SEQUENCE LISTING (602 sequences)

SEQ ID NO: 1 - skipped

SEQ ID NO: 2 - protein, 14 aa, synthetic construct
  REGION 1..14: note = Synthetic Construct
  VARIANT 3: note = where Xaa3 is H, N, or S
  VARIANT 5: note = Xaa5 is F, G, K, R, P, D, Q, H, E, N, T, S, A, I, L, or V
  VARIANT 6: note = Xaa6 is S, Q, E, T, K, or D
  VARIANT 8: note = Xaa8 is L, V, I, F, Y, or W
AIXGXXTXYADSVK

SEQ ID NO: 3 - protein, 12 aa, synthetic construct
  REGION 1..12: note = Synthetic Construct
  VARIANT 5: note = where Xaa5 is D, I, T, K, R, A, E, S, or Y
  VARIANT 9: note = Xaa9 is R, G, L, K, T, Q, S, or N
  VARIANT 10: note = Xaa10 is N, K, E, V, R, M, or D
  VARIANT 12: note = Xaa12 is Y, A, V, K, H, L, M, T, R, Q, C, S, or N
VPWGXYHPXXVX

SEQ ID NO: 4 - protein, 10 aa, synthetic construct
  REGION 1..10: note = Synthetic Construct
TDIFSISPMG

SEQ ID NO: 5 - protein, 10 aa, synthetic construct
  REGION 1..10: note = Synthetic Construct
TNIFSSSPMG

SEQ ID NO: 6 - protein, 10 aa, synthetic construct
  REGION 1..10: note = Synthetic Construct
TNIFSISPGG

SEQ ID NO: 7 - protein, 10 aa, synthetic construct
  REGION 1..10: note = Synthetic Construct
TNIFMISPMG

SEQ ID NO: 8 - protein, 10 aa, synthetic construct
  REGION 1..10: note = Synthetic Construct
TNIFSSSPMG

SEQ ID NO: 9 - protein, 10 aa, synthetic construct
  REGION 1..10: note = Synthetic Construct
TNIFSIRPMG

SEQ ID NO: 10 - protein, 10 aa, synthetic construct
  REGION 1..10: note = Synthetic Construct
TNISSISPMG

SEQ ID NO: 11 - protein, 10 aa, synthetic construct
  REGION 1..10: note = Synthetic Construct
TNIFSSSPMG

SEQ ID NO: 12 - protein, 10 aa, synthetic construct
  REGION 1..10: note = Synthetic Construct
TNIFSITPMG

SEQ ID NO: 13 - protein, 10 aa, synthetic construct
  REGION 1..10: note = Synthetic Construct
TNIPSISPMG

SEQ ID NO: 14 - protein, 10 aa, synthetic construct
  REGION 1..10: note = Synthetic Construct
TNITSISPMG

SEQ ID NO: 15 - protein, 10 aa, synthetic construct
  REGION 1..10: note = Synthetic Construct
TNIFSKSPMG

SEQ ID NO: 16 - protein, 10 aa, synthetic construct
  REGION 1..10: note = Synthetic Construct
TNDFSISPMG

SEQ ID NO: 17 - protein, 10 aa, synthetic construct
  REGION 1..10: note = Synthetic Construct
TNITSISPMG

SEQ ID NO: 18 - protein, 10 aa, synthetic construct
  REGION 1..10: note = Synthetic Construct
TNIFSISPMG

SEQ ID NO: 19 - protein, 10 aa, synthetic construct
  REGION 1..10: note = Synthetic Construct
TNIFSRSPMG

SEQ ID NO: 20 - protein, 10 aa, synthetic construct
  REGION 1..10: note = Synthetic Construct
TNIESISPMG

SEQ ID NO: 21 - protein, 10 aa, synthetic construct
  REGION 1..10: note = Synthetic Construct
SNIFSISPMG

SEQ ID NO: 22 - protein, 10 aa, synthetic construct
  REGION 1..10: note = Synthetic Construct
TNIFSTSPMG

SEQ ID NO: 23 - protein, 10 aa, synthetic construct
  REGION 1..10: note = Synthetic Construct
TNIVSISPMG

SEQ ID NO: 24 - protein, 10 aa, synthetic construct
  REGION 1..10: note = Synthetic Construct
TNIESISPMG

SEQ ID NO: 25 - protein, 10 aa, synthetic construct
  REGION 1..10: note = Synthetic Construct
TNIPSISPMG

SEQ ID NO: 26 - protein, 10 aa, synthetic construct
  REGION 1..10: note = Synthetic Construct
TNIFSSSPMG

SEQ ID NO: 27 - protein, 10 aa, synthetic construct
  REGION 1..10: note = Synthetic Construct
TNIFSISPMG

SEQ ID NO: 28 - protein, 10 aa, synthetic construct
  REGION 1..10: note = Synthetic Construct
TNIFSIYPMG

SEQ ID NO: 29 - protein, 10 aa, synthetic construct
  REGION 1..10: note = Synthetic Construct
TNIFSNSPMG

SEQ ID NO: 30 - protein, 10 aa, synthetic construct
  REGION 1..10: note = Synthetic Construct
TNISSISPMG

SEQ ID NO: 31 - protein, 10 aa, synthetic construct
  REGION 1..10: note = Synthetic Construct
TNIVSISPMG

SEQ ID NO: 32 - protein, 10 aa, synthetic construct
  REGION 1..10: note = Synthetic Construct
TNIFSNSPMG

SEQ ID NO: 33 - protein, 10 aa, synthetic construct
  REGION 1..10: note = Synthetic Construct
TNITSISPMG

SEQ ID NO: 34 - protein, 10 aa, synthetic construct
  REGION 1..10: note = Synthetic Construct
TNIFSDSPMG

SEQ ID NO: 35 - protein, 10 aa, synthetic construct
  REGION 1..10: note = Synthetic Construct
TNIFSRSPMG

SEQ ID NO: 36 - protein, 10 aa, synthetic construct
  REGION 1..10: note = Synthetic Construct
TNIFSASPMG

SEQ ID NO: 37 - protein, 10 aa, synthetic construct
  REGION 1..10: note = Synthetic Construct
TNIFSASPMG

SEQ ID NO: 38 - protein, 10 aa, synthetic construct
  REGION 1..10: note = Synthetic Construct
TNITSISPMG

SEQ ID NO: 39 - protein, 10 aa, synthetic construct
  REGION 1..10: note = Synthetic Construct
TNIASISPMG

SEQ ID NO: 40 - protein, 10 aa, synthetic construct
  REGION 1..10: note = Synthetic Construct
TNIFSRSPMG

SEQ ID NO: 41 - protein, 10 aa, synthetic construct
  REGION 1..10: note = Synthetic Construct
TNIFSLSPMG

SEQ ID NO: 42 - protein, 10 aa, synthetic construct
  REGION 1..10: note = Synthetic Construct
TNIPSISPMG

SEQ ID NO: 43 - protein, 10 aa, synthetic construct
  REGION 1..10: note = Synthetic Construct
TNIFSQSPMG

SEQ ID NO: 44 - protein, 10 aa, synthetic construct
  REGION 1..10: note = Synthetic Construct
TNIESISPMG

SEQ ID NO: 45 - protein, 10 aa, synthetic construct
  REGION 1..10: note = Synthetic Construct
TNIFSHSPMG

SEQ ID NO: 46 - protein, 10 aa, synthetic construct
  REGION 1..10: note = Synthetic Construct
TNIFSESPMG

SEQ ID NO: 47 - protein, 10 aa, synthetic construct
  REGION 1..10: note = Synthetic Construct
TNIDSISPMG

SEQ ID NO: 48 - protein, 10 aa, synthetic construct
  REGION 1..10: note = Synthetic Construct
TNIFSSSPMG

SEQ ID NO: 49 - protein, 10 aa, synthetic construct
  REGION 1..10: note = Synthetic Construct
TNIFSTSPMG

SEQ ID NO: 50 - protein, 10 aa, synthetic construct
  REGION 1..10: note = Synthetic Construct
TNITSVSPMG

SEQ ID NO: 51 - protein, 10 aa, synthetic construct
  REGION 1..10: note = Synthetic Construct
TNISSISPMG

SEQ ID NO: 52 - protein, 10 aa, synthetic construct
  REGION 1..10: note = Synthetic Construct
SNIFSISPMG

SEQ ID NO: 53 - protein, 10 aa, synthetic construct
  REGION 1..10: note = Synthetic Construct
TNIFRISPMG

SEQ ID NO: 54 - protein, 10 aa, synthetic construct
  REGION 1..10: note = Synthetic Construct
TNIVSISPMG

SEQ ID NO: 55 - protein, 10 aa, synthetic construct
  REGION 1..10: note = Synthetic Construct
TNIDSISPMG

SEQ ID NO: 56 - protein, 10 aa, synthetic construct
  REGION 1..10: note = Synthetic Construct
TNIFSRSPMG

SEQ ID NO: 57 - protein, 10 aa, synthetic construct
  REGION 1..10: note = Synthetic Construct
TNIQSISPMG

SEQ ID NO: 58 - protein, 10 aa, synthetic construct
  REGION 1..10: note = Synthetic Construct
TNIFNISPMG

SEQ ID NO: 59 - protein, 10 aa, synthetic construct
  REGION 1..10: note = Synthetic Construct
TNEFSISPMG

SEQ ID NO: 60 - protein, 10 aa, synthetic construct
  REGION 1..10: note = Synthetic Construct
TNIPSISPMG

SEQ ID NO: 61 - protein, 10 aa, synthetic construct
  REGION 1..10: note = Synthetic Construct
TNIGSISPMG

SEQ ID NO: 62 - protein, 10 aa, synthetic construct
  REGION 1..10: note = Synthetic Construct
TNIFSKSPMG

SEQ ID NO: 63 - protein, 10 aa, synthetic construct
  REGION 1..10: note = Synthetic Construct
TNIFSITPMG

SEQ ID NO: 64 - protein, 10 aa, synthetic construct
  REGION 1..10: note = Synthetic Construct
TSDFSISPMG

SEQ ID NO: 65 - protein, 10 aa, synthetic construct
  REGION 1..10: note = Synthetic Construct
TNIMSISPMG

SEQ ID NO: 66 - protein, 10 aa, synthetic construct
  REGION 1..10: note = Synthetic Construct
TNIMSISPMG

SEQ ID NO: 67 - protein, 10 aa, synthetic construct
  REGION 1..10: note = Synthetic Construct
TNIPSISPMG

SEQ ID NO: 68 - protein, 10 aa, synthetic construct
  REGION 1..10: note = Synthetic Construct
TNIFSTSPMG

SEQ ID NO: 69 - protein, 10 aa, synthetic construct
  REGION 1..10: note = Synthetic Construct
TNIFSQSPMG

SEQ ID NO: 70 - protein, 10 aa, synthetic construct
  REGION 1..10: note = Synthetic Construct
TNIASISPMG

SEQ ID NO: 71 - protein, 10 aa, synthetic construct
  REGION 1..10: note = Synthetic Construct
TNIFSKSPMG

SEQ ID NO: 72 - protein, 10 aa, synthetic construct
  REGION 1..10: note = Synthetic Construct
TNIFSRSPMG

SEQ ID NO: 73 - protein, 10 aa, synthetic construct
  REGION 1..10: note = Synthetic Construct
TNHFSISPMG

SEQ ID NO: 74 - protein, 10 aa, synthetic construct
  REGION 1..10: note = Synthetic Construct
TNIFSNSPMG

SEQ ID NO: 75 - protein, 10 aa, synthetic construct
  REGION 1..10: note = Synthetic Construct
TNIFSTSPMG

SEQ ID NO: 76 - protein, 10 aa, synthetic construct
  REGION 1..10: note = Synthetic Construct
TNIFSISPYG

SEQ ID NO: 77 - protein, 10 aa, synthetic construct
  REGION 1..10: note = Synthetic Construct
TNIFSNSPMG

SEQ ID NO: 78 - protein, 10 aa, synthetic construct
  REGION 1..10: note = Synthetic Construct
TNIFSSSPMG

SEQ ID NO: 79 - protein, 10 aa, synthetic construct
  REGION 1..10: note = Synthetic Construct
TNIVSISPMG

SEQ ID NO: 80 - protein, 10 aa, synthetic construct
  REGION 1..10: note = Synthetic Construct
TNISSISPMG

SEQ ID NO: 81 - protein, 10 aa, synthetic construct
  REGION 1..10: note = Synthetic Construct
TNIISISPMG

SEQ ID NO: 82 - protein, 10 aa, synthetic construct
  REGION 1..10: note = Synthetic Construct
TNIASISPMG

SEQ ID NO: 83 - protein, 10 aa, synthetic construct
  REGION 1..10: note = Synthetic Construct
TNIFSESPMG

SEQ ID NO: 84 - protein, 10 aa, synthetic construct
  REGION 1..10: note = Synthetic Construct
TNIFSTSPMG

SEQ ID NO: 85 - protein, 10 aa, synthetic construct
  REGION 1..10: note = Synthetic Construct
TNISSISPMG

SEQ ID NO: 86 - protein, 10 aa, synthetic construct
  REGION 1..10: note = Synthetic Construct
TNVVSISPMG

SEQ ID NO: 87 - protein, 10 aa, synthetic construct
  REGION 1..10: note = Synthetic Construct
TNEFSISPMG

SEQ ID NO: 88 - protein, 10 aa, synthetic construct
  REGION 1..10: note = Synthetic Construct
TNIFSNSPMG

SEQ ID NO: 89 - protein, 10 aa, synthetic construct
  REGION 1..10: note = Synthetic Construct
TNIFSRSPMG

SEQ ID NO: 90 - protein, 10 aa, synthetic construct
  REGION 1..10: note = Synthetic Construct
TNIFSDSPMG

SEQ ID NO: 91 - protein, 10 aa, synthetic construct
  REGION 1..10: note = Synthetic Construct
TNDFSISPMG

SEQ ID NO: 92 - protein, 10 aa, synthetic construct
  REGION 1..10: note = Synthetic Construct
TNIFSKSPMG

SEQ ID NO: 93 - protein, 10 aa, synthetic construct
  REGION 1..10: note = Synthetic Construct
TNIFSIYPMG

SEQ ID NO: 94 - protein, 10 aa, synthetic construct
  REGION 1..10: note = Synthetic Construct
TNIFSSSPMG

SEQ ID NO: 95 - protein, 10 aa, synthetic construct
  REGION 1..10: note = Synthetic Construct
TNIFSVSPMG

SEQ ID NO: 96 - protein, 10 aa, synthetic construct
  REGION 1..10: note = Synthetic Construct
TNIFSITPMG

SEQ ID NO: 97 - protein, 10 aa, synthetic construct
  REGION 1..10: note = Synthetic Construct
TNIESISPMG

SEQ ID NO: 98 - protein, 10 aa, synthetic construct
  REGION 1..10: note = Synthetic Construct
TNIFSTSPMG

SEQ ID NO: 99 - protein, 10 aa, synthetic construct
  REGION 1..10: note = Synthetic Construct
TNIESISPMG

SEQ ID NO: 100 - protein, 10 aa, synthetic construct
  REGION 1..10: note = Synthetic Construct
TNIFSINPMG

SEQ ID NO: 101 - protein, 10 aa, synthetic construct
  REGION 1..10: note = Synthetic Construct
TNIFSITPMG

SEQ ID NO: 102 - protein, 10 aa, synthetic construct
  REGION 1..10: note = Synthetic Construct
TNITSISPMG

SEQ ID NO: 103 - protein, 10 aa, synthetic construct
  REGION 1..10: note = Synthetic Construct
TNIFSGSPMG

SEQ ID NO: 104 - protein, 10 aa, synthetic construct
  REGION 1..10: note = Synthetic Construct
TNIFSITPMG

SEQ ID NO: 105 - protein, 10 aa, synthetic construct
  REGION 1..10: note = Synthetic Construct
TNIDSISPMG

SEQ ID NO: 106 - protein, 10 aa, synthetic construct
  REGION 1..10: note = Synthetic Construct
TNIFSDSPMG

SEQ ID NO: 107 - protein, 10 aa, synthetic construct
  REGION 1..10: note = Synthetic Construct
TNIDSISPMG

SEQ ID NO: 108 - protein, 10 aa, synthetic construct
  REGION 1..10: note = Synthetic Construct
TNIFSKSPMG

SEQ ID NO: 109 - protein, 10 aa, synthetic construct
  REGION 1..10: note = Synthetic Construct
TNIFSVSPMG

SEQ ID NO: 110 - protein, 10 aa, synthetic construct
  REGION 1..10: note = Synthetic Construct
TNQFSISPMG

SEQ ID NO: 111 - protein, 10 aa, synthetic construct
  REGION 1..10: note = Synthetic Construct
TNIRSISPMG

SEQ ID NO: 112 - protein, 10 aa, synthetic construct
  REGION 1..10: note = Synthetic Construct
TNIFSRSPMG

SEQ ID NO: 113 - protein, 10 aa, synthetic construct
  REGION 1..10: note = Synthetic Construct
TNITSISPMG

SEQ ID NO: 114 - protein, 10 aa, synthetic construct
  REGION 1..10: note = Synthetic Construct
TNIFSTSPYG

SEQ ID NO: 115 - protein, 10 aa, synthetic construct
  REGION 1..10: note = Synthetic Construct
TNIFSTSPGG

SEQ ID NO: 116 - protein, 10 aa, synthetic construct
  REGION 1..10: note = Synthetic Construct
TNIFSITPYG

SEQ ID NO: 117 - protein, 10 aa, synthetic construct
  REGION 1..10: note = Synthetic Construct
TNIFSITPGG

SEQ ID NO: 118 - protein, 14 aa, synthetic construct
  REGION 1..14: note = Synthetic Construct
AIHGGSTLYADSVK

SEQ ID NO: 119 - protein, 14 aa, synthetic construct
  REGION 1..14: note = Synthetic Construct
AINGFSTLYADSVK

SEQ ID NO: 120 - protein, 14 aa, synthetic construct
  REGION 1..14: note = Synthetic Construct
AIHGSSTLYADSVK

SEQ ID NO: 121 - protein, 14 aa, synthetic construct
  REGION 1..14: note = Synthetic Construct
AIHGDSTLYADSVK

SEQ ID NO: 122 - protein, 14 aa, synthetic construct
  REGION 1..14: note = Synthetic Construct
AIHGFSTLYADSVK

SEQ ID NO: 123 - protein, 14 aa, synthetic construct
  REGION 1..14: note = Synthetic Construct
AIHGFSTVYADSVK

SEQ ID NO: 124 - protein, 14 aa, synthetic construct
  REGION 1..14: note = Synthetic Construct
AIHGTSTLYADSVK

SEQ ID NO: 125 - protein, 14 aa, synthetic construct
  REGION 1..14: note = Synthetic Construct
AIHGESTLYADSVK

SEQ ID NO: 126 - protein, 14 aa, synthetic construct
  REGION 1..14: note = Synthetic Construct
AIHGRSTLYADSVK

SEQ ID NO: 127 - protein, 14 aa, synthetic construct
  REGION 1..14: note = Synthetic Construct
AIHGESTLYADSVK

SEQ ID NO: 128 - protein, 14 aa, synthetic construct
  REGION 1..14: note = Synthetic Construct
AISGFSTLYADSVK

SEQ ID NO: 129 - protein, 14 aa, synthetic construct
  REGION 1..14: note = Synthetic Construct
AIHGKSTLYADSVK

SEQ ID NO: 130 - protein, 14 aa, synthetic construct
  REGION 1..14: note = Synthetic Construct
AIHGKSTLYADSVK

SEQ ID NO: 131 - protein, 14 aa, synthetic construct
  REGION 1..14: note = Synthetic Construct
AIHGFETLYADSVK

SEQ ID NO: 132 - protein, 14 aa, synthetic construct
  REGION 1..14: note = Synthetic Construct
AIHGDSTLYADSVK

SEQ ID NO: 133 - protein, 14 aa, synthetic construct
  REGION 1..14: note = Synthetic Construct
AIHGNSTLYADSVK

SEQ ID NO: 134 - protein, 14 aa, synthetic construct
  REGION 1..14: note = Synthetic Construct
AIHGSSTLYADSVK

SEQ ID NO: 135 - protein, 14 aa, synthetic construct
  REGION 1..14: note = Synthetic Construct
AIHGSSTLYADSVK

SEQ ID NO: 136 - protein, 14 aa, synthetic construct
  REGION 1..14: note = Synthetic Construct
AIHGFQTLYADSVK

SEQ ID NO: 137 - protein, 14 aa, synthetic construct
  REGION 1..14: note = Synthetic Construct
AIHGHSTLYADSVK

SEQ ID NO: 138 - protein, 14 aa, synthetic construct
  REGION 1..14: note = Synthetic Construct
AIHGNSTLYADSVK

SEQ ID NO: 139 - protein, 14 aa, synthetic construct
  REGION 1..14: note = Synthetic Construct
AIHGDSTLYADSVK

SEQ ID NO: 140 - protein, 14 aa, synthetic construct
  REGION 1..14: note = Synthetic Construct
AIHGFKTLYADSVK

SEQ ID NO: 141 - protein, 14 aa, synthetic construct
  REGION 1..14: note = Synthetic Construct
AIHGDSTLYADSVK

SEQ ID NO: 142 - protein, 14 aa, synthetic construct
  REGION 1..14: note = Synthetic Construct
AIHGFSTYYADSVK

SEQ ID NO: 143 - protein, 14 aa, synthetic construct
  REGION 1..14: note = Synthetic Construct
AIHGGSTLYADSVK

SEQ ID NO: 144 - protein, 14 aa, synthetic construct
  REGION 1..14: note = Synthetic Construct
AIHGFSTLYADSVK

SEQ ID NO: 145 - protein, 14 aa, synthetic construct
  REGION 1..14: note = Synthetic Construct
AIHGKSTLYADSVK

SEQ ID NO: 146 - protein, 14 aa, synthetic construct
  REGION 1..14: note = Synthetic Construct
AIHGDSTLYADSVK

SEQ ID NO: 147 - protein, 14 aa, synthetic construct
  REGION 1..14: note = Synthetic Construct
AIHGVSTLYADSVK

SEQ ID NO: 148 - protein, 14 aa, synthetic construct
  REGION 1..14: note = Synthetic Construct
AIHGTSTLYADSVK

SEQ ID NO: 149 - protein, 14 aa, synthetic construct
  REGION 1..14: note = Synthetic Construct
AIHGDSTLYADSVK

SEQ ID NO: 150 - protein, 14 aa, synthetic construct
  REGION 1..14: note = Synthetic Construct
AIHGSSTLYADSVK

SEQ ID NO: 151 - protein, 14 aa, synthetic construct
  REGION 1..14: note = Synthetic Construct
AIHGSSTLYADSVK

SEQ ID NO: 152 - protein, 14 aa, synthetic construct
  REGION 1..14: note = Synthetic Construct
AIHGDSTLYADSVK

SEQ ID NO: 153 - protein, 14 aa, synthetic construct
  REGION 1..14: note = Synthetic Construct
AIHGTSTLYADSVK

SEQ ID NO: 154 - protein, 14 aa, synthetic construct
  REGION 1..14: note = Synthetic Construct
AIHGTSTLYADSVK

SEQ ID NO: 155 - protein, 14 aa, synthetic construct
  REGION 1..14: note = Synthetic Construct
AIHGDSTLYADSVK

SEQ ID NO: 156 - protein, 14 aa, synthetic construct
  REGION 1..14: note = Synthetic Construct
AIHGQSTLYADSVK

SEQ ID NO: 157 - protein, 14 aa, synthetic construct
  REGION 1..14: note = Synthetic Construct
AIHGDSTLYADSVK

SEQ ID NO: 158 - protein, 14 aa, synthetic construct
  REGION 1..14: note = Synthetic Construct
AIHGKSTLYADSVK

SEQ ID NO: 159 - protein, 14 aa, synthetic construct
  REGION 1..14: note = Synthetic Construct
AIHGTSTLYADSVK

SEQ ID NO: 160 - protein, 14 aa, synthetic construct
  REGION 1..14: note = Synthetic Construct
AIHGNSTLYADSVK

SEQ ID NO: 161 - protein, 14 aa, synthetic construct
  REGION 1..14: note = Synthetic Construct
AIHGFQTLYADSVK

SEQ ID NO: 162 - protein, 14 aa, synthetic construct
  REGION 1..14: note = Synthetic Construct
AIHGFSTWYADSVK

SEQ ID NO: 163 - protein, 14 aa, synthetic construct
  REGION 1..14: note = Synthetic Construct
AIHGFSTIYADSVK

SEQ ID NO: 164 - protein, 14 aa, synthetic construct
  REGION 1..14: note = Synthetic Construct
AIHGPSTLYADSVK

SEQ ID NO: 165 - protein, 14 aa, synthetic construct
  REGION 1..14: note = Synthetic Construct
AIHGHSTLYADSVK

SEQ ID NO: 166 - protein, 14 aa, synthetic construct
  REGION 1..14: note = Synthetic Construct
AIHGESTLYADSVK

SEQ ID NO: 167 - protein, 14 aa, synthetic construct
  REGION 1..14: note = Synthetic Construct
AIHGDSTLYADSVK

SEQ ID NO: 168 - protein, 14 aa, synthetic construct
  REGION 1..14: note = Synthetic Construct
AIHGDSTLYADSVK

SEQ ID NO: 169 - protein, 14 aa, synthetic construct
  REGION 1..14: note = Synthetic Construct
AIHGNSTLYADSVK

SEQ ID NO: 170 - protein, 14 aa, synthetic construct
  REGION 1..14: note = Synthetic Construct
AIHGFETLYADSVK

SEQ ID NO: 171 - protein, 14 aa, synthetic construct
  REGION 1..14: note = Synthetic Construct
AIHGFETLYADSVK

SEQ ID NO: 172 - protein, 14 aa, synthetic construct
  REGION 1..14: note = Synthetic Construct
AIHGFSTYYADSVK

SEQ ID NO: 173 - protein, 14 aa, synthetic construct
  REGION 1..14: note = Synthetic Construct
AIHGLSTLYADSVK

SEQ ID NO: 174 - protein, 14 aa, synthetic construct
  REGION 1..14: note = Synthetic Construct
AIHGASTLYADSVK

SEQ ID NO: 175 - protein, 14 aa, synthetic construct
  REGION 1..14: note = Synthetic Construct
AIHGQSTLYADSVK

SEQ ID NO: 176 - protein, 14 aa, synthetic construct
  REGION 1..14: note = Synthetic Construct
AIHGQSTLYADSVK

SEQ ID NO: 177 - protein, 14 aa, synthetic construct
  REGION 1..14: note = Synthetic Construct
AIHGTSTLYADSVK

SEQ ID NO: 178 - protein, 14 aa, synthetic construct
  REGION 1..14: note = Synthetic Construct
AIHGFETLYADSVK

SEQ ID NO: 179 - protein, 14 aa, synthetic construct
  REGION 1..14: note = Synthetic Construct
AIHGFSTVYADSVK

SEQ ID NO: 180 - protein, 14 aa, synthetic construct
  REGION 1..14: note = Synthetic Construct
AIHGNSTLYADSVK

SEQ ID NO: 181 - protein, 14 aa, synthetic construct
  REGION 1..14: note = Synthetic Construct
AIHGESTLYADSVK

SEQ ID NO: 182 - protein, 14 aa, synthetic construct
  REGION 1..14: note = Synthetic Construct
AIHGFSTLYADSVK

SEQ ID NO: 183 - protein, 14 aa, synthetic construct
  REGION 1..14: note = Synthetic Construct
AIHGKSTLYADSVK

SEQ ID NO: 184 - protein, 14 aa, synthetic construct
  REGION 1..14: note = Synthetic Construct
AIHGKSTLYADSVK

SEQ ID NO: 185 - protein, 14 aa, synthetic construct
  REGION 1..14: note = Synthetic Construct
AIHGNSTLYADSVK

SEQ ID NO: 186 - protein, 14 aa, synthetic construct
  REGION 1..14: note = Synthetic Construct
AIHGESTLYADSVK

SEQ ID NO: 187 - protein, 14 aa, synthetic construct
  REGION 1..14: note = Synthetic Construct
AIHGSSTLYADSVK

SEQ ID NO: 188 - protein, 14 aa, synthetic construct
  REGION 1..14: note = Synthetic Construct
AIHGRSTLYADSVK

SEQ ID NO: 189 - protein, 14 aa, synthetic construct
  REGION 1..14: note = Synthetic Construct
AIHGFSTIYADSVK

SEQ ID NO: 190 - protein, 14 aa, synthetic construct
  REGION 1..14: note = Synthetic Construct
AIHGTSTLYADSVK

SEQ ID NO: 191 - protein, 14 aa, synthetic construct
  REGION 1..14: note = Synthetic Construct
AIHGFSTLYADSVK

SEQ ID NO: 192 - protein, 14 aa, synthetic construct
  REGION 1..14: note = Synthetic Construct
AIHGHSTLYADSVK

SEQ ID NO: 193 - protein, 14 aa, synthetic construct
  REGION 1..14: note = Synthetic Construct
AIHGDSTLYADSVK

SEQ ID NO: 194 - protein, 14 aa, synthetic construct
  REGION 1..14: note = Synthetic Construct
AIHGFDTLYADSVK

SEQ ID NO: 195 - protein, 14 aa, synthetic construct
  REGION 1..14: note = Synthetic Construct
AIHGASTLYADSVK

SEQ ID NO: 196 - protein, 14 aa, synthetic construct
  REGION 1..14: note = Synthetic Construct
AIHGSSTLYADSVK

SEQ ID NO: 197 - protein, 14 aa, synthetic construct
  REGION 1..14: note = Synthetic Construct
AIHGFTTLYADSVK

SEQ ID NO: 198 - protein, 14 aa, synthetic construct
  REGION 1..14: note = Synthetic Construct
AIHGTSTLYADSVK

SEQ ID NO: 199 - protein, 14 aa, synthetic construct
  REGION 1..14: note = Synthetic Construct
AIHGFSTVYADSVK

SEQ ID NO: 200 - protein, 14 aa, synthetic construct
  REGION 1..14: note = Synthetic Construct
AIHGKSTLYADSVK

SEQ ID NO: 201 - protein, 14 aa, synthetic construct
  REGION 1..14: note = Synthetic Construct
AIHGESTLYADSVK

SEQ ID NO: 202 - protein, 14 aa, synthetic construct
  REGION 1..14: note = Synthetic Construct
AIHGPSTLYADSVK

SEQ ID NO: 203 - protein, 14 aa, synthetic construct
  REGION 1..14: note = Synthetic Construct
AIHGISTLYADSVK

SEQ ID NO: 204 - protein, 14 aa, synthetic construct
  REGION 1..14: note = Synthetic Construct
AIHGFSTFYADSVK

SEQ ID NO: 205 - protein, 14 aa, synthetic construct
  REGION 1..14: note = Synthetic Construct
AIHGGSTLYADSVK

SEQ ID NO: 206 - protein, 14 aa, synthetic construct
  REGION 1..14: note = Synthetic Construct
AIHGSSTLYADSVK

SEQ ID NO: 207 - protein, 14 aa, synthetic construct
  REGION 1..14: note = Synthetic Construct
AIHGSSTLYADSVK

SEQ ID NO: 208 - protein, 14 aa, synthetic construct
  REGION 1..14: note = Synthetic Construct
AIHGFSTLYADSVK

SEQ ID NO: 209 - protein, 14 aa, synthetic construct
  REGION 1..14: note = Synthetic Construct
AIHGNSTLYADSVK

SEQ ID NO: 210 - protein, 14 aa, synthetic construct
  REGION 1..14: note = Synthetic Construct
AIHGESTLYADSVK

SEQ ID NO: 211 - protein, 14 aa, synthetic construct
  REGION 1..14: note = Synthetic Construct
AIHGFSTLYADSVK

SEQ ID NO: 212 - protein, 14 aa, synthetic construct
  REGION 1..14: note = Synthetic Construct
AIHGTSTLYADSVK

SEQ ID NO: 213 - protein, 14 aa, synthetic construct
  REGION 1..14: note = Synthetic Construct
AIHGTSTLYADSVK

SEQ ID NO: 214 - protein, 14 aa, synthetic construct
  REGION 1..14: note = Synthetic Construct
AIHGFDTLYADSVK

SEQ ID NO: 215 - protein, 14 aa, synthetic construct
  REGION 1..14: note = Synthetic Construct
AIHGASTLYADSVK

SEQ ID NO: 216 - protein, 14 aa, synthetic construct
  REGION 1..14: note = Synthetic Construct
AIHGNSTLYADSVK

SEQ ID NO: 217 - protein, 14 aa, synthetic construct
  REGION 1..14: note = Synthetic Construct
AIHGNSTLYADSVK

SEQ ID NO: 218 - protein, 14 aa, synthetic construct
  REGION 1..14: note = Synthetic Construct
AIHGESTLYADSVK

SEQ ID NO: 219 - protein, 14 aa, synthetic construct
  REGION 1..14: note = Synthetic Construct
AIHGESTLYADSVK

SEQ ID NO: 220 - protein, 14 aa, synthetic construct
  REGION 1..14: note = Synthetic Construct
AIHGFSTLYADSVK

SEQ ID NO: 221 - protein, 14 aa, synthetic construct
  REGION 1..14: note = Synthetic Construct
AIHGSSTLYADSVK

SEQ ID NO: 222 - protein, 14 aa, synthetic construct
  REGION 1..14: note = Synthetic Construct
AIHGSSTLYADSVK

SEQ ID NO: 223 - protein, 14 aa, synthetic construct
  REGION 1..14: note = Synthetic Construct
AIHGNSTLYADSVK

SEQ ID NO: 224 - protein, 14 aa, synthetic construct
  REGION 1..14: note = Synthetic Construct
AIHGKSTLYADSVK

SEQ ID NO: 225 - protein, 14 aa, synthetic construct
  REGION 1..14: note = Synthetic Construct
AIHGSSTLYADSVK

SEQ ID NO: 226 - protein, 14 aa, synthetic construct
  REGION 1..14: note = Synthetic Construct
AIHGSSTLYADSVK

SEQ ID NO: 227 - protein, 14 aa, synthetic construct
  REGION 1..14: note = Synthetic Construct
AIHGESTLYADSVK

SEQ ID NO: 228 - protein, 14 aa, synthetic construct
  REGION 1..14: note = Synthetic Construct
AIHGFSTIYADSVK

SEQ ID NO: 229 - protein, 14 aa, synthetic construct
  REGION 1..14: note = Synthetic Construct
AIHGFSTIYADSVK

SEQ ID NO: 230 - protein, 14 aa, synthetic construct
  REGION 1..14: note = Synthetic Construct
AIHGASTLYADSVK

SEQ ID NO: 231 - protein, 14 aa, synthetic construct
  REGION 1..14: note = Synthetic Construct
AIHGASTLYADSVK

SEQ ID NO: 232 - protein, 12 aa, synthetic construct
  REGION 1..12: note = Synthetic Construct
VPWGDYHPRNVA

SEQ ID NO: 233 - protein, 12 aa, synthetic construct
  REGION 1..12: note = Synthetic Construct
VPWGDYHPRNVH

SEQ ID NO: 234 - protein, 12 aa, synthetic construct
  REGION 1..12: note = Synthetic Construct
VPWGDYHPRNVY

SEQ ID NO: 235 - protein, 12 aa, synthetic construct
  REGION 1..12: note = Synthetic Construct
VPWGRYHPRNVY

SEQ ID NO: 236 - protein, 12 aa, synthetic construct
  REGION 1..12: note = Synthetic Construct
VPWGDYHPRNVY

SEQ ID NO: 237 - protein, 12 aa, synthetic construct
  REGION 1..12: note = Synthetic Construct
VPWGDYHPRNVY

SEQ ID NO: 238 - protein, 12 aa, synthetic construct
  REGION 1..12: note = Synthetic Construct
VPWGDYHPGNVY

SEQ ID NO: 239 - protein, 12 aa, synthetic construct
  REGION 1..12: note = Synthetic Construct
VPWGDYHPRKVY

SEQ ID NO: 240 - protein, 12 aa, synthetic construct
  REGION 1..12: note = Synthetic Construct
VPWGSYHPRNVY

SEQ ID NO: 241 - protein, 12 aa, synthetic construct
  REGION 1..12: note = Synthetic Construct
VPWGDYHPRNVA

SEQ ID NO: 242 - protein, 12 aa, synthetic construct
  REGION 1..12: note = Synthetic Construct
VPWGDYHPRNVY

SEQ ID NO: 243 - protein, 12 aa, synthetic construct
  REGION 1..12: note = Synthetic Construct
VPWGDYHPRNVV

SEQ ID NO: 244 - protein, 12 aa, synthetic construct
  REGION 1..12: note = Synthetic Construct
VPWGDYHPRNVK

SEQ ID NO: 245 - protein, 12 aa, synthetic construct
  REGION 1..12: note = Synthetic Construct
VPWGDYHPGNVY

SEQ ID NO: 246 - protein, 12 aa, synthetic construct
  REGION 1..12: note = Synthetic Construct
VPWGEYHPRNVY

SEQ ID NO: 247 - protein, 12 aa, synthetic construct
  REGION 1..12: note = Synthetic Construct
VPWGIYHPRNVY

SEQ ID NO: 248 - protein, 12 aa, synthetic construct
  REGION 1..12: note = Synthetic Construct
VPWGRYHPRNVY

SEQ ID NO: 249 - protein, 12 aa, synthetic construct
  REGION 1..12: note = Synthetic Construct
VPWGDYHPGNVY

SEQ ID NO: 250 - protein, 12 aa, synthetic construct
  REGION 1..12: note = Synthetic Construct
VPWGDYHPRNVV

SEQ ID NO: 251 - protein, 12 aa, synthetic construct
  REGION 1..12: note = Synthetic Construct
VPWGDYHPGNVY

SEQ ID NO: 252 - protein, 12 aa, synthetic construct
  REGION 1..12: note = Synthetic Construct
VPWGRYHPRNVY

SEQ ID NO: 253 - protein, 12 aa, synthetic construct
  REGION 1..12: note = Synthetic Construct
VPWGDYHPRNVY

SEQ ID NO: 254 - protein, 12 aa, synthetic construct
  REGION 1..12: note = Synthetic Construct
VPWGDYHPGNVY

SEQ ID NO: 255 - protein, 12 aa, synthetic construct
  REGION 1..12: note = Synthetic Construct
VPWGKYHPRNVY

SEQ ID NO: 256 - protein, 12 aa, synthetic construct
  REGION 1..12: note = Synthetic Construct
VPWGSYHPRNVY

SEQ ID NO: 257 - protein, 12 aa, synthetic construct
  REGION 1..12: note = Synthetic Construct
VPWGDYHPRNVH

SEQ ID NO: 258 - protein, 12 aa, synthetic construct
  REGION 1..12: note = Synthetic Construct
VPWGYYHPRNVY

SEQ ID NO: 259 - protein, 12 aa, synthetic construct
  REGION 1..12: note = Synthetic Construct
VPWGDYHPGNVY

SEQ ID NO: 260 - protein, 12 aa, synthetic construct
  REGION 1..12: note = Synthetic Construct
VPWGDYHPRNVR

SEQ ID NO: 261 - protein, 12 aa, synthetic construct
  REGION 1..12: note = Synthetic Construct
VPWGDYHPRNVQ

SEQ ID NO: 262 - protein, 12 aa, synthetic construct
  REGION 1..12: note = Synthetic Construct
VPWGDYHPRNVY

SEQ ID NO: 263 - protein, 12 aa, synthetic construct
  REGION 1..12: note = Synthetic Construct
VPWGDYHPRNVT

SEQ ID NO: 264 - protein, 12 aa, synthetic construct
  REGION 1..12: note = Synthetic Construct
VPWGDYHPRNVN

SEQ ID NO: 265 - protein, 12 aa, synthetic construct
  REGION 1..12: note = Synthetic Construct
VPWGRYHPRNVY

SEQ ID NO: 266 - protein, 12 aa, synthetic construct
  REGION 1..12: note = Synthetic Construct
VPWGDYHPGNVY

SEQ ID NO: 267 - protein, 12 aa, synthetic construct
  REGION 1..12: note = Synthetic Construct
VPWGDYHPGNVY

SEQ ID NO: 268 - protein, 12 aa, synthetic construct
  REGION 1..12: note = Synthetic Construct
VPWGKYHPRNVY

SEQ ID NO: 269 - protein, 12 aa, synthetic construct
  REGION 1..12: note = Synthetic Construct
VPWGDYHPRNVY

SEQ ID NO: 270 - protein, 12 aa, synthetic construct
  REGION 1..12: note = Synthetic Construct
VPWGDYHPRNVQ

SEQ ID NO: 271 - protein, 12 aa, synthetic construct
  REGION 1..12: note = Synthetic Construct
VPWGDYHPGNVC

SEQ ID NO: 272 - protein, 12 aa, synthetic construct
  REGION 1..12: note = Synthetic Construct
VPWGDYHPRRVY

SEQ ID NO: 273 - protein, 12 aa, synthetic construct
  REGION 1..12: note = Synthetic Construct
VPWGRYHPRNVY

SEQ ID NO: 274 - protein, 12 aa, synthetic construct
  REGION 1..12: note = Synthetic Construct
VPWGTYHPRNVY

SEQ ID NO: 275 - protein, 12 aa, synthetic construct
  REGION 1..12: note = Synthetic Construct
VPWGDYHPGNVY

SEQ ID NO: 276 - protein, 12 aa, synthetic construct
  REGION 1..12: note = Synthetic Construct
VPWGRYHPRNVY

SEQ ID NO: 277 - protein, 12 aa, synthetic construct
  REGION 1..12: note = Synthetic Construct
VPWGDYHPGNVY

SEQ ID NO: 278 - protein, 12 aa, synthetic construct
  REGION 1..12: note = Synthetic Construct
VPWGDYHPTNVY

SEQ ID NO: 279 - protein, 12 aa, synthetic construct
  REGION 1..12: note = Synthetic Construct
VPWGRYHPRNVY

SEQ ID NO: 280 - protein, 12 aa, synthetic construct
  REGION 1..12: note = Synthetic Construct
VPWGDYHPRNVT

SEQ ID NO: 281 - protein, 12 aa, synthetic construct
  REGION 1..12: note = Synthetic Construct
VPWGRYHPRNVY

SEQ ID NO: 282 - protein, 12 aa, synthetic construct
  REGION 1..12: note = Synthetic Construct
VPWGDYHPRRVY

SEQ ID NO: 283 - protein, 12 aa, synthetic construct
  REGION 1..12: note = Synthetic Construct
VPWGDYHPRMVY

SEQ ID NO: 284 - protein, 12 aa, synthetic construct
  REGION 1..12: note = Synthetic Construct
VPWGDYHPRNVL

SEQ ID NO: 285 - protein, 12 aa, synthetic construct
  REGION 1..12: note = Synthetic Construct
VPWGDYHPGNVY

SEQ ID NO: 286 - protein, 12 aa, synthetic construct
  REGION 1..12: note = Synthetic Construct
VPWGRYHPRNVY

SEQ ID NO: 287 - protein, 12 aa, synthetic construct
  REGION 1..12: note = Synthetic Construct
VPWGAYHPRNVY

SEQ ID NO: 288 - protein, 12 aa, synthetic construct
  REGION 1..12: note = Synthetic Construct
VPWGDYHPRNVA

SEQ ID NO: 289 - protein, 12 aa, synthetic construct
  REGION 1..12: note = Synthetic Construct
VPWGDYHPQNVY

SEQ ID NO: 290 - protein, 12 aa, synthetic construct
  REGION 1..12: note = Synthetic Construct
VPWGDYHPRNVT

SEQ ID NO: 291 - protein, 12 aa, synthetic construct
  REGION 1..12: note = Synthetic Construct
VPWGRYHPRNVY

SEQ ID NO: 292 - protein, 12 aa, synthetic construct
  REGION 1..12: note = Synthetic Construct
VPWGDYHPGNVY

SEQ ID NO: 293 - protein, 12 aa, synthetic construct
  REGION 1..12: note = Synthetic Construct
VPWGDYHPGNVY

SEQ ID NO: 294 - protein, 12 aa, synthetic construct
  REGION 1..12: note = Synthetic Construct
VPWGDYHPGNVY

SEQ ID NO: 295 - protein, 12 aa, synthetic construct
  REGION 1..12: note = Synthetic Construct
VPWGDYHPRNVY

SEQ ID NO: 296 - protein, 12 aa, synthetic construct
  REGION 1..12: note = Synthetic Construct
VPWGDYHPGNVY

SEQ ID NO: 297 - protein, 12 aa, synthetic construct
  REGION 1..12: note = Synthetic Construct
VPWGDYHPSNVY

SEQ ID NO: 298 - protein, 12 aa, synthetic construct
  REGION 1..12: note = Synthetic Construct
VPWGDYHPGNVY

SEQ ID NO: 299 - protein, 12 aa, synthetic construct
  REGION 1..12: note = Synthetic Construct
VPWGDYHPREVY

SEQ ID NO: 300 - protein, 12 aa, synthetic construct
  REGION 1..12: note = Synthetic Construct
VPWGRYHPGNVY

SEQ ID NO: 301 - protein, 12 aa, synthetic construct
  REGION 1..12: note = Synthetic Construct
VPWGDYHPRVVY

SEQ ID NO: 302 - protein, 12 aa, synthetic construct
  REGION 1..12: note = Synthetic Construct
VPWGDYHPRNVM

SEQ ID NO: 303 - protein, 12 aa, synthetic construct
  REGION 1..12: note = Synthetic Construct
VPWGDYHPLNVY

SEQ ID NO: 304 - protein, 12 aa, synthetic construct
  REGION 1..12: note = Synthetic Construct
VPWGDYHPGNVY

SEQ ID NO: 305 - protein, 12 aa, synthetic construct
  REGION 1..12: note = Synthetic Construct
VPWGDYHPGNVY

SEQ ID NO: 306 - protein, 12 aa, synthetic construct
  REGION 1..12: note = Synthetic Construct
VPWGDYHPRNVT

SEQ ID NO: 307 - protein, 12 aa, synthetic construct
  REGION 1..12: note = Synthetic Construct
VPWGDYHPRNVS

SEQ ID NO: 308 - protein, 12 aa, synthetic construct
  REGION 1..12: note = Synthetic Construct
VPWGDYHPRNVY

SEQ ID NO: 309 - protein, 12 aa, synthetic construct
  REGION 1..12: note = Synthetic Construct
VPWGDYHPGNVY

SEQ ID NO: 310 - protein, 12 aa, synthetic construct
  REGION 1..12: note = Synthetic Construct
VPWGDYHPGNVY

SEQ ID NO: 311 - protein, 12 aa, synthetic construct
  REGION 1..12: note = Synthetic Construct
VPWGDYHPRNVT

SEQ ID NO: 312 - protein, 12 aa, synthetic construct
  REGION 1..12: note = Synthetic Construct
VPWGDYHPGNVY

SEQ ID NO: 313 - protein, 12 aa, synthetic construct
  REGION 1..12: note = Synthetic Construct
VPWGRYHPRNVY

SEQ ID NO: 314 - protein, 12 aa, synthetic construct
  REGION 1..12: note = Synthetic Construct
VPWGDYHPNNVY

SEQ ID NO: 315 - protein, 12 aa, synthetic construct
  REGION 1..12: note = Synthetic Construct
VPWGDYHPGNVY

SEQ ID NO: 316 - protein, 12 aa, synthetic construct
  REGION 1..12: note = Synthetic Construct
VPWGDYHPRNVT

SEQ ID NO: 317 - protein, 12 aa, synthetic construct
  REGION 1..12: note = Synthetic Construct
VPWGDYHPGNVY

SEQ ID NO: 318 - protein, 12 aa, synthetic construct
  REGION 1..12: note = Synthetic Construct
VPWGDYHPGNVY

SEQ ID NO: 319 - protein, 12 aa, synthetic construct
  REGION 1..12: note = Synthetic Construct
VPWGDYHPRNVA

SEQ ID NO: 320 - protein, 12 aa, synthetic construct
  REGION 1..12: note = Synthetic Construct
VPWGDYHPGNVY

SEQ ID NO: 321 - protein, 12 aa, synthetic construct
  REGION 1..12: note = Synthetic Construct
VPWGDYHPKNVY

SEQ ID NO: 322 - protein, 12 aa, synthetic construct
  REGION 1..12: note = Synthetic Construct
VPWGDYHPGNVY

SEQ ID NO: 323 - protein, 12 aa, synthetic construct
  REGION 1..12: note = Synthetic Construct
VPWGKYHPRNVY

SEQ ID NO: 324 - protein, 12 aa, synthetic construct
  REGION 1..12: note = Synthetic Construct
VPWGRYHPRNVY

SEQ ID NO: 325 - protein, 12 aa, synthetic construct
  REGION 1..12: note = Synthetic Construct
VPWGDYHPGNVY

SEQ ID NO: 326 - protein, 12 aa, synthetic construct
  REGION 1..12: note = Synthetic Construct
VPWGDYHPRNVQ

SEQ ID NO: 327 - protein, 12 aa, synthetic construct
  REGION 1..12: note = Synthetic Construct
VPWGDYHPGNVY

SEQ ID NO: 328 - protein, 12 aa, synthetic construct
  REGION 1..12: note = Synthetic Construct
VPWGDYHPRNVS

SEQ ID NO: 329 - protein, 12 aa, synthetic construct
  REGION 1..12: note = Synthetic Construct
VPWGDYHPGNVY

SEQ ID NO: 330 - protein, 12 aa, synthetic construct
  REGION 1..12: note = Synthetic Construct
VPWGDYHPGNVY

SEQ ID NO: 331 - protein, 12 aa, synthetic construct
  REGION 1..12: note = Synthetic Construct
VPWGDYHPGNVY

SEQ ID NO: 332 - protein, 12 aa, synthetic construct
  REGION 1..12: note = Synthetic Construct
VPWGDYHPRDVY

SEQ ID NO: 333 - protein, 12 aa, synthetic construct
  REGION 1..12: note = Synthetic Construct
VPWGDYHPRNVT

SEQ ID NO: 334 - protein, 12 aa, synthetic construct
  REGION 1..12: note = Synthetic Construct
VPWGDYHPRNVA

SEQ ID NO: 335 - protein, 12 aa, synthetic construct
  REGION 1..12: note = Synthetic Construct
VPWGDYHPRNVT

SEQ ID NO: 336 - protein, 12 aa, synthetic construct
  REGION 1..12: note = Synthetic Construct
VPWGDYHPRNVY

SEQ ID NO: 337 - protein, 12 aa, synthetic construct
  REGION 1..12: note = Synthetic Construct
VPWGDYHPRNVY

SEQ ID NO: 338 - protein, 12 aa, synthetic construct
  REGION 1..12: note = Synthetic Construct
VPWGDYHPRNVV

SEQ ID NO: 339 - protein, 12 aa, synthetic construct
  REGION 1..12: note = Synthetic Construct
VPWGDYHPRNVV

SEQ ID NO: 340 - protein, 12 aa, synthetic construct
  REGION 1..12: note = Synthetic Construct
VPWGDYHPGNVY

SEQ ID NO: 341 - protein, 12 aa, synthetic construct
  REGION 1..12: note = Synthetic Construct
VPWGDYHPRNVY

SEQ ID NO: 342 - protein, 12 aa, synthetic construct
  REGION 1..12: note = Synthetic Construct
VPWGDYHPGNVY

SEQ ID NO: 343 - protein, 12 aa, synthetic construct
  REGION 1..12: note = Synthetic Construct
VPWGDYHPGNVY

SEQ ID NO: 344 - protein, 12 aa, synthetic construct
  REGION 1..12: note = Synthetic Construct
VPWGDYHPGNVY

SEQ ID NO: 345 - protein, 12 aa, synthetic construct
  REGION 1..12: note = Synthetic Construct
VPWGDYHPGNVY

SEQ ID NO: 346 - protein, 119 aa, synthetic construct
  REGION 1..119: note = Synthetic Construct
EVQLVESGGGLVQPGRSLTLSCAASTNIFSISPMGWYRQAPGKQRELVAAIHGFSTLYADSVKGRFTISRDNAKNSIYLQMNSLRPEDTALYYCNKVPWGDYHPRNVYWGQGTQVTVSS

SEQ ID NO: 347 - protein, 119 aa, synthetic construct
  REGION 1..119: note = Synthetic Construct
EVQLVESGGGLVQPGRSLTLSCAASTDIFSISPMGWYRQAPGKQRELVAAIHGGSTLYADSVKGRFTISRDNAKNSIYLQMNSLRPEDTALYYCNKVPWGDYHPRNVAWGQGTQVTVSS

SEQ ID NO: 348 - protein, 119 aa, synthetic construct
  REGION 1..119: note = Synthetic Construct
EVQLVESGGGLVQPGRSLTLSCAASTNDFSISPMGWYRQAPGKQRELVAAIHGGSTLYADSVKGRFTISRDNAKNSIYLQMNSLRPEDTALYYCNKVPWGDYHPRNVAWGQGTQVTVSS

SEQ ID NO: 349 - protein, 119 aa, synthetic construct
  REGION 1..119: note = Synthetic Construct
EVQLVESGGGLVQPGRSLTLSCAASTNIFSKSPMGWYRQAPGKQRELVAAIHGKSTLYADSVKGRFTISRDNAKNSIYLQMNSLRPEDTALYYCNKVPWGDYHPRNVVWGQGTQVTVSS

SEQ ID NO: 350 - protein, 119 aa, synthetic construct
  REGION 1..119: note = Synthetic Construct
EVQLVESGGGLVQPGRSLTLSCAASTNDFSISPMGWYRQAPGKQRELVAAIHGKSTLYADSVKGRFTISRDNAKNSIYLQMNSLRPEDTALYYCNKVPWGDYHPRNVKWGQGTQVTVSS

SEQ ID NO: 351 - protein, 119 aa, synthetic construct
  REGION 1..119: note = Synthetic Construct
EVQLVESGGGLVQPGRSLTLSCAASTNQFSISPMGWYRQAPGKQRELVAAIHGKSTLYADSVKGRFTISRDNAKNSIYLQMNSLRPEDTALYYCNKVPWGDYHPRNVVWGQGTQVTVSS

SEQ ID NO: 352 - protein, 119 aa, synthetic construct
  REGION 1..119: note = Synthetic Construct
EVQLVESGGGLVQPGRSLTLSCAASTNIFSSSPMGWYRQAPGKQRELVAAINGFSTLYADSVKGRFTISRDNAKNSIYLQMNSLRPEDTALYYCNKVPWGDYHPRNVHWGQGTQVTVSS

SEQ ID NO: 353 - protein, 119 aa, synthetic construct
  REGION 1..119: note = Synthetic Construct
EVQLVESGGGLVQPGRSLTLSCAASTNIFSSSPMGWYRQAPGKQRELVAAIHGFSTLYADSVKGRFTISRDNAKNSIYLQMNSLRPEDTALYYCNKVPWGDYHPRNVYWGQGTQVTVSS

SEQ ID NO: 354 - protein, 119 aa, synthetic construct
  REGION 1..119: note = Synthetic Construct
EVQLVESGGGLVQPGRSLTLSCAASTNIFSTSPMGWYRQAPGKQRELVAAIHGFQTLYADSVKGRFTISRDNAKNSIYLQMNSLRPEDTALYYCNKVPWGDYHPRNVVWGQGTQVTVSS

SEQ ID NO: 355 - protein, 119 aa, synthetic construct
  REGION 1..119: note = Synthetic Construct
EVQLVESGGGLVQPGRSLTLSCAASTNIFSRSPMGWYRQAPGKQRELVAAIHGFETLYADSVKGRFTISRDNAKNSIYLQMNSLRPEDTALYYCNKVPWGDYHPRNVLWGQGTQVTVSS

SEQ ID NO: 356 - protein, 119 aa, synthetic construct
  REGION 1..119: note = Synthetic Construct
EVQLVESGGGLVQPGRSLTLSCAASTNIFSESPMGWYRQAPGKQRELVAAIHGFTTLYADSVKGRFTISRDNAKNSIYLQMNSLRPEDTALYYCNKVPWGDYHPRNVTWGQGTQVTVSS

SEQ ID NO: 357 - protein, 119 aa, synthetic construct
  REGION 1..119: note = Synthetic Construct
EVQLVESGGGLVQPGRSLTLSCAASTNIFSDSPMGWYRQAPGKQRELVAAIHGFSTLYADSVKGRFTISRDNAKNSIYLQMNSLRPEDTALYYCNKVPWGDYHPRNVAWGQGTQVTVSS

SEQ ID NO: 358 - protein, 119 aa, synthetic construct
  REGION 1..119: note = Synthetic Construct
EVQLVESGGGLVQPGRSLTLSCAASTNIFSNSPMGWYRQAPGKQRELVAAIHGGSTLYADSVKGRFTISRDNAKNSIYLQMNSLRPEDTALYYCNKVPWGDYHPRNVHWGQGTQVTVSS

SEQ ID NO: 359 - protein, 119 aa, synthetic construct
  REGION 1..119: note = Synthetic Construct
EVQLVESGGGLVQPGRSLTLSCAASTNIFSNSPMGWYRQAPGKQRELVAAIHGRSTLYADSVKGRFTISRDNAKNSIYLQMNSLRPEDTALYYCNKVPWGDYHPRNVMWGQGTQVTVSS

SEQ ID NO: 360 - protein, 119 aa, synthetic construct
  REGION 1..119: note = Synthetic Construct
EVQLVESGGGLVQPGRSLTLSCAASTNIFSNSPMGWYRQAPGKQRELVAAIHGPSTLYADSVKGRFTISRDNAKNSIYLQMNSLRPEDTALYYCNKVPWGDYHPRNVTWGQGTQVTVSS

SEQ ID NO: 361 - protein, 119 aa, synthetic construct
  REGION 1..119: note = Synthetic Construct
EVQLVESGGGLVQPGRSLTLSCAASTNIFSNSPMGWYRQAPGKQRELVAAIHGDSTLYADSVKGRFTISRDNAKNSIYLQMNSLRPEDTALYYCNKVPWGDYHPRNVRWGQGTQVTVSS

SEQ ID NO: 362 - protein, 119 aa, synthetic construct
  REGION 1..119: note = Synthetic Construct
EVQLVESGGGLVQPGRSLTLSCAASTNIFSRSPMGWYRQAPGKQRELVAAIHGDSTLYADSVKGRFTISRDNAKNSIYLQMNSLRPEDTALYYCNKVPWGDYHPRNVTWGQGTQVTVSS

SEQ ID NO: 363 - protein, 119 aa, synthetic construct
  REGION 1..119: note = Synthetic Construct
EVQLVESGGGLVQPGRSLTLSCAASTNIFSKSPMGWYRQAPGKQRELVAAIHGQSTLYADSVKGRFTISRDNAKNSIYLQMNSLRPEDTALYYCNKVPWGDYHPRNVTWGQGTQVTVSS

SEQ ID NO: 364 - protein, 119 aa, synthetic construct
  REGION 1..119: note = Synthetic Construct
EVQLVESGGGLVQPGRSLTLSCAASTNIFSSSPMGWYRQAPGKQRELVAAIHGHSTLYADSVKGRFTISRDNAKNSIYLQMNSLRPEDTALYYCNKVPWGDYHPRNVTWGQGTQVTVSS

SEQ ID NO: 365 - protein, 119 aa, synthetic construct
  REGION 1..119: note = Synthetic Construct
EVQLVESGGGLVQPGRSLTLSCAASTNIFSSSPMGWYRQAPGKQRELVAAIHGESTLYADSVKGRFTISRDNAKNSIYLQMNSLRPEDTALYYCNKVPWGDYHPRKVYWGQGTQVTVSS

SEQ ID NO: 366 - protein, 119 aa, synthetic construct
  REGION 1..119: note = Synthetic Construct
EVQLVESGGGLVQPGRSLTLSCAASTNIFSRSPMGWYRQAPGKQRELVAAIHGNSTLYADSVKGRFTISRDNAKNSIYLQMNSLRPEDTALYYCNKVPWGIYHPRNVYWGQGTQVTVSS

SEQ ID NO: 367 - protein, 119 aa, synthetic construct
  REGION 1..119: note = Synthetic Construct
EVQLVESGGGLVQPGRSLTLSCAASTNIFSESPMGWYRQAPGKQRELVAAIHGNSTLYADSVKGRFTISRDNAKNSIYLQMNSLRPEDTALYYCNKVPWGTYHPRNVYWGQGTQVTVSS

SEQ ID NO: 368 - protein, 119 aa, synthetic construct
  REGION 1..119: note = Synthetic Construct
EVQLVESGGGLVQPGRSLTLSCAASTNIFSVSPMGWYRQAPGKQRELVAAIHGNSTLYADSVKGRFTISRDNAKNSIYLQMNSLRPEDTALYYCNKVPWGKYHPRNVYWGQGTQVTVSS

SEQ ID NO: 369 - protein, 119 aa, synthetic construct
  REGION 1..119: note = Synthetic Construct
EVQLVESGGGLVQPGRSLTLSCAASTNIFSVSPMGWYRQAPGKQRELVAAIHGNSTLYADSVKGRFTISRDNAKNSIYLQMNSLRPEDTALYYCNKVPWGDYHPRNVYWGQGTQVTVSS

SEQ ID NO: 370 - protein, 119 aa, synthetic construct
  REGION 1..119: note = Synthetic Construct
EVQLVESGGGLVQPGRSLTLSCAASTNIFSKSPMGWYRQAPGKQRELVAAIHGNSTLYADSVKGRFTISRDNAKNSIYLQMNSLRPEDTALYYCNKVPWGDYHPREVYWGQGTQVTVSS

SEQ ID NO: 371 - protein, 119 aa, synthetic construct
  REGION 1..119: note = Synthetic Construct
EVQLVESGGGLVQPGRSLTLSCAASTNIFSDSPMGWYRQAPGKQRELVAAIHGTSTLYADSVKGRFTISRDNAKNSIYLQMNSLRPEDTALYYCNKVPWGDYHPRNVYWGQGTQVTVSS

SEQ ID NO: 372 - protein, 119 aa, synthetic construct
  REGION 1..119: note = Synthetic Construct
EVQLVESGGGLVQPGRSLTLSCAASTNIFSRSPMGWYRQAPGKQRELVAAIHGTSTLYADSVKGRFTISRDNAKNSIYLQMNSLRPEDTALYYCNKVPWGKYHPRNVYWGQGTQVTVSS

SEQ ID NO: 373 - protein, 119 aa, synthetic construct
  REGION 1..119: note = Synthetic Construct
EVQLVESGGGLVQPGRSLTLSCAASTNIFSHSPMGWYRQAPGKQRELVAAIHGTSTLYADSVKGRFTISRDNAKNSIYLQMNSLRPEDTALYYCNKVPWGRYHPRNVYWGQGTQVTVSS

SEQ ID NO: 374 - protein, 119 aa, synthetic construct
  REGION 1..119: note = Synthetic Construct
EVQLVESGGGLVQPGRSLTLSCAASTNIFSTSPMGWYRQAPGKQRELVAAIHGTSTLYADSVKGRFTISRDNAKNSIYLQMNSLRPEDTALYYCNKVPWGDYHPGNVYWGQGTQVTVSS

SEQ ID NO: 375 - protein, 119 aa, synthetic construct
  REGION 1..119: note = Synthetic Construct
EVQLVESGGGLVQPGRSLTLSCAASTNIFSTSPMGWYRQAPGKQRELVAAIHGTSTLYADSVKGRFTISRDNAKNSIYLQMNSLRPEDTALYYCNKVPWGDYHPRNVQWGQGTQVTVSS

SEQ ID NO: 376 - protein, 119 aa, synthetic construct
  REGION 1..119: note = Synthetic Construct
EVQLVESGGGLVQPGRSLTLSCAASTNIFSLSPMGWYRQAPGKQRELVAAIHGDSTLYADSVKGRFTISRDNAKNSIYLQMNSLRPEDTALYYCNKVPWGDYHPRNVYWGQGTQVTVSS

SEQ ID NO: 377 - protein, 119 aa, synthetic construct
  REGION 1..119: note = Synthetic Construct
EVQLVESGGGLVQPGRSLTLSCAASTNIFSISPGGWYRQAPGKQRELVAAIHGSSTLYADSVKGRFTISRDNAKNSIYLQMNSLRPEDTALYYCNKVPWGDYHPRNVYWGQGTQVTVSS

SEQ ID NO: 378 - protein, 119 aa, synthetic construct
  REGION 1..119: note = Synthetic Construct
EVQLVESGGGLVQPGRSLTLSCAASTNHFSISPMGWYRQAPGKQRELVAAIHGSSTLYADSVKGRFTISRDNAKNSIYLQMNSLRPEDTALYYCNKVPWGDYHPRVVYWGQGTQVTVSS

SEQ ID NO: 379 - protein, 119 aa, synthetic construct
  REGION 1..119: note = Synthetic Construct
EVQLVESGGGLVQPGRSLTLSCAASTNIFSASPMGWYRQAPGKQRELVAAIHGSSTLYADSVKGRFTISRDNAKNSIYLQMNSLRPEDTALYYCNKVPWGDYHPRNVNWGQGTQVTVSS

SEQ ID NO: 380 - protein, 119 aa, synthetic construct
  REGION 1..119: note = Synthetic Construct
EVQLVESGGGLVQPGRSLTLSCAASTNIFSASPMGWYRQAPGKQRELVAAIHGSSTLYADSVKGRFTISRDNAKNSIYLQMNSLRPEDTALYYCNKVPWGRYHPRNVYWGQGTQVTVSS

SEQ ID NO: 381 - protein, 119 aa, synthetic construct
  REGION 1..119: note = Synthetic Construct
EVQLVESGGGLVQPGRSLTLSCAASTNISSISPMGWYRQAPGKQRELVAAIHGTSTLYADSVKGRFTISRDNAKNSIYLQMNSLRPEDTALYYCNKVPWGDYHPGNVYWGQGTQVTVSS

SEQ ID NO: 382 - protein, 119 aa, synthetic construct
  REGION 1..119: note = Synthetic Construct
EVQLVESGGGLVQPGRSLTLSCAASTNIESISPMGWYRQAPGKQRELVAAIHGTSTLYADSVKGRFTISRDNAKNSIYLQMNSLRPEDTALYYCNKVPWGDYHPGNVYWGQGTQVTVSS

SEQ ID NO: 383 - protein, 119 aa, synthetic construct
  REGION 1..119: note = Synthetic Construct
EVQLVESGGGLVQPGRSLTLSCAASTNIFSISPYGWYRQAPGKQRELVAAIHGTSTLYADSVKGRFTISRDNAKNSIYLQMNSLRPEDTALYYCNKVPWGDYHPGNVYWGQGTQVTVSS

SEQ ID NO: 384 - protein, 119 aa, synthetic construct
  REGION 1..119: note = Synthetic Construct
EVQLVESGGGLVQPGRSLTLSCAASTNIASISPMGWYRQAPGKQRELVAAIHGTSTLYADSVKGRFTISRDNAKNSIYLQMNSLRPEDTALYYCNKVPWGDYHPGNVYWGQGTQVTVSS

SEQ ID NO: 385 - protein, 119 aa, synthetic construct
  REGION 1..119: note = Synthetic Construct
EVQLVESGGGLVQPGRSLTLSCAASTNIASISPMGWYRQAPGKQRELVAAIHGKSTLYADSVKGRFTISRDNAKNSIYLQMNSLRPEDTALYYCNKVPWGDYHPGNVYWGQGTQVTVSS

SEQ ID NO: 386 - protein, 119 aa, synthetic construct
  REGION 1..119: note = Synthetic Construct
EVQLVESGGGLVQPGRSLTLSCAASTNIASISPMGWYRQAPGKQRELVAAIHGSSTLYADSVKGRFTISRDNAKNSIYLQMNSLRPEDTALYYCNKVPWGDYHPGNVYWGQGTQVTVSS

SEQ ID NO: 387 - protein, 119 aa, synthetic construct
  REGION 1..119: note = Synthetic Construct
EVQLVESGGGLVQPGRSLTLSCAASTNITSISPMGWYRQAPGKQRELVAAIHGDSTLYADSVKGRFTISRDNAKNSIYLQMNSLRPEDTALYYCNKVPWGDYHPGNVYWGQGTQVTVSS

SEQ ID NO: 388 - protein, 119 aa, synthetic construct
  REGION 1..119: note = Synthetic Construct
EVQLVESGGGLVQPGRSLTLSCAASTNIMSISPMGWYRQAPGKQRELVAAIHGNSTLYADSVKGRFTISRDNAKNSIYLQMNSLRPEDTALYYCNKVPWGDYHPGNVYWGQGTQVTVSS

SEQ ID NO: 389 - protein, 119 aa, synthetic construct
  REGION 1..119: note = Synthetic Construct
EVQLVESGGGLVQPGRSLTLSCAASTNITSISPMGWYRQAPGKQRELVAAIHGNSTLYADSVKGRFTISRDNAKNSIYLQMNSLRPEDTALYYCNKVPWGDYHPGNVYWGQGTQVTVSS

SEQ ID NO: 390 - protein, 119 aa, synthetic construct
  REGION 1..119: note = Synthetic Construct
EVQLVESGGGLVQPGRSLTLSCAASTNIVSISPMGWYRQAPGKQRELVAAIHGHSTLYADSVKGRFTISRDNAKNSIYLQMNSLRPEDTALYYCNKVPWGDYHPGNVYWGQGTQVTVSS

SEQ ID NO: 391 - protein, 119 aa, synthetic construct
  REGION 1..119: note = Synthetic Construct
EVQLVESGGGLVQPGRSLTLSCAASTNIVSISPMGWYRQAPGKQRELVAAIHGKSTLYADSVKGRFTISRDNAKNSIYLQMNSLRPEDTALYYCNKVPWGDYHPGNVYWGQGTQVTVSS

SEQ ID NO: 392 - protein, 119 aa, synthetic construct
  REGION 1..119: note = Synthetic Construct
EVQLVESGGGLVQPGRSLTLSCAASTNVVSISPMGWYRQAPGKQRELVAAIHGKSTLYADSVKGRFTISRDNAKNSIYLQMNSLRPEDTALYYCNKVPWGDYHPNNVYWGQGTQVTVSS

SEQ ID NO: 393 - protein, 119 aa, synthetic construct
  REGION 1..119: note = Synthetic Construct
EVQLVESGGGLVQPGRSLTLSCAASTNIISISPMGWYRQAPGKQRELVAAIHGASTLYADSVKGRFTISRDNAKNSIYLQMNSLRPEDTALYYCNKVPWGDYHPGNVYWGQGTQVTVSS

SEQ ID NO: 394 - protein, 119 aa, synthetic construct
  REGION 1..119: note = Synthetic Construct
EVQLVESGGGLVQPGRSLTLSCAASTNIFSITPMGWYRQAPGKQRELVAAIHGASTLYADSVKGRFTISRDNAKNSIYLQMNSLRPEDTALYYCNKVPWGDYHPGNVYWGQGTQVTVSS

SEQ ID NO: 395 - protein, 119 aa, synthetic construct
  REGION 1..119: note = Synthetic Construct
EVQLVESGGGLVQPGRSLTLSCAASTNITSISPMGWYRQAPGKQRELVAAIHGFETLYADSVKGRFTISRDNAKNSIYLQMNSLRPEDTALYYCNKVPWGDYHPGNVYWGQGTQVTVSS

SEQ ID NO: 396 - protein, 119 aa, synthetic construct
  REGION 1..119: note = Synthetic Construct
EVQLVESGGGLVQPGRSLTLSCAASTNIQSISPMGWYRQAPGKQRELVAAIHGFETLYADSVKGRFTISRDNAKNSIYLQMNSLRPEDTALYYCNKVPWGDYHPGNVYWGQGTQVTVSS

SEQ ID NO: 397 - protein, 119 aa, synthetic construct
  REGION 1..119: note = Synthetic Construct
EVQLVESGGGLVQPGRSLTLSCAASTSDFSISPMGWYRQAPGKQRELVAAIHGFETLYADSVKGRFTISRDNAKNSIYLQMNSLRPEDTALYYCNKVPWGDYHPGNVYWGQGTQVTVSS

SEQ ID NO: 398 - protein, 119 aa, synthetic construct
  REGION 1..119: note = Synthetic Construct
EVQLVESGGGLVQPGRSLTLSCAASTNIDSISPMGWYRQAPGKQRELVAAIHGFQTLYADSVKGRFTISRDNAKNSIYLQMNSLRPEDTALYYCNKVPWGDYHPGNVYWGQGTQVTVSS

SEQ ID NO: 399 - protein, 119 aa, synthetic construct
  REGION 1..119: note = Synthetic Construct
EVQLVESGGGLVQPGRSLTLSCAASTNIMSISPMGWYRQAPGKQRELVAAIHGFSTVYADSVKGRFTISRDNAKNSIYLQMNSLRPEDTALYYCNKVPWGDYHPGNVYWGQGTQVTVSS

SEQ ID NO: 400 - protein, 119 aa, synthetic construct
  REGION 1..119: note = Synthetic Construct
EVQLVESGGGLVQPGRSLTLSCAASTNIESISPMGWYRQAPGKQRELVAAIHGFSTLYADSVKGRFTISRDNAKNSIYLQMNSLRPEDTALYYCNKVPWGDYHPGNVYWGQGTQVTVSS

SEQ ID NO: 401 - protein, 119 aa, synthetic construct
  REGION 1..119: note = Synthetic Construct
EVQLVESGGGLVQPGRSLTLSCAASTNIFSSSPMGWYRQAPGKQRELVAAIHGFKTLYADSVKGRFTISRDNAKNSIYLQMNSLRPEDTARYYCNKVPWGDYHPGNVYWGQGTQVTVSS

SEQ ID NO: 402 - protein, 119 aa, synthetic construct
  REGION 1..119: note = Synthetic Construct
EVQLVESGGGLVQPGRSLTLSCAASTNIFSSSPMGWYRQAPGKQRELVAAIHGFSTLYADSVKGRFTISRDNAKNSIYLQMNSLRPEDTALYYCNKVPWGDYHPGNVYWGQGTQVTVSS

SEQ ID NO: 403 - protein, 119 aa, synthetic construct
  REGION 1..119: note = Synthetic Construct
EVQLVESGGGLVQPGRSLTLSCAASTNIFSNSPMGWYRQAPGKQRELVAAIHGFSTLYADSVKGRFTISRDNAKNSIYLQMNSLRPEDTALYYCNKVPWGDYHPGNVYWGQGTQVTVSS

SEQ ID NO: 404 - protein, 119 aa, synthetic construct
  REGION 1..119: note = Synthetic Construct
EVQLVESGGGLVQPGRSLTLSCAASTNIFSTSPMGWYRQAPGKQRELVAAIHGFSTIYADSVKGRFTISRDNAKNSIYLQMNSLRPEDTALYYCNKVPWGDYHPGNVYWGQGTQVTVSS

SEQ ID NO: 405 - protein, 119 aa, synthetic construct
  REGION 1..119: note = Synthetic Construct
EVQLVESGGGLVQPGRSLTLSCAASTNIFSTSPMGWYRQAPGKQRELVAAIHGFSTIYADSVKGRFTISRDNAKNSIYLQMNSLRPEDTALYYCNKVPWGDYHPLNVYWGQGTQVTVSS

SEQ ID NO: 406 - protein, 119 aa, synthetic construct
  REGION 1..119: note = Synthetic Construct
EVQLVESGGGLVQPGRSLTLSCVASTNIFSTSPMGWYRQAPGKQRELVAAIHGFSTLYADSVKGRFTISRDNAKNSIYLQMNSLRPEDTALYYCNKVPWGDYHPGNVYWGQGTQVTVSS

SEQ ID NO: 407 - protein, 119 aa, synthetic construct
  REGION 1..119: note = Synthetic Construct
EVQLVESGGGLVQPGRSLTLSCAASTNIFSDSPMGWYRQAPGKQRELVAAIHGFSTFYADSVKGRFTISRDNAKNSIYLQMNSLRPEDTALYYCNKVPWGDYHPGNVYWGQGTQVTVSS

SEQ ID NO: 408 - protein, 119 aa, synthetic construct
  REGION 1..119: note = Synthetic Construct
EVQLVESGGGLVQPGRSLTLSCAASTNIFSQSPMGWYRQAPGKQRELVAAIHGDSTLYADSVKGRFTISRDNAKNSIYLQMNSLRPEDTALYYCNKVPWGDYHPGNVCWGQGTQVTVSS

SEQ ID NO: 409 - protein, 119 aa, synthetic construct
  REGION 1..119: note = Synthetic Construct
EVQLVESGGGLVQPGRSLTLSCAASTNIFSQSPMGWYRQAPGKQRELVAAIHGKSTLYADSVKGRFTISRDNAKNSIYLQMNSLRPEDTALYYCNKVPWGDYHPSNVYWGKGTQVTVSS

SEQ ID NO: 410 - protein, 119 aa, synthetic construct
  REGION 1..119: note = Synthetic Construct
EVQLVESGGGLVQPGRSLTLSCAASTNIFSRSPMGWYRQAPGKQRELVAAIHGESTLYADSVKGRFTISRDNAKNSIYLQMNSLRPEDTALYYCNKVPWGRYHPGNVYWGQGTQVTVSS

SEQ ID NO: 411 - protein, 119 aa, synthetic construct
  REGION 1..119: note = Synthetic Construct
EVQLVESGGGLVQPGRSLTLSCAASTNIFSRSPMGWYRQAPGKQRELVAAIHGISTLYADSVKGRFTISRDNAKNSIYLQMNSLRPEDTALYYCNKVPWGDYHPGNVYWGQGTQVTVSS

SEQ ID NO: 412 - protein, 119 aa, synthetic construct
  REGION 1..119: note = Synthetic Construct
EVQLVESGGGLVQPGRSLTLSCAASTNIFSRSPMGWYRQAPGKQRELVAAIHGSSTLYADSVKGRFTISRDNAKNSIYLQMNSLRPEDTALYYCNKVPWGDYHPGNVYWGQGTQVTVSS

SEQ ID NO: 413 - protein, 119 aa, synthetic construct
  REGION 1..119: note = Synthetic Construct
EVQLVESGGGLVQPGRSLTLSCAASTNIFSGSPMGWYRQAPGKQRELVAAIHGNSTLYADSVKGRFTISRDNAKNSIYLQMNSLRPEDTALYYCNKVPWGDYHPGNVYWGQGTQVTVSS

SEQ ID NO: 414 - protein, 119 aa, synthetic construct
  REGION 1..119: note = Synthetic Construct
EVQLVESGGGLVQPGRSLTLSCAASSNIFSISPMGWYRQAPGKQRELVAAIHGSSTLYADSVKGRFTISRDNAKNSIYLQMNSLRPEDTALYYCNKVPWGDYHPGNVYWGQGTQVTVSS

SEQ ID NO: 415 - protein, 119 aa, synthetic construct
  REGION 1..119: note = Synthetic Construct
EVQLVESGGGLVQPGRSLTLSCAASTNIFSIYPMGWYRQAPGKQRELVAAIHGSSTLYADSVKGRFTISRDNAKNSIYLQMNSLRPEDTALYYCNKVPWGDYHPKNVYWGQGTQVTVSS

SEQ ID NO: 416 - protein, 119 aa, synthetic construct
  REGION 1..119: note = Synthetic Construct
EVQLVESGGGLVQPGRSLTLSCAASTNIFSKSPMGWYRQAPGKQRELVAAIHGSSTLYADSVKGRFTISRDNAKNSIYLQMNSLRPEDTALYYCNKVPWGDYHPGNVYWGQGTQVTVSS

SEQ ID NO: 417 - protein, 119 aa, synthetic construct
  REGION 1..119: note = Synthetic Construct
EVQLVESGGGLVQPGRSLTLSCAASTNIFSKSPMGWYRQAPGKQRELVAAIHGSSTLYADSVKGRFTISRDNAKNSIYLQMNSLRPEDTALYYCNKVPWGDYHPRNVYWGQGTQVTVSS

SEQ ID NO: 418 - protein, 119 aa, synthetic construct
  REGION 1..119: note = Synthetic Construct
EVQLVESGGGLVQPGRSLTLSCAASTNEFSISPMGWYRQAPGKQRELVAAIHGLSTLYADSVKGRFTISRDNAKNSIYLQMNSLRPEDTALYYCNKVPWGAYHPRNVYWGQGTQVTVSS

SEQ ID NO: 419 - protein, 119 aa, synthetic construct
  REGION 1..119: note = Synthetic Construct
EVQLVESGGGLVQPGRSLTLSCAASTNEFSISPMGWYRQAPGKQRELVAAIHGESTLYADSVKGRFTISRDNAKNSIYLQMNSLRPEDTALYYCNKVPWGDYHPGNVYWGQGTQVTVSS

SEQ ID NO: 420 - protein, 119 aa, synthetic construct
  REGION 1..119: note = Synthetic Construct
EVQLVESGGGLVQPGRSLTLSCAASTNIPSISPMGWYRQAPGKQRELVAAIHGESTLYADSVKGRFTISRDNAKNSIYLQMNSLRPEDTALYYCNKVPWGDYHPRNVAWGQGTQVTVSS

SEQ ID NO: 421 - protein, 119 aa, synthetic construct
  REGION 1..119: note = Synthetic Construct
EVQLVESGGGLVQPGRSLTLSCAASTNIPSISPMGWYRQAPGKQRELVAAIHGASTLYADSVKGRFTISRDNAKNSIYLQMNSLRPEDTALYYCNKVPWGDYHPRNVAWGQGTQVTVSS

SEQ ID NO: 422 - protein, 119 aa, synthetic construct
  REGION 1..119: note = Synthetic Construct
EVQLVESGGGLVQPGRSLTLSCAASTNIPSISPMGWYRQAPGKQRELVAAIHGESTLYADSVKGRFTISRDNAKNSIYLQMNSLRPEDTALYYCNKVPWGDYHPRNVYWGQGTQVTVSS

SEQ ID NO: 423 - protein, 119 aa, synthetic construct
  REGION 1..119: note = Synthetic Construct
EVQLVESGGGLVQPGRSLTLSCAASTNIPSISPMGWYRQAPGKQRELVAAIHGDSTLYADSVKGRFTISRDNAKNSIYLQMNSLRPEDTALYYCNKVPWGDYHPRNVYWGQGTQVTVSS

SEQ ID NO: 424 - protein, 119 aa, synthetic construct
  REGION 1..119: note = Synthetic Construct
EVQLVESGGGLVQPGRSLTLSCAASTNITSISPMGWYRQAPGKQRELVAAIHGVSTLYADSVKGRFTISRDNAKNSIYLQMNSLRPEDTALYYCNKVPWGDYHPRNVQWGQGTQVTVSS

SEQ ID NO: 425 - protein, 119 aa, synthetic construct
  REGION 1..119: note = Synthetic Construct
EVQLVESGGGLVQPGRSLTLSCAASTNIPSISPMGWYRQAPGKQRELVAAIHGQSTLYADSVKGRFTISRDNAKNSIYLQMNSLRPEDTALYYCNKVPWGDYHPRNVQWGQGTQVTVSS

SEQ ID NO: 426 - protein, 119 aa, synthetic construct
  REGION 1..119: note = Synthetic Construct
EVQLVESGGGLVQPGRSLTLSCAASTNIVSISPMGWYRQAPGKQRELVAAIHGDSTLYADSVKGRFTISRDNAKNSIYLQMNSLRPEDTALYYCNKVPWGDYHPRNVSWGQGTQVTVSS

SEQ ID NO: 427 - protein, 119 aa, synthetic construct
  REGION 1..119: note = Synthetic Construct
EVQLVESGGGLVQPGRSLTLSCAASSNIFSISPMGWYRQAPGKQRELVAAIHGESTLYADSVKGRFTISRDNAKNSIYLQMNSLRPEDTALYYCNKVPWGDYHPRNVTWGQGTQVTVSS

SEQ ID NO: 428 - protein, 119 aa, synthetic construct
  REGION 1..119: note = Synthetic Construct
EVQLVESGGGLVQPGRSLTLSCAASTNIDSISPMGWYRQAPGKQRELVAAIHGESTLYADSVKGRFTISRDNAKNSIYLQMNSLRPEDTALYYCNKVPWGDYHPRNVTWGQGTQVTVSS

SEQ ID NO: 429 - protein, 119 aa, synthetic construct
  REGION 1..119: note = Synthetic Construct
EVQLVESGGGLVQPGRSLTLSCAASTNIDSISPMGWYRQAPGKQRELVAAIHGSSTLYADSVKGRFTISRDNAKNSIYLQMNSLRPEDTALYYCNKVPWGDYHPRNVTWGQGTQVTVSS

SEQ ID NO: 430 - protein, 119 aa, synthetic construct
  REGION 1..119: note = Synthetic Construct
EVQLVESGGGLVQPGRSLTLSCAASTNIRSISPMGWYRQAPGKQRELVAAIHGSSTLYADSVKGRFTISRDNAKNSIYLQMNSLRPEDTALYYCNKVPWGDYHPRNVVWGQGTQVTVSS

SEQ ID NO: 431 - protein, 119 aa, synthetic construct
  REGION 1..119: note = Synthetic Construct
EVQLVESGGGLVQPGRSLTLSCAASTNITSISPMGWYRQAPGKQRELVAAISGFSTLYADSVKGRFTISRDNAKNSIYLQMNSLRPEDTALYYCNEVPWGDYHPRNVYWGQGTQVTVSS

SEQ ID NO: 432 - protein, 119 aa, synthetic construct
  REGION 1..119: note = Synthetic Construct
EVQLVESGGGLVQPGRSLTLSCAASTNITSISPMGWYRQAPGKQRELVAAIHGESTLYADSVKGRFTISRDNAKNSIYLQMNSLRPEDTALYYCNKVPWGDYHPRNVYWGQGTQVTVSS

SEQ ID NO: 433 - protein, 119 aa, synthetic construct
  REGION 1..119: note = Synthetic Construct
EVQLVESGGGLVQPGRSLTLSCAASTNITSVSPMGWYRQAPGKQRELVAAIHGPSTLYADSVKGRFTISRDNAKNSIYLQMNSLRPEDTALYYCNKVPWGDYHPTNVYWGQGTQVTVSS

SEQ ID NO: 434 - protein, 119 aa, synthetic construct
  REGION 1..119: note = Synthetic Construct
EVQLVESGGGLVQPGRSLTLSCAASTNIGSISPMGWYRQAPGKQRELVAAIHGQSTLYADSVKGRFTISRDNAKNSIYLQMNSLRPEDTALYYCNKVPWGDYHPQNVYWGQGTQVTVSS

SEQ ID NO: 435 - protein, 119 aa, synthetic construct
  REGION 1..119: note = Synthetic Construct
EVQLVESGGGLVQPGRSLTLSCAASTNIESISPMGWYRQAPGKQRELVAAIHGKSTLYADSVKGRFTISRDNAKNSIYLQMNSLRPEDTALYYCNKVPWGDYHPRRVYWGQGTQVTVSS

SEQ ID NO: 436 - protein, 119 aa, synthetic construct
  REGION 1..119: note = Synthetic Construct
EVQLVESGGGLVQPGRSLTLSCAASTNIVSISPMGWYRQAPGKQRELVAAIHGDSTLYADSVKGRFTISRDNAKNSIYLQMNSLRPEDTALYYCNKVPWGDYHPRRVYWGQGTQVTVSS

SEQ ID NO: 437 - protein, 119 aa, synthetic construct
  REGION 1..119: note = Synthetic Construct
EVQLVESGGGLVQPGRSLTLSCAASTNIDSISPMGWYRQAPGKQRELVAAIHGNSTLYADSVKGRFTISRDNAKNSIYLQMNSLRPEDTALYYCNKVPWGDYHPRMVYWGQGTQVTVSS

SEQ ID NO: 438 - protein, 119 aa, synthetic construct
  REGION 1..119: note = Synthetic Construct
EVQLVESGGGLVQPGRSLTLSCAASTNIFMISPMGWYRQAPGKQRELVAAIHGDSTLYADSVKGRFTISRDNAKNSIYLQMNSLRPEDTALYYCNKVPWGRYHPRNVYWGQGTQVTVSS

SEQ ID NO: 439 - protein, 119 aa, synthetic construct
  REGION 1..119: note = Synthetic Construct
EVQLVESGGGLVQPGRSLTLSCAASTNIFRISPMGWYRQAPGKQRELVAAIHGDSTLYADSVKGRFTISRDNAKNSIYLQMNSLRPEDTALYYCNKVPWGRYHPRNVYWGQGTQVTVSS

SEQ ID NO: 440 - protein, 119 aa, synthetic construct
  REGION 1..119: note = Synthetic Construct
EVQLVESGGGLVQPGRSLTLSCAASTNIFSISPMGWYRQAPGKQRELVAAIHGDSTLYADSVKGRFTISRDNAKNSIYLQMNSLRPEDTALYYCNKVPWGEYHPRNVYWGQGTQVTVSS

SEQ ID NO: 441 - protein, 119 aa, synthetic construct
  REGION 1..119: note = Synthetic Construct
EVQLVESGGGLVQPGRSLTLSCAASTNIFSISPMGWYRQAPGKQRELVAAIHGDSTLYADSVKGRFTISRDNAKNSIYLQMNSLRPEDTALYYCNKVPWGKYHPRNVYWGQGTQVTVSS

SEQ ID NO: 442 - protein, 119 aa, synthetic construct
  REGION 1..119: note = Synthetic Construct
EVQLVESGGGLVQPGRSLTLSCAASTNIESISPMGWYRQAPGKQRELVAAIHGSSTLYADSVKGRFTISRDNAKNSIYLQMNSLRPEDTALYYCNKVPWGRYHPRNVYWGQGTQVTVSS

SEQ ID NO: 443 - protein, 119 aa, synthetic construct
  REGION 1..119: note = Synthetic Construct
EVQLVESGGGLVQPGRSLTLSCAASTNIESISPMGWYRQAPGKQRELVAAIHGNSTLYADSVKGRFTISRDNAKNSIYLQMNSLRPEDTALYYCNKVPWGRYHPRNVYWGQGTQVTVSS

SEQ ID NO: 444 - protein, 119 aa, synthetic construct
  REGION 1..119: note = Synthetic Construct
EVQLVESGGGLVQPGRSLTLSCAASTNISSISPMGWYRQAPGKQRELVAAIHGFSTLYADSVKGRFTISRDNAKNSIYLQMNSLRPEDTALYYCNKVPWGYYHPRNVYWGQGTQVTVSS

SEQ ID NO: 445 - protein, 119 aa, synthetic construct
  REGION 1..119: note = Synthetic Construct
EVQLVESGGGLVQPGRSLTLSCAASTNISSISPMGWYRQAPGKQRELVAAIHGHSTLYADSVKGRFTISRDNAKNSIYLQMNSLRPEDTALYYCNKVPWGRYHPRNVYWGQGTQVTVSS

SEQ ID NO: 446 - protein, 119 aa, synthetic construct
  REGION 1..119: note = Synthetic Construct
EVQLVESGGGLVQPGRSLTLSCAASTNISSISPMGWYRQAPGKQRELVAAIHGFSTVYADSVKGRFTISRDNAKNSIYLQMNSLRPEDTALYYCNKVPWGRYHPRNVYWGQGTQVTVSS

SEQ ID NO: 447 - protein, 119 aa, synthetic construct
  REGION 1..119: note = Synthetic Construct
EVQLVESGGGLVQPGRSLTLSCAASTNIFSIRPMGWYRQAPGKQRELVAAIHGFSTVYADSVKGRFTISRDNAKNSIYLQMNSLRPEDTALYYCNKVPWGDYHPRNVYWGQGTQVTVSS

SEQ ID NO: 448 - protein, 119 aa, synthetic construct
  REGION 1..119: note = Synthetic Construct
EVQLVESGGGLVQPGRSLTLSCAASTNIFSIYPMGWYRQAPGKQRELVAAIHGFSTYYADSVKGRFTISRDNAKNSIYLQMNSLRPEDTALYYCNKVPWGSYHPRNVYWGQGTQVTVSS

SEQ ID NO: 449 - protein, 119 aa, synthetic construct
  REGION 1..119: note = Synthetic Construct
EVQLVESGGGLVQPGRSLTLSCAASTNIFNISPMGWYRQAPGKQRELVAAIHGFSTYYADSVKGRFTISRDNAKNSIYLQMNSLRPEDTALYYCNKVPWGRYHPRNVYWGQGTQVTVSS

SEQ ID NO: 450 - protein, 119 aa, synthetic construct
  REGION 1..119: note = Synthetic Construct
EVQLVESGGGLVQPGRSLTLSCAASTNIFSSSPMGWYRQAPGKQRELVAAIHGFSTWYADSVKGRFTISRDNAKNSIYLQMNSLRPEDTALYYCNKVPWGRYHPRNVYWGQGTQVTVSS

SEQ ID NO: 451 - protein, 119 aa, synthetic construct
  REGION 1..119: note = Synthetic Construct
EVQLVESGGGLVQPGRSLTLSCAASTNISSISPMGWYRQAPGKQRELVAAIHGFDTLYADSVKGRFTISRDNAKNSIYLQMNSLRPEDTALYYCNKVPWGDYHPRNVYWGQGTQVTVSS

SEQ ID NO: 452 - protein, 119 aa, synthetic construct
  REGION 1..119: note = Synthetic Construct
EVQLVESGGGLVQPGRSLTLSCAASTNIFSINPMGWYRQAPGKQRELVAAIHGFDTLYADSVKGRFTISRDNAKNSIYLQMNSLRPEDTALYYCNKVPWGDYHPRNVSWGQGTQVTVSS

SEQ ID NO: 453 - protein, 119 aa, synthetic construct
  REGION 1..119: note = Synthetic Construct
EVQLVESGGGLVQPGRSLTLSCAASTNIFSITPMGWYRQAPGKQRELVAAIHGRSTLYADSVKGRFTISRDNAKNSIYLQMNSLRPEDTALYYCNKVPWGSYHPRNVYWGQGTQVTVSS

SEQ ID NO: 454 - protein, 119 aa, synthetic construct
  REGION 1..119: note = Synthetic Construct
EVQLVESGGGLVQPGRSLTLSCAASTNIFSITPMGWYRQAPGKQRELVAAIHGTSTLYADSVKGRFTISRDNAKNSIYLQMNSLRPEDTALYYCNKVPWGRYHPRNVYWGQGTQVTVSS

SEQ ID NO: 455 - protein, 119 aa, synthetic construct
  REGION 1..119: note = Synthetic Construct
EVQLVESGGGLVQPGRSLTLSCAASTNIFSITPMGWYRQAPGKQRELVAAIHGESTLYADSVKGRFTISRDNAKNSIYLQMNSLRPEDTALYYCNKVPWGRYHPRNVYWGQGTQVTVSS

SEQ ID NO: 456 - protein, 119 aa, synthetic construct
  REGION 1..119: note = Synthetic Construct
EVQLVESGGGLVQPGRSLTLSCAASTNIFSITPMGWYRQAPGKQRELVAAIHGESTLYADSVKGRFTISRDNAKNSIYLQMNSLRPEDTALYYCNKVPWGDYHPRDVYWGQGTQVTVSS

SEQ ID NO: 457 - protein, 119 aa, synthetic construct
  REGION 1..119: note = Synthetic Construct
EVQLVESGGGLVQPGRSLTLSCAASTNIFSTSPYGWYRQAPGKQRELVAAIHGFSTIYADSVKGRFTISRDNAKNSIYLQMNSLRPEDTALYYCNKVPWGDYHPGNVYWGQGTQVTVSS

SEQ ID NO: 458 - protein, 119 aa, synthetic construct
  REGION 1..119: note = Synthetic Construct
EVQLVESGGGLVQPGRSLTLSCAASTNIFSTSPGGWYRQAPGKQRELVAAIHGFSTIYADSVKGRFTISRDNAKNSIYLQMNSLRPEDTALYYCNKVPWGDYHPGNVYWGQGTQVTVSS

SEQ ID NO: 459 - protein, 119 aa, synthetic construct
  REGION 1..119: note = Synthetic Construct
EVQLVESGGGLVQPGRSLTLSCAASTNIFSITPYGWYRQAPGKQRELVAAIHGASTLYADSVKGRFTISRDNAKNSIYLQMNSLRPEDTALYYCNKVPWGDYHPGNVYWGQGTQVTVSS

SEQ ID NO: 460 - protein, 119 aa, synthetic construct
  REGION 1..119: note = Synthetic Construct
EVQLVESGGGLVQPGRSLTLSCAASTNIFSITPGGWYRQAPGKQRELVAAIHGASTLYADSVKGRFTISRDNAKNSIYLQMNSLRPEDTALYYCNKVPWGDYHPGNVYWGQGTQVTVSS

SEQ ID NO: 461 - protein, 25 aa, synthetic construct
  REGION 1..25: note = Synthetic Construct
EVQLVESGGGLVQPGRSLTLSCAAS

SEQ ID NO: 462 - protein, 25 aa, synthetic construct
  REGION 1..25: note = Synthetic Construct
EVQLVESGGGLVQPGRSLTLSCVAS

SEQ ID NO: 463 - protein, 14 aa, synthetic construct
  REGION 1..14: note = Synthetic Construct
WYRQAPGKQRELVA

SEQ ID NO: 464 - protein, 33 aa, synthetic construct
  REGION 1..33: note = Synthetic Construct
GRFTISRDNAKNSIYLQMNSLRPEDTALYYCNK

SEQ ID NO: 465 - protein, 33 aa, synthetic construct
  REGION 1..33: note = Synthetic Construct
GRFTISRDNAKNSIYLQMNSLRPEDTALYYCNE

SEQ ID NO: 466 - protein, 11 aa, synthetic construct
  REGION 1..11: note = Synthetic Construct
WGQGTQVTVSS

SEQ ID NO: 467 - protein, 11 aa, synthetic construct
  REGION 1..11: note = Synthetic Construct
WGKGTQVTVSS

SEQ ID NO: 468 - protein, 184 aa, Homo sapiens
MLQMAGQCSQNEYFDSLLHACIPCQLRCSSNTPPLTCQRYCNASVTNSVKGTNAILWTCLGLSLIISLAVFVLMFLLRKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 469 - protein, 185 aa, synthetic construct
  REGION 1..185: note = Synthetic Construct
MAQQCFHSEYFDSLLHACKPCHLRCSNPPATCQPYCDPSVTSSVKGTYTVLWIFLGLTLVLSLALFTISFLLRKMNPEALKDEPQSPGQLDGSAQLDKADTELTRIRAGDDRIFPRSLEYTVEECTCEDCVKSKPKGDSDHFFPLPAMEEGATILVTTKTGDYGKSSVPTALQSVMGMEKPTHTR

SEQ ID NO: 470 - protein, 183 aa, synthetic construct
  REGION 1..183: note = Synthetic Construct
MLQMARQCSQNEYFDSLLHDCKPCQLRCSSTPPLTCQRYCNASMTNSVKGMNAILWTCLGLSLIISLAVFVLTFLLRKMSSEPLKDEFKNTGSGLLGMANIDLEKGRTGDEIVLPRGLEYTVEECTCEDCIKNKPKVDSDHCFPLPAMEEGATILVTTKTNDYCNSLSAALSVTEIEKSISAR

SEQ ID NO: 471 - protein, 6 aa, synthetic construct
  REGION 1..6: note = Synthetic Construct
HHHHHH

SEQ ID NO: 472 - protein, 20 aa, synthetic construct
  REGION 1..20: note = Synthetic Construct
  VARIANT 1..20: note = MISC_FEATURE - This sequence may encompass 1-10 GS repeating units (Amino acids 3 to 20 can be empty or absent)
GSGSGSGSGSGSGSGSGSGS

SEQ ID NO: 473 - protein, 30 aa, synthetic construct
  REGION 1..30: note = Synthetic Construct
  VARIANT 1..30: note = MISC_FEATURE - This sequence may encompass 1-10 GGS repeating units (Amino acids 3 to 30 can be empty or absent)
GGSGGSGGSGGSGGSGGSGGSGGSGGSGGS

SEQ ID NO: 474 - protein, 40 aa, synthetic construct
  REGION 1..40: note = Synthetic Construct
  VARIANT 1..40: note = REPEAT - This sequence may encompass 1-10 GGGS repeating units
GGGSGGGSGGGSGGGSGGGSGGGSGGGSGGGSGGGSGGGS

SEQ ID NO: 475 - protein, 40 aa, synthetic construct
  REGION 1..40: note = Synthetic Construct
  VARIANT 1..40: note = REPEAT - This sequence may encompass 1-10 GGSG repeating units
GGSGGGSGGGSGGGSGGGSGGGSGGGSGGGSGGGSGGGSG

SEQ ID NO: 476 - protein, 50 aa, synthetic construct
  REGION 1..50: note = Synthetic Construct
  VARIANT 1..50: note = REPEAT - This sequence may encompass 1-10 GGSGG repeating units
GGSGGGGSGGGGSGGGGSGGGGSGGGGSGGGGSGGGGSGGGGSGGGGSGG

SEQ ID NO: 477 - protein, 50 aa, synthetic construct
  REGION 1..50: note = Synthetic Construct
  VARIANT 1..50: note = REPEAT - This sequence may encompass 1-10 GGGGS repeating units
GGGGSGGGGSGGGGSGGGGSGGGGSGGGGSGGGGSGGGGSGGGGSGGGGS

SEQ ID NO: 478 - protein, 50 aa, synthetic construct
  REGION 1..50: note = Synthetic Construct
  VARIANT 1..50: note = REPEAT - This sequence may encompass 1-10 GGGGG repeating units
GGGGGGGGGGGGGGGGGGGGGGGGGGGGGGGGGGGGGGGGGGGGGGGGGG

SEQ ID NO: 479 - protein, 30 aa, synthetic construct
  REGION 1..30: note = Synthetic Construct
  VARIANT 1..30: note = MISC_FEATURE - This sequence may encompass 1-10 GGG repeating units (Amino acids 3 to 30 can be empty or absent)
GGGGGGGGGGGGGGGGGGGGGGGGGGGGGG

SEQ ID NO: 480 - protein, 20 aa, synthetic construct
  REGION 1..20: note = Synthetic Construct
GGGGSGGGGSGGGGSGGGGS

SEQ ID NO: 481 - protein, 15 aa, synthetic construct
  REGION 1..15: note = Synthetic Construct
GGGGSGGGGSGGGGS

SEQ ID NO: 482 - protein, 5 aa, synthetic construct
  REGION 1..5: note = Synthetic Construct
LPETG

SEQ ID NO: 483 - protein, 507 aa, synthetic construct
  REGION 1..507: note = Synthetic Construct
EVQLVESGGGLVQPGRSLTLSCAASTNIFSISPMGWYRQAPGKQRELVAAIHGFSTLYADSVKGRFTISRDNAKNSIYLQMNSLRPEDTALYYCNKVPWGDYHPRNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 484 - protein, 507 aa, synthetic construct
  REGION 1..507: note = Synthetic Construct
EVQLVESGGGLVQPGRSLTLSCAASTDIFSISPMGWYRQAPGKQRELVAAIHGGSTLYADSVKGRFTISRDNAKNSIYLQMNSLRPEDTALYYCNKVPWGDYHPRNVAWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 485 - protein, 507 aa, synthetic construct
  REGION 1..507: note = Synthetic Construct
EVQLVESGGGLVQPGRSLTLSCAASTNDFSISPMGWYRQAPGKQRELVAAIHGGSTLYADSVKGRFTISRDNAKNSIYLQMNSLRPEDTALYYCNKVPWGDYHPRNVAWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 486 - protein, 507 aa, synthetic construct
  REGION 1..507: note = Synthetic Construct
EVQLVESGGGLVQPGRSLTLSCAASTNIFSKSPMGWYRQAPGKQRELVAAIHGKSTLYADSVKGRFTISRDNAKNSIYLQMNSLRPEDTALYYCNKVPWGDYHPRNVVWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 487 - protein, 507 aa, synthetic construct
  REGION 1..507: note = Synthetic Construct
EVQLVESGGGLVQPGRSLTLSCAASTNDFSISPMGWYRQAPGKQRELVAAIHGKSTLYADSVKGRFTISRDNAKNSIYLQMNSLRPEDTALYYCNKVPWGDYHPRNVKWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 488 - protein, 507 aa, synthetic construct
  REGION 1..507: note = Synthetic Construct
EVQLVESGGGLVQPGRSLTLSCAASTNQFSISPMGWYRQAPGKQRELVAAIHGKSTLYADSVKGRFTISRDNAKNSIYLQMNSLRPEDTALYYCNKVPWGDYHPRNVVWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 489 - protein, 507 aa, synthetic construct
  REGION 1..507: note = Synthetic Construct
EVQLVESGGGLVQPGRSLTLSCAASTNIFSSSPMGWYRQAPGKQRELVAAINGFSTLYADSVKGRFTISRDNAKNSIYLQMNSLRPEDTALYYCNKVPWGDYHPRNVHWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 490 - protein, 507 aa, synthetic construct
  REGION 1..507: note = Synthetic Construct
EVQLVESGGGLVQPGRSLTLSCAASTNIFSSSPMGWYRQAPGKQRELVAAIHGFSTLYADSVKGRFTISRDNAKNSIYLQMNSLRPEDTALYYCNKVPWGDYHPRNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 491 - protein, 507 aa, synthetic construct
  REGION 1..507: note = Synthetic Construct
EVQLVESGGGLVQPGRSLTLSCAASTNIFSTSPMGWYRQAPGKQRELVAAIHGFQTLYADSVKGRFTISRDNAKNSIYLQMNSLRPEDTALYYCNKVPWGDYHPRNVVWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 492 - protein, 507 aa, synthetic construct
  REGION 1..507: note = Synthetic Construct
EVQLVESGGGLVQPGRSLTLSCAASTNIFSRSPMGWYRQAPGKQRELVAAIHGFETLYADSVKGRFTISRDNAKNSIYLQMNSLRPEDTALYYCNKVPWGDYHPRNVLWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 493 - protein, 507 aa, synthetic construct
  REGION 1..507: note = Synthetic Construct
EVQLVESGGGLVQPGRSLTLSCAASTNIFSESPMGWYRQAPGKQRELVAAIHGFTTLYADSVKGRFTISRDNAKNSIYLQMNSLRPEDTALYYCNKVPWGDYHPRNVTWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 494 - protein, 507 aa, synthetic construct
  REGION 1..507: note = Synthetic Construct
EVQLVESGGGLVQPGRSLTLSCAASTNIFSDSPMGWYRQAPGKQRELVAAIHGFSTLYADSVKGRFTISRDNAKNSIYLQMNSLRPEDTALYYCNKVPWGDYHPRNVAWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 495 - protein, 507 aa, synthetic construct
  REGION 1..507: note = Synthetic Construct
EVQLVESGGGLVQPGRSLTLSCAASTNIFSNSPMGWYRQAPGKQRELVAAIHGGSTLYADSVKGRFTISRDNAKNSIYLQMNSLRPEDTALYYCNKVPWGDYHPRNVHWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 496 - protein, 507 aa, synthetic construct
  REGION 1..507: note = Synthetic Construct
EVQLVESGGGLVQPGRSLTLSCAASTNIFSNSPMGWYRQAPGKQRELVAAIHGRSTLYADSVKGRFTISRDNAKNSIYLQMNSLRPEDTALYYCNKVPWGDYHPRNVMWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 497 - protein, 507 aa, synthetic construct
  REGION 1..507: note = Synthetic Construct
EVQLVESGGGLVQPGRSLTLSCAASTNIFSNSPMGWYRQAPGKQRELVAAIHGPSTLYADSVKGRFTISRDNAKNSIYLQMNSLRPEDTALYYCNKVPWGDYHPRNVTWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 498 - protein, 507 aa, synthetic construct
  REGION 1..507: note = Synthetic Construct
EVQLVESGGGLVQPGRSLTLSCAASTNIFSNSPMGWYRQAPGKQRELVAAIHGDSTLYADSVKGRFTISRDNAKNSIYLQMNSLRPEDTALYYCNKVPWGDYHPRNVRWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 499 - protein, 507 aa, synthetic construct
  REGION 1..507: note = Synthetic Construct
EVQLVESGGGLVQPGRSLTLSCAASTNIFSRSPMGWYRQAPGKQRELVAAIHGDSTLYADSVKGRFTISRDNAKNSIYLQMNSLRPEDTALYYCNKVPWGDYHPRNVTWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 500 - protein, 507 aa, synthetic construct
  REGION 1..507: note = Synthetic Construct
EVQLVESGGGLVQPGRSLTLSCAASTNIFSKSPMGWYRQAPGKQRELVAAIHGQSTLYADSVKGRFTISRDNAKNSIYLQMNSLRPEDTALYYCNKVPWGDYHPRNVTWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 501 - protein, 507 aa, synthetic construct
  REGION 1..507: note = Synthetic Construct
EVQLVESGGGLVQPGRSLTLSCAASTNIFSSSPMGWYRQAPGKQRELVAAIHGHSTLYADSVKGRFTISRDNAKNSIYLQMNSLRPEDTALYYCNKVPWGDYHPRNVTWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 502 - protein, 507 aa, synthetic construct
  REGION 1..507: note = Synthetic Construct
EVQLVESGGGLVQPGRSLTLSCAASTNIFSSSPMGWYRQAPGKQRELVAAIHGESTLYADSVKGRFTISRDNAKNSIYLQMNSLRPEDTALYYCNKVPWGDYHPRKVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 503 - protein, 507 aa, synthetic construct
  REGION 1..507: note = Synthetic Construct
EVQLVESGGGLVQPGRSLTLSCAASTNIFSRSPMGWYRQAPGKQRELVAAIHGNSTLYADSVKGRFTISRDNAKNSIYLQMNSLRPEDTALYYCNKVPWGIYHPRNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 504 - protein, 507 aa, synthetic construct
  REGION 1..507: note = Synthetic Construct
EVQLVESGGGLVQPGRSLTLSCAASTNIFSESPMGWYRQAPGKQRELVAAIHGNSTLYADSVKGRFTISRDNAKNSIYLQMNSLRPEDTALYYCNKVPWGTYHPRNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 505 - protein, 507 aa, synthetic construct
  REGION 1..507: note = Synthetic Construct
EVQLVESGGGLVQPGRSLTLSCAASTNIFSVSPMGWYRQAPGKQRELVAAIHGNSTLYADSVKGRFTISRDNAKNSIYLQMNSLRPEDTALYYCNKVPWGKYHPRNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 506 - protein, 507 aa, synthetic construct
  REGION 1..507: note = Synthetic Construct
EVQLVESGGGLVQPGRSLTLSCAASTNIFSVSPMGWYRQAPGKQRELVAAIHGNSTLYADSVKGRFTISRDNAKNSIYLQMNSLRPEDTALYYCNKVPWGDYHPRNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 507 - protein, 507 aa, synthetic construct
  REGION 1..507: note = Synthetic Construct
EVQLVESGGGLVQPGRSLTLSCAASTNIFSKSPMGWYRQAPGKQRELVAAIHGNSTLYADSVKGRFTISRDNAKNSIYLQMNSLRPEDTALYYCNKVPWGDYHPREVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 508 - protein, 507 aa, synthetic construct
  REGION 1..507: note = Synthetic Construct
EVQLVESGGGLVQPGRSLTLSCAASTNIFSDSPMGWYRQAPGKQRELVAAIHGTSTLYADSVKGRFTISRDNAKNSIYLQMNSLRPEDTALYYCNKVPWGDYHPRNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 509 - protein, 507 aa, synthetic construct
  REGION 1..507: note = Synthetic Construct
EVQLVESGGGLVQPGRSLTLSCAASTNIFSRSPMGWYRQAPGKQRELVAAIHGTSTLYADSVKGRFTISRDNAKNSIYLQMNSLRPEDTALYYCNKVPWGKYHPRNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 510 - protein, 507 aa, synthetic construct
  REGION 1..507: note = Synthetic Construct
EVQLVESGGGLVQPGRSLTLSCAASTNIFSHSPMGWYRQAPGKQRELVAAIHGTSTLYADSVKGRFTISRDNAKNSIYLQMNSLRPEDTALYYCNKVPWGRYHPRNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 511 - protein, 507 aa, synthetic construct
  REGION 1..507: note = Synthetic Construct
EVQLVESGGGLVQPGRSLTLSCAASTNIFSTSPMGWYRQAPGKQRELVAAIHGTSTLYADSVKGRFTISRDNAKNSIYLQMNSLRPEDTALYYCNKVPWGDYHPGNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 512 - protein, 507 aa, synthetic construct
  REGION 1..507: note = Synthetic Construct
EVQLVESGGGLVQPGRSLTLSCAASTNIFSTSPMGWYRQAPGKQRELVAAIHGTSTLYADSVKGRFTISRDNAKNSIYLQMNSLRPEDTALYYCNKVPWGDYHPRNVQWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 513 - protein, 507 aa, synthetic construct
  REGION 1..507: note = Synthetic Construct
EVQLVESGGGLVQPGRSLTLSCAASTNIFSLSPMGWYRQAPGKQRELVAAIHGDSTLYADSVKGRFTISRDNAKNSIYLQMNSLRPEDTALYYCNKVPWGDYHPRNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 514 - protein, 507 aa, synthetic construct
  REGION 1..507: note = Synthetic Construct
EVQLVESGGGLVQPGRSLTLSCAASTNIFSISPGGWYRQAPGKQRELVAAIHGSSTLYADSVKGRFTISRDNAKNSIYLQMNSLRPEDTALYYCNKVPWGDYHPRNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 515 - protein, 507 aa, synthetic construct
  REGION 1..507: note = Synthetic Construct
EVQLVESGGGLVQPGRSLTLSCAASTNHFSISPMGWYRQAPGKQRELVAAIHGSSTLYADSVKGRFTISRDNAKNSIYLQMNSLRPEDTALYYCNKVPWGDYHPRVVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 516 - protein, 507 aa, synthetic construct
  REGION 1..507: note = Synthetic Construct
EVQLVESGGGLVQPGRSLTLSCAASTNIFSASPMGWYRQAPGKQRELVAAIHGSSTLYADSVKGRFTISRDNAKNSIYLQMNSLRPEDTALYYCNKVPWGDYHPRNVNWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 517 - protein, 507 aa, synthetic construct
  REGION 1..507: note = Synthetic Construct
EVQLVESGGGLVQPGRSLTLSCAASTNIFSASPMGWYRQAPGKQRELVAAIHGSSTLYADSVKGRFTISRDNAKNSIYLQMNSLRPEDTALYYCNKVPWGRYHPRNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 518 - protein, 507 aa, synthetic construct
  REGION 1..507: note = Synthetic Construct
EVQLVESGGGLVQPGRSLTLSCAASTNISSISPMGWYRQAPGKQRELVAAIHGTSTLYADSVKGRFTISRDNAKNSIYLQMNSLRPEDTALYYCNKVPWGDYHPGNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 519 - protein, 507 aa, synthetic construct
  REGION 1..507: note = Synthetic Construct
EVQLVESGGGLVQPGRSLTLSCAASTNIESISPMGWYRQAPGKQRELVAAIHGTSTLYADSVKGRFTISRDNAKNSIYLQMNSLRPEDTALYYCNKVPWGDYHPGNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 520 - protein, 507 aa, synthetic construct
  REGION 1..507: note = Synthetic Construct
EVQLVESGGGLVQPGRSLTLSCAASTNIFSISPYGWYRQAPGKQRELVAAIHGTSTLYADSVKGRFTISRDNAKNSIYLQMNSLRPEDTALYYCNKVPWGDYHPGNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 521 - protein, 507 aa, synthetic construct
  REGION 1..507: note = Synthetic Construct
EVQLVESGGGLVQPGRSLTLSCAASTNIASISPMGWYRQAPGKQRELVAAIHGTSTLYADSVKGRFTISRDNAKNSIYLQMNSLRPEDTALYYCNKVPWGDYHPGNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 522 - protein, 507 aa, synthetic construct
  REGION 1..507: note = Synthetic Construct
EVQLVESGGGLVQPGRSLTLSCAASTNIASISPMGWYRQAPGKQRELVAAIHGKSTLYADSVKGRFTISRDNAKNSIYLQMNSLRPEDTALYYCNKVPWGDYHPGNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 523 - protein, 507 aa, synthetic construct
  REGION 1..507: note = Synthetic Construct
EVQLVESGGGLVQPGRSLTLSCAASTNIASISPMGWYRQAPGKQRELVAAIHGSSTLYADSVKGRFTISRDNAKNSIYLQMNSLRPEDTALYYCNKVPWGDYHPGNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 524 - protein, 507 aa, synthetic construct
  REGION 1..507: note = Synthetic Construct
EVQLVESGGGLVQPGRSLTLSCAASTNITSISPMGWYRQAPGKQRELVAAIHGDSTLYADSVKGRFTISRDNAKNSIYLQMNSLRPEDTALYYCNKVPWGDYHPGNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 525 - protein, 507 aa, synthetic construct
  REGION 1..507: note = Synthetic Construct
EVQLVESGGGLVQPGRSLTLSCAASTNIMSISPMGWYRQAPGKQRELVAAIHGNSTLYADSVKGRFTISRDNAKNSIYLQMNSLRPEDTALYYCNKVPWGDYHPGNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 526 - protein, 507 aa, synthetic construct
  REGION 1..507: note = Synthetic Construct
EVQLVESGGGLVQPGRSLTLSCAASTNITSISPMGWYRQAPGKQRELVAAIHGNSTLYADSVKGRFTISRDNAKNSIYLQMNSLRPEDTALYYCNKVPWGDYHPGNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 527 - protein, 507 aa, synthetic construct
  REGION 1..507: note = Synthetic Construct
EVQLVESGGGLVQPGRSLTLSCAASTNIVSISPMGWYRQAPGKQRELVAAIHGHSTLYADSVKGRFTISRDNAKNSIYLQMNSLRPEDTALYYCNKVPWGDYHPGNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 528 - protein, 507 aa, synthetic construct
  REGION 1..507: note = Synthetic Construct
EVQLVESGGGLVQPGRSLTLSCAASTNIVSISPMGWYRQAPGKQRELVAAIHGKSTLYADSVKGRFTISRDNAKNSIYLQMNSLRPEDTALYYCNKVPWGDYHPGNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 529 - protein, 507 aa, synthetic construct
  REGION 1..507: note = Synthetic Construct
EVQLVESGGGLVQPGRSLTLSCAASTNVVSISPMGWYRQAPGKQRELVAAIHGKSTLYADSVKGRFTISRDNAKNSIYLQMNSLRPEDTALYYCNKVPWGDYHPNNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 530 - protein, 507 aa, synthetic construct
  REGION 1..507: note = Synthetic Construct
EVQLVESGGGLVQPGRSLTLSCAASTNIISISPMGWYRQAPGKQRELVAAIHGASTLYADSVKGRFTISRDNAKNSIYLQMNSLRPEDTALYYCNKVPWGDYHPGNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 531 - protein, 507 aa, synthetic construct
  REGION 1..507: note = Synthetic Construct
EVQLVESGGGLVQPGRSLTLSCAASTNIFSITPMGWYRQAPGKQRELVAAIHGASTLYADSVKGRFTISRDNAKNSIYLQMNSLRPEDTALYYCNKVPWGDYHPGNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 532 - protein, 507 aa, synthetic construct
  REGION 1..507: note = Synthetic Construct
EVQLVESGGGLVQPGRSLTLSCAASTNITSISPMGWYRQAPGKQRELVAAIHGFETLYADSVKGRFTISRDNAKNSIYLQMNSLRPEDTALYYCNKVPWGDYHPGNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 533 - protein, 507 aa, synthetic construct
  REGION 1..507: note = Synthetic Construct
EVQLVESGGGLVQPGRSLTLSCAASTNIQSISPMGWYRQAPGKQRELVAAIHGFETLYADSVKGRFTISRDNAKNSIYLQMNSLRPEDTALYYCNKVPWGDYHPGNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 534 - protein, 507 aa, synthetic construct
  REGION 1..507: note = Synthetic Construct
EVQLVESGGGLVQPGRSLTLSCAASTSDFSISPMGWYRQAPGKQRELVAAIHGFETLYADSVKGRFTISRDNAKNSIYLQMNSLRPEDTALYYCNKVPWGDYHPGNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 535 - protein, 507 aa, synthetic construct
  REGION 1..507: note = Synthetic Construct
EVQLVESGGGLVQPGRSLTLSCAASTNIDSISPMGWYRQAPGKQRELVAAIHGFQTLYADSVKGRFTISRDNAKNSIYLQMNSLRPEDTALYYCNKVPWGDYHPGNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 536 - protein, 507 aa, synthetic construct
  REGION 1..507: note = Synthetic Construct
EVQLVESGGGLVQPGRSLTLSCAASTNIMSISPMGWYRQAPGKQRELVAAIHGFSTVYADSVKGRFTISRDNAKNSIYLQMNSLRPEDTALYYCNKVPWGDYHPGNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 537 - protein, 507 aa, synthetic construct
  REGION 1..507: note = Synthetic Construct
EVQLVESGGGLVQPGRSLTLSCAASTNIESISPMGWYRQAPGKQRELVAAIHGFSTLYADSVKGRFTISRDNAKNSIYLQMNSLRPEDTALYYCNKVPWGDYHPGNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 538 - protein, 507 aa, synthetic construct
  REGION 1..507: note = Synthetic Construct
EVQLVESGGGLVQPGRSLTLSCAASTNIFSSSPMGWYRQAPGKQRELVAAIHGFKTLYADSVKGRFTISRDNAKNSIYLQMNSLRPEDTARYYCNKVPWGDYHPGNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 539 - protein, 507 aa, synthetic construct
  REGION 1..507: note = Synthetic Construct
EVQLVESGGGLVQPGRSLTLSCAASTNIFSSSPMGWYRQAPGKQRELVAAIHGFSTLYADSVKGRFTISRDNAKNSIYLQMNSLRPEDTALYYCNKVPWGDYHPGNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 540 - protein, 507 aa, synthetic construct
  REGION 1..507: note = Synthetic Construct
EVQLVESGGGLVQPGRSLTLSCAASTNIFSNSPMGWYRQAPGKQRELVAAIHGFSTLYADSVKGRFTISRDNAKNSIYLQMNSLRPEDTALYYCNKVPWGDYHPGNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 541 - protein, 507 aa, synthetic construct
  REGION 1..507: note = Synthetic Construct
EVQLVESGGGLVQPGRSLTLSCAASTNIFSTSPMGWYRQAPGKQRELVAAIHGFSTIYADSVKGRFTISRDNAKNSIYLQMNSLRPEDTALYYCNKVPWGDYHPGNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 542 - protein, 507 aa, synthetic construct
  REGION 1..507: note = Synthetic Construct
EVQLVESGGGLVQPGRSLTLSCAASTNIFSTSPMGWYRQAPGKQRELVAAIHGFSTIYADSVKGRFTISRDNAKNSIYLQMNSLRPEDTALYYCNKVPWGDYHPLNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 543 - protein, 507 aa, synthetic construct
  REGION 1..507: note = Synthetic Construct
EVQLVESGGGLVQPGRSLTLSCVASTNIFSTSPMGWYRQAPGKQRELVAAIHGFSTLYADSVKGRFTISRDNAKNSIYLQMNSLRPEDTALYYCNKVPWGDYHPGNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 544 - protein, 507 aa, synthetic construct
  REGION 1..507: note = Synthetic Construct
EVQLVESGGGLVQPGRSLTLSCAASTNIFSDSPMGWYRQAPGKQRELVAAIHGFSTFYADSVKGRFTISRDNAKNSIYLQMNSLRPEDTALYYCNKVPWGDYHPGNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 545 - protein, 507 aa, synthetic construct
  REGION 1..507: note = Synthetic Construct
EVQLVESGGGLVQPGRSLTLSCAASTNIFSQSPMGWYRQAPGKQRELVAAIHGDSTLYADSVKGRFTISRDNAKNSIYLQMNSLRPEDTALYYCNKVPWGDYHPGNVCWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 546 - protein, 507 aa, synthetic construct
  REGION 1..507: note = Synthetic Construct
EVQLVESGGGLVQPGRSLTLSCAASTNIFSQSPMGWYRQAPGKQRELVAAIHGKSTLYADSVKGRFTISRDNAKNSIYLQMNSLRPEDTALYYCNKVPWGDYHPSNVYWGKGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 547 - protein, 507 aa, synthetic construct
  REGION 1..507: note = Synthetic Construct
EVQLVESGGGLVQPGRSLTLSCAASTNIFSRSPMGWYRQAPGKQRELVAAIHGESTLYADSVKGRFTISRDNAKNSIYLQMNSLRPEDTALYYCNKVPWGRYHPGNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 548 - protein, 507 aa, synthetic construct
  REGION 1..507: note = Synthetic Construct
EVQLVESGGGLVQPGRSLTLSCAASTNIFSRSPMGWYRQAPGKQRELVAAIHGISTLYADSVKGRFTISRDNAKNSIYLQMNSLRPEDTALYYCNKVPWGDYHPGNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 549 - protein, 507 aa, synthetic construct
  REGION 1..507: note = Synthetic Construct
EVQLVESGGGLVQPGRSLTLSCAASTNIFSRSPMGWYRQAPGKQRELVAAIHGSSTLYADSVKGRFTISRDNAKNSIYLQMNSLRPEDTALYYCNKVPWGDYHPGNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 550 - protein, 507 aa, synthetic construct
  REGION 1..507: note = Synthetic Construct
EVQLVESGGGLVQPGRSLTLSCAASTNIFSGSPMGWYRQAPGKQRELVAAIHGNSTLYADSVKGRFTISRDNAKNSIYLQMNSLRPEDTALYYCNKVPWGDYHPGNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 551 - protein, 507 aa, synthetic construct
  REGION 1..507: note = Synthetic Construct
EVQLVESGGGLVQPGRSLTLSCAASSNIFSISPMGWYRQAPGKQRELVAAIHGSSTLYADSVKGRFTISRDNAKNSIYLQMNSLRPEDTALYYCNKVPWGDYHPGNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 552 - protein, 507 aa, synthetic construct
  REGION 1..507: note = Synthetic Construct
EVQLVESGGGLVQPGRSLTLSCAASTNIFSIYPMGWYRQAPGKQRELVAAIHGSSTLYADSVKGRFTISRDNAKNSIYLQMNSLRPEDTALYYCNKVPWGDYHPKNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 553 - protein, 507 aa, synthetic construct
  REGION 1..507: note = Synthetic Construct
EVQLVESGGGLVQPGRSLTLSCAASTNIFSKSPMGWYRQAPGKQRELVAAIHGSSTLYADSVKGRFTISRDNAKNSIYLQMNSLRPEDTALYYCNKVPWGDYHPGNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 554 - protein, 507 aa, synthetic construct
  REGION 1..507: note = Synthetic Construct
EVQLVESGGGLVQPGRSLTLSCAASTNIFSKSPMGWYRQAPGKQRELVAAIHGSSTLYADSVKGRFTISRDNAKNSIYLQMNSLRPEDTALYYCNKVPWGDYHPRNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 555 - protein, 507 aa, synthetic construct
  REGION 1..507: note = Synthetic Construct
EVQLVESGGGLVQPGRSLTLSCAASTNEFSISPMGWYRQAPGKQRELVAAIHGLSTLYADSVKGRFTISRDNAKNSIYLQMNSLRPEDTALYYCNKVPWGAYHPRNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 556 - protein, 507 aa, synthetic construct
  REGION 1..507: note = Synthetic Construct
EVQLVESGGGLVQPGRSLTLSCAASTNEFSISPMGWYRQAPGKQRELVAAIHGESTLYADSVKGRFTISRDNAKNSIYLQMNSLRPEDTALYYCNKVPWGDYHPGNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 557 - protein, 507 aa, synthetic construct
  REGION 1..507: note = Synthetic Construct
EVQLVESGGGLVQPGRSLTLSCAASTNIPSISPMGWYRQAPGKQRELVAAIHGESTLYADSVKGRFTISRDNAKNSIYLQMNSLRPEDTALYYCNKVPWGDYHPRNVAWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 558 - protein, 507 aa, synthetic construct
  REGION 1..507: note = Synthetic Construct
EVQLVESGGGLVQPGRSLTLSCAASTNIPSISPMGWYRQAPGKQRELVAAIHGASTLYADSVKGRFTISRDNAKNSIYLQMNSLRPEDTALYYCNKVPWGDYHPRNVAWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 559 - protein, 507 aa, synthetic construct
  REGION 1..507: note = Synthetic Construct
EVQLVESGGGLVQPGRSLTLSCAASTNIPSISPMGWYRQAPGKQRELVAAIHGESTLYADSVKGRFTISRDNAKNSIYLQMNSLRPEDTALYYCNKVPWGDYHPRNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 560 - protein, 507 aa, synthetic construct
  REGION 1..507: note = Synthetic Construct
EVQLVESGGGLVQPGRSLTLSCAASTNIPSISPMGWYRQAPGKQRELVAAIHGDSTLYADSVKGRFTISRDNAKNSIYLQMNSLRPEDTALYYCNKVPWGDYHPRNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 561 - protein, 507 aa, synthetic construct
  REGION 1..507: note = Synthetic Construct
EVQLVESGGGLVQPGRSLTLSCAASTNITSISPMGWYRQAPGKQRELVAAIHGVSTLYADSVKGRFTISRDNAKNSIYLQMNSLRPEDTALYYCNKVPWGDYHPRNVQWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 562 - protein, 507 aa, synthetic construct
  REGION 1..507: note = Synthetic Construct
EVQLVESGGGLVQPGRSLTLSCAASTNIPSISPMGWYRQAPGKQRELVAAIHGQSTLYADSVKGRFTISRDNAKNSIYLQMNSLRPEDTALYYCNKVPWGDYHPRNVQWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 563 - protein, 507 aa, synthetic construct
  REGION 1..507: note = Synthetic Construct
EVQLVESGGGLVQPGRSLTLSCAASTNIVSISPMGWYRQAPGKQRELVAAIHGDSTLYADSVKGRFTISRDNAKNSIYLQMNSLRPEDTALYYCNKVPWGDYHPRNVSWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 564 - protein, 507 aa, synthetic construct
  REGION 1..507: note = Synthetic Construct
EVQLVESGGGLVQPGRSLTLSCAASSNIFSISPMGWYRQAPGKQRELVAAIHGESTLYADSVKGRFTISRDNAKNSIYLQMNSLRPEDTALYYCNKVPWGDYHPRNVTWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 565 - protein, 507 aa, synthetic construct
  REGION 1..507: note = Synthetic Construct
EVQLVESGGGLVQPGRSLTLSCAASTNIDSISPMGWYRQAPGKQRELVAAIHGESTLYADSVKGRFTISRDNAKNSIYLQMNSLRPEDTALYYCNKVPWGDYHPRNVTWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 566 - protein, 507 aa, synthetic construct
  REGION 1..507: note = Synthetic Construct
EVQLVESGGGLVQPGRSLTLSCAASTNIDSISPMGWYRQAPGKQRELVAAIHGSSTLYADSVKGRFTISRDNAKNSIYLQMNSLRPEDTALYYCNKVPWGDYHPRNVTWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 567 - protein, 507 aa, synthetic construct
  REGION 1..507: note = Synthetic Construct
EVQLVESGGGLVQPGRSLTLSCAASTNIRSISPMGWYRQAPGKQRELVAAIHGSSTLYADSVKGRFTISRDNAKNSIYLQMNSLRPEDTALYYCNKVPWGDYHPRNVVWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 568 - protein, 507 aa, synthetic construct
  REGION 1..507: note = Synthetic Construct
EVQLVESGGGLVQPGRSLTLSCAASTNITSISPMGWYRQAPGKQRELVAAISGFSTLYADSVKGRFTISRDNAKNSIYLQMNSLRPEDTALYYCNEVPWGDYHPRNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 569 - protein, 507 aa, synthetic construct
  REGION 1..507: note = Synthetic Construct
EVQLVESGGGLVQPGRSLTLSCAASTNITSISPMGWYRQAPGKQRELVAAIHGESTLYADSVKGRFTISRDNAKNSIYLQMNSLRPEDTALYYCNKVPWGDYHPRNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 570 - protein, 507 aa, synthetic construct
  REGION 1..507: note = Synthetic Construct
EVQLVESGGGLVQPGRSLTLSCAASTNITSVSPMGWYRQAPGKQRELVAAIHGPSTLYADSVKGRFTISRDNAKNSIYLQMNSLRPEDTALYYCNKVPWGDYHPTNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 571 - protein, 507 aa, synthetic construct
  REGION 1..507: note = Synthetic Construct
EVQLVESGGGLVQPGRSLTLSCAASTNIGSISPMGWYRQAPGKQRELVAAIHGQSTLYADSVKGRFTISRDNAKNSIYLQMNSLRPEDTALYYCNKVPWGDYHPQNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 572 - protein, 507 aa, synthetic construct
  REGION 1..507: note = Synthetic Construct
EVQLVESGGGLVQPGRSLTLSCAASTNIESISPMGWYRQAPGKQRELVAAIHGKSTLYADSVKGRFTISRDNAKNSIYLQMNSLRPEDTALYYCNKVPWGDYHPRRVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 573 - protein, 507 aa, synthetic construct
  REGION 1..507: note = Synthetic Construct
EVQLVESGGGLVQPGRSLTLSCAASTNIVSISPMGWYRQAPGKQRELVAAIHGDSTLYADSVKGRFTISRDNAKNSIYLQMNSLRPEDTALYYCNKVPWGDYHPRRVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 574 - protein, 507 aa, synthetic construct
  REGION 1..507: note = Synthetic Construct
EVQLVESGGGLVQPGRSLTLSCAASTNIDSISPMGWYRQAPGKQRELVAAIHGNSTLYADSVKGRFTISRDNAKNSIYLQMNSLRPEDTALYYCNKVPWGDYHPRMVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 575 - protein, 507 aa, synthetic construct
  REGION 1..507: note = Synthetic Construct
EVQLVESGGGLVQPGRSLTLSCAASTNIFMISPMGWYRQAPGKQRELVAAIHGDSTLYADSVKGRFTISRDNAKNSIYLQMNSLRPEDTALYYCNKVPWGRYHPRNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 576 - protein, 507 aa, synthetic construct
  REGION 1..507: note = Synthetic Construct
EVQLVESGGGLVQPGRSLTLSCAASTNIFRISPMGWYRQAPGKQRELVAAIHGDSTLYADSVKGRFTISRDNAKNSIYLQMNSLRPEDTALYYCNKVPWGRYHPRNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 577 - protein, 507 aa, synthetic construct
  REGION 1..507: note = Synthetic Construct
EVQLVESGGGLVQPGRSLTLSCAASTNIFSISPMGWYRQAPGKQRELVAAIHGDSTLYADSVKGRFTISRDNAKNSIYLQMNSLRPEDTALYYCNKVPWGEYHPRNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 578 - protein, 507 aa, synthetic construct
  REGION 1..507: note = Synthetic Construct
EVQLVESGGGLVQPGRSLTLSCAASTNIFSISPMGWYRQAPGKQRELVAAIHGDSTLYADSVKGRFTISRDNAKNSIYLQMNSLRPEDTALYYCNKVPWGKYHPRNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 579 - protein, 507 aa, synthetic construct
  REGION 1..507: note = Synthetic Construct
EVQLVESGGGLVQPGRSLTLSCAASTNIESISPMGWYRQAPGKQRELVAAIHGSSTLYADSVKGRFTISRDNAKNSIYLQMNSLRPEDTALYYCNKVPWGRYHPRNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 580 - protein, 507 aa, synthetic construct
  REGION 1..507: note = Synthetic Construct
EVQLVESGGGLVQPGRSLTLSCAASTNIESISPMGWYRQAPGKQRELVAAIHGNSTLYADSVKGRFTISRDNAKNSIYLQMNSLRPEDTALYYCNKVPWGRYHPRNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 581 - protein, 507 aa, synthetic construct
  REGION 1..507: note = Synthetic Construct
EVQLVESGGGLVQPGRSLTLSCAASTNISSISPMGWYRQAPGKQRELVAAIHGFSTLYADSVKGRFTISRDNAKNSIYLQMNSLRPEDTALYYCNKVPWGYYHPRNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 582 - protein, 507 aa, synthetic construct
  REGION 1..507: note = Synthetic Construct
EVQLVESGGGLVQPGRSLTLSCAASTNISSISPMGWYRQAPGKQRELVAAIHGHSTLYADSVKGRFTISRDNAKNSIYLQMNSLRPEDTALYYCNKVPWGRYHPRNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 583 - protein, 507 aa, synthetic construct
  REGION 1..507: note = Synthetic Construct
EVQLVESGGGLVQPGRSLTLSCAASTNISSISPMGWYRQAPGKQRELVAAIHGFSTVYADSVKGRFTISRDNAKNSIYLQMNSLRPEDTALYYCNKVPWGRYHPRNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 584 - protein, 507 aa, synthetic construct
  REGION 1..507: note = Synthetic Construct
EVQLVESGGGLVQPGRSLTLSCAASTNIFSIRPMGWYRQAPGKQRELVAAIHGFSTVYADSVKGRFTISRDNAKNSIYLQMNSLRPEDTALYYCNKVPWGDYHPRNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 585 - protein, 507 aa, synthetic construct
  REGION 1..507: note = Synthetic Construct
EVQLVESGGGLVQPGRSLTLSCAASTNIFSIYPMGWYRQAPGKQRELVAAIHGFSTYYADSVKGRFTISRDNAKNSIYLQMNSLRPEDTALYYCNKVPWGSYHPRNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 586 - protein, 507 aa, synthetic construct
  REGION 1..507: note = Synthetic Construct
EVQLVESGGGLVQPGRSLTLSCAASTNIFNISPMGWYRQAPGKQRELVAAIHGFSTYYADSVKGRFTISRDNAKNSIYLQMNSLRPEDTALYYCNKVPWGRYHPRNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 587 - protein, 507 aa, synthetic construct
  REGION 1..507: note = Synthetic Construct
EVQLVESGGGLVQPGRSLTLSCAASTNIFSSSPMGWYRQAPGKQRELVAAIHGFSTWYADSVKGRFTISRDNAKNSIYLQMNSLRPEDTALYYCNKVPWGRYHPRNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 588 - protein, 507 aa, synthetic construct
  REGION 1..507: note = Synthetic Construct
EVQLVESGGGLVQPGRSLTLSCAASTNISSISPMGWYRQAPGKQRELVAAIHGFDTLYADSVKGRFTISRDNAKNSIYLQMNSLRPEDTALYYCNKVPWGDYHPRNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 589 - protein, 507 aa, synthetic construct
  REGION 1..507: note = Synthetic Construct
EVQLVESGGGLVQPGRSLTLSCAASTNIFSINPMGWYRQAPGKQRELVAAIHGFDTLYADSVKGRFTISRDNAKNSIYLQMNSLRPEDTALYYCNKVPWGDYHPRNVSWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 590 - protein, 507 aa, synthetic construct
  REGION 1..507: note = Synthetic Construct
EVQLVESGGGLVQPGRSLTLSCAASTNIFSITPMGWYRQAPGKQRELVAAIHGRSTLYADSVKGRFTISRDNAKNSIYLQMNSLRPEDTALYYCNKVPWGSYHPRNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 591 - protein, 507 aa, synthetic construct
  REGION 1..507: note = Synthetic Construct
EVQLVESGGGLVQPGRSLTLSCAASTNIFSITPMGWYRQAPGKQRELVAAIHGTSTLYADSVKGRFTISRDNAKNSIYLQMNSLRPEDTALYYCNKVPWGRYHPRNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 592 - protein, 507 aa, synthetic construct
  REGION 1..507: note = Synthetic Construct
EVQLVESGGGLVQPGRSLTLSCAASTNIFSITPMGWYRQAPGKQRELVAAIHGESTLYADSVKGRFTISRDNAKNSIYLQMNSLRPEDTALYYCNKVPWGRYHPRNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 593 - protein, 507 aa, synthetic construct
  REGION 1..507: note = Synthetic Construct
EVQLVESGGGLVQPGRSLTLSCAASTNIFSITPMGWYRQAPGKQRELVAAIHGESTLYADSVKGRFTISRDNAKNSIYLQMNSLRPEDTALYYCNKVPWGDYHPRDVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 594 - protein, 507 aa, synthetic construct
  REGION 1..507: note = Synthetic Construct
EVQLVESGGGLVQPGRSLTLSCAASTNIFSTSPYGWYRQAPGKQRELVAAIHGFSTIYADSVKGRFTISRDNAKNSIYLQMNSLRPEDTALYYCNKVPWGDYHPGNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 595 - protein, 507 aa, synthetic construct
  REGION 1..507: note = Synthetic Construct
EVQLVESGGGLVQPGRSLTLSCAASTNIFSTSPGGWYRQAPGKQRELVAAIHGFSTIYADSVKGRFTISRDNAKNSIYLQMNSLRPEDTALYYCNKVPWGDYHPGNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 596 - protein, 507 aa, synthetic construct
  REGION 1..507: note = Synthetic Construct
EVQLVESGGGLVQPGRSLTLSCAASTNIFSITPYGWYRQAPGKQRELVAAIHGASTLYADSVKGRFTISRDNAKNSIYLQMNSLRPEDTALYYCNKVPWGDYHPGNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 597 - protein, 507 aa, synthetic construct
  REGION 1..507: note = Synthetic Construct
EVQLVESGGGLVQPGRSLTLSCAASTNIFSITPGGWYRQAPGKQRELVAAIHGASTLYADSVKGRFTISRDNAKNSIYLQMNSLRPEDTALYYCNKVPWGDYHPGNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 598 - protein, 119 aa, Homo sapiens
QVQLVESGGGLVQPGESLRLSCAASTNIFSISPMGWYRQAPGKQRELVAAIHGFSTLYADSVKGRFTISRDNAKNTIYLQMNSLKPEDTAVYYCNKVPWGDYHPRNVYWGQGTQVTVSS

SEQ ID NO: 599 - protein, 10 aa, synthetic construct
  REGION 1..10: note = Synthetic Construct
TNIFSISPMG

SEQ ID NO: 600 - protein, 14 aa, synthetic construct
  REGION 1..14: note = Synthetic Construct
AIHGFSTLYADSVK

SEQ ID NO: 601 - protein, 12 aa, synthetic construct
  REGION 1..12: note = Synthetic Construct
VPWGDYHPRNVY

SEQ ID NO: 602 - protein, 9 aa, synthetic construct
  REGION 1..9: note = Synthetic Construct
GGGGSGGGS

(19) United States
(12) Patent Application Publication
(10) Pub. No.: US 2024/0254250 A1
(43) Pub. Date: Aug. 1, 2024

(54) BCMA TARGETING TRISPECIFIC PROTEINS AND METHODS OF USE

(71) Applicant: Harpoon Therapeutics, Inc., South San Francisco, CA (US)

(72) Inventors: Holger WESCHE, San Francisco, CA (US); Liping SUN, San Ramon, CA (US)

(21) Appl. No.: 18/524,822

(22) Filed: Nov. 30, 2023

Related U.S. Application Data
(63) Continuation of application No. PCT/US2022/031917, filed on Jun. 2, 2022.
(60) Provisional application No. 63/288,124, filed on Dec. 10, 2021.
(60) Provisional application No. 63/196,595, filed on Jun. 3, 2021.

(51) Int. Cl. C07K 16/28; A61P 35/00

(52) U.S. Cl. CPC C07K 16/2878; A61P 35/00; C07K 16/2809; C07K 2317/31; C07K 2317/569; C07K 2317/622

(57) ABSTRACT

Provided herein are B cell maturation agent (BCMA) targeting trispecific proteins comprising a domain binding to CD3, a half-life extension domain, and a domain binding to BCMA. Also provided are pharmaceutical compositions thereof, as well as nucleic acids, recombinant expression vectors and host cells for making such BCMA targeting trispecific proteins. Also disclosed are methods of using the disclosed BCMA targeting trispecific proteins in the prevention, and/or treatment diseases, conditions and disorders.

### CROSS-REFERENCE

This patent application is a continuation application of International Application No. PCT/US2022/031917, filed Jun. 2, 2022; and claims the benefit of U.S. Provisional Patent Application No. 63/196,595 filed Jun. 3, 2021; and U.S. Provisional Patent Application No. 63/288,124 filed Dec. 10, 2021; each of which is incorporated herein by reference in its entirety.

### SEQUENCE LISTING

The instant application contains a Sequence Listing which has been submitted electronically in XML file format and is hereby incorporated by reference in its entirety. Said XML copy, created on Feb. 13, 2024, is named 47517-757_301_SL.xml and is 828.642 bytes in size.

### BACKGROUND OF THE INVENTION

Cancer is the second leading cause of human death next to coronary disease. Worldwide, millions of people die from cancer every year. In the United States alone, cancer causes the death of well over a half-million people each year, with some 1.4 million new cases diagnosed per year. While deaths from heart disease have been declining significantly, those resulting from cancer generally are on the rise. In the early part of the next century, cancer is predicted to become the leading cause of death.

Moreover, even for those cancer patients that initially survive their primary cancers, common experience has shown that their lives are dramatically altered. Many cancer patients experience strong anxieties driven by the awareness of the potential for recurrence or treatment failure. Many cancer patients experience significant physical debilitations following treatment.

Generally speaking, the fundamental problem in the management of the deadliest cancers is the lack of effective and non-toxic systemic therapies. Cancer is a complex disease characterized by genetic mutations that lead to uncontrolled cell growth. Cancerous cells are present in all organisms and, under normal circumstances, their excessive growth is tightly regulated by various physiological factors.

### SUMMARY OF THE INVENTION

Described herein is a method of treating cancer, the method comprising administration of an effective amount of a B cell maturation agent (BCMA) targeting trispecific protein to a subject, wherein said protein comprises (a) a first domain (A) which specifically binds to human CD3; (b) a second domain (B) which is a half-life extension domain; and (c) a third domain (C) which specifically binds to BCMA, wherein the domains are linked in the order H₂N-(C)-(B)-(A)-COOH, or by linkers L1 and L2, and wherein the BCMA targeting trispecific protein is administered at a dosage of about 1 μg to about 100 mg. In some embodiments, the BCMA targeting trispecific protein is administered at a dosage of about 1 μg to about 5 mg. In some embodiments, the BCMA targeting trispecific protein is administered at a dosage of about 1 μg to about 3 mg. In some embodiments, the BCMA targeting trispecific protein is administered at a dosage of about 1 μg to about 2 mg. In some embodiments, the BCMA targeting trispecific protein is administered at a dosage of about 1 μg to about 1 mg. In some embodiments, the BCMA targeting trispecific protein is administered at a dosage of about 5 μg to about 2150 μg. In some embodiments, the BCMA targeting trispecific protein is administered at a dosage of from about 5 μg to about 2860 μg. In some embodiments, the BCMA targeting trispecific protein is administered at a dosage of 2860 μg. In some embodiments, the BCMA targeting trispecific protein is administered at a dosage of 2150 μg. In some embodiments, the BCMA targeting trispecific protein is administered at a dosage of 1620 μg. In some embodiments, the BCMA targeting trispecific protein is administered at a dosage of 810 μg. In some embodiments, the BCMA targeting trispecific protein is administered at a dosage of 270 μg. In some embodiments, the BCMA targeting trispecific protein is administered once a week. In some embodiments, the BCMA targeting trispecific protein is administered twice per week. In some embodiments, the BCMA targeting trispecific protein is administered every other week. In some embodiments, the BCMA targeting trispecific protein is administered every three weeks. In some embodiments, the BCMA targeting trispecific protein is administered intravenously, intraperitoneally, subcutaneously, intramuscularly, topically or intradermally.

Described herein is a method of treating cancer, the method comprising administration of an effective amount of a B cell maturation agent (BCMA) targeting trispecific protein to a subject, wherein said protein comprises (a) a first domain (A) which specifically binds to human CD3; (b) a second domain (B) which is a half-life extension domain; and (c) a third domain (C) which specifically binds to BCMA, wherein the domains are linked in the order H₂N-(C)-(B)-(A)-COOH, or by linkers L1 and L2, and wherein the BCMA targeting trispecific protein is administered according to a schedule comprising the following steps: (i) administration of a first dose of the BCMA targeting trispecific protein, and (ii) administration of a second dose of the BCMA targeting trispecific protein, wherein the second dose is higher than the first dose. In some embodiments, the first dose is from about 1 μg to about 10 mg. In some embodiments, the first dose is about 1 μg to about 5 mg. In some embodiments, the first dose is about 1 μg to about 4 mg. In some embodiments, the first dose is about 1 μg to about 3 mg. In some embodiments, the first dose is about 1 μg to about 2 mg. In some embodiments, the first dose is about 1 mg. In some embodiments, the first dose is about 1.62 mg. In some embodiments, the first dose is about 1.5 mg. In some embodiments, the first dose is about 2.15 mg. In some embodiments, the first dose is about 2.86 mg. In some embodiments, the first dose is about 3.24 mg. In some embodiments, the first dose is administered for about 1 week to about 36 weeks. In some embodiments, the first dose is administered for about 1 week to about 27 weeks. In some embodiments, first dose is administered for about 1 week to about 18 weeks. In some embodiments, first dose is administered for about 1 week to about 9 weeks. In some embodiments, the first dose is administered once a day. In some embodiments, the first dose is administered twice a day. In some embodiments, the first dose is administered three times a day. In some embodiments, the first dose is administered five times a day. In some embodiments, the first dose is administered once a week. In some embodiments, the first dose is administered twice per week. In some embodiments, the first dose is administered every other week. In some embodiments, the first dose is administered every three weeks. In some embodiments, the first dose is administered intravenously, intraperitoneally, subcutaneously, intramuscularly, topically or intradermally. In some embodiments, the second dose is from about 1 mg to about 12 mg. In some embodiments, the second dose is from about 1 mg to about 24 mg. In some embodiments, the second dose is from about 1 mg to about 48 mg. In some embodiments, the second dose is from about 5 mg to about 12 mg. In some embodiments, the second dose is from about 10 mg to about 48 mg. In some embodiments, the second dose is about 10 mg. In some embodiments, the second dose is about 12 mg. In some embodiments, the second dose is about 24 mg. In some embodiments, the second dose is about 36 mg. In some embodiments, the second dose is about 48 mg. In some embodiments, the second dose is administered for from about 1 week to about 36 weeks. In some embodiments, the second dose is administered for from about 1 week to about 27 weeks. In some embodiments, the second dose is administered for from about 1 week to about 18 weeks. In some embodiments, the second dose is administered for from about 1 week to about 9 weeks. In some embodiments, the second dose is administered once a day. In some embodiments, the second dose is administered twice a day. In some embodiments, the second dose is administered three times a day. In some embodiments, the second dose is administered five times a day. In some embodiments, the second dose is administered once a week. In some embodiments, the second dose is administered twice per week. In some embodiments, the second dose is administered every other week. In some embodiments, the second dose is administered every three weeks. In some embodiments, the second dose is maintained to the end of the schedule after the administration of the first dose. In some embodiments, the second dose is administered intravenously, intraperitoneally, subcutaneously, intramuscularly, topically or intradermally.

For any of the methods described herein, the BCMA targeting trispecific protein has an elimination half-time of at least 12 hours, at least 20 hours, at least 25 hours, at least 30 hours, at least 35 hours, at least 40 hours, at least 45 hours, at least 50 hours, or at least 100 hours. In some embodiments, the third domain comprises a VHH domain. In some embodiments, the VHH domain is human, humanized, affinity matured, or a combination thereof. In some embodiments, the third domain comprises one or more sequences selected from the group consisting of SEQ ID NO: 346-460. In some embodiments, the first domain comprises a variable light chain and variable heavy chain each of which is capable of specifically binding to human CD3. In some embodiments, the first domain is humanized or human. In some embodiments, the second domain binds human serum albumin. In some embodiments, the second domain comprises a scFv, a variable heavy domain (VH), a variable light domain (VL), a peptide, a ligand, or a small molecule. In some embodiments, the linkers L1 and L2 are each independently selected from (GS)ₙ (SEQ ID NO: 472), (GGS)ₙ (SEQ ID NO: 473), (GGGS)ₙ (SEQ ID NO: 474), (GGSG)ₙ (SEQ ID NO: 475), (GGSGG)ₙ (SEQ ID NO: 476), (GGGGS)ₙ (SEQ ID NO: 477), or GGGGSGGGS (SEQ ID NO: 602), wherein n is 1, 2, 3, 4, 5, 6, 7, 8, 9, or 10. In some embodiments, the linkers L1 and L2 are each independently (GGGGS)₄ (SEQ ID NO: 480), (GGGGS); (SEQ ID NO: 481) or GGGGSGGGS (SEQ ID NO: 602). In some embodiments, the domains are linked in the order H₂N-(C)-L1-(B)-L2-(A)-COOH. In some embodiments, the BCMA targeting trispecific protein is less than about 80 kDa. In some embodiments, the BCMA targeting trispecific protein is about 50 to about 75 kDa. In some embodiments, the BCMA targeting trispecific protein is less than about 60 kDa. In some embodiments, the BCMA targeting trispecific protein comprises a sequence selected from the group consisting of SEQ ID NO: 483-597. In some embodiments, the BCMA targeting trispecific protein comprises a sequence as set forth in SEQ ID NO: 520. In some embodiments, the cancer is a tumorous disease, an autoimmune disease or an infection disease associated with BCMA. In some embodiments, the cancer is a multiple myeloma, a leukemia, a lymphoma, or a metastasis thereof. In some embodiments, the cancer is a multiple myeloma.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is schematic representation of an exemplary BCMA targeting trispecific antigen-binding protein where the protein has a constant core element comprising an anti-CD3ε single chain variable fragment (scFv) and an anti-ALB variable heavy chain region; and an anti-BCMA binding domain that can be a VHH, a VH, scFv, a non-Ig binder, or a ligand.

FIG. 2 illustrates the effect of exemplary BCMA targeting molecules (01H08, 01F07, (2F02, and BH253), containing an anti-BCMA binding protein according to the present disclosure, in killing of purified human T cells that expresses BCMA compared to a negative control.

FIG. 3 is an image of an SDS-PAGE of representative purified BCMA trispecific molecules. Lane 1: 01F07-M34Y TriTAC non-reduced; Lane 2: 01F07-M34G-TriTAC non-reduced; Lane 3: 02B05 TriTAC non-reduced; Lane 4: 02G02-M34Y TriTAC non-reduced; Lane 5: 02G02 M34G TriTAC non-reduced; Lane 6: Broad Range SDS-PAGE Standard (Bio-Rad #1610317); Lane 7: 01F07-M34Y TriTAC non-reduced; Lane 8:01F07-M34G-TriTAC non-reduced; Lane 9: 02B05 TriTAC non-reduced; Lane 10: 02G02-M34Y TriTAC non-reduced; Lane 11: 02G02 M34G TriTAC non-reduced; Lane 12: Broad Range SDS-PAGE Standard (Bio-Rad #1610317)

FIGS. 4A-41 illustrate the effect of exemplary BCMA trispecific targeting molecules containing an anti-BCMA binding protein according to the present disclosure in killing of Jeko1, MOLP-8 or OPM-2 cells that express BCMA compared to a negative control.

FIGS. 5A-5D illustrate binding of an exemplary BCMA trispecific targeting protein (02B05) to purified T Cells from four different human donors, donor 02 (FIG. 5A), donor 35 (FIG. 5B), donor 81 (FIG. 5C), donor 86 (FIG. 5D).

FIGS. 6A-6F illustrate binding of an exemplary BCMA trispecific targeting protein (02B05) to cells expressing BCMA, NCI-H929 (FIG. 6A), EJM (FIG. 6B), OPM2 (FIG. 6D), RPMI8226 (FIG. 6E); or cell lines lacking expression of BCMA, NCI-H510A (FIG. 6C) and DMS-153 (FIG. 6F).

FIG. 7 illustrates the results of a TDCC assay using an exemplary BCMA trispecific targeting protein (02B05) and BCMA expressing EJM cells, in presence or absence of human serum albumin (HSA).

FIG. 8 illustrates the results of a TDCC assay using an exemplary BCMA trispecific targeting protein (02B05) and BCMA expressing EJM cells, using a varying effector cells to target cells ratio.

FIG. 9 illustrates the results of a TDCC assay using an exemplary BCMA trispecific targeting protein (02B05) and BCMA expressing OPM2 cells, using a varying effector cells to target cells ratio.

FIG. 10 illustrates the results of a TDCC assay using an exemplary BCMA trispecific targeting protein (02B05) and BCMA expressing NCI-H929 cells, using varying time-points and a 1:1 effector cells to target cells ratio.

FIG. 11 illustrates the results of a TDCC assay using an exemplary BCMA trispecific targeting protein (02B05), BCMA expressing EJM cells, and T cells from four different donors, in presence of human serum albumin (HSA).

FIG. 12 illustrates the results of a TDCC assay using an exemplary BCMA trispecific targeting protein (02B05), BCMA expressing NCI-H929 cells, and T cells from four different donors, in presence of human serum albumin (HSA).

FIG. 13 illustrates the results of a TDCC assay using an exemplary BCMA trispecific targeting protein (02B05), BCMA expressing OPM2 cells, and T cells from four different donors, in presence of human serum albumin (HSA).

FIG. 14 illustrates the results of a TDCC assay using an exemplary BCMA trispecific targeting protein (02B05), BCMA expressing RPMI8226 cells, and T cells from four different donors, in presence of human serum albumin (HSA).

FIG. 15 illustrates the results of a TDCC assay using an exemplary BCMA trispecific targeting protein (02B05), BCMA non-expressing OVCAR8 cells, and T cells from four different donors, in presence of human serum albumin (HSA).

FIG. 16 illustrates the results of a TDCC assay using an exemplary BCMA trispecific targeting protein (02B05), BCMA non-expressing NCI-H510A cells, and T cells from four different donors, in presence of human serum albumin (HSA).

FIG. 17 illustrates the results of a TDCC assay using an exemplary BCMA trispecific targeting protein (02B05), BCMA expressing NCI-H929 cells, and peripheral blood mononuclear cells (PBMC) from two different cynomolgus donors, in presence of human serum albumin (HSA).

FIG. 18 illustrates the results of a TDCC assay using an exemplary BCMA trispecific targeting protein (02B05), BCMA expressing RPMI8226 cells, and peripheral blood mononuclear cells (PBMC) from two different cynomolgus donors, in presence of human serum albumin (HSA).

FIG. 19 illustrates the expression level of T cell activation biomarker CD69, following a TDCC assay using an exemplary BCMA targeting trispecific protein (02B05) and BCMA expressing cells EJM.

FIG. 20 illustrates the expression level of T cell activation biomarker CD25, following a TDCC assay using an exemplary BCMA targeting trispecific protein (02B05) and BCMA expressing cells EJM.

FIG. 21 illustrates the expression level of T cell activation biomarker CD69, following a TDCC assay using an exemplary BCMA targeting trispecific protein (02B05) and BCMA expressing cells OPM2.

FIG. 22 illustrates the expression level of T cell activation biomarker CD25, following a TDCC assay using an exemplary BCMA targeting trispecific protein (02B05) and BCMA expressing cells OPM2.

FIG. 23 illustrates the expression level of T cell activation biomarker CD69, following a TDCC assay using an exemplary BCMA targeting trispecific protein (02B05) and BCMA expressing cells RPMI8226.

FIG. 24 illustrates the expression level of T cell activation biomarker CD25, following a TDCC assay using an exemplary BCMA targeting trispecific protein (02B05) and BCMA expressing cells RPMI8226.

FIG. 25 illustrates the expression level of T cell activation biomarker CD69, following a TDCC assay using an exemplary BCMA targeting trispecific protein (02B05) and BCMA non-expressing cells OVCAR8.

FIG. 26 illustrates the expression level of T cell activation biomarker CD25, following a TDCC assay using an exemplary BCMA targeting trispecific protein (02B05) and BCMA non-expressing cells OVCAR8.

FIG. 27 illustrates the expression level of T cell activation biomarker CD69, following a TDCC assay using an exemplary BCMA targeting trispecific protein (02B05) and BCMA non-expressing cells NCI-H510A.

FIG. 28 illustrates the expression level of T cell activation biomarker CD25, following a TDCC assay using an exemplary BCMA targeting trispecific protein (02B05) and BCMA non-expressing cells NCI-H510A.

FIG. 29 illustrates the expression level a cytokine, TNF-α, in co-cultures of T cells and BCMA expressing target cells (EJM cells) treated with increasing concentrations of an exemplary BCMA targeting trispecific (02B05) protein or with a negative control GFP trispecific protein.

FIG. 30 illustrates tumor growth reduction in RPMI8226 xenograft model, treated with an exemplary BCMA targeting trispecific (02B05) protein, at varying concentrations, or with a control vehicle.

FIG. 31 illustrates tumor growth reduction in Jeko1 xenograft model, treated with an exemplary BCMA targeting trispecific (02B05) protein, at varying concentrations, or with a control vehicle.

FIG. 32 illustrates concentration of BCMA targeting trispecific protein in serum samples from cynomolgus monkeys dosed with varying concentrations of an exemplary BCMA targeting trispecific (02B05) protein.

FIG. 33 the results of a TDCC assay using BCMA trispecific targeting protein obtained from serum samples of cynomolgus monkeys collected 168 hours after dosing with varying concentrations of an exemplary BCMA targeting trispecific (02B05) protein, BCMA expressing EJM cells and purified human T cells, in presence of serum from cynomolgus monkeys that were not exposed to a BCMA targeting trispecific protein.

FIG. 34 illustrates BCMA trispecific antigen-binding protein Phase ½ trial design.

FIG. 35 shows the time on treatment for all patients treated.

FIG. 36 shows the overall response rate.

FIG. 37A illustrates the pharmacokinetic data of the BCMA trispecific antigen-binding protein for the different dosing cohorts. Evidence for BCMA trispecific accumulation is shown by comparing C1D1 and C2D15: about 1.5-2-fold increase in Cmax (FIG. 37B) and AUC (FIG. 37C) and about 2-3-fold increase in Clast (FIG. 37D).

FIG. 38A shows serum cytokine concentrations 5 hours after first (C1D1) and second (C1D8) dose for serum IL-6. FIG. 38B shows serum cytokine concentrations 5 hours after first (C1D1) and second (C1D8) dose for serum TNFα.

FIG. 39 shows on-treatment changes in serum BCMA. On-treatment changes observed in serum BCMA (sBCMA) from Baseline to C1D15 Predose (after 2 doses).

FIG. 40 shows the concentration-time profiles of the BCMA trispecific antigen-binding protein. FIG. 40A shows the concentration-time profiles after the sixth dose and FIG. 40B shows the concentration-time profiles after the first dose.

FIGS. 41A and 41B show the serum cytokine and chemokine levels 5 hours after the first infusion. Serum samples were collected before (baseline) and 5 hours after the first infusion (5 h EOI) on C1D1. FIG. 41A illustrates the median increase in concentration above baseline, of each cytokine at 5 h EOI represented by a histogram bar, and the value from each patient is represented by a marker symbol. FIG. 41B illustrates the median increase in concentration above baseline, of each chemokine at 5 h EOI represented by a histogram bar, and the value from each patient is represented by a marker symbol.

FIGS. 42A and 42B show the kinetics of changes in serum cytokines and chemokines in fixed-dose and step-dose cohorts. FIG. 42A shows that cytokine concentrations in sera collected before the first dose (Pre) and at 5 h EOI on the indicated dosing day are shown with the dose administered. FIG. 42B shows that chemokine concentrations in sera collected before the first dose (Pre) and at 5 h EOI on the indicated dosing day are shown with the dose administered. The median cytokine or chemokine concentration is represented by a histogram bar, and the value from each patient is represented by a marker symbol.

FIG. 43 shows cytokine induction and clinical responses in the 2150 and 2860 μg dose cohorts. Serum samples were collected at baseline and 5 hours after the first infusion. The increase in cytokine or chemokine concentration above baseline for each subject in the 2150 and 2860 μg dose cohorts is indicated by a marker (triangle or circle). Subjects are divided into two groups according to the latest clinical responses - - - those with progressive disease (PD) or stable disease (SD), and those who had a partial response (PR), very good partial response (VGPR), or a complete response (CR) to the treatment. The significance of the difference in cytokine or chemokine concentration between these two groups is determined using an unpaired two-tailed Student's t-test.

FIGS. 44A and 44B show changes in circulating T cell counts after the BCMA trispecific antigen-binding protein infusions for fixed-dose cohorts. FIG. 44A shows CD4+ T cells and FIG. 44B shows CD8+ T cells. Flow cytometry analysis was conducted on whole blood to determine the cell count of each T cell subset before and 5, 24, or 48 hours after the indicated infusions. The cell counts at the post-dose time points are expressed as % of pre-dose. The median % of pre-dose cell count for each cohort is represented by the histogram bar and the value for each patient is indicated by a marker symbol.

FIG. 45 shows changes in circulating T cell counts after the BCMA trispecific antigen-binding protein infusions for step-dose cohorts. Flow cytometry analysis was conducted on whole blood to determine the cell count of each T cell subset before and 5, 24, or 48 hours after the indicated infusions. The cell counts at the post-dose time points are expressed as % of pre-dose. The median % of pre-dose cell count for each cohort is represented by the histogram bar and the value for each patient is indicated by a marker symbol.

FIGS. 46A and 46B shows upregulation of CD69 expression on T cells after the BCMA trispecific antigen-binding protein infusions for fixed-dose cohorts. FIG. 46A shows CD4+ T cells and FIG. 46B shows CD8+ T cells. Flow cytometry analysis was conducted on whole blood to determine the percent of CD4+ and CD8+ T cells that expressed CD69 before and after the indicated the BCMA trispecific antigen-binding protein administration. The median % CD69+ for each cohort is represented by the histogram bar and the value for each patient is indicated by a marker symbol.

FIG. 47 shows T cell activation and clinical responses in the 2150 and 2860 μg dose cohorts. Flow cytometry analysis was conducted on whole blood to measure the expression of CD69 on T cells at baseline and 24 hours after the first BCMA trispecific antigen-binding protein administration. The fold change in percent CD69+ T cells for each subject in the 2150 and 2860 μg dose cohorts is indicated by a marker (triangle or circle). Subjects are divided into two groups according to the latest clinical responses, those with progressive disease (PD) or stable disease (SD), and those who had a partial response (PR), very good partial response (VGPR), or a complete response (CR) to the treatment. The significance of the difference in cytokine or chemokine concentration between these two groups is determined using an unpaired two-tailed Student's t-test.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

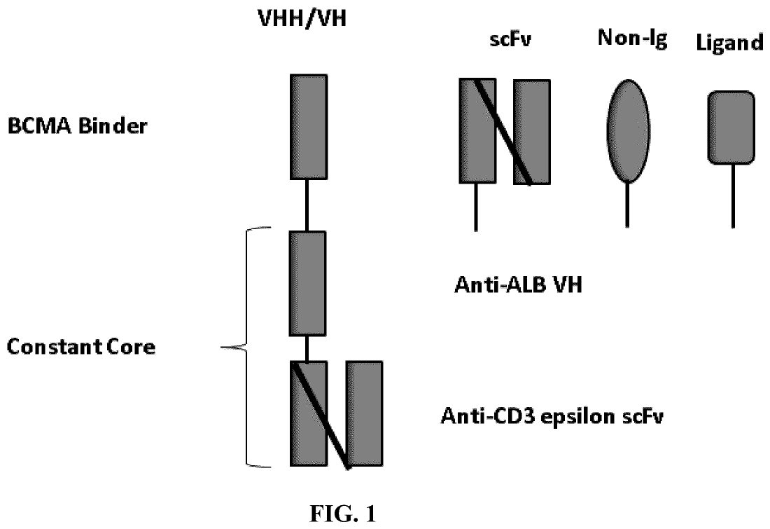

Described herein are trispecific proteins that target B cell maturation antigen (BCMA), pharmaceutical compositions thereof (referred to herein as BCMA binding trispecific protein. BCMA targeting trispecific protein, or BCMA trispecific antigen-binding protein) as well as nucleic acids, recombinant expression vectors and host cells for making such proteins thereof. Also provided are methods of using the disclosed BCMA targeting trispecific proteins in the prevention, and/or treatment of diseases, conditions and disorders. The BCMA targeting trispecific proteins are capable of specifically binding to BCMA as well as CD3 and have a half-life extension domain, such as a domain binding to human albumin (ALB). FIG. 1 depicts a non-limiting example of a trispecific BCMA-binding protein.

An “antibody” typically refers to a Y-shaped tetrameric protein comprising two heavy (H) and two light (L) polypeptide chains held together by covalent disulfide bonds and noncovalent interactions. Human light chains comprise a variable domain (VL) and a constant domain (CL) wherein the constant domain may be readily classified as kappa or lambda based on amino acid sequence and gene loci. Each heavy chain comprises one variable domain (VH) and a constant region, which in the case of IgG, IgA, and IgD, comprises three domains termed CH1, CH2, and CH3 (IgM and IgE have a fourth domain, CH4). In IgG, IgA, and IgD classes the CH1 and CH2 domains are separated by a flexible hinge region, which is a proline and cysteine rich segment of variable length (generally from about 10 to about 60 amino acids in IgG). The variable domains in both the light and heavy chains are joined to the constant domains by a “J” region of about 12 or more amino acids and the heavy chain also has a “D” region of about 10 additional amino acids. Each class of antibody further comprises inter-chain and intra-chain disulfide bonds formed by paired cysteine residues. There are two types of native disulfide bridges or bonds in immunoglobulin molecules: inter-chain and intra-chain disulfide bonds. The location and number of inter-chain disulfide bonds vary according to the immunoglobulin class and species. Inter-chain disulfide bonds are located on the surface of the immunoglobulin, are accessible to solvent and are usually relatively easily reduced. In the human IgG1 isotype there are four inter-chain disulfide bonds, one from each heavy chain to the light chain and two between the heavy chains. The inter-chain disulfide bonds are not required for chain association. As is well known the cysteine rich IgG1 hinge region of the heavy chain has generally been held to consist of three parts: an upper hinge, a core hinge, and a lower hinge. Those skilled in the art will appreciate that the IgG1 hinge region contains the cysteines in the heavy chain that comprise the inter-chain disulfide bonds (two heavy/heavy, two heavy/light), which provide structural flexibility that facilitates Fab movements. The inter-chain disulfide bond between the light and heavy chain of IgG1 are formed between C214 of the kappa or lambda light chain and C220 in the upper hinge region of the heavy chain. The inter-chain disulfide bonds between the heavy chains are at positions C226 and C229 (all numbered per the EU index according to Kabat, et al., infra.).

As used herein the term “antibody” includes polyclonal antibodies, multiclonal antibodies, monoclonal antibodies, chimeric antibodies, humanized and primatized antibodies, CDR grafted antibodies, human antibodies, recombinantly produced antibodies, intrabodies, multispecific antibodies, bispecific antibodies, monovalent antibodies, multivalent antibodies, anti-idiotypic antibodies, synthetic antibodies, including muteins and variants thereof, immunospecific antibody fragments such as Fd, Fab, F(ab′)₂, F(ab′) fragments, single-chain fragments (e.g., ScFv and ScFvFc), disulfide-linked Fvs (sdFv), a Fd fragment consisting of the VH and CH1 domains, linear antibodies, single domain antibodies such as sdAb (VH, VL, or VHH domains); and derivatives thereof including Fc fusions and other modifications, and any other immunoreactive molecule so long as it comprises a domain having a binding site for preferential association or binding with a BCMA protein. Moreover, unless dictated otherwise by contextual constraints the term further comprises all classes of antibodies (i.e., IgA, IgD, IgE, IgG, and IgM) and all subclasses (i.e., IgG1, IgG2, IgG3, IgG4, IgA1, and IgA2). Heavy-chain constant domains that correspond to the different classes of antibodies are typically denoted by the corresponding lower case Greek letter alpha, delta, epsilon, gamma, and mu, respectively. Light chains of the antibodies from any vertebrate species can be assigned to one of two clearly distinct types, called kappa (kappa) and lambda (lambda), based on the amino acid sequences of their constant domains.

In some embodiments, the BCMA binding domain of the BCMA targeting trispecific proteins of this disclosure comprise a heavy chain only antibody, such as a VH or a VHH domain. In some cases, the BCMA binding proteins comprise a heavy chain only antibody that is an engineered human VH domain. In some examples, the engineered human VH domain is produced by panning of phage display libraries. In some embodiments, the BCMA binding domain of the BCMA targeting trispecific proteins of this disclosure comprise a VHH. The term “VHH,” as used herein, refers to single chain antibody binding domain devoid of light chain. In some cases, a VHH is derived from an antibody of the type that can be found in Camelidae or cartilaginous fish which are naturally devoid of light chains or to a synthetic and non-immunized VHH which can be constructed accordingly. Each heavy chain comprises a variable region encoded by V-, D- and J exons. A VHH, in some cases, is a natural VHH, such as a Camelid-derived VHH, or a recombinant protein comprising a heavy chain variable domain. In some embodiments, the VHH is derived from a species selected from the group consisting of camels, llamas, vicugnas, guanacos, and cartilaginous fish (such as, but not limited to, sharks). In another embodiment, the VHH is derived from an alpaca (such as, but not limited to, a Huacaya Alpaca or a Suri alpaca).

As used herein. “Variable region” or “variable domain” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the light-chain (VL) and the heavy-chain (VH) variable domains. The more highly conserved portions of variable domains are called the framework (FR). The variable domains of native heavy and light chains each comprise four FR regions, largely adopting a β-sheet configuration, connected by three CDRs, which form loops connecting, and in some cases forming part of, the β sheet structure. The CDRs in each chain are held together in close proximity by the FR regions and, with the CDRs from the other chain, contribute to the formation of the antigen-binding site of antibodies (see Kabat et al., Sequences of Proteins of Immunological Interest, Fifth Edition, National Institute of Health, Bethesda. Md. (1991)). The constant domains are not involved directly in binding an antibody to an antigen, but exhibit various effector functions, such as participation of the antibody in antibody-dependent cellular toxicity. ScFv fragments (or single chain fragment variable), which in some cases are obtained by genetic engineering, associate in a single polypeptide chain, the VH and the VL region of an antibody, separated by a peptide linker.

In some embodiments of this disclosure, the BCMA binding domain of the BCMA targeting trispecific proteins comprise heavy chain only antibodies, such as VH or VHH domains, and comprise three CDRs. Such heavy chain only antibodies, in some embodiments, bind BCMA as a monomer with no dependency on dimerization with a VL (light chain variable) region for optimal binding affinity. In some embodiments of this disclosure, the CD3 binding domain of the BCMA targeting trispecific proteins comprises a scFv. In some embodiments of this disclosure, the albumin binding domain of the BCMA targeting trispecific proteins comprise a heavy chain only antibody, such as a single domain antibody comprising a VH domain or a VHH domain.

The assignment of amino acids to each domain, framework region and CDR is, in some embodiments, in accordance with one of the numbering schemes provided by Kabat et al. (1991) Sequences of Proteins of Immunological Interest (5th Ed.). US Dept, of Health and Human Services, PHS, NIH, NIH Publication no. 91-3242: Chothia et al., 1987, PMID: 3681981; Chothia et al., 1989. PMID: 2687698: MacCallum et al., 1996. PMID: 8876650; or Dubel. Ed. (2007) Handbook of Therapeutic Antibodies. 3rd Ed., Wily-VCH Verlag GmbH and Co or AbM (Oxford Molecular/MSI Pharmacopia) unless otherwise noted. It is not intended that CDRs of the present disclosure necessarily correspond to the Kabat numbering convention.

The term “Framework” or “FR” residues (or regions) refer to variable domain residues other than the CDR or hypervariable region residues as herein defined. A “human consensus framework” is a framework which represents the most commonly occurring amino acid residue in a selection of human immunoglobulin VL or VH framework sequences.

As used herein, the term “Percent (%) amino acid sequence identity” with respect to a sequence is defined as the percentage of amino acid residues in a candidate sequence that are identical with the amino acid residues in the specific sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as EMBOSS MATCHER, EMBOSS WATER, EMBOSS STRETCHER, EMBOSS NEEDLE, EMBOSS LALIGN, BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

As used herein. “elimination half-time” is used in its ordinary sense, as is described in Goodman and Gillman's The Pharmaceutical Basis of Therapeutics 21-25 (Alfred Goodman Gilman, Louis S. Goodman, and Alfred Gilman, eds., 6th ed. 1980). Briefly, the term is meant to encompass a quantitative measure of the time course of drug elimination. The elimination of most drugs is exponential (i.e., follows first-order kinetics), since drug concentrations usually do not approach those required for saturation of the elimination process. The rate of an exponential process may be expressed by its rate constant, k, which expresses the fractional change per unit of time, or by its half-time, t1/2 the time required for 50% completion of the process. The units of these two constants are time−1 and time, respectively. A first-order rate constant and the half-time of the reaction are simply related (k×t1/2=0.693) and may be interchanged accordingly. Since first-order elimination kinetics dictates that a constant fraction of drug is lost per unit time, a plot of the log of drug concentration versus time is linear at all times following the initial distribution phase (i.e., after drug absorption and distribution are complete). The half-time for drug elimination can be accurately determined from such a graph.

As used herein, the term “binding affinity” refers to the affinity of the proteins described in the disclosure to their binding targets, and is expressed numerically using “Kd” values. If two or more proteins are indicated to have comparable binding affinities towards their binding targets, then the Kd values for binding of the respective proteins towards their binding targets, are within ±2-fold of each other. If two or more proteins are indicated to have comparable binding affinities towards single binding target, then the Kd values for binding of the respective proteins towards said single binding target, are within ±2-fold of each other. If a protein is indicated to bind two or more targets with comparable binding affinities, then the Kd values for binding of said protein to the two or more targets are within ±2-fold of each other. In general, a higher Kd value corresponds to a weaker binding. In some embodiments, the “Kd” is measured by a radiolabeled antigen binding assay (RIA) or surface plasmon resonance assays using a BIACORE™-2000 or a BIACORE™-3000 (BIAcore, Inc., Piscataway. N.J.). In certain embodiments, an “on-rate” or “rate of association” or “association rate” or “kon” and an “off-rate” or “rate of dissociation” or “dissociation rate” or “koff” are also determined with the surface plasmon resonance technique using a BIACORE™-2000 or a BIACORE™-3000 (BIAcore, Inc., Piscataway. N.J.). In additional embodiments, the “Kd”. “kon”, and “koff” are measured using the OCTET® Systems (Pall Life Sciences). In an exemplary method for measuring binding affinity using the OCTET® Systems, the ligand, e.g., biotinylated human or cynomolgus BCMA, is immobilized on the OCTET® streptavidin capillary sensor tip surface which streptavidin tips are then activated according to manufacturer's instructions using about 20-50 μg/ml human or cynomolgus BCMA protein. A solution of PBS/Casein is also introduced as a blocking agent. For association kinetic measurements. BCMA binding protein variants are introduced at a concentration ranging from about 10 ng/ml to about 100 μg/mL, from about 50 ng/ml to about 5 μg/mL, or from about 2 ng/mL to about 20 μg/mL. In some embodiments, the BCMA binding single domain proteins are used at a concentration ranging from about 2 ng/ml to about 20 μg/mL. Complete dissociation is observed in case of the negative control, assay buffer without the binding proteins. The kinetic parameters of the binding reactions are then determined using an appropriate tool, e.g., ForteBio software.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” should be assumed to mean an acceptable error range for the particular value.

The terms “individual,” “patient,” or “subject” are used interchangeably. None of the terms require or are limited to situations characterized by the supervision (e.g., constant or intermittent) of a health care worker (e.g., a doctor, a registered nurse, a nurse practitioner, a physician's assistant, an orderly, or a hospice worker).

The terminology used herein is for the purpose of describing particular cases only and is not intended to be limiting. As used herein, the singular forms “a,” “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

In one aspect, the BCMA targeting trispecific proteins comprise a domain (A) which specifically binds to CD3, a domain (B) which specifically binds to human albumin (ALB), and a domain (C) which specifically binds to BCMA. The three domains in BCMA targeting trispecific proteins are arranged in any order. Thus, it is contemplated that the domain order of the BCMA targeting trispecific proteins are: H₂N-(A)-(B)-(C)-COOH, H₂N-(A)-(C)-(B)-COOH, H₂N-(B)-(A)-(C)-COOH, H₂N-(B)-(C)-(A)-COOH, H₂N-(C)-(B)-(A)-COOH, or H₂N-(C)-(A)-(B)-COOH.

In some embodiments, the BCMA targeting trispecific proteins have a domain order of H₂N-(A)-(B)-(C)-COOH. In some embodiments, the BCMA targeting trispecific proteins have a domain order of H₂N-(A)-(C)-(B)-COOH. In some embodiments, the BCMA targeting trispecific proteins have a domain order of H₂N-(B)-(A)-(C)-COOH. In some embodiments, the BCMA targeting trispecific proteins have a domain order of H₂N-(B)-(C)-(A)-COOH. In some embodiments, the BCMA targeting trispecific proteins have a domain order of H₂N-(C)-(B)-(A)-COOH. In some embodiments, the BCMA targeting trispecific proteins have a domain order of H₂N-(C)-(A)-(B)-COOH. In some embodiments, the anti-BCMA domain (the anti-target domain, T), the anti-CD3 domain (C), and the anti-ALB domain (A) are in an anti-CD3: anti-ALB: anti-BCMA (CAT) orientation. In some embodiments, the anti-BCMA domain (the anti-target domain (T), the anti-CD3 domain (C), and the anti-ALB domain (A) are in an anti-BCMA: anti-ALB: anti-CD3 (TAC) orientation.

In some embodiments, the BCMA targeting trispecific proteins have the HSA binding domain as the middle domain, such that the domain order is H₂N-(A)-(B)-(C)-COOH or H₂N-(C)-(B)-(A)-COOH. It is contemplated that in such embodiments where the ALB binding domain as the middle domain, the CD3 and BCMA binding domains are afforded additional flexibility to bind to their respective targets.

In some embodiments, the BCMA targeting trispecific proteins described herein comprise a polypeptide having a sequence described in the Sequence Table (SEQ ID NO: 483-597) and subsequences thereof. In some embodiments, the trispecific antigen binding protein comprises a polypeptide having at least 70%-95% or more homology to a sequence described in the Sequence Table (SEQ ID NO: 483-597). In some embodiments, the trispecific antigen binding protein comprises a polypeptide having at least 70%, 75%, 80%, 85%, 90%, 95%, or more homology to a sequence described in the Sequence Table 1 (SEQ ID NO: 483-597).

The BCMA targeting trispecific proteins described herein are designed to allow specific targeting of cells expressing BCMA by recruiting cytotoxic T cells. This improves efficacy compared to ADCC (antibody dependent cell-mediated cytotoxicity), which is using full length antibodies directed to a sole antigen and is not capable of directly recruiting cytotoxic T cells. In contrast, by engaging CD3 molecules expressed specifically on these cells, the BCMA targeting trispecific proteins can crosslink cytotoxic T cells with cells expressing BCMA in a highly specific fashion, thereby directing the cytotoxic potential of the T cell towards the target cell. The BCMA targeting trispecific proteins described herein engage cytotoxic T cells via binding to the surface-expressed CD3 proteins, which form part of the TCR. Simultaneous binding of several BCMA trispecific antigen-binding protein to CD3 and to BCMA expressed on the surface of particular cells causes T cell activation and mediates the subsequent lysis of the particular BCMA expressing cell. Thus. BCMA targeting trispecific proteins are contemplated to display strong, specific and efficient target cell killing. In some embodiments, the BCMA targeting trispecific proteins described herein stimulate target cell killing by cytotoxic T cells to eliminate pathogenic cells (e.g., tumor cells expressing BCMA). In some of such embodiments, cells are eliminated selectively, thereby reducing the potential for toxic side effects.

The BCMA targeting trispecific proteins described herein confer further therapeutic advantages over traditional monoclonal antibodies and other smaller bispecific molecules. Generally, the effectiveness of recombinant protein pharmaceuticals depends heavily on the intrinsic pharmacokinetics of the protein itself. One such benefit here is that the BCMA targeting trispecific proteins described herein have extended pharmacokinetic elimination half-time due to having a half-life extension domain such as a domain specific to HSA. In this respect, the BCMA targeting trispecific proteins described herein have an extended serum elimination half-time of about two, three, about five, about seven, about 10, about 12, or about 14 days in some embodiments. This contrasts to other binding proteins such as BiTE or DART molecules which have relatively much shorter elimination half-times. For example, the BiTE CD19×CD3 bispecific scFv-scFv fusion molecule requires continuous intravenous infusion (i.v.) drug delivery due to its short elimination half-time. The longer intrinsic half-times of the BCMA targeting trispecific proteins solve this issue thereby allowing for increased therapeutic potential such as low-dose pharmaceutical formulations, decreased periodic administration and/or novel pharmaceutical compositions.

The BCMA targeting trispecific proteins described herein also have an optimal size for enhanced tissue penetration and tissue distribution. Larger sizes limit or prevent penetration or distribution of the protein in the target tissues. The BCMA targeting trispecific proteins described herein avoid this by having a small size that allows enhanced tissue penetration and distribution. Accordingly, the BCMA targeting trispecific proteins described herein, in some embodiments have a size of about 50 kD to about 80 kD, about 50 kD to about 75 kD, about 50 kD to about 70 kD, or about 50 kD to about 65 kD. Thus, the size of the BCMA targeting trispecific proteins is advantageous over IgG antibodies which are about 150 kD and the BiTE and DART diabody molecules which are about 55 kD but are not half-life extended and therefore cleared quickly through the kidney.

In further embodiments, the BCMA targeting trispecific proteins described herein have an optimal size for enhanced tissue penetration and distribution. In these embodiments, the BCMA targeting trispecific proteins are constructed to be as small as possible, while retaining specificity toward its targets. Accordingly, in these embodiments, the BCMA targeting trispecific proteins described herein have a size of about 20 kD to about 40 kD or about 25 kD to about 35 kD to about 40 kD, to about 45 kD, to about 50 kD, to about 55 kD, to about 60 kD, to about 65 kD. In some embodiments, the BCMA targeting trispecific proteins described herein have a size of about 50 kD, 49, kD, 48 kD, 47 kD, 46 kD, 45 kD, 44 kD, 43 kD, 42 kD, 41 kD, 40 kD, about 39 kD, about 38 kD, about 37 kD, about 36 kD, about 35 kD, about 34 kD, about 33 kD, about 32 kD, about 31 kD, about 30 kD, about 29 kD, about 28 kD, about 27 kD, about 26 kD, about 25 kD, about 24 kD, about 23 kD, about 22 kD, about 21 kD, or about 20 kD. An exemplary approach to the small size is through the use of single domain antibody (sdAb) fragments for each of the domains. For example, a particular BCMA trispecific antigen-binding protein has an anti-CD3 sdAb, anti-ALB sdAb and an sdAb for BCMA. This reduces the size of the exemplary BCMA trispecific antigen-binding protein to under 40 kD. Thus, in some embodiments, the domains of the BCMA targeting trispecific proteins are all single domain antibody (sdAb) fragments. In other embodiments, the BCMA targeting trispecific proteins described herein comprise small molecule entity (SME) binders for ALB and/or the BCMA. SME binders are small molecules averaging about 500 to 2000 Da in size and are attached to the BCMA targeting trispecific proteins by known methods, such as sortase ligation or conjugation. In these instances, one of the domains of BCMA trispecific antigen-binding protein is a sortase recognition sequence, e.g., LPETG (SEQ ID NO: 482). To attach a SME binder to BCMA trispecific antigen-binding protein with a sortase recognition sequence, the protein is incubated with a sortase and a SME binder whereby the sortase attaches the SME binder to the recognition sequence. Known SME binders include MIP-1072 and MIP-1095 which bind to BCMA.

In yet other embodiments, the domain which binds to BCMA of BCMA targeting trispecific proteins described herein comprise a knottin peptide for binding BCMA. Knottins are disulfide-stabilized peptides with a cysteine knot scaffold and have average sizes about 3.5 kD. Knottins have been contemplated for binding to certain tumor molecules such as BCMA. In further embodiments, domain which binds to BCMA of BCMA targeting trispecific proteins described herein comprise a natural BCMA ligand.

Another feature of the BCMA targeting trispecific proteins described herein is that they are of a single-polypeptide design with flexible linkage of their domains. This allows for facile production and manufacturing of the BCMA targeting trispecific proteins as they can be encoded by single cDNA molecule to be easily incorporated into a vector. Further, because the BCMA targeting trispecific proteins described herein are a monomeric single polypeptide chain, there are no chain pairing issues or a requirement for dimerization. It is contemplated that the BCMA targeting trispecific proteins described herein have a reduced tendency to aggregate unlike other reported molecules such as bispecific proteins with Fc-gamma immunoglobulin domains.

In the BCMA targeting trispecific proteins described herein, the domains are linked by internal linkers L1 and L2, where L1 links the first and second domain of the BCMA targeting trispecific proteins and L2 links the second and third domains of the BCMA targeting trispecific proteins. Linkers L1 and L2 have an optimized length and/or amino acid composition. In some embodiments, linkers L1 and L2 are the same length and amino acid composition. In other embodiments. L1 and L2 are different. In certain embodiments, internal linkers L1 and/or L2 are “short”, i.e., consist of 0, 1, 2, 3, 4, 5, 6, 7, 8, 9, 10, 11 or 12 amino acid residues. Thus, in certain instances, the internal linkers consist of about 12 or less amino acid residues. In the case of 0 amino acid residues, the internal linker is a peptide bond. In certain embodiments, internal linkers L1 and/or L2 are “long”, i.e., “consist of” 15, 20 or 25 amino acid residues. In some embodiments, these internal linkers consist of about 3 to about 15, for example 8, 9 or 10 contiguous amino acid residues. Regarding the amino acid composition of the internal linkers L1 and L2, peptides are selected with properties that confer flexibility to the BCMA targeting trispecific proteins, do not interfere with the binding domains as well as resist cleavage from proteases. For example, glycine and serine residues generally provide protease resistance. Examples of internal linkers suitable for linking the domains in the BCMA targeting trispecific proteins include but are not limited to (GS)ₙ (SEQ ID NO: 472), (GGS)ₙ (SEQ ID NO: 473), (GGGS)ₙ (SEQ ID NO: 474), (GGSG)ₙ (SEQ ID NO: 475), (GGSGG)ₙ (SEQ ID NO: 476), (GGGGS)ₙ (SEQ ID NO: 477), (GGGGG)ₙ (SEQ ID NO: 478), or (GGG)ₙ (SEQ ID NO: 479), wherein n is 1, 2, 3, 4, 5, 6, 7, 8, 9, or 10. In one embodiment, internal linker L1 and/or L2 is (GGGGS)4 (SEQ ID NO: 480) or (GGGGS)3 (SEQ ID NO: 481).

### CD3 Binding Domain

The specificity of the response of T cells is mediated by the recognition of an antigen (displayed in context of a major histocompatibility complex, MHC) by the TCR. As part of the TCR, CD3 is a protein complex that includes a CD3γ (gamma) chain, a CD3δ (delta) chain, and two CD3ε (epsilon) chains which are present on the cell surface. CD3 associates with the α (alpha) and β (beta) chains of the TCR as well as CD3 ζ (zeta) altogether to comprise the complete TCR. Clustering of CD3 on T cells, such as by immobilized anti-CD3 antibodies leads to T cell activation similar to the engagement of the T cell receptor but independent of its clone-typical specificity.

In one aspect, the BCMA targeting trispecific proteins described herein comprise a domain which specifically binds to CD3. In one aspect, the BCMA targeting trispecific proteins described herein comprise a domain which specifically binds to human CD3. In some embodiments, the BCMA targeting trispecific proteins described herein comprise a domain which specifically binds to CD3γ. In some embodiments, the BCMA targeting trispecific proteins described herein comprise a domain which specifically binds to CD3δ. In some embodiments, the BCMA targeting trispecific proteins described herein comprise a domain which specifically binds to CD3ε.

In further embodiments, the BCMA targeting trispecific proteins described herein comprise a domain which specifically binds to the TCR. In certain instances, the BCMA targeting trispecific proteins described herein comprise a domain which specifically binds the α chain of the TCR. In certain instances, the BCMA targeting trispecific proteins described herein comprise a domain which specifically binds the β chain of the TCR.

In some embodiments, the CD3 binding domain of the BCMA trispecific antigen-binding protein can be any domain that binds to CD3 including but not limited to domains from a monoclonal antibody, a polyclonal antibody, a recombinant antibody, a human antibody, a humanized antibody. In some instances, it is beneficial for the CD3 binding domain to be derived from the same species in which the BCMA trispecific antigen-binding protein will ultimately be used in. For example, for use in humans, it may be beneficial for the CD3 binding domain of the BCMA trispecific antigen-binding protein to comprise human or humanized residues from the antigen binding domain of an antibody or antibody fragment.

Thus, in one aspect, the antigen-binding domain comprises a humanized or human antibody or an antibody fragment, or a murine antibody or antibody fragment. In one embodiment, the humanized or human anti-CD3 binding domain comprises one or more (e.g., all three) light chain complementary determining region 1 (LC CDR1), light chain complementary determining region 2 (LC CDR2), and light chain complementary determining region 3 (LC CDR3) of a humanized or human anti-CD3 binding domain described herein, and/or one or more (e.g., all three) heavy chain complementary determining region 1 (HC CDR1), heavy chain complementary determining region 2 (HC CDR2), and heavy chain complementary determining region 3 (HC CDR3) of a humanized or human anti-CD3 binding domain described herein, e.g., a humanized or human anti-CD3 binding domain comprising one or more, e.g., all three. LC CDRs and one or more, e.g., all three, HC CDRs.

In some embodiments, the humanized or human anti-CD3 binding domain comprises a humanized or human light chain variable region specific to CD3 where the light chain variable region specific to CD3 comprises human or non-human light chain CDRs in a human light chain framework region. In certain instances, the light chain framework region is a λ (lamda) light chain framework. In other instances, the light chain framework region is a κ (kappa) light chain framework.

In some embodiments, the humanized or human anti-CD3 binding domain comprises a humanized or human heavy chain variable region specific to CD3 where the heavy chain variable region specific to CD3 comprises human or non-human heavy chain CDRs in a human heavy chain framework region.

In certain instances, the complementary determining regions of the heavy chain and/or the light chain are derived from known anti-CD3 antibodies, such as, for example, muromonab-CD3 (OKT3), otelixizumab (TRX4), teplizumab (MGA031), visilizumab (NUVION®), SP34, TR-66 or X35-3, VIT3, BMA030 (BW264/56), CLB-T3/3, CRIS7, YTH12.5, F111-409, CLB-T3.4.2, TR-66, WT32, SPv-T3b, 11D8, XIII-141, XIII-46, XIII-87, 12F6, T3/RW2-8C8, T3/RW2-4B6, OKT3D, M-T301, SMC2, F101.01, UCHT-1 and WT-31.

In one embodiment, the anti-CD3 binding domain is a single chain variable fragment (scFv) comprising a light chain and a heavy chain of an amino acid sequence provided herein. As used herein. “single chain variable fragment” or “scFv” refers to an antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single polypeptide chain, and wherein the scFv retains the specificity of the intact antibody from which it is derived. In an embodiment, the anti-CD3 binding domain comprises: a light chain variable region comprising an amino acid sequence having at least one, two or three modifications (e.g., substitutions) but not more than 30, 20 or 10 modifications (e.g., substitutions) of an amino acid sequence of a light chain variable region provided herein, or a sequence with 95-99% identity with an amino acid sequence provided herein; and/or a heavy chain variable region comprising an amino acid sequence having at least one, two or three modifications (e.g., substitutions) but not more than 30, 20 or 10 modifications (e.g., substitutions) of an amino acid sequence of a heavy chain variable region provided herein, or a sequence with 95-99% identity to an amino acid sequence provided herein. In one embodiment, the humanized or human anti-CD3 binding domain is a scFv, and a light chain variable region comprising an amino acid sequence described herein, is attached to a heavy chain variable region comprising an amino acid sequence described herein, via a scFv linker. The light chain variable region and heavy chain variable region of a scFv can be, e.g., in any of the following orientations: light chain variable region—scFv linker-heavy chain variable region or heavy chain variable region—scFv linker-light chain variable region.

In some instances, scFvs which bind to CD3 are prepared according to known methods. For example, scFv molecules can be produced by linking VH and VL regions together using flexible polypeptide linkers. The scFv molecules comprise a scFv linker (e.g., a Ser-Gly linker) with an optimized length and/or amino acid composition. Accordingly, in some embodiments, the length of the scFv linker is such that the VH or VL domain can associate intermolecularly with the other variable domain to form the CD3 binding site. In certain embodiments, such scFv linkers are “short”, i.e, consist of 0, 1, 2, 3, 4, 5, 6, 7, 8, 9, 10, 11 or 12 amino acid residues. Thus, in certain instances, the scFv linkers consist of about 12 or less amino acid residues. In the case of 0 amino acid residues, the scFv linker is a peptide bond. In some embodiments, these scFv linkers consist of about 3 to about 15, for example 8, 9 or 10 contiguous amino acid residues. Regarding the amino acid composition of the scFv linkers, peptides are selected that confer flexibility, do not interfere with the variable domains as well as allow inter-chain folding to bring the two variable domains together to form a functional CD3 binding site. For example, scFv linkers comprising glycine and serine residues generally provide protease resistance. In some embodiments, linkers in a scFv comprise glycine and serine residues. The amino acid sequence of the scFv linkers can be optimized, for example, by phage-display methods to improve the CD3 binding and production yield of the scFv. Examples of peptide scFv linkers suitable for linking a variable light domain and a variable heavy domain in a scFv include but are not limited to (GS)ₙ (SEQ ID NO: 472), (GGS)ₙ (SEQ ID NO: 473), (GGGS)ₙ (SEQ ID NO: 474), (GGSG)ₙ (SEQ ID NO: 475), (GGSGG)ₙ (SEQ ID NO: 476), (GGGGS)ₙ (SEQ ID NO: 477), (GGGGG)ₙ (SEQ ID NO: 478), or (GGG)ₙ (SEQ ID NO: 479), wherein n is 1, 2, 3, 4, 5, 6, 7, 8, 9, or 10. In one embodiment, internal linker L1 and/or L2 is (GGGGS)₄ (SEQ ID NO: 480) or (GGGGS)₃ (SEQ ID NO: 481). Variation in the linker length may retain or enhance activity, giving rise to superior efficacy in activity studies.

In some embodiments. CD3 binding domain of BCMA trispecific antigen-binding protein has an affinity to CD3 on CD3 expressing cells with a K_{D} of 1000 nM or less, 500 nM or less, 200 nM or less, 100 nM or less, 80 nM or less, 50 nM or less, 20 nM or less, 10 nM or less, 5 nM or less, 1 nM or less, or 0.5 nM or less. In some embodiments, the CD3 binding domain of BCMA trispecific antigen-binding protein has an affinity to CD3ε, γ, or δ with a K_{D} of 1000 nM or less, 500 nM or less, 200 nM or less, 100 nM or less, 80 nM or less, 50 nM or less, 20 nM or less, 10 nM or less, 5 nM or less, 1 nM or less, or 0.5 nM or less. In further embodiments. CD3 binding domain of BCMA trispecific antigen-binding protein has low affinity to CD3, i.e., about 100 nM or greater.

The affinity to bind to CD3 can be determined, for example, by the ability of the BCMA trispecific antigen-binding protein itself or its CD3 binding domain to bind to CD3 coated on an assay plate; displayed on a microbial cell surface: in solution; etc. The binding activity of the BCMA trispecific antigen-binding protein itself or its CD3 binding domain of the present disclosure to CD3 can be as saved by immobilizing the ligand (e.g., CD3) or the BCMA trispecific antigen-binding protein itself or its CD3 binding domain, to a bead, substrate, cell, etc. Agents can be added in an appropriate buffer and the binding partners incubated for a period of time at a given temperature. After washes to remove unbound material, the bound protein can be released with, for example, SDS, buffers with a high pH, and the like and analyzed, for example, by Surface Plasmon Resonance (SPR).

### Half-Life Extension Domain

Contemplated herein are domains which extend the half-life of an antigen-binding domain. Such domains are contemplated to include but are not limited to Albumin binding domains. Fc domains, small molecules, and other half-life extension domains known in the art.

Human albumin (ALB) (molecular mass of about 67 kDa) is the most abundant protein in plasma, present at about 50 mg/ml (600 μM), and has a half-life of around 20 days in humans. ALB serves to maintain plasma pH, contributes to colloidal blood pressure, functions as carrier of many metabolites and fatty acids, and serves as a major drug transport protein in plasma.

Noncovalent association with albumin extends the elimination half-time of short-lived proteins. For example, a recombinant fusion of an albumin binding domain to a Fab fragment resulted in an in vivo clearance of 25- and 58-fold and a half-life extension of 26- and 37-fold when administered intravenously to mice and rabbits respectively as compared to the administration of the Fab fragment alone. In another example, when insulin is acylated with fatty acids to promote association with albumin, a protracted effect was observed when injected subcutaneously in rabbits or pigs. Together, these studies demonstrate a linkage between albumin binding and prolonged action.

In one aspect, the BCMA targeting trispecific proteins described herein comprise a half-life extension domain, for example a domain which specifically binds to ALB. In some embodiments, the ALB binding domain of BCMA trispecific antigen-binding protein can be any domain that binds to ALB including but not limited to domains from a monoclonal antibody, a polyclonal antibody, a recombinant antibody, a human antibody, a humanized antibody. In some embodiments, the ALB binding domain is a single chain variable fragments (scFv), single-domain antibody such as a heavy chain variable domain (VH), a light chain variable domain (VL) and a variable domain (VHH) of camelid derived single domain antibody, peptide, ligand or small molecule entity specific for HSA. In certain embodiments, the ALB binding domain is a single-domain antibody. In other embodiments, the HSA binding domain is a peptide. In further embodiments, the HSA binding domain is a small molecule. It is contemplated that the HSA binding domain of BCMA trispecific antigen-binding protein is fairly small and no more than 25 kD, no more than 20 kD, no more than 15 kD, or no more than 10 kD in some embodiments. In certain instances, the ALB binding is 5 kD or less if it is a peptide or small molecule entity.

The half-life extension domain of BCMA trispecific antigen-binding protein provides for altered pharmacodynamics and pharmacokinetics of the BCMA trispecific antigen-binding protein itself. As above, the half-life extension domain extends the elimination half-time. The half-life extension domain also alters pharmacodynamic properties including alteration of tissue distribution, penetration, and diffusion of the trispecific antigen-binding protein. In some embodiments, the half-life extension domain provides for improved tissue (including tumor) targeting, tissue distribution, tissue penetration, diffusion within the tissue, and enhanced efficacy as compared with a protein without a half-life extension domain. In one embodiment, therapeutic methods effectively and efficiently utilize a reduced amount of the trispecific antigen-binding protein, resulting in reduced side effects, such as reduced non-tumor cell cytotoxicity.

Further, the binding affinity of the half-life extension domain can be selected so as to target a specific elimination half-time in a particular trispecific antigen-binding protein. Thus, in some embodiments, the half-life extension domain has a high binding affinity. In other embodiments, the half-life extension domain has a medium binding affinity. In vet other embodiments, the half-life extension domain has a low or marginal binding affinity. Exemplary binding affinities include KD concentrations at 10 nM or less (high), between 10 nM and 100 nM (medium), and greater than 100 nM (low). As above, binding affinities to ALB are determined by known methods such as Surface Plasmon Resonance (SPR).

In some embodiments. ALB binding domains described herein comprise a single domain antibody.

### B Cell Maturation Antigen (BCMA) Binding Domain

B cell maturation antigen (BCMA, TNFRSF17, CD269) is a transmembrane protein belonging to the tumor necrosis family receptor (TNFR) super family that is primarily expressed on terminally differentiated B cells. BCMA expression is restricted to the B cell lineage and mainly present on plasma cells and plasmablasts and to some extent on memory B cells, but virtually absent on peripheral and naive B cells. BCMA is also expressed on multiple myeloma (MM) cells, on leukemia cells and lymphoma cells.

BCMA was identified through molecular analysis of a t(4:16)(q26:p13) translocation found in a human intestinal T cell lymphoma and an in-frame sequence was mapped to the 16p13.1 chromosome band.

Human BCMA cDNA has an open reading frame of 552 bp that encodes a 184 amino acid polypeptide. The BCMA gene is organized into three exons that are separated by two introns, each flanked by GT donor and AG acceptor consensus splicing sites, and codes for a transcript of 1.2 kb. The structure of BCMA protein includes an integral transmembrane protein based on a central 24 amino acid hydrophobic region in an alpha-helix structure.

The murine BCMA gene is located on chromosome 16 syntenic to the human 16p13 region, and also includes three exons that are separated by two introns. The gene encodes a 185 amino acid protein. Murine BCMA mRNA is expressed as a 404 bp transcript at the highest levels in plasmacytoma cells (J558) and at modest levels in the A20 B cell lymphoma line. Murine BCMA mRNA transcripts have also been detected at low levels in T cell lymphoma (EL4, BW5147) and dendritic cell (CB1D6, D2SC1) lines in contrast to human cell lines of T cell and dendritic cell origin. The murine BCMA cDNA sequence has 69.3% nucleotide identity with the human BCMA cDNA sequence and slightly higher identity (73.7%) when comparing the coding regions between these two cDNA sequences. Mouse BCMA protein is 62% identical to human BCMA protein and, like human BCMA, contains a single hydrophobic region, which may be an internal transmembrane segment. The N-terminal 40 amino acid domain of both murine and human BCMA protein have six conserved cysteine residues, consistent with the formation of a cysteine repeat motif found in the extracellular domain of TNFRs. Similar to members of the TNFR superfamily. BCMA protein contains a conserved aromatic residue four to six residues C-terminal from the first cysteine.

BCMA is not expressed at the cell surface, but rather, is located on the Golgi apparatus. The amount of BCMA expression is proportional to the stage of cellular differentiation (highest in plasma cells).

It is involved in B cell development and homeostasis due to its interaction with its ligands BAFF (B cell activating factor, also designated as TALL-1 or TNFSF13B) and APRIL (A proliferation inducing ligand).

BCMA regulates different aspects of humoral immunity. B cell development and homeostasis along with its family members TACI (transmembrane activator and cyclophylin ligand interactor) and BAFF-R (B cell activation factor receptor, also known as tumor necrosis factor receptor superfamily member 13C). Expression of BCMA appears rather late in B cell differentiation and contributes to the long-term survival of plasmablasts and plasma cells in the bone marrow. BCMA also supports growth and survival of multiple myeloma (MM) cells.

BCMA is mostly known for its functional activity in mediating the survival of plasma cells that maintain long-term humoral immunity.

There is a need for having treatment options for solid tumor diseases related to the overexpression of BCMA, such as cancer multiple myeloma, leukemias and lymphomas. The present disclosure provides, in certain embodiments, single domain proteins which specifically bind to BCMA on the surface of tumor target cells.

The design of the BCMA targeting trispecific proteins described herein allows the binding domain to BCMA to be flexible in that the binding domain to BCMA can be any type of binding domain, including but not limited to, domains from a monoclonal antibody, a polyclonal antibody, a recombinant antibody, a human antibody, a humanized antibody. In some embodiments, the binding domain to BCMA is a single chain variable fragments (scFv), single-domain antibody such as a heavy chain variable domain (VH), a light chain variable domain (VL) and a variable domain (VHH) of camelid derived single domain antibody. In other embodiments, the binding domain to BCMA is a non-Ig binding domain, i.e., antibody mimetic, such as anticalins, affilins, affibody molecules, AFFIMERS®, affitins, alphabodies, avimers, DARPINS®, fynomers, kunitz domain peptides, and monobodies. In further embodiments, the binding domain to BCMA is a ligand or peptide that binds to or associates with BCMA. In yet further embodiments, the binding domain to BCMA is a knottin. In yet further embodiments, the binding domain to BCMA is a small molecular entity.

In some embodiments, the BCMA binding domain binds to a protein comprising the sequence of SEQ ID NO: 469, 470 or 471. In some embodiments, the BCMA binding domain binds to a protein comprising a truncated sequence compared to SEQ ID NO: 469, 470 or 471.

In some embodiments, the BCMA binding domain is an anti-BCMA antibody or an antibody variant. As used herein, the term “antibody variant” refers to variants and derivatives of an antibody described herein. In certain embodiments, amino acid sequence variants of the anti-BCMA antibodies described herein are contemplated. For example, in certain embodiments amino acid sequence variants of anti-BCMA antibodies described herein are contemplated to improve the binding affinity and/or other biological properties of the antibodies. Exemplary method for preparing amino acid variants include, but are not limited to, introducing appropriate modifications into the nucleotide sequence encoding the antibody, or by peptide synthesis. Such modifications include, for example, deletions from, and/or insertions into and/or substitutions of residues within the amino acid sequences of the antibody.

Any combination of deletion, insertion, and substitution can be made to arrive at the final construct, provided that the final construct possesses the desired characteristics, e.g., antigen-binding. In certain embodiments, antibody variants having one or more amino acid substitutions are provided. Sites of interest for substitution mutagenesis include the CDRs and framework regions. Examples of such substitutions are described below. Amino acid substitutions may be introduced into an antibody of interest and the products screened for a desired activity, e.g., retained/improved antigen binding, decreased immunogenicity, or improved T-cell mediated cytotoxicity (TDCC). Both conservative and non-conservative amino acid substitutions are contemplated for preparing the antibody variants.

In another example of a substitution to create a variant anti-BCMA antibody, one or more hypervariable region residues of a parent antibody are substituted. In general, variants are then selected based on improvements in desired properties compared to a parent antibody, for example, increased affinity, reduced affinity, reduced immunogenicity, increased pH dependence of binding.

In some embodiments, the BCMA binding domain of the BCMA targeting trispecific protein is a single domain antibody such as a heavy chain variable domain (VH), a variable domain (VHH) of a llama derived sdAb, a peptide, a ligand or a small molecule entity specific for BCMA. In some embodiments, the BCMA binding domain of the BCMA targeting trispecific protein described herein is any domain that binds to BCMA including but not limited to domains from a monoclonal antibody, a polyclonal antibody, a recombinant antibody, a human antibody, a humanized antibody. In certain embodiments, the BCMA binding domain is a single-domain antibody. In other embodiments, the BCMA binding domain is a peptide. In further embodiments, the BCMA binding domain is a small molecule.

Generally, it should be noted that the term single domain antibody as used herein in its broadest sense is not limited to a specific biological source or to a specific method of preparation. Single domain antibodies are antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, goat, rabbit, bovine. For example, in some embodiments, the single domain antibodies of the disclosure are obtained: (1) by isolating the VHH domain of a naturally occurring heavy chain antibody; (2) by expression of a nucleotide sequence encoding a naturally occurring VHH domain; (3) by “humanization” of a naturally occurring VHH domain or by expression of a nucleic acid encoding a such humanized VHH domain; (4) by “camelization” of a naturally occurring VH domain from any animal species, and in particular from a species of mammal, such as from a human being, or by expression of a nucleic acid encoding such a camelized VH domain; (5) by “camelisation” of a “domain antibody” or “Dab”, or by expression of a nucleic acid encoding such a camelized VH domain; (6) by using synthetic or semi-synthetic techniques for preparing proteins, polypeptides or other amino acid sequences; (7) by preparing a nucleic acid encoding a single domain antibody using techniques for nucleic acid synthesis known in the field, followed by expression of the nucleic acid thus obtained; and/or (8) by any combination of one or more of the foregoing.

In one embodiment, a single domain antibody corresponds to the VHH domains of naturally occurring heavy chain antibodies directed against BCMA. As further described herein, such VHH sequences can generally be generated or obtained by suitably immunizing a species of Llama with BCMA. (i.e., so as to raise an immune response and/or heavy chain antibodies directed against BCMA), by obtaining a suitable biological sample from said Llama (such as a blood sample, serum sample or sample of B-cells), and by generating VHH sequences directed against BCMA, starting from said sample, using any suitable technique known in the field.

In another embodiment, such naturally occurring VHH domains against BCMA, are obtained from naïve libraries of Camelid VHH sequences, for example by screening such a library using BCMA, or at least one part, fragment, antigenic determinant or epitope thereof using one or more screening techniques known in the field. Such libraries and techniques are for example described in WO 99/37681. WO 01/90190. WO 03/025020 and WO 03/035694. Alternatively, improved synthetic or semi-synthetic libraries derived from naïve VHH libraries are used, such as VHH libraries obtained from naïve VHH libraries by techniques such as random mutagenesis and/or CDR shuffling, as for example described in WO 00/43507.

In a further embodiment, yet another technique for obtaining VHH sequences directed against BCMA, involves suitably immunizing a transgenic mammal that is capable of expressing heavy chain antibodies (i.e., so as to raise an immune response and/or heavy chain antibodies directed against BCMA), obtaining a suitable biological sample from said transgenic mammal (such as a blood sample, serum sample or sample of B-cells), and then generating VHH sequences directed against BCMA, starting from said sample, using any suitable technique known in the field. For example, for this purpose, the heavy chain antibody-expressing rats or mice and the further methods and techniques described in WO 02/085945 and in WO 04/049794 can be used.

In some embodiments, an anti-BCMA single domain antibody of the BCMA targeting trispecific protein comprises a single domain antibody with an amino acid sequence that corresponds to the amino acid sequence of a naturally occurring VHH domain, but that has been “humanized”, i.e., by replacing one or more amino acid residues in the amino acid sequence of said naturally occurring VHH sequence (and in particular in the framework sequences) by one or more of the amino acid residues that occur at the corresponding position(s) in a VH domain from a conventional 4-chain antibody from a human being (e.g., as indicated above). This can be performed in a manner known in the field, which will be clear to the skilled person, for example on the basis of the further description herein. Again, it should be noted that such humanized anti-BCMA single domain antibodies of the disclosure are obtained in any suitable manner known per se (i.e., as indicated under points (1)-(8) above) and thus are not strictly limited to polypeptides that have been obtained using a polypeptide that comprises a naturally occurring VHH domain as a starting material. In some additional embodiments, a single domain anti-BCMA antibody, as described herein, comprises a single domain antibody with an amino acid sequence that corresponds to the amino acid sequence of a naturally occurring VH domain, but that has been “camelized”, i.e., by replacing one or more amino acid residues in the amino acid sequence of a naturally occurring VH domain from a conventional 4-chain antibody by one or more of the amino acid residues that occur at the corresponding position(s) in a VHH domain of a heavy chain antibody. Such “camelizing” substitutions are preferably inserted at amino acid positions that form and/or are present at the VH-VL interface, and/or at the so-called Camelidae hallmark residues (see, for example, WO 94/04678 and Davies and Riechmann (1994 and 1996)). Preferably, the VH sequence that is used as a starting material or starting point for generating or designing the camelized single domain is preferably a VH sequence from a mammal, more preferably the VH sequence of a human being, such as a VH3 sequence. However, it should be noted that such camelized anti-BCMA single domain antibodies of the disclosure, in certain embodiments, are obtained in any suitable manner known in the field (i.e., as indicated under points (1)-(8) above) and thus are not strictly limited to polypeptides that have been obtained using a polypeptide that comprises a naturally occurring VH domain as a starting material. For example, as further described herein, both “humanization” and “camelization” is performed by providing a nucleotide sequence that encodes a naturally occurring VHH domain or VH domain, respectively, and then changing, one or more codons in said nucleotide sequence in such a way that the new nucleotide sequence encodes a “humanized” or “camelized” single domain antibody, respectively. This nucleic acid can then be expressed, so as to provide a desired anti-BCMA single domain antibody of the disclosure. Alternatively, in other embodiments, based on the amino acid sequence of a naturally occurring VHH domain or VH domain, respectively, the amino acid sequence of the desired humanized or camelized anti-BCMA single domain antibody of the disclosure, respectively, are designed and then synthesized de novo using known techniques for peptide synthesis. In some embodiments, based on the amino acid sequence or nucleotide sequence of a naturally occurring VHH domain or VH domain, respectively, a nucleotide sequence encoding the desired humanized or camelized anti-BCMA single domain antibody of the disclosure, respectively, is designed and then synthesized de novo using known techniques for nucleic acid synthesis, after which the nucleic acid thus obtained is expressed in using known expression techniques, so as to provide the desired anti-BCMA single domain antibody of the disclosure.

Other suitable methods and techniques for obtaining the anti-BCMA single domain antibody of the disclosure and/or nucleic acids encoding the same, starting from naturally occurring VH sequences or VHH sequences for example comprises combining one or more parts of one or more naturally occurring VH sequences (such as one or more framework (FR) sequences and/or complementarity determining region (CDR) sequences), one or more parts of one or more naturally occurring VHH sequences (such as one or more FR sequences or CDR sequences), and/or one or more synthetic or semi-synthetic sequences, in a suitable manner, so as to provide an anti-BCMA single domain antibody of the disclosure or a nucleotide sequence or nucleic acid encoding the same.

In some embodiments, the BCMA binding domain is an anti-BCMA specific antibody comprising a heavy chain variable complementarity determining region CDR1, a heavy chain variable CDR2, a heavy chain variable CDR3, a light chain variable CDR1, a light chain variable CDR2, and a light chain variable CDR3. In some embodiments, the BCMA binding domain comprises any domain that binds to BCMA including but not limited to domains from a monoclonal antibody, a polyclonal antibody, a recombinant antibody, a human antibody, a humanized antibody, or antigen binding fragments such as single domain antibodies (sdAb), Fab, Fab′, F(ab)₂, and Fv fragments, fragments comprised of one or more CDRs, single-chain antibodies (e.g., single chain Fv fragments (scFv)), disulfide stabilized (dsFv) Fv fragments, heteroconjugate antibodies (e.g., bispecific antibodies), pFv fragments, heavy chain monomers or dimers, light chain monomers or dimers, and dimers consisting of one heavy chain and one light chain. In some embodiments, the BCMA binding domain is a single domain antibody. In some embodiments, the anti-BCMA single domain antibody comprises heavy chain variable complementarity determining regions (CDR), CDR1, CDR2, and CDR3.

In some embodiments, the BCMA binding protein of the present disclosure is a polypeptide comprising an amino acid sequence that is comprised of four framework regions/sequences (f1-f4) interrupted by three complementarity determining regions/sequences, as represented by the formula: f1-r1-f2-r2-f3-r3-f4, wherein r1, r2, and r3 are complementarity determining regions CDR1, CDR2, and CDR3, respectively, and f1, f2, f3, and f4 are framework residues. The r1 residues of the BCMA binding protein of the present disclosure comprise, for example, amino acid residues 26, 27, 28, 29, 30, 31, 32, 33 and 34; the r2 residues of the BCMA binding protein of the present disclosure comprise, for example, amino acid residues, for example, 50, 51, 52, 53, 54, 55, 56, 57, 58, 59, 60, 61, 62 and 63; and the r3 residues of the BCMA binding protein of the present disclosure comprise, for example, amino acid residues, for example, 97, 98, 99, 100, 101, 102, 103, 104, 105, 106, 107 and 108. In some embodiments, the BCMA binding protein comprises an amino acid sequence selected from SEQ ID NOS: 346-460.

In one embodiment, the CDR1 does not comprise an amino acid sequence of SEQ ID NO: 599. In one embodiment, the CDR2 does not comprise an amino acid sequence of SEQ ID NO: 600. In one embodiment, the CDR3 does not comprise an amino acid sequence of SEQ ID NO: 601.

In some embodiments, the CDR1 comprises the amino acid sequence as set forth in SEQ ID NO: 1 or a variant thereof having one, two, three, four, five, six, seven, eight, nine, or ten amino acid substitutions. An exemplary CDR1 comprises the amino acid sequence as set forth in SEQ ID NO: 4. Another exemplary CDR1 comprises the amino acid sequence as set forth in SEQ ID NO: 5. Another exemplary CDR1 comprises the amino acid sequence as set forth in SEQ ID NO: 6. Another exemplary CDR1 comprises the amino acid sequence as set forth in SEQ ID NO: 7. Another exemplary CDR1 comprises the amino acid sequence as set forth in SEQ ID NO: 8. Another exemplary CDR1 comprises the amino acid sequence as set forth in SEQ ID NO: 9. Another exemplary CDR1 comprises the amino acid sequence as set forth in SEQ ID NO: 10. Another exemplary CDR1 comprises the amino acid sequence as set forth in SEQ ID NO: 11. Another exemplary CDR1 comprises the amino acid sequence as set forth in SEQ ID NO: 12. Another exemplary CDR1 comprises the amino acid sequence as set forth in SEQ ID NO: 13. Another exemplary CDR1 comprises the amino acid sequence as set forth in SEQ ID NO: 14. Another exemplary CDR1 comprises the amino acid sequence as set forth in SEQ ID NO: 15. Another exemplary CDR1 comprises the amino acid sequence as set forth in SEQ ID NO: 16. Another exemplary CDR1 comprises the amino acid sequence as set forth in SEQ ID NO: 17. Another exemplary CDR1 comprises the amino acid sequence as set forth in SEQ ID NO: 18. Another exemplary CDR1 comprises the amino acid sequence as set forth in SEQ ID NO: 19. Another exemplary CDR1 comprises the amino acid sequence as set forth in SEQ ID NO: 20. Another exemplary CDR1 comprises the amino acid sequence as set forth in SEQ ID NO: 21. Another exemplary CDR1 comprises the amino acid sequence as set forth in SEQ ID NO: 22. Another exemplary CDR1 comprises the amino acid sequence as set forth in SEQ ID NO: 23. Another exemplary CDR1 comprises the amino acid sequence as set forth in SEQ ID NO: 24. Another exemplary CDR1 comprises the amino acid sequence as set forth in SEQ ID NO: 25. Another exemplary CDR1 comprises the amino acid sequence as set forth in SEQ ID NO: 26. Another exemplary CDR1 comprises the amino acid sequence as set forth in SEQ ID NO: 27. Another exemplary CDR1 comprises the amino acid sequence as set forth in SEQ ID NO: 28. Another exemplary CDR1 comprises the amino acid sequence as set forth in SEQ ID NO: 29. Another exemplary CDR1 comprises the amino acid sequence as set forth in SEQ ID NO: 30. Another exemplary CDR1 comprises the amino acid sequence as set forth in SEQ ID NO: 31. Another exemplary CDR1 comprises the amino acid sequence as set forth in SEQ ID NO: 32. Another exemplary CDR1 comprises the amino acid sequence as set forth in SEQ ID NO: 33. Another exemplary CDR1 comprises the amino acid sequence as set forth in SEQ ID NO: 34. Another exemplary CDR1 comprises the amino acid sequence as set forth in SEQ ID NO: 35. Another exemplary CDR1 comprises the amino acid sequence as set forth in SEQ ID NO: 36. Another exemplary CDR1 comprises the amino acid sequence as set forth in SEQ ID NO: 37. Another exemplary CDR1 comprises the amino acid sequence as set forth in SEQ ID NO: 38. Another exemplary CDR1 comprises the amino acid sequence as set forth in SEQ ID NO: 39. Another exemplary CDR1 comprises the amino acid sequence as set forth in SEQ ID NO: 40. Another exemplary CDR1 comprises the amino acid sequence as set forth in SEQ ID NO: 41. Another exemplary CDR1 comprises the amino acid sequence as set forth in SEQ ID NO: 42. Another exemplary CDR1 comprises the amino acid sequence as set forth in SEQ ID NO: 43. Another exemplary CDR1 comprises the amino acid sequence as set forth in SEQ ID NO: 44. Another exemplary CDR1 comprises the amino acid sequence as set forth in SEQ ID NO: 45. Another exemplary CDR1 comprises the amino acid sequence as set forth in SEQ ID NO: 46. Another exemplary CDR1 comprises the amino acid sequence as set forth in SEQ ID NO: 47. Another exemplary CDR1 comprises the amino acid sequence as set forth in SEQ ID NO: 48. Another exemplary CDR1 comprises the amino acid sequence as set forth in SEQ ID NO: 49. Another exemplary CDR1 comprises the amino acid sequence as set forth in SEQ ID NO: 50. Another exemplary CDR1 comprises the amino acid sequence as set forth in SEQ ID NO: 51. Another exemplary CDR1 comprises the amino acid sequence as set forth in SEQ ID NO: 52. Another exemplary CDR1 comprises the amino acid sequence as set forth in SEQ ID NO: 53. Another exemplary CDR1 comprises the amino acid sequence as set forth in SEQ ID NO: 54. Another exemplary CDR1 comprises the amino acid sequence as set forth in SEQ ID NO: 55. Another exemplary CDR1 comprises the amino acid sequence as set forth in SEQ ID NO: 56. Another exemplary CDR1 comprises the amino acid sequence as set forth in SEQ ID NO: 57. Another exemplary CDR1 comprises the amino acid sequence as set forth in SEQ ID NO: 58. Another exemplary CDR1 comprises the amino acid sequence as set forth in SEQ ID NO: 59. Another exemplary CDR1 comprises the amino acid sequence as set forth in SEQ ID NO: 60. Another exemplary CDR1 comprises the amino acid sequence as set forth in SEQ ID NO: 61. Another exemplary CDR1 comprises the amino acid sequence as set forth in SEQ ID NO: 62. Another exemplary CDR1 comprises the amino acid sequence as set forth in SEQ ID NO: 63. Another exemplary CDR1 comprises the amino acid sequence as set forth in SEQ ID NO: 64. Another exemplary CDR1 comprises the amino acid sequence as set forth in SEQ ID NO: 65. Another exemplary CDR1 comprises the amino acid sequence as set forth in SEQ ID NO: 66. Another exemplary CDR1 comprises the amino acid sequence as set forth in SEQ ID NO: 67. Another exemplary CDR1 comprises the amino acid sequence as set forth in SEQ ID NO: 68. Another exemplary CDR1 comprises the amino acid sequence as set forth in SEQ ID NO: 69. Another exemplary CDR1 comprises the amino acid sequence as set forth in SEQ ID NO: 70. Another exemplary CDR1 comprises the amino acid sequence as set forth in SEQ ID NO: 71. Another exemplary CDR1 comprises the amino acid sequence as set forth in SEQ ID NO: 72. Another exemplary CDR1 comprises the amino acid sequence as set forth in SEQ ID NO: 73. Another exemplary CDR1 comprises the amino acid sequence as set forth in SEQ ID NO: 74. Another exemplary CDR1 comprises the amino acid sequence as set forth in SEQ ID NO: 75. Another exemplary CDR1 comprises the amino acid sequence as set forth in SEQ ID NO: 76. Another exemplary CDR1 comprises the amino acid sequence as set forth in SEQ ID NO: 77. Another exemplary CDR1 comprises the amino acid sequence as set forth in SEQ ID NO: 78. Another exemplary CDR1 comprises the amino acid sequence as set forth in SEQ ID NO: 79. Another exemplary CDR1 comprises the amino acid sequence as set forth in SEQ ID NO: 80. Another exemplary CDR1 comprises the amino acid sequence as set forth in SEQ ID NO: 81. Another exemplary CDR1 comprises the amino acid sequence as set forth in SEQ ID NO: 82. Another exemplary CDR1 comprises the amino acid sequence as set forth in SEQ ID NO: 83. Another exemplary CDR1 comprises the amino acid sequence as set forth in SEQ ID NO: 84. Another exemplary CDR1 comprises the amino acid sequence as set forth in SEQ ID NO: 85. Another exemplary CDR1 comprises the amino acid sequence as set forth in SEQ ID NO: 86. Another exemplary CDR1 comprises the amino acid sequence as set forth in SEQ ID NO: 87. Another exemplary CDR1 comprises the amino acid sequence as set forth in SEQ ID NO: 88. Another exemplary CDR1 comprises the amino acid sequence as set forth in SEQ ID NO: 89. Another exemplary CDR1 comprises the amino acid sequence as set forth in SEQ ID NO: 90. Another exemplary CDR1 comprises the amino acid sequence as set forth in SEQ ID NO: 91. Another exemplary CDR1 comprises the amino acid sequence as set forth in SEQ ID NO: 92. Another exemplary CDR1 comprises the amino acid sequence as set forth in SEQ ID NO: 93. Another exemplary CDR1 comprises the amino acid sequence as set forth in SEQ ID NO: 94. Another exemplary CDR1 comprises the amino acid sequence as set forth in SEQ ID NO: 95. Another exemplary CDR1 comprises the amino acid sequence as set forth in SEQ ID NO: 96. Another exemplary CDR1 comprises the amino acid sequence as set forth in SEQ ID NO: 97. Another exemplary CDR1 comprises the amino acid sequence as set forth in SEQ ID NO: 98. Another exemplary CDR1 comprises the amino acid sequence as set forth in SEQ ID NO: 99. Another exemplary CDR1 comprises the amino acid sequence as set forth in SEQ ID NO: 100. Another exemplary CDR1 comprises the amino acid sequence as set forth in SEQ ID NO: 101. Another exemplary CDR1 comprises the amino acid sequence as set forth in SEQ ID NO: 102. Another exemplary CDR1 comprises the amino acid sequence as set forth in SEQ ID NO: 103. Another exemplary CDR1 comprises the amino acid sequence as set forth in SEQ ID NO: 104. Another exemplary CDR1 comprises the amino acid sequence as set forth in SEQ ID NO: 105. Another exemplary CDR1 comprises the amino acid sequence as set forth in SEQ ID NO: 106. Another exemplary CDR1 comprises the amino acid sequence as set forth in SEQ ID NO: 107. Another exemplary CDR1 comprises the amino acid sequence as set forth in SEQ ID NO: 108. Another exemplary CDR1 comprises the amino acid sequence as set forth in SEQ ID NO: 109. Another exemplary CDR1 comprises the amino acid sequence as set forth in SEQ ID NO: 110. Another exemplary CDR1 comprises the amino acid sequence as set forth in SEQ ID NO: 111. Another exemplary CDR1 comprises the amino acid sequence as set forth in SEQ ID NO: 112. Another exemplary CDR1 comprises the amino acid sequence as set forth in SEQ ID NO: 113. Another exemplary CDR1 comprises the amino acid sequence as set forth in SEQ ID NO: 114. Another exemplary CDR1 comprises the amino acid sequence as set forth in SEQ ID NO: 115. Another exemplary CDR1 comprises the amino acid sequence as set forth in SEQ ID NO: 116. Another exemplary CDR1 comprises the amino acid sequence as set forth in SEQ ID NO: 117.

In some embodiments, the CDR2 comprises a sequence as set forth in SEQ ID NO: 2 or a variant having one, two, three, four, five, six, seven, eight, nine, or ten amino acid substitutions in SEQ ID NO: 2. Another exemplary CDR2 comprises the amino acid sequence as set forth in SEQ ID NO: 118. Another exemplary CDR2 comprises the amino acid sequence as set forth in SEQ ID NO: 119. Another exemplary CDR2 comprises the amino acid sequence as set forth in SEQ ID NO: 120. Another exemplary CDR2 comprises the amino acid sequence as set forth in SEQ ID NO: 121. Another exemplary CDR2 comprises the amino acid sequence as set forth in SEQ ID NO: 122. Another exemplary CDR2 comprises the amino acid sequence as set forth in SEQ ID NO: 123. Another exemplary CDR2 comprises the amino acid sequence as set forth in SEQ ID NO: 124. Another exemplary CDR2 comprises the amino acid sequence as set forth in SEQ ID NO: 125. Another exemplary CDR2 comprises the amino acid sequence as set forth in SEQ ID NO: 126. Another exemplary CDR2 comprises the amino acid sequence as set forth in SEQ ID NO: 127. Another exemplary CDR2 comprises the amino acid sequence as set forth in SEQ ID NO: 128. Another exemplary CDR2 comprises the amino acid sequence as set forth in SEQ ID NO: 129. Another exemplary CDR2 comprises the amino acid sequence as set forth in SEQ ID NO: 130. Another exemplary CDR2 comprises the amino acid sequence as set forth in SEQ ID NO: 131. Another exemplary CDR2 comprises the amino acid sequence as set forth in SEQ ID NO: 132. Another exemplary CDR2 comprises the amino acid sequence as set forth in SEQ ID NO: 133. Another exemplary CDR2 comprises the amino acid sequence as set forth in SEQ ID NO: 134. Another exemplary CDR2 comprises the amino acid sequence as set forth in SEQ ID NO: 135. Another exemplary CDR2 comprises the amino acid sequence as set forth in SEQ ID NO: 136. Another exemplary CDR2 comprises the amino acid sequence as set forth in SEQ ID NO: 137. Another exemplary CDR2 comprises the amino acid sequence as set forth in SEQ ID NO: 138. Another exemplary CDR2 comprises the amino acid sequence as set forth in SEQ ID NO: 139. Another exemplary CDR2 comprises the amino acid sequence as set forth in SEQ ID NO: 140. Another exemplary CDR2 comprises the amino acid sequence as set forth in SEQ ID NO: 141. Another exemplary CDR2 comprises the amino acid sequence as set forth in SEQ ID NO: 142. Another exemplary CDR2 comprises the amino acid sequence as set forth in SEQ ID NO: 143. Another exemplary CDR2 comprises the amino acid sequence as set forth in SEQ ID NO: 144. Another exemplary CDR2 comprises the amino acid sequence as set forth in SEQ ID NO: 145. Another exemplary CDR2 comprises the amino acid sequence as set forth in SEQ ID NO: 146. Another exemplary CDR2 comprises the amino acid sequence as set forth in SEQ ID NO: 147. Another exemplary CDR2 comprises the amino acid sequence as set forth in SEQ ID NO: 148. Another exemplary CDR2 comprises the amino acid sequence as set forth in SEQ ID NO: 149. Another exemplary CDR2 comprises the amino acid sequence as set forth in SEQ ID NO: 150. Another exemplary CDR2 comprises the amino acid sequence as set forth in SEQ ID NO: 151. Another exemplary CDR2 comprises the amino acid sequence as set forth in SEQ ID NO: 152. Another exemplary CDR2 comprises the amino acid sequence as set forth in SEQ ID NO: 153. Another exemplary CDR2 comprises the amino acid sequence as set forth in SEQ ID NO: 154. Another exemplary CDR2 comprises the amino acid sequence as set forth in SEQ ID NO: 155. Another exemplary CDR2 comprises the amino acid sequence as set forth in SEQ ID NO: 156. Another exemplary CDR2 comprises the amino acid sequence as set forth in SEQ ID NO: 157. Another exemplary CDR2 comprises the amino acid sequence as set forth in SEQ ID NO: 158. Another exemplary CDR2 comprises the amino acid sequence as set forth in SEQ ID NO: 159. Another exemplary CDR2 comprises the amino acid sequence as set forth in SEQ ID NO: 160. Another exemplary CDR2 comprises the amino acid sequence as set forth in SEQ ID NO: 161. Another exemplary CDR2 comprises the amino acid sequence as set forth in SEQ ID NO: 162. Another exemplary CDR2 comprises the amino acid sequence as set forth in SEQ ID NO: 163. Another exemplary CDR2 comprises the amino acid sequence as set forth in SEQ ID NO: 164. Another exemplary CDR2 comprises the amino acid sequence as set forth in SEQ ID NO: 165. Another exemplary CDR2 comprises the amino acid sequence as set forth in SEQ ID NO: 166. Another exemplary CDR2 comprises the amino acid sequence as set forth in SEQ ID NO: 167. Another exemplary CDR2 comprises the amino acid sequence as set forth in SEQ ID NO: 168. Another exemplary CDR2 comprises the amino acid sequence as set forth in SEQ ID NO: 169. Another exemplary CDR2 comprises the amino acid sequence as set forth in SEQ ID NO: 170. Another exemplary CDR2 comprises the amino acid sequence as set forth in SEQ ID NO: 171. Another exemplary CDR2 comprises the amino acid sequence as set forth in SEQ ID NO: 172. Another exemplary CDR2 comprises the amino acid sequence as set forth in SEQ ID NO: 173. Another exemplary CDR2 comprises the amino acid sequence as set forth in SEQ ID NO: 174. Another exemplary CDR2 comprises the amino acid sequence as set forth in SEQ ID NO: 175. Another exemplary CDR2 comprises the amino acid sequence as set forth in SEQ ID NO: 176. Another exemplary CDR2 comprises the amino acid sequence as set forth in SEQ ID NO: 177. Another exemplary CDR2 comprises the amino acid sequence as set forth in SEQ ID NO: 178. Another exemplary CDR2 comprises the amino acid sequence as set forth in SEQ ID NO: 179. Another exemplary CDR2 comprises the amino acid sequence as set forth in SEQ ID NO: 180. Another exemplary CDR2 comprises the amino acid sequence as set forth in SEQ ID NO: 181. Another exemplary CDR2 comprises the amino acid sequence as set forth in SEQ ID NO: 182. Another exemplary CDR2 comprises the amino acid sequence as set forth in SEQ ID NO: 183. Another exemplary CDR2 comprises the amino acid sequence as set forth in SEQ ID NO: 184. Another exemplary CDR2 comprises the amino acid sequence as set forth in SEQ ID NO: 185. Another exemplary CDR2 comprises the amino acid sequence as set forth in SEQ ID NO: 186. Another exemplary CDR2 comprises the amino acid sequence as set forth in SEQ ID NO: 187. Another exemplary CDR2 comprises the amino acid sequence as set forth in SEQ ID NO: 188. Another exemplary CDR2 comprises the amino acid sequence as set forth in SEQ ID NO: 189. Another exemplary CDR2 comprises the amino acid sequence as set forth in SEQ ID NO: 190. Another exemplary CDR2 comprises the amino acid sequence as set forth in SEQ ID NO: 191. Another exemplary CDR2 comprises the amino acid sequence as set forth in SEQ ID NO: 192. Another exemplary CDR2 comprises the amino acid sequence as set forth in SEQ ID NO: 193. Another exemplary CDR2 comprises the amino acid sequence as set forth in SEQ ID NO: 194. Another exemplary CDR2 comprises the amino acid sequence as set forth in SEQ ID NO: 195. Another exemplary CDR2 comprises the amino acid sequence as set forth in SEQ ID NO: 196. Another exemplary CDR2 comprises the amino acid sequence as set forth in SEQ ID NO: 197. Another exemplary CDR2 comprises the amino acid sequence as set forth in SEQ ID NO: 198. Another exemplary CDR2 comprises the amino acid sequence as set forth in SEQ ID NO: 199. Another exemplary CDR2 comprises the amino acid sequence as set forth in SEQ ID NO: 200. Another exemplary CDR2 comprises the amino acid sequence as set forth in SEQ ID NO: 201. Another exemplary CDR2 comprises the amino acid sequence as set forth in SEQ ID NO: 202. Another exemplary CDR2 comprises the amino acid sequence as set forth in SEQ ID NO: 203. Another exemplary CDR2 comprises the amino acid sequence as set forth in SEQ ID NO: 204. Another exemplary CDR2 comprises the amino acid sequence as set forth in SEQ ID NO: 205. Another exemplary CDR2 comprises the amino acid sequence as set forth in SEQ ID NO: 206. Another exemplary CDR2 comprises the amino acid sequence as set forth in SEQ ID NO: 207. Another exemplary CDR2 comprises the amino acid sequence as set forth in SEQ ID NO: 208. Another exemplary CDR2 comprises the amino acid sequence as set forth in SEQ ID NO: 209. Another exemplary CDR2 comprises the amino acid sequence as set forth in SEQ ID NO: 210. Another exemplary CDR2 comprises the amino acid sequence as set forth in SEQ ID NO: 211. Another exemplary CDR2 comprises the amino acid sequence as set forth in SEQ ID NO: 212. Another exemplary CDR2 comprises the amino acid sequence as set forth in SEQ ID NO: 213. Another exemplary CDR2 comprises the amino acid sequence as set forth in SEQ ID NO: 214. Another exemplary CDR2 comprises the amino acid sequence as set forth in SEQ ID NO: 215. Another exemplary CDR2 comprises the amino acid sequence as set forth in SEQ ID NO: 216. Another exemplary CDR2 comprises the amino acid sequence as set forth in SEQ ID NO: 217. Another exemplary CDR2 comprises the amino acid sequence as set forth in SEQ ID NO: 218. Another exemplary CDR2 comprises the amino acid sequence as set forth in SEQ ID NO: 219. Another exemplary CDR2 comprises the amino acid sequence as set forth in SEQ ID NO: 220. Another exemplary CDR2 comprises the amino acid sequence as set forth in SEQ ID NO: 221. Another exemplary CDR2 comprises the amino acid sequence as set forth in SEQ ID NO: 222. Another exemplary CDR2 comprises the amino acid sequence as set forth in SEQ ID NO: 223. Another exemplary CDR2 comprises the amino acid sequence as set forth in SEQ ID NO: 224. Another exemplary CDR2 comprises the amino acid sequence as set forth in SEQ ID NO: 225. Another exemplary CDR2 comprises the amino acid sequence as set forth in SEQ ID NO: 226. Another exemplary CDR2 comprises the amino acid sequence as set forth in SEQ ID NO: 227. Another exemplary CDR2 comprises the amino acid sequence as set forth in SEQ ID NO: 228. Another exemplary CDR2 comprises the amino acid sequence as set forth in SEQ ID NO: 229. Another exemplary CDR2 comprises the amino acid sequence as set forth in SEQ ID NO: 230. Another exemplary CDR3 comprises the amino acid sequence as set forth in SEQ ID NO: 231.

In some embodiments, the CDR3 comprises a sequence as set forth in SEQ ID NO: 3 or a variant having one, two, three, four, five, six, seven, eight, nine, or ten amino acid substitutions in SEQ ID NO: 3. Another exemplary CDR3 comprises the amino acid sequence as set forth in SEQ ID NO: 232. Another exemplary CDR3 comprises the amino acid sequence as set forth in SEQ ID NO: 233. Another exemplary CDR3 comprises the amino acid sequence as set forth in SEQ ID NO: 234. Another exemplary CDR3 comprises the amino acid sequence as set forth in SEQ ID NO: 235. Another exemplary CDR3 comprises the amino acid sequence as set forth in SEQ ID NO: 236. Another exemplary CDR3 comprises the amino acid sequence as set forth in SEQ ID NO: 237. Another exemplary CDR3 comprises the amino acid sequence as set forth in SEQ ID NO: 238. Another exemplary CDR3 comprises the amino acid sequence as set forth in SEQ ID NO: 239. Another exemplary CDR3 comprises the amino acid sequence as set forth in SEQ ID NO: 240. Another exemplary CDR3 comprises the amino acid sequence as set forth in SEQ ID NO: 241. Another exemplary CDR3 comprises the amino acid sequence as set forth in SEQ ID NO: 242. Another exemplary CDR3 comprises the amino acid sequence as set forth in SEQ ID NO: 243. Another exemplary CDR3 comprises the amino acid sequence as set forth in SEQ ID NO: 244. Another exemplary CDR3 comprises the amino acid sequence as set forth in SEQ ID NO: 245. Another exemplary CDR3 comprises the amino acid sequence as set forth in SEQ ID NO: 246. Another exemplary CDR3 comprises the amino acid sequence as set forth in SEQ ID NO: 247. Another exemplary CDR3 comprises the amino acid sequence as set forth in SEQ ID NO: 248. Another exemplary CDR3 comprises the amino acid sequence as set forth in SEQ ID NO: 249. Another exemplary CDR3 comprises the amino acid sequence as set forth in SEQ ID NO: 250. Another exemplary CDR3 comprises the amino acid sequence as set forth in SEQ ID NO: 251. Another exemplary CDR3 comprises the amino acid sequence as set forth in SEQ ID NO: 252. Another exemplary CDR3 comprises the amino acid sequence as set forth in SEQ ID NO: 253. Another exemplary CDR3 comprises the amino acid sequence as set forth in SEQ ID NO: 254. Another exemplary CDR3 comprises the amino acid sequence as set forth in SEQ ID NO: 255. Another exemplary CDR3 comprises the amino acid sequence as set forth in SEQ ID NO: 256. Another exemplary CDR3 comprises the amino acid sequence as set forth in SEQ ID NO: 257. Another exemplary CDR3 comprises the amino acid sequence as set forth in SEQ ID NO: 258. Another exemplary CDR3 comprises the amino acid sequence as set forth in SEQ ID NO: 259. Another exemplary CDR3 comprises the amino acid sequence as set forth in SEQ ID NO: 260. Another exemplary CDR3 comprises the amino acid sequence as set forth in SEQ ID NO: 261. Another exemplary CDR3 comprises the amino acid sequence as set forth in SEQ ID NO: 262. Another exemplary CDR3 comprises the amino acid sequence as set forth in SEQ ID NO: 263. Another exemplary CDR3 comprises the amino acid sequence as set forth in SEQ ID NO: 264. Another exemplary CDR3 comprises the amino acid sequence as set forth in SEQ ID NO: 265. Another exemplary CDR3 comprises the amino acid sequence as set forth in SEQ ID NO: 266. Another exemplary CDR3 comprises the amino acid sequence as set forth in SEQ ID NO: 267. Another exemplary CDR3 comprises the amino acid sequence as set forth in SEQ ID NO: 268. Another exemplary CDR3 comprises the amino acid sequence as set forth in SEQ ID NO: 269. Another exemplary CDR3 comprises the amino acid sequence as set forth in SEQ ID NO: 270. Another exemplary CDR3 comprises the amino acid sequence as set forth in SEQ ID NO: 271. Another exemplary CDR3 comprises the amino acid sequence as set forth in SEQ ID NO: 272. Another exemplary CDR3 comprises the amino acid sequence as set forth in SEQ ID NO: 273. Another exemplary CDR3 comprises the amino acid sequence as set forth in SEQ ID NO: 274. Another exemplary CDR3 comprises the amino acid sequence as set forth in SEQ ID NO: 275. Another exemplary CDR3 comprises the amino acid sequence as set forth in SEQ ID NO: 276. Another exemplary CDR3 comprises the amino acid sequence as set forth in SEQ ID NO: 277. Another exemplary CDR3 comprises the amino acid sequence as set forth in SEQ ID NO: 278. Another exemplary CDR3 comprises the amino acid sequence as set forth in SEQ ID NO: 279. Another exemplary CDR3 comprises the amino acid sequence as set forth in SEQ ID NO: 280. Another exemplary CDR3 comprises the amino acid sequence as set forth in SEQ ID NO: 281. Another exemplary CDR3 comprises the amino acid sequence as set forth in SEQ ID NO: 282. Another exemplary CDR3 comprises the amino acid sequence as set forth in SEQ ID NO: 283. Another exemplary CDR3 comprises the amino acid sequence as set forth in SEQ ID NO: 284. Another exemplary CDR3 comprises the amino acid sequence as set forth in SEQ ID NO: 285. Another exemplary CDR3 comprises the amino acid sequence as set forth in SEQ ID NO: 286. Another exemplary CDR3 comprises the amino acid sequence as set forth in SEQ ID NO: 287. Another exemplary CDR3 comprises the amino acid sequence as set forth in SEQ ID NO: 288. Another exemplary CDR3 comprises the amino acid sequence as set forth in SEQ ID NO: 289. Another exemplary CDR3 comprises the amino acid sequence as set forth in SEQ ID NO: 290. Another exemplary CDR3 comprises the amino acid sequence as set forth in SEQ ID NO: 291. Another exemplary CDR3 comprises the amino acid sequence as set forth in SEQ ID NO: 292. Another exemplary CDR3 comprises the amino acid sequence as set forth in SEQ ID NO: 293. Another exemplary CDR3 comprises the amino acid sequence as set forth in SEQ ID NO: 294. Another exemplary CDR3 comprises the amino acid sequence as set forth in SEQ ID NO: 295. Another exemplary CDR3 comprises the amino acid sequence as set forth in SEQ ID NO: 296. Another exemplary CDR3 comprises the amino acid sequence as set forth in SEQ ID NO: 297. Another exemplary CDR3 comprises the amino acid sequence as set forth in SEQ ID NO: 298. Another exemplary CDR3 comprises the amino acid sequence as set forth in SEQ ID NO: 299. Another exemplary CDR3 comprises the amino acid sequence as set forth in SEQ ID NO: 300. Another exemplary CDR3 comprises the amino acid sequence as set forth in SEQ ID NO: 301. Another exemplary CDR3 comprises the amino acid sequence as set forth in SEQ ID NO: 302. Another exemplary CDR3 comprises the amino acid sequence as set forth in SEQ ID NO: 303. Another exemplary CDR3 comprises the amino acid sequence as set forth in SEQ ID NO: 304. Another exemplary CDR3 comprises the amino acid sequence as set forth in SEQ ID NO: 305. Another exemplary CDR3 comprises the amino acid sequence as set forth in SEQ ID NO: 306. Another exemplary CDR3 comprises the amino acid sequence as set forth in SEQ ID NO: 307. Another exemplary CDR3 comprises the amino acid sequence as set forth in SEQ ID NO: 308. Another exemplary CDR3 comprises the amino acid sequence as set forth in SEQ ID NO: 309. Another exemplary CDR3 comprises the amino acid sequence as set forth in SEQ ID NO: 310. Another exemplary CDR3 comprises the amino acid sequence as set forth in SEQ ID NO: 311. Another exemplary CDR3 comprises the amino acid sequence as set forth in SEQ ID NO: 312. Another exemplary CDR3 comprises the amino acid sequence as set forth in SEQ ID NO: 313. Another exemplary CDR3 comprises the amino acid sequence as set forth in SEQ ID NO: 314. Another exemplary CDR3 comprises the amino acid sequence as set forth in SEQ ID NO: 315. Another exemplary CDR3 comprises the amino acid sequence as set forth in SEQ ID NO: 316. Another exemplary CDR3 comprises the amino acid sequence as set forth in SEQ ID NO: 317. Another exemplary CDR3 comprises the amino acid sequence as set forth in SEQ ID NO: 318. Another exemplary CDR3 comprises the amino acid sequence as set forth in SEQ ID NO: 319. Another exemplary CDR3 comprises the amino acid sequence as set forth in SEQ ID NO: 320. Another exemplary CDR3 comprises the amino acid sequence as set forth in SEQ ID NO: 321. Another exemplary CDR3 comprises the amino acid sequence as set forth in SEQ ID NO: 322. Another exemplary CDR3 comprises the amino acid sequence as set forth in SEQ ID NO: 323. Another exemplary CDR3 comprises the amino acid sequence as set forth in SEQ ID NO: 324. Another exemplary CDR3 comprises the amino acid sequence as set forth in SEQ ID NO: 325. Another exemplary CDR3 comprises the amino acid sequence as set forth in SEQ ID NO: 326. Another exemplary CDR3 comprises the amino acid sequence as set forth in SEQ ID NO: 327. Another exemplary CDR3 comprises the amino acid sequence as set forth in SEQ ID NO: 328. Another exemplary CDR3 comprises the amino acid sequence as set forth in SEQ ID NO: 329. Another exemplary CDR3 comprises the amino acid sequence as set forth in SEQ ID NO: 330. Another exemplary CDR3 comprises the amino acid sequence as set forth in SEQ ID NO: 331. Another exemplary CDR3 comprises the amino acid sequence as set forth in SEQ ID NO: 332. Another exemplary CDR3 comprises the amino acid sequence as set forth in SEQ ID NO: 333. Another exemplary CDR3 comprises the amino acid sequence as set forth in SEQ ID NO: 334. Another exemplary CDR3 comprises the amino acid sequence as set forth in SEQ ID NO: 335. Another exemplary CDR3 comprises the amino acid sequence as set forth in SEQ ID NO: 336. Another exemplary CDR3 comprises the amino acid sequence as set forth in SEQ ID NO: 337. Another exemplary CDR3 comprises the amino acid sequence as set forth in SEQ ID NO: 338. Another exemplary CDR3 comprises the amino acid sequence as set forth in SEQ ID NO: 339. Another exemplary CDR3 comprises the amino acid sequence as set forth in SEQ ID NO: 340. Another exemplary CDR3 comprises the amino acid sequence as set forth in SEQ ID NO: 341. Another exemplary CDR3 comprises the amino acid sequence as set forth in SEQ ID NO: 342. Another exemplary CDR3 comprises the amino acid sequence as set forth in SEQ ID NO: 343. Another exemplary CDR3 comprises the amino acid sequence as set forth in SEQ ID NO: 344. Another exemplary CDR3 comprises the amino acid sequence as set forth in SEQ ID NO: 345.

In various embodiments, the BCMA binding protein of the present disclosure has a CDR1 that has an amino acid sequence that is at least about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to an amino acid sequence selected from SEQ ID NOS: 4-117.

In various embodiments, the BCMA binding protein of the present disclosure has a CDR2 that has an amino acid sequence that is at least about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to an amino acid sequence selected from SEQ ID NOS: 118-231.

In various embodiments, a complementarity determining region of the BCMA binding protein of the present disclosure has a CDR3 that has an amino acid sequence that is at least about 10%, about 20%, about 30%, about 40%, about 50%, about 60%, about 70%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to an amino acid sequence selected from SEQ ID NOS: 232-345.

In various embodiments, a BCMA binding protein of the present disclosure has an amino acid sequence that is at least about 10%, about 20%, about 30%, about 40%, about 50%, about 60%, about 70%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to an amino acid sequence selected from SEQ ID NOS: 346-460.

In various embodiments, a BCMA binding protein of the present disclosure has a framework 1 (f1) that has an amino acid sequence that is at least about 10%, about 20%, about 30%, about 40%, about 50%, about 60%, about 70%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to the amino acid sequence set forth in SEQ ID NO: 461 or SEQ ID NO: 462.

In various embodiments, a BCMA binding protein of the present disclosure has a framework 2 (f2) that has an amino acid sequence that is at least about 10%, about 20%, about 30%, about 40%, about 50%, about 60%, about 70%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to the amino acid sequence set forth in SEQ ID NO: 463.

In various embodiments, a BCMA binding protein of the present disclosure has a framework 3 (f3) that has an amino acid sequence that is at least about 10%, about 20%, about 30%, about 40%, about 50%, about 60%, about 70%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to the amino acid sequence set forth in SEQ ID NO: 464 or SEQ ID NO: 465.

In various embodiments, a BCMA binding protein of the present disclosure has a framework 4 (f4) that has an amino acid sequence that is at least about 10%, about 20%, about 30%, about 40%, about 50%, about 60%, about 70%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to the amino acid sequence set forth in SEQ ID NO: 466 or SEQ ID NO: 467.

In some embodiments, the BCMA binding protein is a single domain antibody comprising the sequence of SEQ ID NO: 346. In some embodiments, the BCMA binding protein is a single domain antibody comprising the sequence of SEQ ID NO: 347. In some embodiments, the BCMA binding protein is a single domain antibody comprising the sequence of SEQ ID NO: 348. In some embodiments, the BCMA binding protein is a single domain antibody comprising the sequence of SEQ ID NO: 349. In some embodiments, the BCMA binding protein is a single domain antibody comprising the sequence of SEQ ID NO: 350. In some embodiments, the BCMA binding protein is a single domain antibody comprising the sequence of SEQ ID NO: 351. In some embodiments, the BCMA binding protein is a single domain antibody comprising the sequence of SEQ ID NO: 352. In some embodiments, the BCMA binding protein is a single domain antibody comprising the sequence of SEQ ID NO: 353. In some embodiments, the BCMA binding protein is a single domain antibody comprising the sequence of SEQ ID NO: 354. In some embodiments, the BCMA binding protein is a single domain antibody comprising the sequence of SEQ ID NO: 355. In some embodiments, the BCMA binding protein is a single domain antibody comprising the sequence of SEQ ID NO: 356. In some embodiments, the BCMA binding protein is a single domain antibody comprising the sequence of SEQ ID NO: 357. In some embodiments, the BCMA binding protein is a single domain antibody comprising the sequence of SEQ ID NO: 358. In some embodiments, the BCMA binding protein is a single domain antibody comprising the sequence of SEQ ID NO: 359.

In some embodiments, the BCMA binding protein is a single domain antibody comprising the sequence of SEQ ID NO: 360. In some embodiments, the BCMA binding protein is a single domain antibody comprising the sequence of SEQ ID NO: 361. In some embodiments, the BCMA binding protein is a single domain antibody comprising the sequence of SEQ ID NO: 362. In some embodiments, the BCMA binding protein is a single domain antibody comprising the sequence of SEQ ID NO: 363. In some embodiments, the BCMA binding protein is a single domain antibody comprising the sequence of SEQ ID NO: 364. In some embodiments, the BCMA binding protein is a single domain antibody comprising the sequence of SEQ ID NO: 365. In some embodiments, the BCMA binding protein is a single domain antibody comprising the sequence of SEQ ID NO: 366. In some embodiments, the BCMA binding protein is a single domain antibody comprising the sequence of SEQ ID NO: 367. In some embodiments, the BCMA binding protein is a single domain antibody comprising the sequence of SEQ ID NO: 368. In some embodiments, the BCMA binding protein is a single domain antibody comprising the sequence of SEQ ID NO: 369.

In some embodiments, the BCMA binding protein is a single domain antibody comprising the sequence of SEQ ID NO: 370. In some embodiments, the BCMA binding protein is a single domain antibody comprising the sequence of SEQ ID NO: 371. In some embodiments, the BCMA binding protein is a single domain antibody comprising the sequence of SEQ ID NO: 372. In some embodiments, the BCMA binding protein is a single domain antibody comprising the sequence of SEQ ID NO: 373. In some embodiments, the BCMA binding protein is a single domain antibody comprising the sequence of SEQ ID NO: 374. In some embodiments, the BCMA binding protein is a single domain antibody comprising the sequence of SEQ ID NO: 375. In some embodiments, the BCMA binding protein is a single domain antibody comprising the sequence of SEQ ID NO: 376. In some embodiments, the BCMA binding protein is a single domain antibody comprising the sequence of SEQ ID NO: 377. In some embodiments, the BCMA binding protein is a single domain antibody comprising the sequence of SEQ ID NO: 378. In some embodiments, the BCMA binding protein is a single domain antibody comprising the sequence of SEQ ID NO: 379.

In some embodiments, the BCMA binding protein is a single domain antibody comprising the sequence of SEQ ID NO: 380. In some embodiments, the BCMA binding protein is a single domain antibody comprising the sequence of SEQ ID NO: 381. In some embodiments, the BCMA binding protein is a single domain antibody comprising the sequence of SEQ ID NO: 382. In some embodiments, the BCMA binding protein is a single domain antibody comprising the sequence of SEQ ID NO: 383. In some embodiments, the BCMA binding protein is a single domain antibody comprising the sequence of SEQ ID NO: 384. In some embodiments, the BCMA binding protein is a single domain antibody comprising the sequence of SEQ ID NO: 385. In some embodiments, the BCMA binding protein is a single domain antibody comprising the sequence of SEQ ID NO: 386. In some embodiments, the BCMA binding protein is a single domain antibody comprising the sequence of SEQ ID NO: 387. In some embodiments, the BCMA binding protein is a single domain antibody comprising the sequence of SEQ ID NO: 388. In some embodiments, the BCMA binding protein is a single domain antibody comprising the sequence of SEQ ID NO: 389.

In some embodiments, the BCMA binding protein is a single domain antibody comprising the sequence of SEQ ID NO: 390. In some embodiments, the BCMA binding protein is a single domain antibody comprising the sequence of SEQ ID NO: 391. In some embodiments, the BCMA binding protein is a single domain antibody comprising the sequence of SEQ ID NO: 392. In some embodiments, the BCMA binding protein is a single domain antibody comprising the sequence of SEQ ID NO: 393. In some embodiments, the BCMA binding protein is a single domain antibody comprising the sequence of SEQ ID NO: 394. In some embodiments, the BCMA binding protein is a single domain antibody comprising the sequence of SEQ ID NO: 395. In some embodiments, the BCMA binding protein is a single domain antibody comprising the sequence of SEQ ID NO: 396. In some embodiments, the BCMA binding protein is a single domain antibody comprising the sequence of SEQ ID NO: 397. In some embodiments, the BCMA binding protein is a single domain antibody comprising the sequence of SEQ ID NO: 398. In some embodiments, the BCMA binding protein is a single domain antibody comprising the sequence of SEQ ID NO: 399.

In some embodiments, the BCMA binding protein is a single domain antibody comprising the sequence of SEQ ID NO: 400. In some embodiments, the BCMA binding protein is a single domain antibody comprising the sequence of SEQ ID NO: 401. In some embodiments, the BCMA binding protein is a single domain antibody comprising the sequence of SEQ ID NO: 402. In some embodiments, the BCMA binding protein is a single domain antibody comprising the sequence of SEQ ID NO: 403. In some embodiments, the BCMA binding protein is a single domain antibody comprising the sequence of SEQ ID NO: 404. In some embodiments, the BCMA binding protein is a single domain antibody comprising the sequence of SEQ ID NO: 405. In some embodiments, the BCMA binding protein is a single domain antibody comprising the sequence of SEQ ID NO: 406. In some embodiments, the BCMA binding protein is a single domain antibody comprising the sequence of SEQ ID NO: 407. In some embodiments, the BCMA binding protein is a single domain antibody comprising the sequence of SEQ ID NO: 408. In some embodiments, the BCMA binding protein is a single domain antibody comprising the sequence of SEQ ID NO: 409.

In some embodiments, the BCMA binding protein is a single domain antibody comprising the sequence of SEQ ID NO: 410. In some embodiments, the BCMA binding protein is a single domain antibody comprising the sequence of SEQ ID NO: 411. In some embodiments, the BCMA binding protein is a single domain antibody comprising the sequence of SEQ ID NO: 412. In some embodiments, the BCMA binding protein is a single domain antibody comprising the sequence of SEQ ID NO: 413. In some embodiments, the BCMA binding protein is a single domain antibody comprising the sequence of SEQ ID NO: 414. In some embodiments, the BCMA binding protein is a single domain antibody comprising the sequence of SEQ ID NO: 415. In some embodiments, the BCMA binding protein is a single domain antibody comprising the sequence of SEQ ID NO: 416. In some embodiments, the BCMA binding protein is a single domain antibody comprising the sequence of SEQ ID NO: 417. In some embodiments, the BCMA binding protein is a single domain antibody comprising the sequence of SEQ ID NO: 418. In some embodiments, the BCMA binding protein is a single domain antibody comprising the sequence of SEQ ID NO: 419.

In some embodiments, the BCMA binding protein is a single domain antibody comprising the sequence of SEQ ID NO: 420. In some embodiments, the BCMA binding protein is a single domain antibody comprising the sequence of SEQ ID NO: 421. In some embodiments, the BCMA binding protein is a single domain antibody comprising the sequence of SEQ ID NO: 422. In some embodiments, the BCMA binding protein is a single domain antibody comprising the sequence of SEQ ID NO: 423. In some embodiments, the BCMA binding protein is a single domain antibody comprising the sequence of SEQ ID NO: 424. In some embodiments, the BCMA binding protein is a single domain antibody comprising the sequence of SEQ ID NO: 425. In some embodiments, the BCMA binding protein is a single domain antibody comprising the sequence of SEQ ID NO: 426. In some embodiments, the BCMA binding protein is a single domain antibody comprising the sequence of SEQ ID NO: 427. In some embodiments, the BCMA binding protein is a single domain antibody comprising the sequence of SEQ ID NO: 428. In some embodiments, the BCMA binding protein is a single domain antibody comprising the sequence of SEQ ID NO: 429.

In some embodiments, the BCMA binding protein is a single domain antibody comprising the sequence of SEQ ID NO: 430. In some embodiments, the BCMA binding protein is a single domain antibody comprising the sequence of SEQ ID NO: 431. In some embodiments, the BCMA binding protein is a single domain antibody comprising the sequence of SEQ ID NO: 432. In some embodiments, the BCMA binding protein is a single domain antibody comprising the sequence of SEQ ID NO: 433. In some embodiments, the BCMA binding protein is a single domain antibody comprising the sequence of SEQ ID NO: 434. In some embodiments, the BCMA binding protein is a single domain antibody comprising the sequence of SEQ ID NO: 435. In some embodiments, the BCMA binding protein is a single domain antibody comprising the sequence of SEQ ID NO: 436. In some embodiments, the BCMA binding protein is a single domain antibody comprising the sequence of SEQ ID NO: 437. In some embodiments, the BCMA binding protein is a single domain antibody comprising the sequence of SEQ ID NO: 438. In some embodiments, the BCMA binding protein is a single domain antibody comprising the sequence of SEQ ID NO: 439.

In some embodiments, the BCMA binding protein is a single domain antibody comprising the sequence of SEQ ID NO: 440. In some embodiments, the BCMA binding protein is a single domain antibody comprising the sequence of SEQ ID NO: 441. In some embodiments, the BCMA binding protein is a single domain antibody comprising the sequence of SEQ ID NO: 442. In some embodiments, the BCMA binding protein is a single domain antibody comprising the sequence of SEQ ID NO: 443. In some embodiments, the BCMA binding protein is a single domain antibody comprising the sequence of SEQ ID NO: 444. In some embodiments, the BCMA binding protein is a single domain antibody comprising the sequence of SEQ ID NO: 445. In some embodiments, the BCMA binding protein is a single domain antibody comprising the sequence of SEQ ID NO: 446. In some embodiments, the BCMA binding protein is a single domain antibody comprising the sequence of SEQ ID NO: 447. In some embodiments, the BCMA binding protein is a single domain antibody comprising the sequence of SEQ ID NO: 448. In some embodiments, the BCMA binding protein is a single domain antibody comprising the sequence of SEQ ID NO: 449.

In some embodiments, the BCMA binding protein is a single domain antibody comprising the sequence of SEQ ID NO: 450. In some embodiments, the BCMA binding protein is a single domain antibody comprising the sequence of SEQ ID NO: 451. In some embodiments, the BCMA binding protein is a single domain antibody comprising the sequence of SEQ ID NO: 452. In some embodiments, the BCMA binding protein is a single domain antibody comprising the sequence of SEQ ID NO: 453. In some embodiments, the BCMA binding protein is a single domain antibody comprising the sequence of SEQ ID NO: 454. In some embodiments, the BCMA binding protein is a single domain antibody comprising the sequence of SEQ ID NO: 455. In some embodiments, the BCMA binding protein is a single domain antibody comprising the sequence of SEQ ID NO: 456. In some embodiments, the BCMA binding protein is a single domain antibody comprising the sequence of SEQ ID NO: 457. In some embodiments, the BCMA binding protein is a single domain antibody comprising the sequence of SEQ ID NO: 458. In some embodiments, the BCMA binding protein is a single domain antibody comprising the sequence of SEQ ID NO: 459. In some embodiments, the BCMA binding protein is a single domain antibody comprising the sequence of SEQ ID NO: 460.

A BCMA binding protein described herein can bind to human BCMA with a hKd ranges from about 0.1 nM to about 500 nM. In some embodiments, the hKd ranges from about 0.1 nM to about 450 nM. In some embodiments, the hKd ranges from about 0.1 nM to about 400 nM. In some embodiments, the hKd ranges from about 0.1 nM to about 350 nM. In some embodiments, the hKd ranges from about 0.1 nM to about 300 nM. In some embodiments, the hKd ranges from about 0.1 nM to about 250 nM. In some embodiments, the hKd ranges from about 0.1 nM to about 200 nM. In some embodiments, the hKd ranges from about 0.1 nM to about 150 nM. In some embodiments, the hKd ranges from about 0.1 nM to about 100 nM. In some embodiments, the hKd ranges from about 0.1 nM to about 90 nM. In some embodiments, the hKd ranges from about 0.2 nM to about 80 nM. In some embodiments, the hKd ranges from about 0.3 nM to about 70 nM. In some embodiments, the hKd ranges from about 0.4 nM to about 50 nM. In some embodiments, the hKd ranges from about 0.5 nM to about 30 nM. In some embodiments, the hKd ranges from about 0.6 nM to about 10 nM. In some embodiments, the hKd ranges from about 0.7 nM to about 8 nM. In some embodiments, the hKd ranges from about 0.8 nM to about 6 nM. In some embodiments, the hKd ranges from about 0.9 nM to about 4 nM. In some embodiments, the hKd ranges from about 1 nM to about 2 nM.

In some embodiments, any of the foregoing BCMA binding domains are affinity peptide tagged for ease of purification. In some embodiments, the affinity peptide tag is six consecutive histidine residues, also referred to as a His tag or 6×-his (His-His-His-His-His-His; SEQ ID NO: 471).

In certain embodiments, the BCMA binding domains of the present disclosure preferentially bind membrane bound BCMA over soluble BCMA. Membrane bound BCMA refers to the presence of BCMA in or on the cell membrane surface of a cell that expresses BCMA. Soluble BCMA refers to BCMA that is no longer on in or on the cell membrane surface of a cell that expresses or expressed BCMA. In certain instances, the soluble BCMA is present in the blood and/or lymphatic circulation in a subject. In one embodiment, the BCMA binding domains bind membrane-bound BCMA at least 5 fold, 10 fold, 15 fold, 20 fold, 25 fold, 30 fold, 40 fold, 50 fold, 100 fold, 500 fold, or 1000 fold greater than soluble BCMA. In one embodiment, the BCMA targeting trispecific antigen binding proteins of the present disclosure preferentially bind membrane-bound BCMA 30 fold greater than soluble BCMA. Determining the preferential binding of an antigen binding protein to membrane bound BCMA over soluble BCMA can be readily determined using assays well known in the art.

### Trispecific Proteins

A BCMA binding trispecific protein comprises an amino acid sequence selected from the group consisting of SEQ ID NO: 483-597.

In one example, a BCMA binding trispecific protein comprises an amino acid sequence of SEQ ID NO: 483. In one example, a BCMA binding trispecific protein comprises an amino acid sequence of SEQ ID NO: 484. In one example, a BCMA binding trispecific protein comprises an amino acid sequence of SEQ ID NO: 485. In one example, a BCMA binding trispecific protein comprises an amino acid sequence of SEQ ID NO: 486. In one example, a BCMA binding trispecific protein comprises an amino acid sequence of SEQ ID NO: 487. In one example, a BCMA binding trispecific protein comprises an amino acid sequence of SEQ ID NO: 488. In one example, a BCMA binding trispecific protein comprises an amino acid sequence of SEQ ID NO: 489. In one example, a BCMA binding trispecific protein comprises an amino acid sequence of SEQ ID NO: 490. In one example, a BCMA binding trispecific protein comprises an amino acid sequence of SEQ ID NO: 491. In one example, a BCMA binding trispecific protein comprises an amino acid sequence of SEQ ID NO: 492. In one example, a BCMA binding trispecific protein comprises an amino acid sequence of SEQ ID NO: 493. In one example, a BCMA binding trispecific protein comprises an amino acid sequence of SEQ ID NO: 494. In one example, a BCMA binding trispecific protein comprises an amino acid sequence of SEQ ID NO: 495. In one example, a BCMA binding trispecific protein comprises an amino acid sequence of SEQ ID NO: 496. In one example, a BCMA binding trispecific protein comprises an amino acid sequence of SEQ ID NO: 497. In one example, a BCMA binding trispecific protein comprises an amino acid sequence of SEQ ID NO: 498. In one example, a BCMA binding trispecific protein comprises an amino acid sequence of SEQ ID NO: 499.

In one example, a BCMA binding trispecific protein comprises an amino acid sequence of SEQ ID NO: 500. In one example, a BCMA binding trispecific protein comprises an amino acid sequence of SEQ ID NO: 501. In one example, a BCMA binding trispecific protein comprises an amino acid sequence of SEQ ID NO: 502. In one example, a BCMA binding trispecific protein comprises an amino acid sequence of SEQ ID NO: 503. In one example, a BCMA binding trispecific protein comprises an amino acid sequence of SEQ ID NO: 504. In one example, a BCMA binding trispecific protein comprises an amino acid sequence of SEQ ID NO: 505. In one example, a BCMA binding trispecific protein comprises an amino acid sequence of SEQ ID NO: 506. In one example, a BCMA binding trispecific protein comprises an amino acid sequence of SEQ ID NO: 507. In one example, a BCMA binding trispecific protein comprises an amino acid sequence of SEQ ID NO: 508. In one example, a BCMA binding trispecific protein comprises an amino acid sequence of SEQ ID NO: 509.

In one example, a BCMA binding trispecific protein comprises an amino acid sequence of SEQ ID NO: 510. In one example, a BCMA binding trispecific protein comprises an amino acid sequence of SEQ ID NO: 511. In one example, a BCMA binding trispecific protein comprises an amino acid sequence of SEQ ID NO: 512. In one example, a BCMA binding trispecific protein comprises an amino acid sequence of SEQ ID NO: 513. In one example, a BCMA binding trispecific protein comprises an amino acid sequence of SEQ ID NO: 514. In one example, a BCMA binding trispecific protein comprises an amino acid sequence of SEQ ID NO: 515. In one example, a BCMA binding trispecific protein comprises an amino acid sequence of SEQ ID NO: 516. In one example, a BCMA binding trispecific protein comprises an amino acid sequence of SEQ ID NO: 517. In one example, a BCMA binding trispecific protein comprises an amino acid sequence of SEQ ID NO: 518. In one example, a BCMA binding trispecific protein comprises an amino acid sequence of SEQ ID NO: 519.

In one example, a BCMA binding trispecific protein comprises an amino acid sequence of SEQ ID NO: 520. In one example, a BCMA binding trispecific protein comprises an amino acid sequence of SEQ ID NO: 521. In one example, a BCMA binding trispecific protein comprises an amino acid sequence of SEQ ID NO: 522. In one example, a BCMA binding trispecific protein comprises an amino acid sequence of SEQ ID NO: 523. In one example, a BCMA binding trispecific protein comprises an amino acid sequence of SEQ ID NO: 524. In one example, a BCMA binding trispecific protein comprises an amino acid sequence of SEQ ID NO: 525. In one example, a BCMA binding trispecific protein comprises an amino acid sequence of SEQ ID NO: 526. In one example, a BCMA binding trispecific protein comprises an amino acid sequence of SEQ ID NO: 527. In one example, a BCMA binding trispecific protein comprises an amino acid sequence of SEQ ID NO: 528. In one example, a BCMA binding trispecific protein comprises an amino acid sequence of SEQ ID NO: 529.

In one example, a BCMA binding trispecific protein comprises an amino acid sequence of SEQ ID NO: 530. In one example, a BCMA binding trispecific protein comprises an amino acid sequence of SEQ ID NO: 531. In one example, a BCMA binding trispecific protein comprises an amino acid sequence of SEQ ID NO: 532. In one example, a BCMA binding trispecific protein comprises an amino acid sequence of SEQ ID NO: 533. In one example, a BCMA binding trispecific protein comprises an amino acid sequence of SEQ ID NO: 534. In one example, a BCMA binding trispecific protein comprises an amino acid sequence of SEQ ID NO: 535. In one example, a BCMA binding trispecific protein comprises an amino acid sequence of SEQ ID NO: 536. In one example, a BCMA binding trispecific protein comprises an amino acid sequence of SEQ ID NO: 537. In one example, a BCMA binding trispecific protein comprises an amino acid sequence of SEQ ID NO: 538. In one example, a BCMA binding trispecific protein comprises an amino acid sequence of SEQ ID NO: 539. In one example, a BCMA binding trispecific protein comprises an amino acid sequence of SEQ ID NO: 540.

In one example, a BCMA binding trispecific protein comprises an amino acid sequence of SEQ ID NO: 541. In one example, a BCMA binding trispecific protein comprises an amino acid sequence of SEQ ID NO: 542. In one example, a BCMA binding trispecific protein comprises an amino acid sequence of SEQ ID NO: 543. In one example, a BCMA binding trispecific protein comprises an amino acid sequence of SEQ ID NO: 544. In one example, a BCMA binding trispecific protein comprises an amino acid sequence of SEQ ID NO: 545. In one example, a BCMA binding trispecific protein comprises an amino acid sequence of SEQ ID NO: 546. In one example, a BCMA binding trispecific protein comprises an amino acid sequence of SEQ ID NO: 547. In one example, a BCMA binding trispecific protein comprises an amino acid sequence of SEQ ID NO: 5048. In one example, a BCMA binding trispecific protein comprises an amino acid sequence of SEQ ID NO: 549. In one example, a BCMA binding trispecific protein comprises an amino acid sequence of SEQ ID NO: 550.

In one example, a BCMA binding trispecific protein comprises an amino acid sequence of SEQ ID NO: 551. In one example, a BCMA binding trispecific protein comprises an amino acid sequence of SEQ ID NO: 552. In one example, a BCMA binding trispecific protein comprises an amino acid sequence of SEQ ID NO: 553. In one example, a BCMA binding trispecific protein comprises an amino acid sequence of SEQ ID NO: 554. In one example, a BCMA binding trispecific protein comprises an amino acid sequence of SEQ ID NO: 555. In one example, a BCMA binding trispecific protein comprises an amino acid sequence of SEQ ID NO: 556. In one example, a BCMA binding trispecific protein comprises an amino acid sequence of SEQ ID NO: 557. In one example, a BCMA binding trispecific protein comprises an amino acid sequence of SEQ ID NO: 558. In one example, a BCMA binding trispecific protein comprises an amino acid sequence of SEQ ID NO: 559.

In one example, a BCMA binding trispecific protein comprises an amino acid sequence of SEQ ID NO: 560. In one example, a BCMA binding trispecific protein comprises an amino acid sequence of SEQ ID NO: 561. In one example, a BCMA binding trispecific protein comprises an amino acid sequence of SEQ ID NO: 562. In one example, a BCMA binding trispecific protein comprises an amino acid sequence of SEQ ID NO: 563. In one example, a BCMA binding trispecific protein comprises an amino acid sequence of SEQ ID NO: 564. In one example, a BCMA binding trispecific protein comprises an amino acid sequence of SEQ ID NO: 565. In one example, a BCMA binding trispecific protein comprises an amino acid sequence of SEQ ID NO: 566. In one example, a BCMA binding trispecific protein comprises an amino acid sequence of SEQ ID NO: 567. In one example, a BCMA binding trispecific protein comprises an amino acid sequence of SEQ ID NO: 568. In one example, a BCMA binding trispecific protein comprises an amino acid sequence of SEQ ID NO: 569.

In one example, a BCMA binding trispecific protein comprises an amino acid sequence of SEQ ID NO: 570. In one example, a BCMA binding trispecific protein comprises an amino acid sequence of SEQ ID NO: 571. In one example, a BCMA binding trispecific protein comprises an amino acid sequence of SEQ ID NO: 572. In one example, a BCMA binding trispecific protein comprises an amino acid sequence of SEQ ID NO: 573. In one example, a BCMA binding trispecific protein comprises an amino acid sequence of SEQ ID NO: 574. In one example, a BCMA binding trispecific protein comprises an amino acid sequence of SEQ ID NO: 575. In one example, a BCMA binding trispecific protein comprises an amino acid sequence of SEQ ID NO: 576. In one example, a BCMA binding trispecific protein comprises an amino acid sequence of SEQ ID NO: 577. In one example, a BCMA binding trispecific protein comprises an amino acid sequence of SEQ ID NO: 578. In one example, a BCMA binding trispecific protein comprises an amino acid sequence of SEQ ID NO: 579.

In one example, a BCMA binding trispecific protein comprises an amino acid sequence of SEQ ID NO: 580. In one example, a BCMA binding trispecific protein comprises an amino acid sequence of SEQ ID NO: 581. In one example, a BCMA binding trispecific protein comprises an amino acid sequence of SEQ ID NO: 582. In one example, a BCMA binding trispecific protein comprises an amino acid sequence of SEQ ID NO: 583. In one example, a BCMA binding trispecific protein comprises an amino acid sequence of SEQ ID NO: 584. In one example, a BCMA binding trispecific protein comprises an amino acid sequence of SEQ ID NO: 585. In one example, a BCMA binding trispecific protein comprises an amino acid sequence of SEQ ID NO: 586. In one example, a BCMA binding trispecific protein comprises an amino acid sequence of SEQ ID NO: 587. In one example, a BCMA binding trispecific protein comprises an amino acid sequence of SEQ ID NO: 588. In one example, a BCMA binding trispecific protein comprises an amino acid sequence of SEQ ID NO: 589.

In one example, a BCMA binding trispecific protein comprises an amino acid sequence of SEQ ID NO: 590. In one example, a BCMA binding trispecific protein comprises an amino acid sequence of SEQ ID NO: 591. In one example, a BCMA binding trispecific protein comprises an amino acid sequence of SEQ ID NO: 592. In one example, a BCMA binding trispecific protein comprises an amino acid sequence of SEQ ID NO: 593. In one example, a BCMA binding trispecific protein comprises an amino acid sequence of SEQ ID NO: 594. In one example, a BCMA binding trispecific protein comprises an amino acid sequence of SEQ ID NO: 595. In one example, a BCMA binding trispecific protein comprises an amino acid sequence of SEQ ID NO: 596. In one example, a BCMA binding trispecific protein comprises an amino acid sequence of SEQ ID NO: 597.

### Polynucleotides Encoding BCMA Targeting Trispecific Proteins

Also provided, in some embodiments, are polynucleotide molecules encoding an anti-BCMA trispecific binding protein described herein. In some embodiments, the polynucleotide molecules are provided as a DNA construct. In other embodiments, the polynucleotide molecules are provided as a messenger RNA transcript.

The polynucleotide molecules are constructed by known methods such as by combining the genes encoding the three binding domains either separated by peptide linkers or, in other embodiments, directly linked by a peptide bond, into a single genetic construct operably linked to a suitable promoter, and optionally a suitable transcription terminator, and expressing it in bacteria or other appropriate expression system such as, for example CHO cells. In the embodiments where the BCMA binding domain is a small molecule, the polynucleotides contain genes encoding the CD3 binding domain and the half-life extension domain. In the embodiments where the half-life extension domain is a small molecule, the polynucleotides contain genes encoding the domains that bind to CD3 and BCMA. Depending on the vector system and host utilized, any number of suitable transcription and translation elements, including constitutive and inducible promoters, may be used. The promoter is selected such that it drives the expression of the polynucleotide in the respective host cell.

In some embodiments, the polynucleotide is inserted into a vector, preferably an expression vector, which represents a further embodiment. This recombinant vector can be constructed according to known methods. Vectors of particular interest include plasmids, phagemids, phage derivatives, virii (e.g., retroviruses, adenoviruses, adeno-associated viruses, herpes viruses, lentiviruses, and the like), and cosmids.

A variety of expression vector/host systems may be utilized to contain and express the polynucleotide encoding the polypeptide of the described trispecific antigen-binding protein. Examples of expression vectors for expression in E. coli are pSKK (Le Gall et al., J Immunol Methods. (2004) 285(1): 111-27) or pcDNA5 (Invitrogen) for expression in mammalian cells.

Thus, the BCMA targeting trispecific proteins as described herein, in some embodiments, are produced by introducing a vector encoding the protein as described above into a host cell and culturing said host cell under conditions whereby the protein domains are expressed, may be isolated and, optionally, further purified.

Integration into Chimeric Antigen Receptors (CAR)

The BCMA targeting trispecific antigen binding proteins of the present disclosure can, in certain examples, be incorporated into a chimeric antigen receptor (CAR). An engineered immune effector cell, e.g., a T cell or NK cell, can be used to express a CAR that includes an anti-BCMA targeting trispecific protein containing an anti-BCMA single domain antibody as described herein. In one embodiment, the CAR including an anti-BCMA targeting trispecific protein as described herein is connected to a transmembrane domain via a hinge region, and further a costimulatory domain, e.g., a functional signaling domain obtained from OX40, CD27, CD28, CD5, ICAM-1, LFA-1 (CD11a/CD18), ICOS (CD278), or 4-1BB. In some embodiments, the CAR further comprises a sequence encoding a intracellular signaling domain, such as 4-1BB and/or CD3 zeta.

### BCMA Trispecific Protein Modifications

The BCMA targeting trispecific proteins described herein encompass derivatives or analogs in which (i) an amino acid is substituted with an amino acid residue that is not one encoded by the genetic code. (ii) the mature polypeptide is fused with another compound such as polyethylene glycol, or (iii) additional amino acids are fused to the protein, such as a leader or secretory sequence or a sequence for purification of the protein.

Typical modifications include, but are not limited to, acetylation, acylation. ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

Modifications are made anywhere in BCMA targeting trispecific proteins described herein, including the peptide backbone, the amino acid side-chains, and the amino or carboxyl termini. Certain common peptide modifications that are useful for modification of BCMA targeting trispecific proteins include glycosylation, lipid attachment, sulfation, gamma-carboxylation of glutamic acid residues, hydroxylation, blockage of the amino or carboxyl group in a polypeptide, or both, by a covalent modification, and ADP-ribosylation.

### Pharmaceutical Compositions

Also provided, in some embodiments, are pharmaceutical compositions comprising an anti-BCMA trispecific binding protein described herein, a vector comprising the polynucleotide encoding the polypeptide of the BCMA targeting trispecific proteins or a host cell transformed by this vector and at least one pharmaceutically acceptable carrier. The term “pharmaceutically acceptable carrier” includes, but is not limited to, any carrier that does not interfere with the effectiveness of the biological activity of the ingredients and that is not toxic to the patient to whom it is administered. Examples of suitable pharmaceutical carriers are well known in the art and include phosphate buffered saline solutions, water, emulsions, such as oil/water emulsions, various types of wetting agents, sterile solutions etc. Such carriers can be formulated by conventional methods and can be administered to the subject at a suitable dose. Preferably, the compositions are sterile. These compositions may also contain adjuvants such as preservative, emulsifying agents and dispersing agents. Prevention of the action of microorganisms may be ensured by the inclusion of various antibacterial and antifungal agents. A further embodiment provides one or more of the above described BCMA targeting trispecific proteins packaged in lyophilized form, or packaged in an aqueous medium.

In some embodiments of the pharmaceutical compositions, the BCMA targeting trispecific proteins described herein are encapsulated in nanoparticles. In some embodiments, the nanoparticles are fullerenes, liquid crystals, liposome, quantum dots, superparamagnetic nanoparticles, dendrimers, or nanorods. In other embodiments of the pharmaceutical compositions, the BCMA trispecific antigen-binding protein is attached to liposomes. In some instances, the BCMA trispecific antigen-binding proteins are conjugated to the surface of liposomes. In some instances, the BCMA trispecific antigen-binding proteins are encapsulated within the shell of a liposome. In some instances, the liposome is a cationic liposome.

The BCMA targeting trispecific proteins described herein are contemplated for use as a medicament. Administration is effected by different ways, e.g. by intravenous, intraperitoneal, subcutaneous, intramuscular, topical or intradermal administration. In some embodiments, the route of administration depends on the kind of therapy and the kind of compound contained in the pharmaceutical composition. The dosage regimen will be determined by the attending physician and other clinical factors. Dosages for any one patient depends on many factors, including the patient's size, body surface area, age, sex, the particular compound to be administered, time and route of administration, the kind of therapy, general health and other drugs being administered concurrently. An “effective dose” refers to amounts of the active ingredient that are sufficient to affect the course and the severity of the disease, leading to the reduction or remission of such pathology and may be determined using known methods.

In some embodiments, the BCMA targeting trispecific proteins of this disclosure are administered at a dosage of up to 10 mg/kg at a frequency of once a week. In some cases, the dosage ranges from about 1 ng/kg to about 10 mg/kg. In some embodiments, the dose is from about 1 ng/kg to about 10 ng/kg, about 5 ng/kg to about 15 ng/kg, about 12 ng/kg to about 20 ng/kg, about 18 ng/kg to about 30 ng/kg, about 25 ng/kg to about 50 ng/kg, about 35 ng/kg to about 60 ng/kg, about 45 ng/kg to about 70 ng/kg, about 65 ng/kg to about 85 ng/kg, about 80 ng/kg to about 1 μg/kg, about 0.5 μg/kg to about 5 μg/kg, about 2 μg/kg to about 10 μg/kg, about 7 μg/kg to about 15 μg/kg, about 12 μg/kg to about 25 μg/kg, about 20 μg/kg to about 50 μg/kg, about 35 μg/kg to about 70 μg/kg, about 45 μg/kg to about 80 μg/kg, about 65 μg/kg to about 90 μg/kg, about 85 μg/kg to about 0.1 mg/kg, about 0.095 mg/kg to about 10 mg/kg. In some cases, the dosage is about 0.1 mg/kg to about 0.2 mg/kg; about 0.25 mg/kg to about 0.5 mg/kg, about 0.45 mg/kg to about 1 mg/kg, about 0.75 mg/kg to about 3 mg/kg, about 2.5 mg/kg to about 4 mg/kg, about 3.5 mg/kg to about 5 mg/kg, about 4.5 mg/kg to about 6 mg/kg, about 5.5 mg/kg to about 7 mg/kg, about 6.5 mg/kg to about 8 mg/kg, about 7.5 mg/kg to about 9 mg/kg, or about 8.5 mg/kg to about 10 mg/kg. The frequency of administration. In some embodiments, is about less than daily, every other day, less than once a day, twice a week, weekly, once in 7 days, once in two weeks, once in two weeks, once in three weeks, once in four weeks, or once a month. In some cases, the frequency of administration is weekly. In some cases, the frequency of administration is weekly and the dosage is up to 10 mg/kg. In some cases, duration of administration is from about 1 day to about 4 weeks or longer.

In some embodiments, the BCMA targeting trispecific proteins of this disclosure are administered at a dosage of about 1 μg to about 100 μg, about 1 μg to about 500 μg, about 1 μg to about 1 mg, about 1 μg to about 2 mg, about 1 μg to about 5 mg, about 1 μg to about 10 mg, about 1 μg to about 100 mg, about 100 μg to about 500 μg, about 100 μg to about 1 mg, about 100 μg to about 2 mg, about 100 μg to about 5 mg, about 100 μg to about 10 mg, about 100 μg to about 100 mg, about 500 μg to about 1 mg, about 500 μg to about 2 mg, about 500 μg to about 5 mg, about 500 μg to about 10 mg, about 500 μg to about 100 mg, about 1 mg to about 2 mg, about 1 mg to about 5 mg, about 1 mg to about 10 mg, about 1 mg to about 100 mg, about 2 mg to about 5 mg, about 2 mg to about 10 mg, about 2 mg to about 100 mg, about 5 mg to about 10 mg, about 5 mg to about 100 mg, or about 10 mg to about 100 mg.

In some embodiments, the BCMA targeting trispecific proteins of this disclosure are administered at a dosage of about 5 μg to about 15 μg, about 5 μg to about 30 μg, about 5 μg to about 90 μg, about 5 μg to about 270 μg, about 5 μg to about 810 μg, about 5 μg to about 1620 μg, about 5 μg to about 2150 μg, about 5 μg to about 2860 μg, about 15 μg to about 30 μg, about 15 μg to about 90 μg, about 15 μg to about 270 μg, about 15 μg to about 810 μg, about 15 μg to about 1620 μg, about 15 μg to about 2150 μg, about 15 μg to about 2860 μg, about 30 μg to about 90 μg, about 30 μg to about 270 μg, about 30 μg to about 810 μg, about 30 μg to about 1620 μg, about 30 μg to about 2150 μg, about 30 μg to about 2860 μg, about 90 μg to about 270 μg, about 90 μg to about 810 μg, about 90 μg to about 1620 μg, about 90 μg to about 2150 μg, about 90 μg to about 2860 μg, about 270 μg to about 810 μg, about 270 μg to about 1620 μg, about 270 μg to about 2150 μg, about 270 μg to about 2860 μg, about 810 μg to about 1620 μg, about 810 μg to about 2150 μg, about 270 μg to about 2860 μg, about 1620 μg to about 2150 μg, or about 1620 μg to about 2860 μg.

The BCMA targeting trispecific protein described herein can be administered using different dosages. In some embodiments, the BCMA targeting trispecific protein of this disclosure is administered according to a schedule comprising the following steps: (i) administration of a first dose of the BCMA targeting trispecific protein, and (ii) administration of a second dose of the BCMA targeting trispecific protein, wherein the second dose is higher than the first dose. In some embodiments, the schedule further comprises step (iii) administration of a third dose of the BCMA targeting trispecific protein, wherein the third dose is higher than the second dose. In some embodiments, the schedule further comprises step (iv) administration of a fourth dose of the BCMA targeting trispecific protein, wherein the fourth dose is higher than the third dose. In some embodiments, the schedule further comprises step (v) administration of a fifth dose of the BCMA targeting trispecific protein, wherein the fifth dose is higher than the fourth dose.

In some embodiments, the first dose is about 1 μg to about 100 μg, about 1 μg to about 500 μg, about 1 μg to about 1 mg, about 1 μg to about 2 mg, about 1 μg to about 5 mg, about 1 μg to about 5 mg, about 1 μg to about 8 mg, about 1 μg to about 10 mg, about 1 μg to about 50 mg, about 1 μg to about 100 mg about 100 μg to about 500 μg, about 100 μg to about 1 mg, about 100 μg to about 2 mg, about 100 μg to about 5 mg, about 100 μg to about 5 mg, about 100 μg to about 8 mg, about 100 μg to about 10 mg, about 100 μg to about 50 mg, about 100 μg to about 100 mg, about 500 μg to about 1 mg, about 500 μg to about 2 mg, about 500 μg to about 5 mg, about 500 μg to about 5 mg, about 500 μg to about 8 mg, about 500 μg to about 10 mg, about 500 μg to about 50 mg, about 500 μg to about 100 mg, about 1 mg to about 2 mg, about 1 mg to about 5 mg, about 1 mg to about 8 mg, about 1 mg to about 10 mg, about 1 mg to about 50 mg, about 1 mg to about 100 mg, about 2 mg to about 5 mg, about 2 mg to about 8 mg, about 2 mg to about 10 mg, about 2 mg to about 50 mg, about 2 mg to about 100 mg, about 5 mg to about 8 mg, about 5 mg to about 10 mg, about 5 mg to about 50 mg, about 5 mg to about 100 mg, about 8 mg to about 10 mg, about 8 mg to about 50 mg, about 8 mg to about 100 mg, about 10 mg to about 50 mg, or about 50 mg to about 100 mg. In some embodiments, the first dose is about 5 μg. In some embodiments, the first dose is about 15 μg. In some embodiments, the first dose is about 30 μg. In some embodiments, the first dose is about 90 μg. In some embodiments, the first dose is about 270 μg. In some embodiments, the first dose is about 810 μg. In some embodiments, the first dose is about 1500 μg. In some embodiments, the first dose is about 1620 μg. In some embodiments, the first dose is about 2150 μg. In some embodiments, the first dose is about 2860 μg. In some embodiments, the first dose is about 3240 μg.

In some embodiments, the first dose is administered for about 1 week to about 5 weeks, about 1 week to about 10 weeks, about 1 week to about 20 weeks, about 1 week to about 50 weeks, about 1 week to about 80 weeks, about 1 week to about 100 weeks, about 5 weeks to about 10 weeks, about 5 weeks to about 20 weeks, about 5 weeks to about 50 weeks, about 5 weeks to about 80 weeks, about 5 weeks to about 100 weeks, about 10 weeks to about 20 weeks, about 10 weeks to about 50 weeks, about 10 weeks to about 80 weeks, about 10 weeks to about 100 weeks, about 20 weeks to about 50 weeks, about 20 weeks to about 80 weeks, about 20 weeks to about 100 weeks, about 50 weeks to about 80 weeks, about 50 weeks to about 100 weeks, about 80 weeks to about 100 weeks, about 1 week to about 9 weeks, about 1 week to about 18 weeks, about 1 week to about 27 weeks, about 1 week to about 36 weeks, about 9 weeks to about 18 weeks, about 9 weeks to about 27 weeks, about 9 weeks to about 36 weeks, about 18 weeks to about 27 weeks, about 18 weeks to about 36 weeks, or about 27 weeks to about 36 weeks.

In some embodiments, the first dose is administered once per day, twice per day, three times per day, four times per day, five times per day, six times per day, seven times per day, eight times per day, nine times per day or ten times per day. In some embodiments, the first dose is administered once per week, twice per week, three times per week, four times per week, five times per week, six times per week, once every other week, once every three weeks, once every four week or once every five weeks.

In some embodiments, the second dose is about 1 μg to about 100 μg, about 1 μg to about 500 μg, about 1 μg to about 1 mg, about 1 μg to about 2 mg, about 1 μg to about 5 mg, about 1 μg to about 5 mg, about 1 μg to about 8 mg, about 1 μg to about 10 mg, about 1 μg to about 50 mg, about 1 μg to about 100 mg about 100 μg to about 500 μg, about 100 μg to about 1 mg, about 100 μg to about 2 mg, about 100 μg to about 5 mg, about 100 μg to about 5 mg, about 100 μg to about 8 mg, about 100 μg to about 10 mg, about 100 μg to about 50 mg, about 100 μg to about 100 mg, about 500 μg to about 1 mg, about 500 μg to about 2 mg, about 500 μg to about 5 mg, about 500 μg to about 5 mg, about 500 μg to about 8 mg, about 500 μg to about 10 mg, about 500 μg to about 50 mg, about 500 μg to about 100 mg, about 1 mg to about 2 mg, about 1 mg to about 5 mg, about 1 mg to about 8 mg, about 1 mg to about 10 mg, about 1 mg to about 50 mg, about 1 mg to about 100 mg, about 2 mg to about 5 mg, about 2 mg to about 8 mg, about 2 mg to about 10 mg, about 2 mg to about 50 mg, about 2 mg to about 100 mg, about 5 mg to about 8 mg, about 5 mg to about 10 mg, about 5 mg to about 50 mg, about 5 mg to about 100 mg, about 8 mg to about 10 mg, about 8 mg to about 50 mg, about 8 mg to about 100 mg, about 10 mg to about 50 mg, or about 50 mg to about 100 mg. In some embodiments, the second dose is about 1 mg to about 6 mg, about 1 mg to about 12 mg, about 1 to about 24 mg, about 1 mg to about 36 mg, about 1 to about 48 mg, about 6 mg to about 12 mg, about 6 to about 24 mg, about 6 mg to about 36 mg, about 6 to about 48 mg, about 12 to about 24 mg, about 12 mg to about 36 mg, about 12 to about 48 mg, about 24 mg to about 36 mg, about 24 to about 48 mg, or about 36 to about 48 mg. In some embodiments, the second dose is about 5 μg. In some embodiments, the second dose is about 15 μg. In some embodiments, the second dose is about 30 μg. In some embodiments, the second dose is about 90 μg. In some embodiments, the second dose is about 270 μg. In some embodiments, the second dose is about 810 μg. In some embodiments, the second dose is about 1620 μg. In some embodiments, the second dose is about 2150 μg. In some embodiments, the second dose is about 2860 μg. In some embodiments, the second dose is about 3240 μg. In some embodiments, the second dose is about 5 mg. In some embodiments, the second dose is about 10 mg. In some embodiments, the second dose is about 12 mg. In some embodiments, the second dose is about 24 mg. In some embodiments, the second dose is about 36 mg. In some embodiments, the second dose is about 48 mg.

In some embodiments, the second dose is administered for about 1 week to about 5 weeks, about 1 week to about 10 weeks, about 1 week to about 20 weeks, about 1 week to about 50 weeks, about 1 week to about 80 weeks, about 1 week to about 100 weeks, about 5 weeks to about 10 weeks, about 5 weeks to about 20 weeks, about 5 weeks to about 50 weeks, about 5 weeks to about 80 weeks, about 5 weeks to about 100 weeks, about 10 weeks to about 20 weeks, about 10 weeks to about 50 weeks, about 10 weeks to about 80 weeks, about 10 weeks to about 100 weeks, about 20 weeks to about 50 weeks, about 20 weeks to about 80 weeks, about 20 weeks to about 100 weeks, about 50 weeks to about 80 weeks, about 50 weeks to about 100 weeks, about 80 weeks to about 100 weeks, about 1 week to about 9 weeks, about 1 week to about 18 weeks, about 1 week to about 27 weeks, about 1 week to about 36 weeks, about 9 weeks to about 18 weeks, about 9 weeks to about 27 weeks, about 9 weeks to about 36 weeks, about 18 weeks to about 27 weeks, about 18 weeks to about 36 weeks, or about 27 weeks to about 36 weeks.

In some embodiments, the second dose is administered once per day, twice per day, three times per day, four times per day, five times per day, six times per day, seven times per day, eight times per day, nine times per day or ten times per day. In some embodiments, the first dose is administered once per week, twice per week, three times per week, four times per week, five times per week, six times per week, once every other week, once every three weeks, once every four week or once every five weeks.

### Methods of Treatment

In certain embodiments, the BCMA targeting trispecific proteins of the disclosure reduce the growth of tumor cells in vivo when administered to a subject who has tumor cells that express BCMA. Measurement of the reduction of the growth of tumor cells can be determined by multiple different methodologies well known in the art. Non-limiting examples include direct measurement of tumor dimension, measurement of excised tumor mass and comparison to control subjects, measurement via imaging techniques (e.g., CT or MRI) that may or may not use isotopes or luminescent molecules (e.g., luciferase) for enhanced analysis, and the like. In specific embodiments, administration of the trispecific proteins of the disclosure results in a reduction of in vivo growth of tumor cells as compared to a control antigen binding agent by at least about 10%, 20%, 30%, 40%, 50%, 60%, 70%, 80%, 90% or 100%, with an about 100% reduction in tumor growth indicating a complete response and disappearance of the tumor. In further embodiments, administration of the trispecific proteins of the disclosure results in a reduction of in vivo growth of tumor cells as compared to a control antigen binding agent by about 50-100%, about 75-100% or about 90-100%. In further embodiments, administration of the trispecific proteins of the disclosure results in a reduction of in vivo growth of tumor cells as compared to a control antigen binding agent by about 50-60%, about 60-70%, about 70-80%, about 80-90%, or about 90-100%.

Also provided herein, in some embodiments, are methods and uses for stimulating the immune system of an individual in need thereof comprising administration of an anti-BCMA targeting trispecific protein as described herein. In some instances, the administration of an anti-BCMA targeting trispecific protein described herein induces and/or sustains cytotoxicity towards a cell expressing a target antigen.

Also provided herein, in some embodiments, are methods and uses for stimulating the immune system of an individual in need thereof comprising administration of a BCMA binding protein as described herein. In some instances, the administration of a BCMA binding protein described herein induces and/or sustains cytotoxicity towards a cell expressing a target antigen. In some instances, the cell expressing a target antigen is a terminally differentiated B cell that is a cancer or tumor cell, or a metastatic cancer or tumor cell.

Also provided herein are methods and uses for a treatment of a disease, disorder or condition associated with BCMA comprising administering to an individual in need thereof a BCMA binding protein or a multispecific binding protein comprising the BCMA binding protein described herein.

Diseases, disorders or conditions associated with BCMA include, but are not limited to, a cancer or a metastasis that is of a B cell lineage.

Cancers that can be treated, prevented, or managed by the BCMA binding proteins of the present disclosure, and methods of using them, include but are not limited to a primary cancer or a metastatic cancer.

Examples of such leukemias include, but are not limited to: acute lymphoblastic leukemia (ALL), acute myeloid leukemia (AML), chronic lymphocytic leukemia (CLL) and chronic myeloid leukemia (CML), as well as a number of less common types such as, for example, Hairy cell leukemia (HCL), T-cell prolymphocytic leukemia (T-PLL), Large granular lymphocytic leukemia and Adult T-cell leukemia, etc. Acute lymphoblastic leukemia (ALL) subtypes to be treated include, but are not limited to, precursor B acute lymphoblastic leukemia, precursor T acute lymphoblastic leukemia, Burkitt's leukemia, and acute biphenotypic leukemia, Chronic lymphocytic leukemia (CLL) subtypes to be treated include, but are not limited to, B-cell prolymphocytic leukemia. Acute myelogenous leukemia (AML) subtypes to be treated include, but are not limited to, acute promyelocytic leukemia, acute myeloblastic leukemia and acute megakaryoblastic leukemia. Chronic myelogenous leukemia (CML) subtypes to be treated include, but are not limited to, chronic myelomonocytic leukemia.

Examples of a lymphoma to be treated with the subject methods include, but not limited to Hodgkin's disease, non-Hodgkin's disease, or any subtype of lymphoma.

Examples of such multiple myelomas include, but are not limited to, a multiple myeloma of the bone or other tissues including, for example, a smoldering multiple myeloma, a non-secretory myeloma, am osteosclerotic myeloma, etc.

For a review of such disorders, see Fishman et al., 1985. Medicine. 2d Ed., J.B. Lippincott Co., Philadelphia and Murphy et al., 1997. Informed Decisions: The Complete Book of Cancer Diagnosis. Treatment, and Recovery. Viking Penguin. Penguin Books U.S.A., Inc., United States of America).

As used herein, in some embodiments. “treatment” or “treating” or “treated” refers to therapeutic treatment wherein the object is to slow (lessen) an undesired physiological condition, disorder or disease, or to obtain beneficial or desired clinical results. For the purposes described herein, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment. In other embodiments. “treatment” or “treating” or “treated” refers to prophylactic measures, wherein the object is to delay onset of or reduce severity of an undesired physiological condition, disorder or disease, such as, for example is a person who is predisposed to a disease (e.g., an individual who carries a genetic marker for a disease such as breast cancer).

In some embodiments of the methods described herein, the BCMA targeting trispecific proteins as described herein are administered in combination with an agent for treatment of the particular disease, disorder or condition. Agents include, but are not limited to, therapies involving antibodies, small molecules (e.g., chemotherapeutics), hormones (steroidal, peptide, and the like), radiotherapies (y-rays, X-rays, and/or the directed delivery of radioisotopes, microwaves, UV radiation and the like), gene therapies (e.g., antisense, retroviral therapy and the like) and other immunotherapies. In some embodiments, an anti-BCMA targeting trispecific protein as described herein is administered in combination with anti-diarrheal agents, anti-emetic agents, analgesics, opioids and/or non-steroidal anti-inflammatory agents. In some embodiments, an anti-BCMA targeting trispecific protein as described herein is administered in combination with anti-cancer agents.

Non-limiting examples of anti-cancer agents that can be used in the various embodiments of the disclosure, including pharmaceutical compositions and dosage forms and kits of the disclosure, include: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alpha-2a; interferon alpha-2b; interferon alpha-n1 interferon alpha-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinzolidine sulfate; vinzolidine sulfate; vorozole; zeniplatin; zinostatin; zorubicin hydrochloride. Other examples of anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; avinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-I receptor inhibitor; interferon agonists; interferons; interleukins; iobenguane; iododoxorubicin; ipomeanol. 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; HMG-COA reductase inhibitor (such as but not limited to, Lovastatin, Pravastatin, Fluvastatin, Statin, Simvastatin, and Atorvastatin); loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; VITAXIN®; vorozole; zanoterone; zeniplatin; zilascorb; and zinostatin stimalamer. Additional anti-cancer drugs are 5-fluorouracil and leucovorin. These two agents are particularly useful when used in methods employing thalidomide and a topoisomerase inhibitor. In some embodiments, the anti-BCMA targeting trispecific protein of the present disclosure is used in combination with gemcitabine.

In some embodiments, the anti-BCMA targeting trispecific protein as described herein is administered before, during, or after surgery.

In some embodiments, the anti-cancer agent is conjugated via any suitable means to the trispecific protein.

### Methods of Detection of BCMA Expression and Diagnosis of BCMA Associated Cancer

According to another embodiment of the disclosure, kits for detecting expression of BCMA in vitro and/or in vivo are provided. The kits include the foregoing BCMA targeting trispecific proteins (e.g., a trispecific protein containing a labeled anti-BCMA single domain antibody or antigen binding fragments thereof), and one or more compounds for detecting the label. In some embodiments, the label is selected from the group consisting of a fluorescent label, an enzyme label, a radioactive label, a nuclear magnetic resonance active label, a luminescent label, and a chromophore label.

In some cases. BCMA expression is detected in a biological sample. The sample can be any sample, including, but not limited to, tissue from biopsies, autopsies and pathology specimens. Biological samples also include sections of tissues, for example, frozen sections taken for histological purposes. Biological samples further include body fluids, such as blood, serum, plasma, sputum, spinal fluid or urine. A biological sample is typically obtained from a mammal, such as a human or non-human primate.

Samples to be obtained for use in an assay described herein include tissues and bodily fluids may be processed using conventional means in the art (e.g., homogenization, serum isolation, etc.). Accordingly, a sample obtained from a patient is transformed prior to use in an assay described herein. BCMA, if present in the sample, is further transformed in the methods described herein by virtue of binding to, for example, an antibody.

In one embodiment, provided is a method of determining if a subject has cancer by contacting a sample from the subject with an anti-BCMA single domain antibody as disclosed herein; and detecting binding of the single domain antibody to the sample. An increase in binding of the antibody to the sample as compared to binding of the antibody to a control sample identifies the subject as having cancer.

In another embodiment, provided is a method of confirming a diagnosis of cancer in a subject by contacting a sample from a subject diagnosed with cancer with an anti-BCMA single domain antibody as disclosed herein; and detecting binding of the antibody to the sample. An increase in binding of the antibody to the sample as compared to binding of the antibody to a control sample confirms the diagnosis of cancer in the subject.

In some examples of the disclosed methods, the BCMA single domain antibody of the trispecific protein is directly labeled.

In some examples, the methods further include contacting a second antibody that specifically binds the anti-BCMA single domain antibody with the sample; and detecting the binding of the second antibody. An increase in binding of the second antibody to the sample as compared to binding of the second antibody to a control sample detects cancer in the subject or confirms the diagnosis of cancer in the subject.

In some cases, the cancer is a leukemia, a lymphoma, a multiple myeloma, or any other type of cancer that expresses BCMA.

In some examples, the control sample is a sample from a subject without cancer. In particular examples, the sample is a blood or tissue sample.

In some cases, the antibody that binds (for example specifically binds) BCMA is directly labeled with a detectable label. In another embodiment, the antibody that binds (for example, specifically binds) BCMA (the first antibody) is unlabeled and a second antibody or other molecule that can bind the antibody that specifically binds BCMA is labeled. A second antibody is chosen such that it is able to specifically bind the specific species and class of the first antibody. For example, if the first antibody is a llama IgG, then the secondary antibody may be an anti-llama-IgG. Other molecules that can bind to antibodies include, without limitation. Protein A and Protein G, both of which are available commercially. Suitable labels for the antibody or secondary antibody are described above, and include various enzymes, prosthetic groups, fluorescent materials, luminescent materials, magnetic agents and radioactive materials. Non-limiting examples of suitable enzymes include horseradish peroxidase, alkaline phosphatase, beta-galactosidase, or acetylcholinesterase. Non-limiting examples of suitable prosthetic group complexes include streptavidin/biotin and avidin/biotin. Non-limiting examples of suitable fluorescent materials include umbelliferone, fluorescein, fluorescein isothiocyanate, rhodamine, dichlorotriazinylamine fluorescein, dansyl chloride or phycoerythrin. A non-limiting exemplary luminescent material is luminol; a non-limiting exemplary a magnetic agent is gadolinium, and non-limiting exemplary radioactive labels include 125I, 131I, 35S or 3H.

In an alternative embodiment. BCMA can be assayed in a biological sample by a competition immunoassay utilizing BCMA standards labeled with a detectable substance and an unlabeled antibody that specifically binds BCMA. In this assay, the biological sample, the labeled BCMA standards and the antibody that specifically bind BCMA are combined and the amount of labeled BCMA standard bound to the unlabeled antibody is determined. The amount of BCMA in the biological sample is inversely proportional to the amount of labeled BCMA standard bound to the antibody that specifically binds BCMA.

The immunoassays and method disclosed herein can be used for a number of purposes. In one embodiment, the antibody that specifically binds BCMA may be used to detect the production of BCMA in cells in cell culture. In another embodiment, the antibody can be used to detect the amount of BCMA in a biological sample, such as a tissue sample, or a blood or serum sample. In some examples, the BCMA is cell-surface BCMA. In other examples, the BCMA is soluble BCMA (e.g., BCMA in a cell culture supernatant or soluble BCMA in a body fluid sample, such as a blood or serum sample).

In one embodiment, a kit is provided for detecting BCMA in a biological sample, such as a blood sample or tissue sample. For example, to confirm a cancer diagnosis in a subject, a biopsy can be performed to obtain a tissue sample for histological examination. Alternatively, a blood sample can be obtained to detect the presence of soluble BCMA protein or fragment. Kits for detecting a polypeptide will typically comprise a single domain antibody, according to the present disclosure, that specifically binds BCMA. In some embodiments, an antibody fragment, such as a scFv fragment, a VH domain, or a Fab is included in the kit. In a further embodiment, the antibody is labeled (for example, with a fluorescent, radioactive, or an enzymatic label).

In one embodiment, a kit includes instructional materials disclosing means of use of an antibody that binds BCMA. The instructional materials may be written, in an electronic form (such as a computer diskette or compact disk) or may be visual (such as video files), or provided through an electronic network, for example, over the internet. World Wide Web, an intranet, or other network. The kits may also include additional components to facilitate the particular application for which the kit is designed. Thus, for example, the kit may additionally contain means of detecting a label (such as enzyme substrates for enzymatic labels, filter sets to detect fluorescent labels, appropriate secondary labels such as a secondary antibody, or the like). The kits may additionally include buffers and other reagents routinely used for the practice of a particular method. Such kits and appropriate contents are well known to those of skill in the art.

In one embodiment, the diagnostic kit comprises an immunoassay. Although the details of the immunoassays may vary with the particular format employed, the method of detecting BCMA in a biological sample generally includes the steps of contacting the biological sample with an antibody which specifically reacts, under immunologically reactive conditions, to a BCMA polypeptide. The antibody is allowed to specifically bind under immunologically reactive conditions to form an immune complex, and the presence of the immune complex (bound antibody) is detected directly or indirectly.

Methods of determining the presence or absence of a cell surface marker are well known in the art. For example, the antibodies can be conjugated to other compounds including, but not limited to, enzymes, magnetic beads, colloidal magnetic beads, haptens, fluorochromes, metal compounds, radioactive compounds or drugs. The antibodies can also be utilized in immunoassays such as but not limited to radioimmunoassays (RIAs), ELISA, or immunohistochemical assays. The antibodies can also be used for fluorescence activated cell sorting (FACS). FACS employs a plurality of color channels, low angle and obtuse light-scattering detection channels, and impedance channels, among other more sophisticated levels of detection, to separate or sort cells (see. U.S. Pat. No. 5,061,620). Any of the single domain antibodies that bind BCMA, as disclosed herein, can be used in these assays. Thus, the antibodies can be used in a conventional immunoassay, including, without limitation, an ELISA, an RIA, FACS, tissue immunohistochemistry, western blot or immunoprecipitation.

### EXAMPLES

The application may be better understood by reference to the following non-limiting examples, which are provided as exemplary embodiments of the application. The following examples are presented in order to more fully illustrate embodiments and should in no way be construed, however, as limiting the broad scope of the application.

### Example 1

### Protein Production

Sequences of BCMA targeting trispecific molecules, containing a BCMA binding protein according to the present disclosure, were cloned into mammalian expression vector pcDNA 3.4 (Invitrogen) preceded by a leader sequence and followed by a 6× Histidine Tag (SEQ ID NO: 471). Expi293 cells (Life Technologies A14527) were maintained in suspension in Optimum Growth Flasks (Thomson) between 0.2 to 8×1e6 cells/mL in Expi293 media. Purified plasmid DNA was transfected into Expi293 cells in accordance with Expi293 Expression System Kit (Life Technologies. A14635) protocols, and maintained for 4-6 days post transfection. The amount of the exemplary trispecific proteins being tested, in the conditioned media, from the transfected Expi293 cells was quantitated using an Octet instrument with Protein A tips and using a control trispecific protein for a standard curve.

### T Cell Dependent Cellular Cytotoxicity Assays

Titrations of conditioned media was added to TDCC assays (T cell Dependent Cell Cytotoxicity assays) to assess whether the anti-BCMA single domain antibody is capable of forming a synapse between T cells and a BCMA-expressing cell line and direct the T cells to kill the BCMA-expressing cell line. In this assay (Nazarian et al., 2015. J. Biomol. Screen., 20:519-27). T cells and target cancer cell line cells were mixed together at a 10:1 ratio in a 384-well plate, and varying amounts of the trispecific proteins being tested were added. The tumor cell lines were engineered to express luciferase protein. After 48 hours, to quantitate the remaining viable tumor cells. STEADY-GLO® Luminescent Assay (Promega) was used.

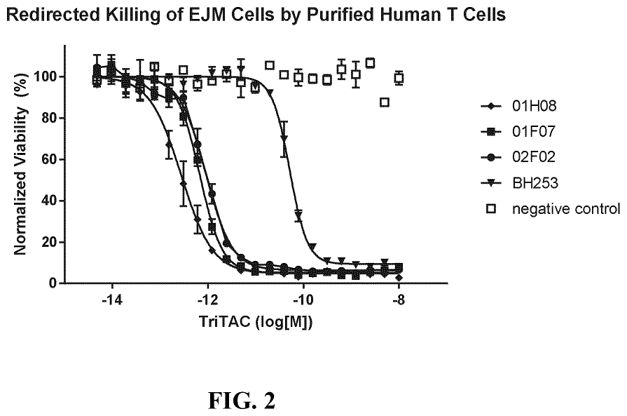

In this example EJM cells were used, which is a cell line that serves as an in vitro model for multiple myeloma and plasma cell leukemia. Viability of the EJM cells is measured after 48 hours. It was seen that the trispecific proteins mediated T cell killing. FIG. 2 shows an example cell viability assay with test proteins 01H08, 01F07, 02F02 and BH253 compared to a negative control. The EC₅₀ for the TDCC activity of several other test trispecific proteins are listed below in Table 1.

### Binding Affinity

In the instant study, the binding affinity to human BCMA protein of the BCMA targeting trispecific proteins containing a BCMA binding protein according to the present disclosure was determined. The affinity measurements are listed in Table 1.

TABLE 1
Binding affinity and TDCC Activity of several
BCMA targeting trispecific proteins.

| | Human | |
|---|---|---|
| Construct | BCMA | TDCC |
| Name | KD (M) | EC50 (M) |
| 253BH10 | 2.77E−08 | 5.29E−11 |
| 01H08 | 2.86E−09 | 3.41E−13 |
| 01F07 | 4.18E−09 | 7.02E−13 |
| 01H06 | ND | 1.00E−12 |
| 02G02 | 5.26E−09 | 1.08E−12 |
| 02B05 | 5.39E−09 | 1.22E−12 |
| 01C01 | 6.52E−09 | 1.33E−12 |
| 02F02 | 6.73E−09 | 1.36E−12 |
| 02E05 | 6.53E−09 | 1.37E−12 |
| 02C01 | 5.31E−09 | 1.55E−12 |
| 02E06 | 6.31E−09 | 1.57E−12 |
| 02B06 | 6.77E−09 | 1.65E−12 |
| 02F04 | 6.75E−09 | 1.72E−12 |
| 01G08 | 6.27E−09 | 1.91E−12 |
| 02C06 | 6.90E−09 | 1.95E−12 |
| 01H09 | 5.44E−09 | 2.21E−12 |
| 01F04 | 6.55E−09 | 2.21E−12 |
| 01D02 | 7.35E−09 | 2.25E−12 |
| 02D11 | 6.71E−09 | 2.35E−12 |
| 01A07 | 6.95E−09 | 2.49E−12 |
| 02C03 | 7.09E−09 | 2.52E−12 |
| 02F07 | 7.06E−09 | 2.59E−12 |
| 01E04 | 7.29E−09 | 2.67E−12 |
| 02H09 | 6.83E−09 | 2.88E−12 |
| 01E03 | 6.36E−09 | 2.98E−12 |
| 02F05 | 7.15E−09 | 3.00E−12 |
| 01B05 | 6.52E−09 | 3.01E−12 |
| 01C05 | 6.09E−09 | 3.07E−12 |
| 02F12 | 7.76E−09 | 3.14E−12 |
| 01H11 | 7.06E−09 | 3.17E−12 |
| 02G06 | 7.50E−09 | 3.39E−12 |
| 01E06 | 8.91E−09 | 3.77E−12 |
| 01G11 | 9.70E−09 | 3.98E−12 |
| 02A05 | 7.06E−09 | 4.21E−12 |
| 01A08 | 1.17E−08 | 4.25E−12 |
| 02G05 | 7.12E−09 | 4.33E−12 |
| 01B09 | 1.12E−08 | 5.27E−12 |
| 01G01 | 1.46E−08 | 5.83E−12 |
| 01B06 | 9.10E−09 | 6.97E−12 |
| 01F10 | 1.44E−08 | 7.44E−12 |
| 01E05 | 1.17E−08 | 1.08E−11 |
| 02G01 | 1.63E−08 | 1.08E−11 |
| 01A06 | 1.58E−08 | 1.10E−11 |
| 02B04 | 1.52E−08 | 1.13E−11 |
| 01D06 | 1.49E−08 | 1.35E−11 |
| 02B07 | 1.58E−08 | 1.42E−11 |
| 02B11 | 1.33E−08 | 1.44E−11 |
| 01H04 | 1.74E−08 | 1.47E−11 |
| 01D03 | 2.09E−08 | 1.49E−11 |
| 01A05 | 1.70E−08 | 1.51E−11 |
| 02F11 | 2.00E−08 | 1.52E−11 |
| 01D04 | 1.89E−08 | 1.60E−11 |
| 01B04 | 1.86E−08 | 1.61E−11 |
| 02C05 | 1.56E−08 | 1.62E−11 |
| 02E03 | 1.68E−08 | 1.65E−11 |
| 01D05 | 1.78E−08 | 1.66E−11 |
| 01C04 | 2.16E−08 | 1.75E−11 |
| 01E07 | 1.99E−08 | 1.92E−11 |
| 01G06 | 1.70E−08 | 1.92E−11 |
| 02F06 | 2.19E−08 | 1.93E−11 |
| 01B01 | 1.99E−08 | 1.95E−11 |
| 01D07 | 1.93E−08 | 1.96E−11 |
| 02A08 | 9.51E−09 | 2.01E−11 |
| 01A02 | 2.15E−08 | 2.18E−11 |
| 02G11 | 2.05E−08 | 2.38E−11 |
| 01G04 | 1.17E−08 | 2.41E−11 |
| 02F03 | 2.57E−08 | 2.45E−11 |
| 01C06 | 1.88E−08 | 2.51E−11 |
| 01A01 | 2.13E−08 | 2.64E−11 |
| 01B12 | 2.07E−08 | 2.73E−11 |
| 02A07 | 1.84E−08 | 2.79E−11 |
| 02G08 | 1.80E−08 | 2.86E−11 |
| 02E09 | 2.09E−08 | 3.11E−11 |
| 02H06 | 2.33E−08 | 3.19E−11 |
| 01H10 | 2.48E−08 | 3.52E−11 |
| 01F05 | 1.67E−08 | 3.72E−11 |
| 01C02 | 2.00E−08 | 3.73E−11 |
| 02A04 | 1.76E−08 | 3.82E−11 |
| 02H05 | 1.96E−08 | 3.89E−11 |
| 02G09 | 3.44E−08 | 3.96E−11 |
| 02D06 | 2.33E−08 | 4.28E−11 |
| 02G07 | 1.93E−08 | 4.46E−11 |
| 01H05 | 2.74E−08 | 4.54E−11 |
| 01C08 | 2.83E−08 | 4.57E−11 |
| 01A03 | 3.08E−08 | 4.61E−11 |
| 01A09 | 2.39E−08 | 4.84E−11 |
| 02B01 | 2.14E−08 | 5.18E−11 |
| 02H01 | 3.56E−08 | 5.42E−11 |
| 02H04 | 3.11E−08 | 5.99E−11 |
| 02A11 | 2.52E−08 | 6.06E−11 |
| 01E10 | 1.85E−08 | 6.23E−11 |
| 02D09 | 2.89E−08 | 6.73E−11 |
| 01F08 | 2.14E−08 | 7.12E−11 |
| 01F03 | 1.50E−08 | 7.64E−11 |
| 02H11 | 2.75E−08 | 7.75E−11 |
| 01C07 | 1.98E−08 | 8.33E−11 |
| 01B08 | 2.56E−08 | 8.76E−11 |
| 01B03 | 2.62E−08 | 9.64E−11 |
| 01H01 | 3.59E−08 | 1.18E−10 |
| 02B12 | 2.52E−08 | 1.24E−10 |
| 01G10 | 4.19E−08 | 1.43E−10 |
| 01A04 | 3.75E−08 | 1.59E−10 |
| 01B07 | 4.39E−08 | 1.74E−10 |
| 01C10 | 4.64E−08 | 2.08E−10 |
| 01F02 | 4.13E−08 | 2.25E−10 |
| 01B02 | 1.88E−08 | 3.59E−10 |
| 01F12 | 4.05E−08 | 3.92E−10 |
| 01G09 | 8.78E−08 | 4.41E−10 |
| 01D10 | 5.39E−08 | 4.53E−10 |
| 01F09 | 5.28E−08 | 9.45E−10 |

ND: Not determined.

Molecules 01H08, 01F07, 01H06, 02G02, 02B05, 01C01, 02F02, 02E05, 01E08, 02C01, 02E06, 02B06, 02F04, 01G08, 02C06, 01H09, 01F04, 01D02, 02D11, 01A07, 02C03, 02F07, 01E04, 02H09, 01E03, 02F05, 01B05, 01C05, 02F12, 01H11, 02G06, 01E06, 01G11, 02A05, 01A08, 02G05, 01B09, 01G01, 01B06, 01F10, 01E05, 02G01, 01A06, 02B04, 01D06, 02B07, 02B11, 01H04, 01D03, 01A05, 02F11, 01D04, 01B04, 02C05, 02E03, 01D05, 01C04, 01E07, 01G06, 02F06, 01B01, 01D07, 02A08, 01A02, 02G11, 01G04, 02F03, 01C06, 01A01 have at least two fold increase TDCC potency and also show increase affinity compared to a molecule with the parental CDRs. 253BH10.

Molecules 01H08, 01F07, 01H06, 02G02, 02B05, 01C01, 02F02, 02E05, 01E08, 02C01, 02E06, 02B06, 02F04, 01G08, 02C06, 01H09, 01F04, 01D02, 02D11, 01A07, 02C03, 02F07, 01E04, 02H09, 01E03, 02F05, 01B05, 01C05, 02F12, 01H11, 02G06, 01E06, 01G11, 02A05, 01A08, 02G05, 01B09 have at least ten-fold increase TDCC potency and also show increase affinity compared to a molecule with the parental CDRs, 253BH10.

An anti-GFP trispecific molecule, included in these assays as a negative control, had no detectable BCMA binding and no effect on cell viability in the TDCC assay (data not shown).

### Example 2: Methods to Assess Binding and Cytotoxic Activities of Exemplary BCMA Targeting Trispecific Proteins According to the Present Disclosure Against Jeko1, MOLP8 and OPM2 Cells

### Protein Production

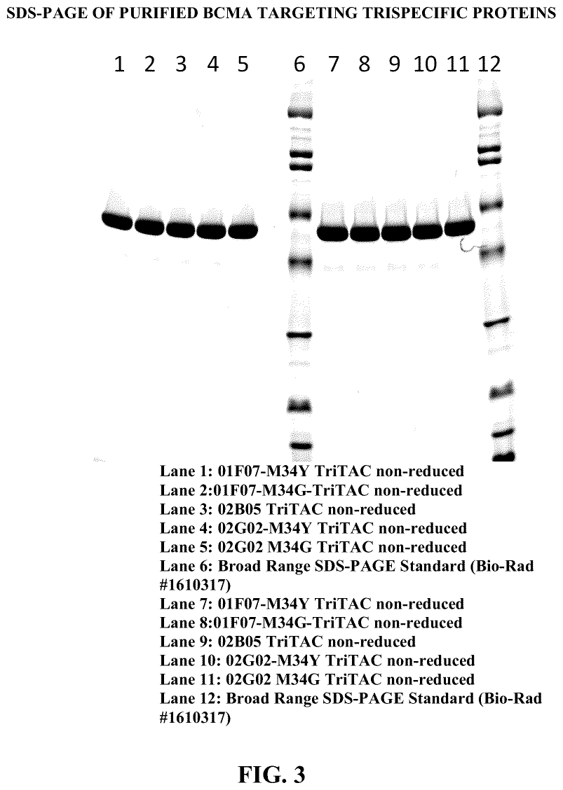

Sequences of BCMA targeting trispecific molecules, containing a BCMA binding protein according to the present disclosure, preceded by a leader sequence and followed by a 6× Histidine Tag (SEQ ID NO: 471), were expressed using the vectors and methods previously described (Running Deer and Allison, 2004, Biotechnol Prog. 20:880-9) except lipid-based reagents and non-linearized plasmid DNA were used for cell transfection. Recombinant trispecific proteins were purified using affinity chromatography, ion exchange, and/or size exclusion chromatography. Purified protein was quantitated using theoretical extinction coefficients and absorption spectroscopy. An image of a Coomassie stained SDS-PAGE demonstrates the purity of the proteins (FIG. 3).

### Cytotoxicity Assays

A human T-cell dependent cellular cytotoxicity (TDCC) assay was used to measure the ability of T cell engagers, including trispecific molecules, to direct T cells to kill tumor cells (Nazarian et al., 2015, J. Biomol. Screen., 20:519-27). In this assay, T cells and target cancer cell line cells are mixed together at a 10:1 ratio in a 384-well plate, and varying amounts of the trispecific proteins being tested are added. The tumor cell lines are engineered to express luciferase protein. After 48 hours, to quantitate the remaining viable tumor cells, Steady-Glo® Luminescent Assay (Promega) was used.

In the instant study, titrations of purified protein were added to TDCC assays (T cell Dependent Cell Cytotoxicity assays) to assess whether the anti-BCMA single domain antibody was capable of forming a synapse between T cells and BCMA-expressing Jeko1, MOLP8 and OPM2 cancer cell lines. Jeko1 is a B cell lymphoma cell line. MOLP-8 is a myeloma cell line. OPM-2 is a human myeloma cell line.

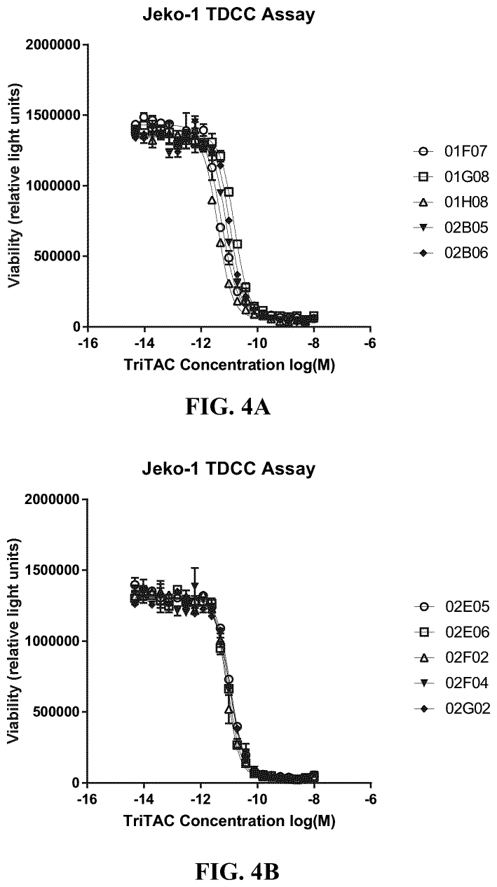

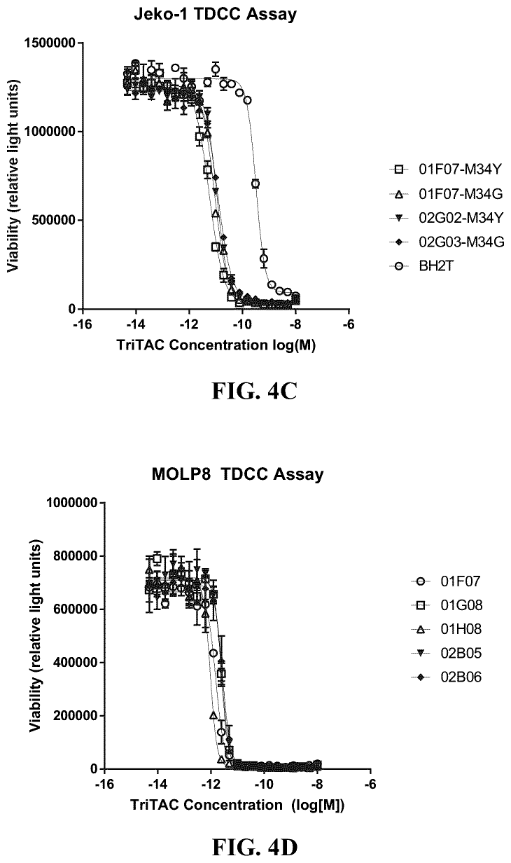

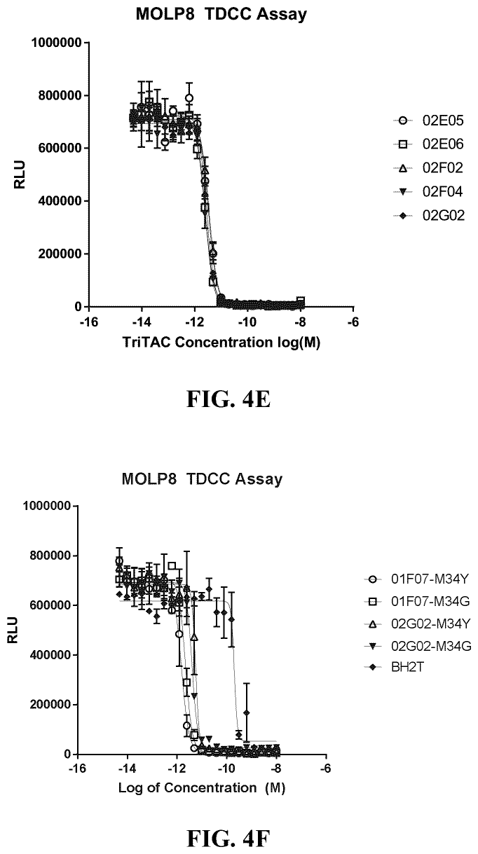

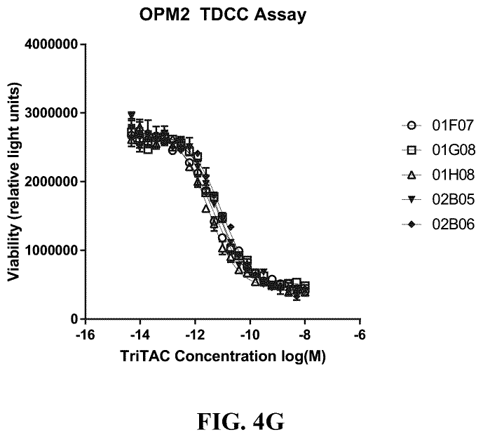

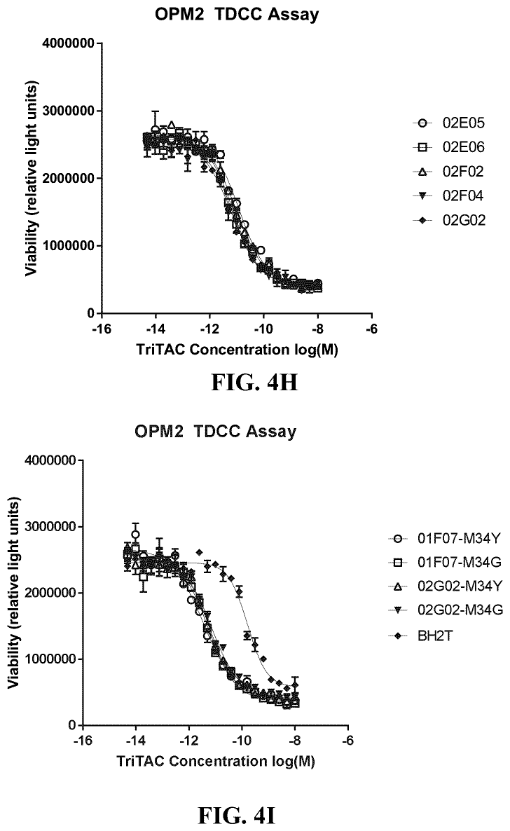

Viability of the cells was measured after 48 hours. It was seen that the trispecific proteins mediated T cell killing. FIG. 4 shows an example cell viability assay with test proteins compared to a negative control. The EC₅₀ for the TDCC activity of several other test trispecific proteins are listed below in Table 2. An anti-GFP trispecific molecule, included in these assays as a negative control, had no effect on cell viability (data not shown).

TABLE 2
TDCC EC₅₀ Values for 3 Cell Lines for Select BCMA targeting
trispecific proteins in TriTAC format (anti-target
(BCMA): anti-albumin: anti-CD3 binding domains).

| | Jeko1 | MOLP-8 | OPM-2 |
|---|---|---|---|
| Construct name | EC50 (M) | EC50 (M) | EC50 (M) |
| BH2T TriTAC | 3.2E−10 | 2.0E−10 | 1.6E−10 |
| 01F07 TriTAC | 5.3E−12 | 1.5E−12 | 4.4E−12 |
| 01F07-M34Y TriTAC | 5.6E−12 | 1.5E−12 | 3.6E−12 |
| 01F07-M34G TriTAC | 9.0E−12 | 2.2E−12 | 5.6E−12 |
| 01G08 TriTAC | 1.5E−11 | 2.5E−12 | 6.9E−12 |
| 01H08 TriTAC | 4.0E−12 | 9.4E−13 | 3.1E−12 |
| 02B05 TriTAC | 8.3E−12 | 2.5E−12 | 6.5E−12 |
| 02B06 TriTAC | 1.1E−11 | 2.8E−12 | 9.7E−12 |
| 02E05 TriTAC | 1.1E−11 | 3.3E−12 | 1.2E−11 |
| 02E06 TriTAC | 9.1E−12 | 2.4E−12 | 7.4E−12 |
| 02F02 TriTAC | 8.2E−12 | 3.5E−12 | 1.0E−11 |
| 02F04 TriTAC | 1.0E−11 | 2.5E−12 | 7.3E−12 |
| 02G02 TriTAC | 1.1E−11 | 2.8E−12 | 6.6E−12 |
| 02G02-M34Y TriTAC | 1.1E−11 | 5.6E−12 | 6.2E−12 |
| 02G02-M34G TriTAC | 1.2E−11 | 4.0E−12 | 7.1E−12 |

### Binding Affinity

In the instant study, the binding affinity to human BCMA protein of the BCMA targeting trispecific proteins containing a BCMA binding protein according to the present disclosure was determined.

TABLE 3
Binding affinity of purified targeting trispecific proteins containing
a BCMA binding protein according to the present disclosure.

| Construct name | Human BCMA K_{D} (M) |
|---|---|
| 01F07-M34Y TriTAC | 3.0E−09 |
| 01F07-M34G TriTAC | 6.0E−09 |
| 02B05 TriTAC | 6.0E−09 |
| 02G02-M34Y TriTAC | 5.0E−09 |
| 02G02-M34G TriTAC | 7.0E−09 |

The data in FIG. 3, FIG. 4, Table 2 and Table 3 indicate the BCMA targeting trispecific proteins can be expressed and purified to greater than 90% purity. The purified proteins exhibit about 13 fold to 213 fold more potent TDCC activity compared to a trispecific protein with the parent BCMA targeting sequence. The purified trispecific proteins bind to BCMA with affinity of about 3 to 7 nM.

### Example 3L Xenograft Tumor Model

An exemplary BCMA targeting trispecific protein described herein was evaluated in a xenograft model.

On day 0, NCG mice were subcutaneously inoculated with RPMI-8226 cells, and also intraperitoneally implanted with normal human peripheral blood mononuclear cells (PBMCs). Treatment with an exemplary BCMA targeting trispecific protein (02B05) (SEQ ID NO: 520) was also started on day 0 (qdx10) (once daily for 10 days). The dosage of administration was 5 μg/kg, 50 μg/kg, or 500 μg/kg of the BCMA targeting trispecific protein 02B05, or a vehicle as control. Tumor volumes were determined for 25 days. As shown in FIG. 30, the mean tumor volumes were significantly lower in mice treated with the exemplary BCMA targeting trispecific protein (02B05) (at 50 μg/kg, or 500 μg/kg), as compared to the mice treated with the vehicle or the lower dose of BCMA targeting trispecific protein (02B05) (at 5 μg/kg).

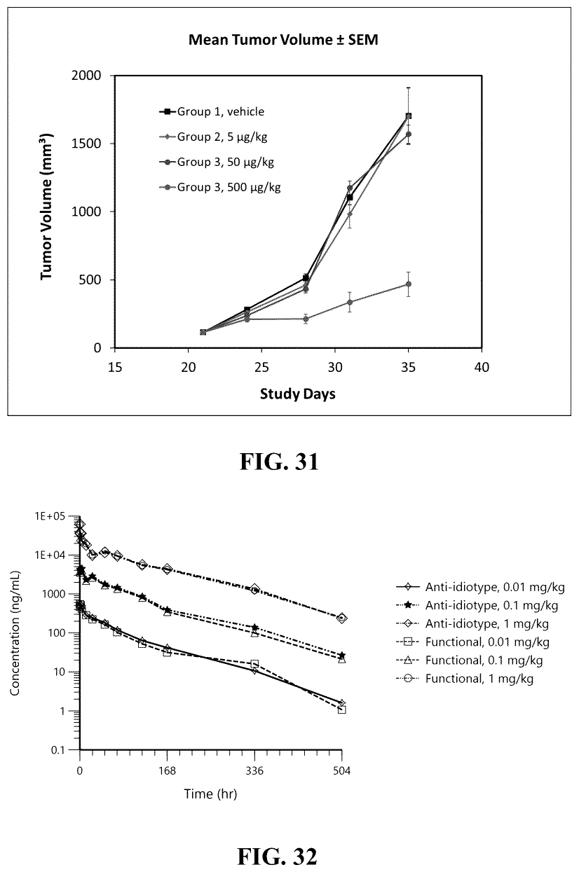

On day 0, NCG mice were subcutaneously inoculated with Jeko 1 cells, and also intraperitoneally implanted with normal human peripheral blood mononuclear cells (PBMCs). Treatment with an exemplary BCMA targeting trispecific protein (02B05) (SEQ ID NO: 520) was started on day 3 (qdx10) (once daily for 10 days). The dosage of administration was 5 μg/kg, 50 μg/kg, or 500 μg/kg of the BCMA targeting trispecific protein 02B05, or a vehicle as control. Tumor volumes were determined for 25 days. As shown in FIG. 31, the mean tumor volumes were significantly lower in mice treated with the exemplary BCMA targeting trispecific protein (02B05) (at 500 μg/kg), as compared to the mice treated with the vehicle or the lower doses of BCMA targeting trispecific protein (02B05) (at 5 μg/kg or 50 μg/kg).

### Example 4: Proof-of-Concept Clinical Trial Protocol for Administration of a BCMA Trispecific Antigen-Binding Protein of this Disclosure Multiple Myeloma Patients

This is a Phase I/II clinical trial for studying the BCMA trispecific antigen-binding protein of Example 1 as a treatment for Multiple Myeloma.

Study Outcomes: Primary: Maximum tolerated dose of BCMA targeting trispecific proteins of the previous examples. Secondary: To determine whether in vitro response of BCMA targeting trispecific proteins of is the previous examples are associated with clinical response.

### Phase I

The maximum tolerated dose (MTD) will be determined in the phase I section of the trial. 1.1: The maximum tolerated dose (MTD) will be determined in the phase I section of the trial. 1.2: Patients who fulfill eligibility criteria will be entered into the trial to BCMA targeting trispecific proteins of the previous examples. 1.3: The goal is to identify the highest dose of BCMA targeting trispecific proteins of the previous examples that can be administered safely without severe or unmanageable side effects in participants. The dose given will depend on the number of participants who have been enrolled in the study prior and how well the dose was tolerated. Not all participants will receive the same dose.

### Phase II

2.1 A subsequent phase II section will be treated at the MTD with a goal of determining if therapy with therapy of BCMA targeting trispecific proteins of the previous examples results in at least a 20% response rate. Primary Outcome for the Phase II - - - To determine if therapy of BCMA targeting trispecific proteins of the previous examples results in at least 20% of patients achieving a clinical response (blast response, minor response, partial response, or complete response)

### Eligibility

Eligibility criteria for inclusion in the studies are as follows: Previously untreated patients with multiple myeloma and without serious or imminent complications (e.g., impending pathologic fracture, hypercalcemia, renal insufficiency). All asymptomatic patients with low or intermediate tumor mass will qualify. Patients with high tumor mass, symptomatic or impending fractures, hypercalcemia (corrected calcium >11.5 mg %), anemia (Hgb<8.5 gm/dl), renal failure (creatinine >2.0 mg/dl), high serum lactate dehydrogenase (>300 U/L) or plasma cell leukemia (>1000/μl) are ineligible. Overt infections or unexplained fever should be resolved before treatment. Adequate liver function (including SGPT, bilirubin and LDH) is required. Patients must have Zubrod performance of 1 or less. Patients must provide written informed consent indicating that they are aware of the investigational nature of this study. Life expectancy should exceed 1 year. Patients with idiopathic monoclonal gammopathy and non-secretory multiple myeloma are ineligible. Patients whose only prior therapy has been with local radiotherapy, alpha-IFN, or ATRA are eligible. Patients exposed to prior high-dose glucocorticoid or alkylating agent are not eligible.

### Example 5: Affinity Measurements for Human and Cynomolgus BCMA, CD3ε, and Albumin, Using an Exemplary BCMA Targeting Trispecific Protein of this Disclosure

The aim of this study was to assess the affinity of an exemplary BCMA targeting trispecific protein of this disclosure (02B05) (SEQ ID NO: 520), toward human BCMA, cynomolgus BCMA, human CD3ε, cynomolgus CD3ε, human albumin, cynomolgus albumin, and mouse albumin. The affinities were measured using an Octet instrument. For these measurements, streptavidin tips were first loaded with 2.5 nM human BCMA-Fc, 2.5 nM cynomologus BCMA-Fc, 2.5 nM human CD3ε-Fc, 2.5 nM cynomolgus CD3ε-Fc, 50 nM human serum albumin (HSA), 50 nM cynomolgus serum albumin, or 50 nM mouse serum albumin. Subsequently, the exemplary BCMA targeting trispecific protein 02B05 was incubated with the tips, and following an association period, the tips were moved to a buffer solution to allow the exemplary BCMA targeting trispecific protein (02B05) to disassociate. The affinities for binding to human and cynomolgus BCMA and CD3ε were measured in the presence of 15 mg/ml human serum albumin. Average calculated K_{D} values from these studies are provided in Table 4 (n indicates the number of independent measurements, n/d indicates no binding detected under the conditions tested). Binding was detected to human BCMA, human CD3ε, cynomolgus CD3ε, human serum albumin, cynomolgus serum albumin, and mouse serum albumin. Under the conditions tested, no binding was detected to cynomolgus BCMA.

TABLE 4
Measured K_{D} values for exemplary BCMA targeting
trispecific protein 02B05 to protein ligands.

| Protein | | | |
|---|---|---|---|
| ligand | Species | K_{D} (nM) | n |
| BCMA | human | 2.4 ± 0.2 | 2 |
| | cynomolgus | n/d | 2 |
| CD38 | human | 8 ± 1 | 2 |
| | cynomolgus | 7.8 ± 0.4 | 2 |
| Albumin | human | 6 ± 1 | 3 |
| | cynomolgus | 7.5 | 1 |
| | mouse | 76 | 1 |

### Example 6: Human T Cell Binding Ability of an Exemplary BCMA Targeting Trispecific Protein of this Disclosure

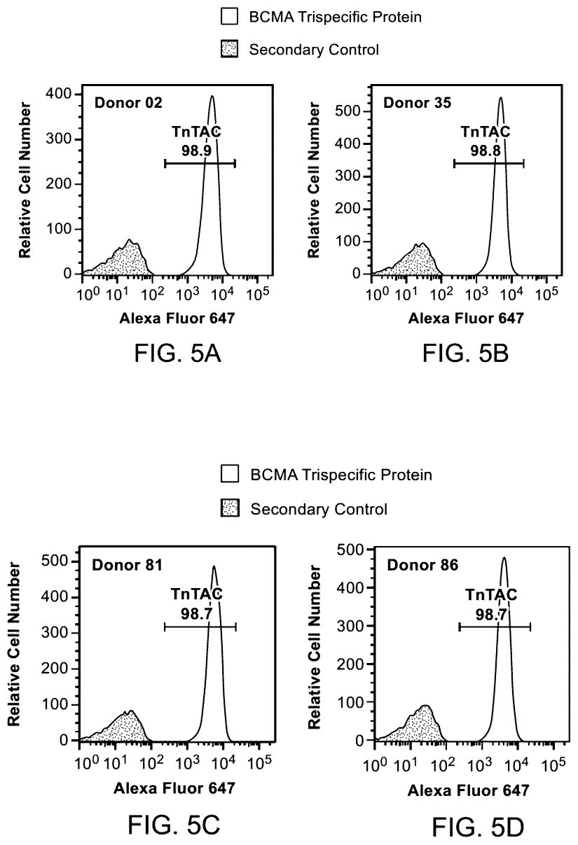

Exemplary BCMA targeting trispecific protein 02B05 (SEQ ID NO: 520) was tested for its ability to bind to purified T cells. Briefly, the BCMA trispecific protein or phosphate buffered saline (PBS) were incubated with purified T cells from 4 different anonymous human donors. After washing unbound protein, the T cells were then incubated with an Alexa Fluor 647 conjugated antibody that recognizes the anti-albumin domain in the 02B05 BCMA trispecific antigen-binding protein. The T cells were then analyzed by flow cytometry. It was observed that human T cells incubated with the 02B05 BCMA trispecific antigen-binding protein had notable shifts associated with Alexa Fluor 647 staining compared to cells that were incubated with PBS. The results are shown in FIGS. 5A, 5B, 5C, and 5D. In conclusion, this study indicated that the exemplary BCMA targeting trispecific protein was able to bind human T cells.

### Example 7: Ability of an Exemplary BCMA Targeting Trispecific Protein of this Disclosure to Bind BCMA Expressing Cells

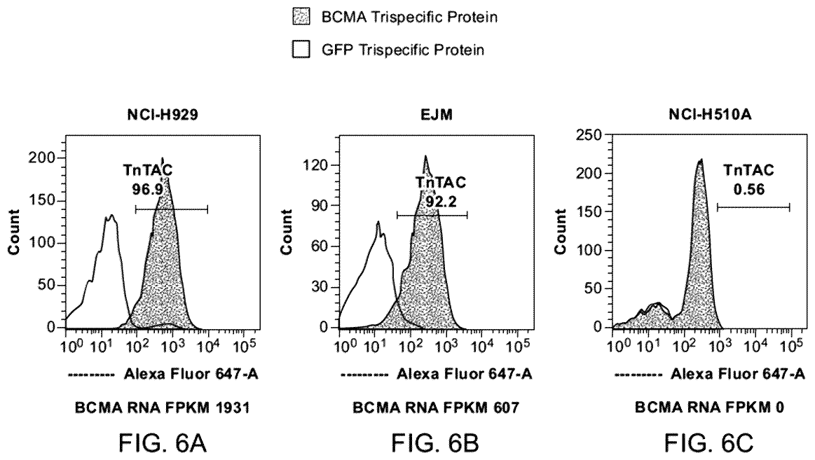

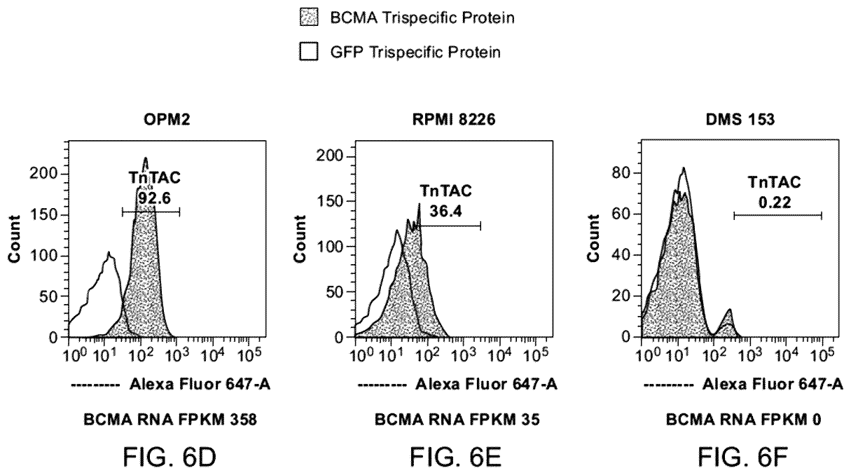

Exemplary BCMA targeting trispecific protein 02B05 (SEQ ID NO: 520) was tested for its ability to bind to BCMA expressing cells. Briefly, the 02B05 BCMA trispecific antigen-binding protein was incubated with cell lines expressing BCMA (NCI-H929; EJM; RPMI-8226; OPM2) or lacking BCMA (NCI-H510A; DMS-153). Expression of BCMA RNA in these cells is indicated by the FPKM (fragments per kilobase million) values listed in FIGS. 6A-F: the RNA FPKM values are from the Cancer Cell Line Encyclopedia (Broad Institute, Cambridge, MA USA). After washing unbound protein, the cells were then incubated with an Alexa Fluor 647 conjugated antibody that recognizes the anti-albumin domain in the 02B05 BCMA trispecific antigen-binding protein. The cells were then analyzed by flow cytometry. As a negative control, cells were incubated with a trispecific protein targeting GFP. Cells expressing BCMA RNA and incubated with the BCMA trispecific protein had notable shifts associated with Alexa Fluor 647 staining compared to cells that were incubated with GFP trispecific protein (as in FIGS. 6A, 6B, 6D, and 6E). Whereas cells lacking BCMA RNA produced equivalent Alexa Fluor 647 staining with the BCMA trispecific protein and the GFP trispecific protein (as seen in FIGS. 6C, and 6F). Thus, this study indicated that the exemplary BCMA trispecific antigen-binding was able to selectively bind to cells expressing BCMA.

### Example 8: Ability of an Exemplary BCMA Targeting Trispecific Protein to Mediate T Cell Killing of Cancer Cells Expressing BCMA

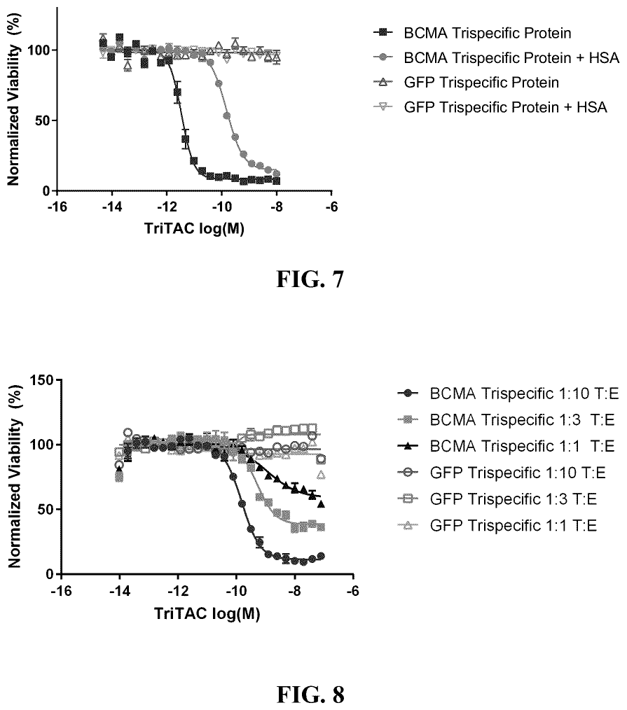

Exemplary BCMA trispecific protein 02B05 (SEQ ID NO: 520) was tested for its ability to direct T cells to kill BCMA expressing cells in the presence and absence of human serum albumin (HSA) using a standard TDCC assay as described in Example 1. Because the exemplary BCMA trispecific protein contains an anti-albumin domain, this experiment was performed to confirm that binding to albumin would not prevent the BCMA trispecific antigen-binding protein from directing T cells to kill BCMA expressing cells. Five BCMA expressing cell lines were tested: EJM, Jeko, OPM2, MOLP8, and NCI-H929. Representative data for an experiment with the EJM cells are shown in FIG. 7. It was observed that viability of the EJM cells decreased with increasing amount of the exemplary 02B05 BCMA trispecific antigen-binding protein in the presence or absence of human serum albumin (HSA), whereas a control GFP targeting trispecific protein did not affect cell viability. In the presence of albumin. higher concentrations of BCMA trispecific protein were needed to reduce viability of the EJM cells. The EC₅₀ values for cell killing by BCMA trispecific protein for the EJM cells as well as the Jeko, OPM2, MOL8, and NCI-H929 cells in the absence or presence of HSA are provided in Table 5. With all five cell lines, the exemplary 02B05 BCMA trispecific antigen-binding protein directed T cells to kill target cells in the presence of HSA.

TABLE 5
TDCC EC₅₀ Values for an exemplary
BCMA targeting trispecific protein in
the presence or absence of human serum albumin
with five different BCMA expressing cell lines.

| | EC₅₀ without | EC₅₀ with |
|---|---|---|
| Cell Line | HSA (pM) | HSA (pM) |
| EJM | 1.0 | 53 |
| Jeko | 8.3 | 662 |
| OPM2 | 6.5 | 328 |
| MOLP8 | 2.5 | 388 |
| NCI-H929 | 6.7 | 194 |

### Example 9: Ability of an Exemplary BCMA Targeting Trispecific Protein to Mediate T Cell Killing of Cancer Cells Expressing BCMA, Using a Smaller Target Cell to Effector Cell Ratio

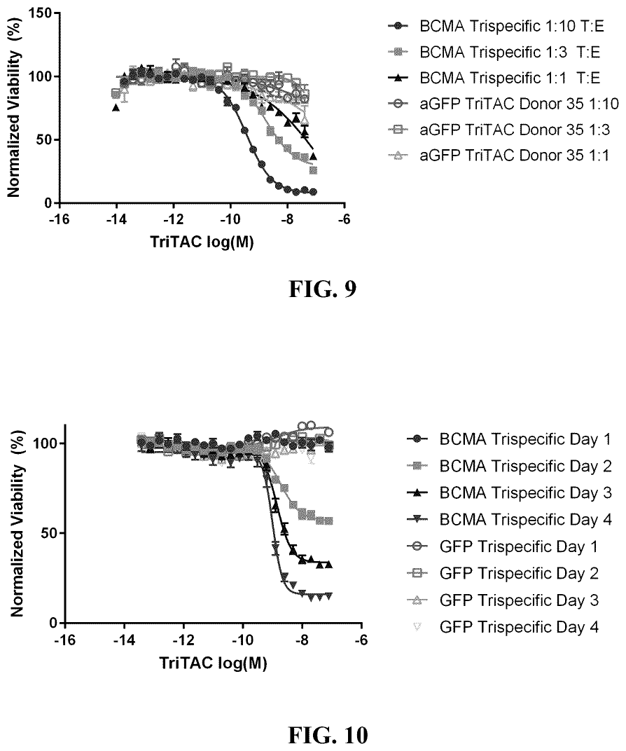

In the standard TDCC assay (as described in Example 1), a ratio 1 target cell (EJM cells or OPM2 cells) per 10 effector cells (T cells) is used in a 48 hour assay. In this experiment, the ability of exemplary BCMA trispecific protein 02B05 (SEQ ID NO: 520) to direct T cells to kill target cells with smaller target cell to effector ratios was tested. The expectation was that less killing would be observed when fewer effector cells were used. Two BCMA expressing cell lines were tested. EJM and OPM2, using target to effector cell ratios of 1:1, 1:3, and 1:10, and the experiment was performed in the presence of 15 mg/ml HSA. A GFP targeting trispecific protein was used as a negative control. Data from this experiment is shown in FIG. 8 (TDCC assay with EJM cells) and FIG. 9 (TDCC assay with OPM2 cells). As expected, near complete killing of the target cells was observed with a 1:10 target to effector cell ratio. The amount of killing was reduced with decreasing effector cells. The EC₅₀ values for cell killing with each ratio are listed in Table 6 (n/d indicates insufficient killing was observed to calculate an EC₅₀ value). The EC₅₀ values increased when fewer effector cells were present. Thus, as expected, reducing the number of effector cells to target cells reduced TDCC activity of the BCMA trispecific protein.

TABLE 6
TDCC EC₅₀ values for an exemplary BCMA
targeting trispecific protein (02B05) with
varied target cell (EJM cells) to effector cell
(T cells) ratios (tested in presence of 15 mg/ml HSA)

| Target cell:T | EJM | OPM2 |
|---|---|---|
| Cell ratio | EC₅₀ (pM) | EC₅₀ (pM) |
| 1:10 | 154 | 371 |
| 1:3 | 523 | 1896 |
| 1:1 | 1147 | n/d |

### Example 10: Ability of an Exemplary BCMA Targeting Trispecific Protein to Mediate T Cell Killing of Cancer Cells Expressing BCMA, in a Time Course Study, Using a Smaller Target Cell to Effector Cell Ratio

In the standard TDCC assay (Example 1), a ratio 1 target cell per 10 effector cells (T cells) is used in a 48-hour assay. In this experiment, a time course was performed using a 1 to 1 ratio of target cells (EJM cells) to effector cells (T cells). The expectation was that with increased time, a 1 to 1 ratio would result in target cell killing. The experiment was performed in the presence of 15 mg/ml HSA. A GFP targeting trispecific protein was used as a negative control. Target cell viability was measured on days 1, 2, 3, and 4 following incubation of the target cells and effector cells, at a 1:1 ratio, in presence of the exemplary 02B05 BCMA trispecific antigen-binding protein and 15 mg/ml HSA, or the GFP targeting trispecific protein and 15 mg/ml HSA. While no target cell killing was observed on day 1, killing was observed at all other time points in the presence of the BCMA trispecific antigen-binding protein, with the amount of killing increasing with time (FIG. 10). Killing with not observed with the GFP targeting trispecific protein. The EC₅₀ values calculated for cell killing on each day are provided in Table 7 (n/d indicates insufficient killing to determine an EC₅₀ value). From this study it was concluded that the exemplary 02B05 BCMA trispecific protein was able to direct T cell killing with lower numbers of effector cells, but more time was needed to achieve more complete killing.

TABLE 7
TDCC EC₅₀ values for an exemplary
BCMA targeting trispecific
protein (02B05) with a 1to 1 target
cell (EJM cells) to effector
cell (T cells) ratios (tested
in presence of 15 mg/ml
HSA), at varied time points.

| | EC₅₀ (pM) |
|---|---|
| Day 1 | n/d |
| Day 2 | 1859 |
| Day 3 | 1420 |
| Day 4 | 1012 |

### Example 11: Ability of an Exemplary BCMA Targeting Trispecific Protein to Direct Human T Cells to Kill BCMA Expressing Cells

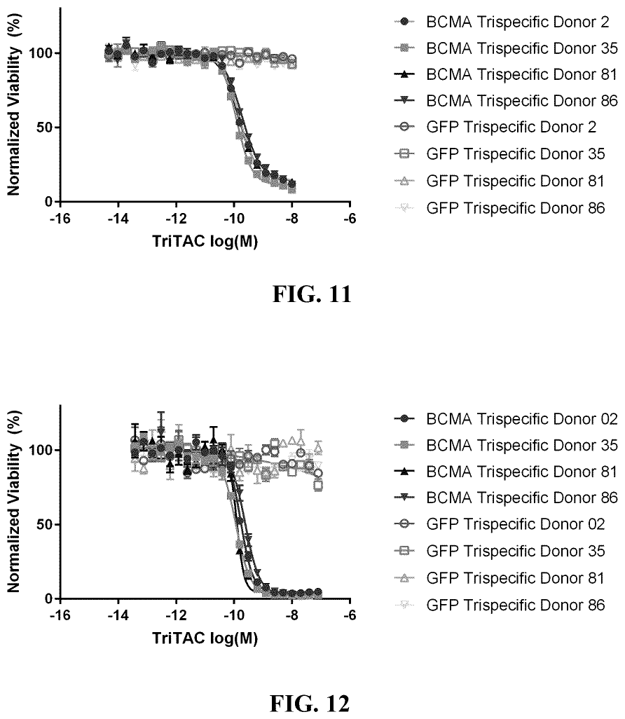

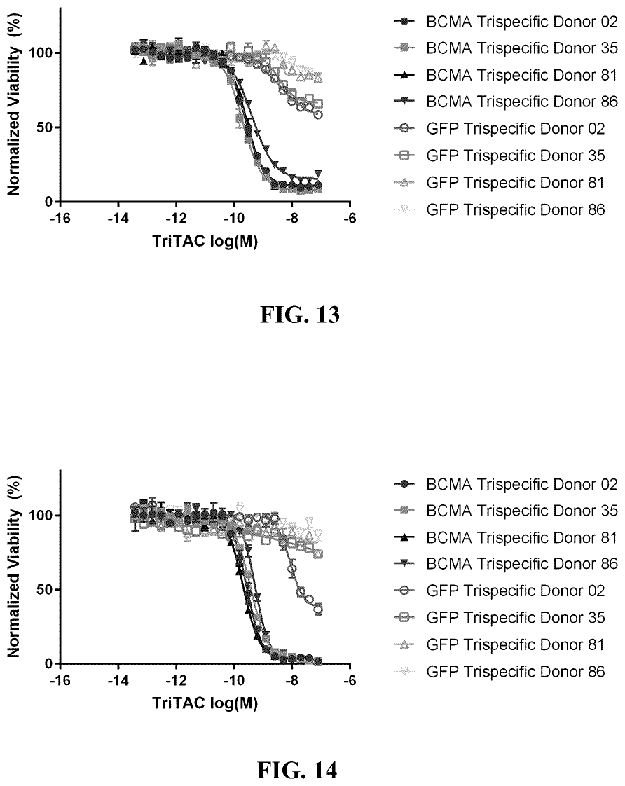

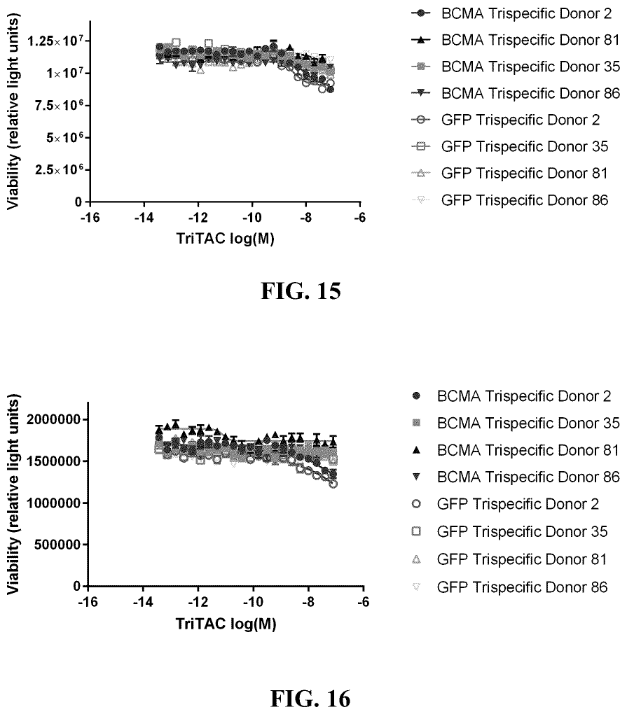

Exemplary BCMA trispecific protein 02B05 (SEQ ID NO: 520) was tested for its ability to direct T cells from four different anonymous human donors to kill four different BCMA expressing cells in the presence of 15 mg/ml human serum albumin (HSA) using a standard TDCC assay as described in Example 1. The BCMA expressing cell lines were EJM, NCI-H929, OPM2, and RPMI8226. As negative controls, two cell lines that lack BCMA expression, OVCAR8 and NCI-H510A, were also tested in the TDCC assays. A control GFP targeting trispecific protein was also used as a negative control. With the four BCMA expressing cell lines and all four T cell donors, cell viability decreased with increasing amounts of the BCMA trispecific protein but not with the GFP trispecific protein (FIGS. 11, 12, 13, and 14). The EC₅₀ values for cell killing are provided in Table 8. The exemplary 02B05 BCMA trispecific antigen-binding protein did not direct killing of the cell lines lacking BCMA expression (FIGS. 15 and 16). Thus, it was inferred that the exemplary 02B05 BCMA trispecific antigen-binding protein was able to direct T cells from multiple donors to kill a spectrum of BCMA expressing cell lines.

TABLE 8
Exemplary 02B05 BCMA trispecific protein EC₅₀
values from TDCC assays with four BCMA expressing
cell lines and four T cell donors in presence of 15 mg/ml HAS.

| | EC50 (pM) | | | |
|---|---|---|---|---|
| | H929 | OPM2 | RPMI8226 | EJM |
| Donor 02 | 169 | 250 | 275 | 151 |
| Donor 35 | 113 | 199 | 371 | 121 |
| Donor 81 | 124 | 265 | 211 | 143 |
| Donor 86 | 239 | 416 | 543 | 191 |

### Example 12: Ability of an Exemplary BCMA Targeting Trispecific Protein to Direct Cynomolgus T Cells to Kill BCMA Expressing Cells

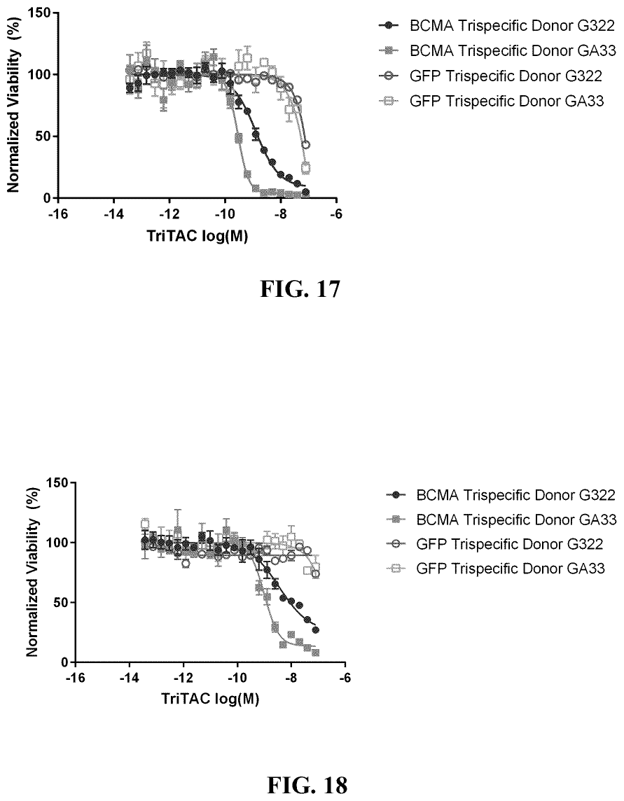

Exemplary BCMA targeting trispecific protein 02B05 (SEQ ID NO: 520) was tested for its ability to direct T cells from cynomolgus monkeys to kill BCMA expressing cells in the presence of 15 mg/ml human serum albumin (HSA). The experimental conditions were the same as described in Example 1 except peripheral blood mononuclear cells (PBMC) from cynomolgus monkeys were used as a source of T cells. Two BCMA expressing cell lines were tested. RPMI8226 and NCI-H929. As shown in FIGS. 17 and 18, the BCMA trispecific protein was able to direct T cells present in the cynomolgus PBMCs to kill the two BCMA expressing cell lines. The EC₅₀ values for the cell killing are listed in Table 9. A GFP trispecific protein did not affect viability of the BCMA expressing cells. Thus, the BCMA expressing trispecific protein, which can bind cynomolgus CD3ε (as shown in Example 5), can direct cynomolgus T cells to kill cells expressing human BCMA.

TABLE 9
BCMA trispecific protein EC₅₀ values from TDCC Assays
with two cell lines and two cynomolgusy PMBC
donors in the presence of 15 mg/ml HSA.

| | EC₅₀ (pM) | |
|---|---|---|
| | RPMI8226 | NCI-H929 |
| Donor G322 | 3654 | 1258 |
| Donor GA33 | 1003 | 288 |

### Example 13: Exemplary BCMA Trispecifc Antigen-Binding Protein and Target Tumor Cell-Mediated Induction of T Cell Activation

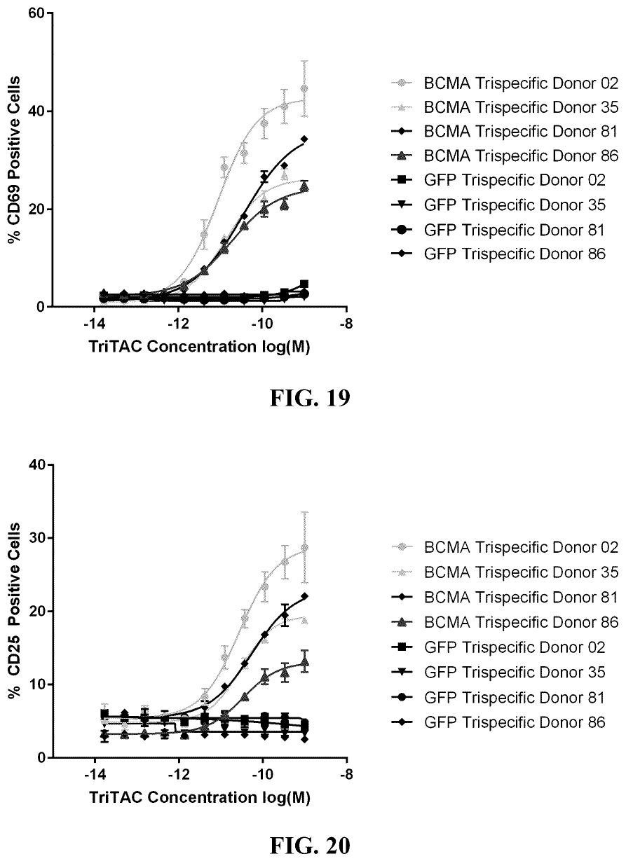

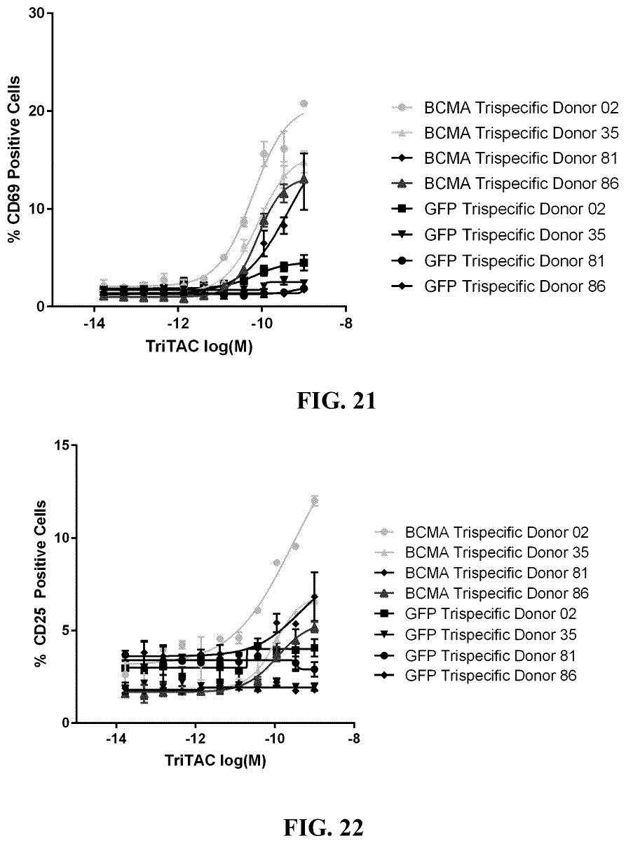

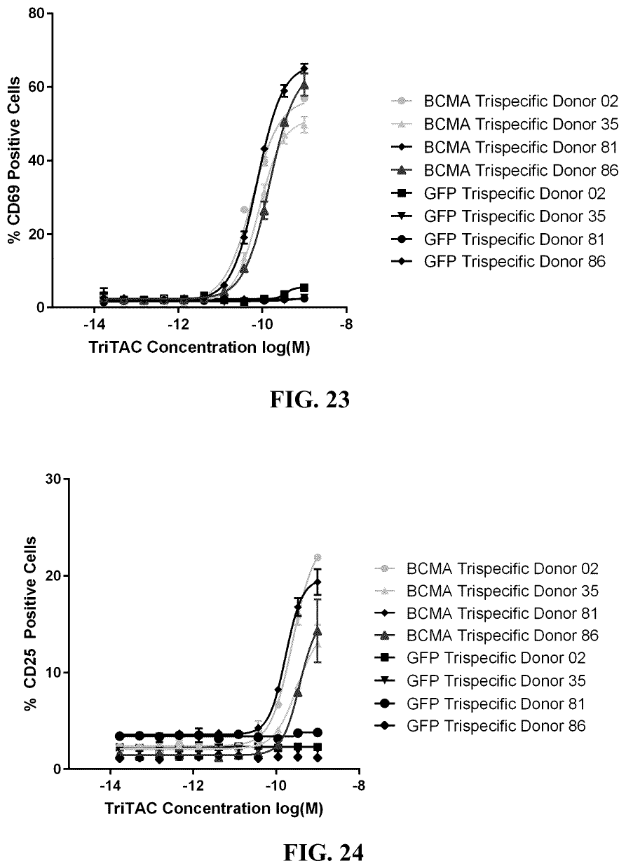

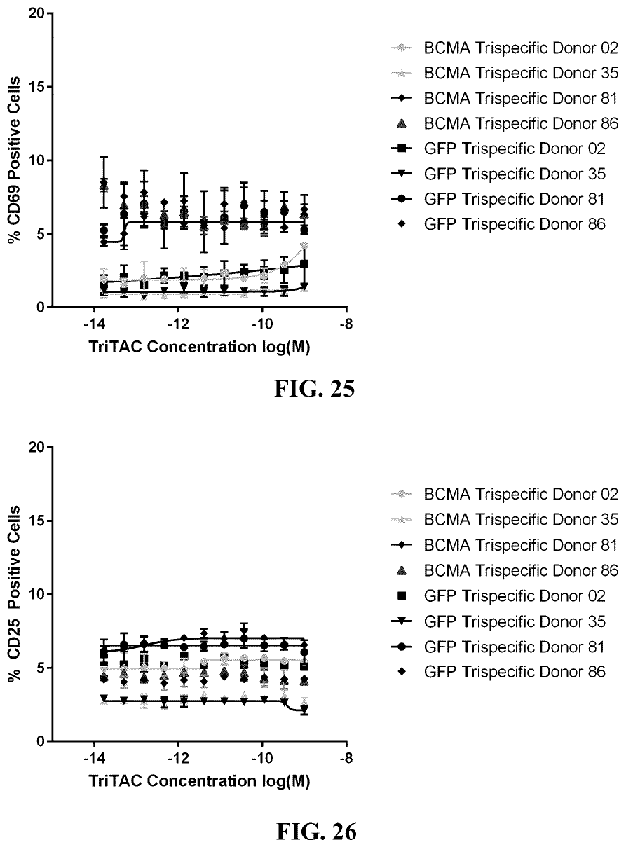

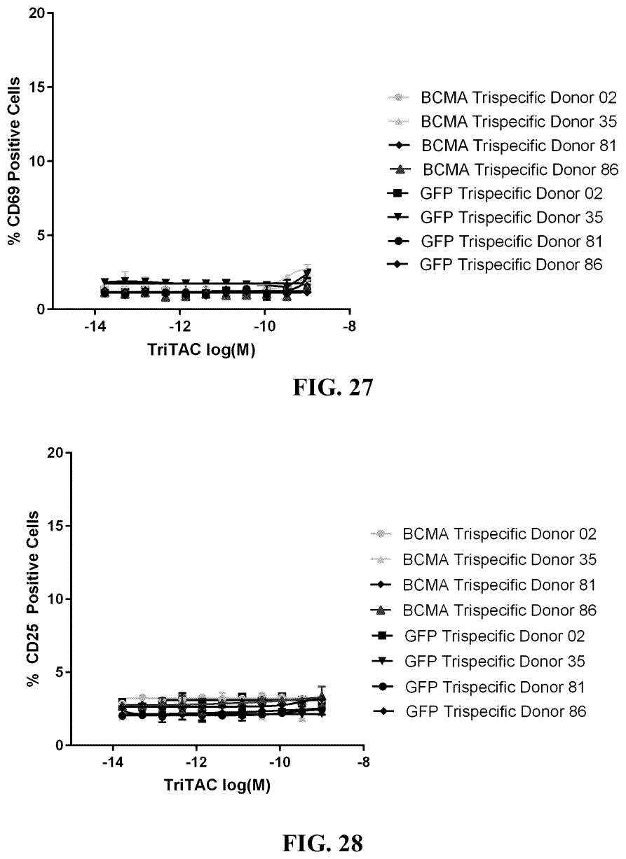

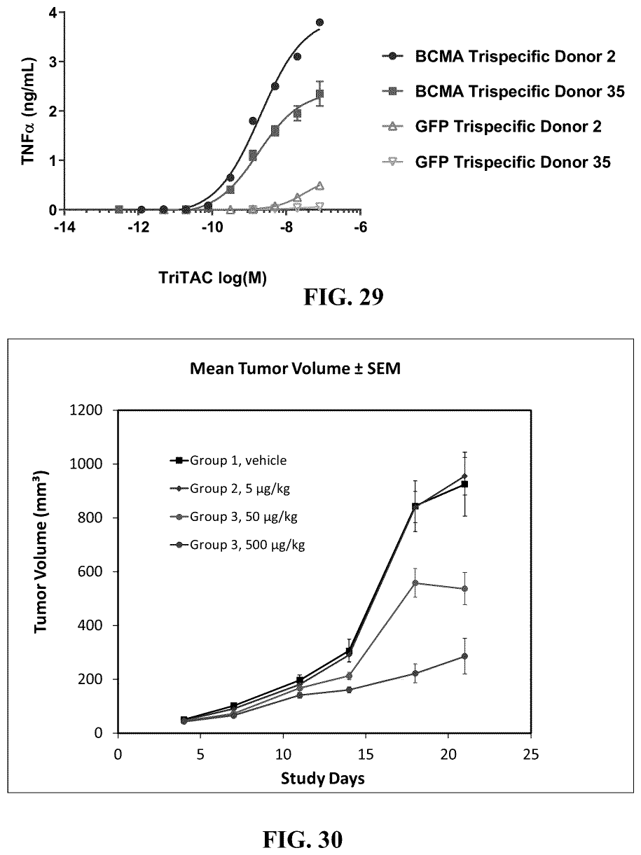

Exemplary BCMA targeting trispecific protein 02B05 (SEQ ID NO: 520) was tested for its ability to activate T cells in the presence of BCMA expressing cells. The BCMA expressing cell lines were EJM, OPM2, and RPMI8226. As negative controls, two cells lines that lack BCMA expression were also included. OVCAR8 and NCI-H510A. T cells were obtained from four different anonymous human donors. The assays were set up using the conditions of a standard TDCC assay as described in Example 1 except the assay was adapted to 96 well format and the assay was carried out in the presence of 15 mg/ml HSA. After the 48-hour assay. T cell activation was assessed by using flow cytometry to measure expression of T cell activation biomarkers CD25 and CD69 on the surface of the T cells. With increasing concentrations of the exemplary 02B05 BCMA trispecific antigen-binding protein increased expression of CD69 and CD25 was observed on T cells when co-cultured with the BCMA expressing cells (as shown in FIGS. 19-24). Thus, the observed increased expression was dependent on interaction of the BCMA binding sequence within the exemplary 02B05 BCMA trispecific antigen-binding protein with BCMA, as little to no activation was observed with a control GFP trispecific protein (as shown in FIGS. 19-24) or with target cells with no BCMA expression (as shown in FIGS. 25-28). Therefore, the exemplary 02B05 BCMA trispecific antigen-binding protein activated T cells in co-cultures containing BCMA expressing cells. This conclusion is bolstered by additional data. For instance, expression of a cytokine, TNFα, was measured in the medium collected from a co-culture of T cells and BCMA expressing target cells treated with increasing concentrations of the exemplary 02B05 BCMA trispecific antigen-binding protein or with the negative control GFP trispecific protein. The co-cultures were set up using the conditions of a standard TDCC assay (as described in Example 1) supplemented with 15 mg/ml HSA. TNFα was measured using an electrochemiluminescent assay (Meso Scale Discovery). Robust induction of TNFα expression was observed with the 02B05 exemplary BCMA targeting trispecific protein and not the GFP trispecific protein (FIG. 29). This result further supports that the 02B05 exemplary BCMA targeting trispecific protein activated T cells in co-cultures containing BCMA expressing cells.

### Example 14: Pharmacokinetics of an Exemplary BCMA Targeting Trispecific Protein of this Disclosure

Cynomolgus monkeys were administered single intravenous doses of an exemplary BCMA targeting trispecific protein (02B05) (SEQ ID NO: 520), at 0.01 mg/kg. 0.1 mg/kg, or 1 mg/kg. Two animals were included per dose group. Following the administration, serum samples were collected and analyzed by two different electrochemiluminescent assays. One assay used biotinylated CD3ε as a capture reagent and detected with sulfo-tagged BCMA (termed the functional assay). Another assay used as a capture reagent a biotinylated antibody recognizing the anti-albumin domain in the exemplary BCMA targeting trispecific protein and used as a detection reagent a sulfo-tagged antibody recognizing the anti-CD3 binding domain in the exemplary BCMA targeting trispecific protein (i.e., an anti-idiotype antibody). The results from the electrochemiluminescent assays are plotted in FIG. 32. As seen in FIG. 32, the exemplary BCMA targeting trispecific protein was detected in the cynomolgus serum samples, even after 504 hours after the administration. The exemplary BCMA targeting trispecific protein was identified using both the sulfo-tagged BCMA (lines labeled using the term “functional” in FIG. 32) and by the anti-idiotype antibody (lines labeled using the term “anti-idiotype” in FIG. 32).

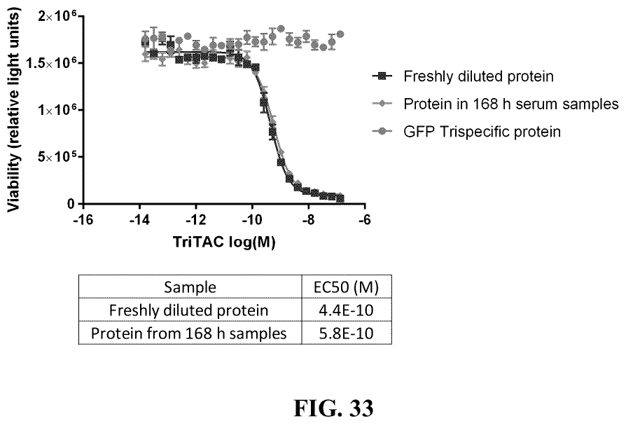

To confirm that the exemplary BCMA targeting trispecific protein retained the ability to direct T cells to kill BCMA expressing EJM cells, after in vivo administration, serum samples from the 168 hour time point were tested in a TDCC assay (as described in Example 1) in the presence of 16.7% serum from a cynomolgus monkey that has not been exposed to a BCMA targeting trispecific protein, titrating the exemplary BCMA targeting trispecific protein using the protein concentrations determined using the electrochemiluminescent assays (shown in FIG. 33). Fresh diluted exemplary 02B05 BCMA trispecific protein was compared to the BCMA trispecific protein collected from the test cynomolgus monkeys at 168 h. A GFP trispecific protein was included as a negative control. This study demonstrated that the exemplary BCMA targeting trispecific protein collected from the test cynomolgus monkeys' serum had identical activity as freshly diluted protein, and that the protein in the serum samples retained the ability to direct T cells to kill BCMA expressing target cells.

### Example 15: BCMA Trispecific Antigen-Binding Protein Phase 1/2a Dose Escalation, Expansion, Safety and Pharmacokinetics Study

Target population is patients with relapsed/refractory multiple myeloma (R/R MM); disease progression on the prior systemic regimen; at least three prior therapies including a proteasome inhibitor, an immunomodulatory agent, and an anti-CD38 antibody.

### Trial Design

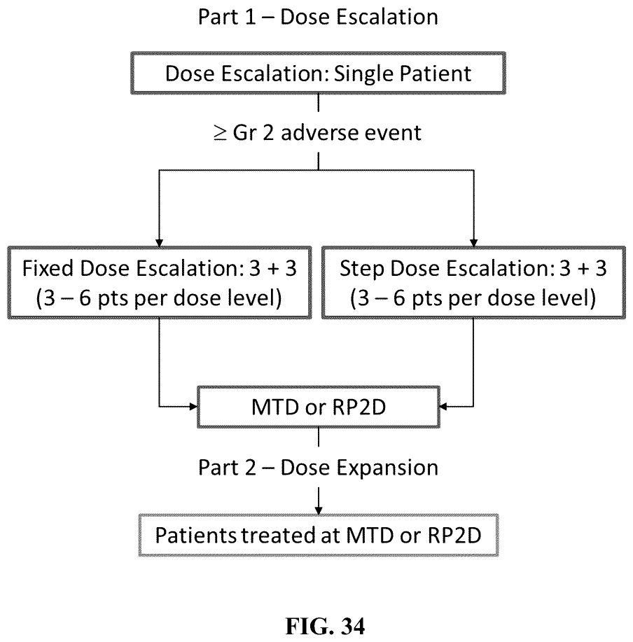

BCMA trispecific antigen-binding protein (SEQ ID NO: 520) Phase 1/2 trial design is shown in FIG. 34. Trial objectives are characterization of safety, PK, immunogenicity, and pharmacodynamics, identification of the maximum tolerated dose (MTD) or the recommended phase 2 dose (RP2D) and tumor assessments based on IMWG Response Criteria (International Myeloma Working Group Uniform Response Criteria For Multiple Myeloma and Minimal Residual Disease Assessment in Multiple Myeloma).

The study will also test the clinical activity of the BCMA trispecific antigen-binding protein in both BCMA exposed (received an agent targeted against BCMA treatment) and BCMA naïve (no prior exposure to an agent targeted against BCMA) patients.

Dosing, administration: BCMA trispecific antigen-binding protein was administered weekly through one hour IV infusion. Premedication was used to manage cytokine release syndrome (CRS). Table 10 shows the dosing cohorts and number of subjects.

TABLE 10
BCMA trispecific antigen-binding
protein dosing cohorts

| Cohort | Dose μg | N |
|---|---|---|
| 1 | 5 | 1 |
| 2 | 15 | 1 |
| 3 | 30 | 2 |
| 4 | 90 | 1 |
| 5 | 270 | 2 |
| 6 | 810 | 4 |
| 7 | 1620 | 6 |
| 8 | 2150 | 11 |
| 9 | 2860 | 6 |
| 10 | 1620 (priming) → 3240 (target) | 4 |
| 11 | 1500 (priming) → 6000 (target) | 1 |
| 12 | 1000 (priming) → 3000 → 6000 | 3 |
| | target | |
| Total | | 42 |

### Baseline Characteristics

Table 11 shows the baseline characteristics and demographics of the subjects. Table 12 shows prior systemic therapies of the subjects.

TABLE 11
Baseline Characteristics and Demographics

| Baseline Characteristics | Total N = 37 | |
|---|---|---|
| Age (yr), Median (range) | 71 | (38-78) |
| Duration of Disease (yr), Median (range) | 8 | (1-20) |
| Prior Transplantation, N (%) | 28 | (76%) |
| Prior Systemic Therapies (N), Median (range) | 7 | (2-16) |
| Best Response To Most Recent Regimen | N | (%) |
| Complete Response | 1 | (3%) |
| Very Good Partial Response | 4 | (11%) |
| Partial Response | 5 | (14%) |
| Minimal Response | 1 | (3%) |
| Stable Disease | 10 | (27%) |
| Progressive Disease | 12 | (32%) |
| Missing | 4 | (11%) |

TABLE 12
Prior Systemic Therapies

| Therapeutic Class and Agents | | Total N = 37, N (%) | |
|---|---|---|---|
| Proteasome Inhibitor | Bortezomib | 34 | (92%) |
| | Carfilzomib | 30 | (81%) |
| | Ixazomib | 10 | (27%) |
| Anti-CD38 | Daratumumab | 33 | (89%) |
| | Isatuximab | 3 | (8%) |
| IMID | Lenalidomide | 34 | (92%) |
| | Pomalidomide | 31 | (84%) |
| | Thalidomide | 11 | (30%) |
| BCMA-Targeted | Belantamab Mafodotin | 6 | (16%) |
| Therapies | SEA-BCMA | 2 | (5%) |
| | Bispecific TCE | 1 | (3%) |
| | CAR T-Cell Therapy | 1 | (3%) |

### Adverse Events

Based on available preliminary data, the most common treatment-related emergent adverse events (TEAEs) were CRS, fatigue, alanine aminotransferase (ALT) increased, and aspartate aminotransferase (AST) increased. TEAEs that occurred in ≥10% of patients are summarized in Table 13. Related TEAEs are summarized in Table 14.

TEAEs irrespective of causality assessed by the Investigator as ≥ Grade 3 in severity (per Common Terminology Criteria for Adverse Events [CTCAE] v.5.0) and occurring in ≥5% of patients included anaemia (38%), AST increased (12%), neutrophil count decreased (12%), neutropenia (10%), hypophosphataemia (10%), thrombocytopenia (7%), ALT increased (7%), platelet count decreased (7%), and hypertension (7%).

Serious adverse events (SAEs) irrespective of causality were reported for 16 (38%) patients. The most common SAEs (>2 patients) include, ALT increased (7%), AST increased (7%), CRS (7%), pneumonia (5%), general physical health deterioration (5%), and dyspnoea (5%). A summary of SAEs assessed by the Investigator as related to investigational product is provided in Table 15. As of the most recent data cut-off date, the most common SAEs assessed by the Investigator as related to investigational product (>1 treated patient) were ALT increased (7%), AST increased (7%), and CRS (7%).

One patient experienced an SAE with a fatal outcome; it was assessed by the Investigator as not related to investigational product: Patient 102-120 (2150 μg/week fixed dosing cohort) experienced general physical health deterioration and died 7 days after receiving the first dose of investigational product. The patient died of multiple organ failure in the context of documented progression of disease.

TABLE 13
Treatment-Emergent Adverse Events with an Overall Incidence of ≥10% of Treated Patients

| | Fixed Dosing | | | | | Step Dosing (Priming Dose-Target Dose) | | | |
|---|---|---|---|---|---|---|---|---|---|
| | Cohort 1-5 | Cohort 6 | Cohort 7 | Cohort 8 | Cohort 109 | Cohort 301 | Cohort 302 | Cohort 501 | |
| | (5-270 μg) | (810 μg) | (1620 μg) | (2150 μg) | (2860 μg) | (1620-3240 μg) | (1.5-6 mg) | (1-3-6 mg) | Total |
| Preferred Term | (N = 7) | (N = 4) | (N = 6) | (N = 11) | (N = 6) | (N = 4) | (N = 1) | (N = 3) | (N = 42) |
| Subjects with at least one | 6 (85.7%) | 4 (100.0%) | 6 (100.0%) | 11 (100.0%) | 6 (100.0%) | 3 (75.0%) | 0 | 3 (100.0%) | 39 (92.9%) |
| TEAE | | | | | | | | | |
| Anaemia | 4 (57.1%) | 2 (50.0%) | 4 (66.7%) | 4 (36.4%) | 4 (66.7%) | 0 | 0 | 1 (33.3%) | 19 (45.2%) |
| Fatigue | 2 (28.6%) | 1 (25.0%) | 4 (66.7%) | 4 (36.4%) | 1 (16.7%) | 1 (25.0%) | 0 | 1 (33.3%) | 14 (33.3%) |
| Cytokine release syndrome | 0 | 0 | 1 (16.7%) | 6 (54.5%) | 2 (33.3%) | 0 | 0 | 0 | 9 (21.4%) |
| Arthralgia | 0 | 1 (25.0%) | 1 (16.7%) | 3 (27.3%) | 3 (50.0%) | 0 | 0 | 0 | 8 (19.0%) |
| Aspartate aminotransferase | 1 (14.3%) | 1 (25.0%) | 0 | 2 (18.2%) | 2 (33.3%) | 1 (25.0%) | 0 | 0 | 7 (16.7%) |
| increased | | | | | | | | | |
| Cough | 0 | 1 (25.0%) | 1 (16.7%) | 4 (36.4%) | 0 | 1 (25.0%) | 0 | 0 | 7 (16.7%) |
| Diarrhoea | 0 | 1 (25.0%) | 1 (16.7%) | 2 (18.2%) | 1 (16.7%) | 1 (25.0%) | 0 | 1 (33.3%) | 7 (16.7%) |
| Headache | 0 | 2 (50.0%) | 1 (16.7%) | 1 (9.1%) | 1 (16.7%) | 1 (25.0%) | 0 | 1 (33.3%) | 7 (16.7%) |
| Hypercalcaemia | 1 (14.3%) | 1 (25.0%) | 2 (33.3%) | 1 (9.1%) | 1 (16.7%) | 1 (25.0%) | 0 | 0 | 7 (16.7%) |
| Nausea | 0 | 1 (25.0%) | 0 | 1 (9.1%) | 3 (50.0%) | 2 (50.0%) | 0 | 0 | 7 (16.7%) |
| Alanine aminotransferase | 0 | 1 (25.0%) | 0 | 2 (18.2%) | 2 (33.3%) | 1 (25.0%) | 0 | 0 | 6 (14.3%) |
| increased | | | | | | | | | |
| Dyspnoea | 3 (42.9%) | 0 | 1 (16.7%) | 1 (9.1%) | 0 | 1 (25.0%) | 0 | 0 | 6 (14.3%) |
| Epistaxis | 2 (28.6%) | 1 (25.0%) | 2 (33.3%) | 1 (9.1%) | 0 | 0 | 0 | 0 | 6 (14.3%) |
| Hypokalaemia | 1 (14.3%) | 0 | 1 (16.7%) | 2 (18.2%) | 1 (16.7%) | 1 (25.0%) | 0 | 0 | 6 (14.3%) |
| Hypomagnesaemia | 2 (28.6%) | 1 (25.0%) | 1 (16.7%) | 0 | 0 | 1 (25.0%) | 0 | 1 (33.3%) | 6 (14.3%) |
| Neutrophil count decreased | 0 | 1 (25.0%) | 1 (16.7%) | 2 (18.2%) | 1 (16.7%) | 1 (25.0%) | 0 | 0 | 6 (14.3%) |
| Back pain | 0 | 2 (50.0%) | 2 (33.3%) | 0 | 1 (16.7%) | 0 | 0 | 0 | 5 (11.9%) |
| Chills | 0 | 1 (25.0%) | 1 (16.7%) | 3 (27.3%) | 0 | 0 | 0 | 0 | 5 (11.9%) |
| Hypophosphataemia | 1 (14.3%) | 1 (25.0%) | 1 (16.7%) | 1 (9.1%) | 1 (16.7%) | 0 | 0 | 0 | 5 (11.9%) |
| Platelet count decreased | 1 (14.3%) | 1 (25.0%) | 1 (16.7%) | 0 | 1 (16.7%) | 1 (25.0%) | 0 | 0 | 5 (11.9%) |

TABLE 14
Treatment-Related Emergent Adverse Events as per Investigators' Assessment by System Organ Class and Preferred Term

| | Fixed Dosing | | | | | Step Dosing (Priming Dose-Target Dose) | | | |
|---|---|---|---|---|---|---|---|---|---|
| | Cohort 1-5 | Cohort 6 | Cohort 7 | Cohort 8 | Cohort 109 | Cohort 301 | Cohort 302 | Cohort 501 | |
| System Order Class | (5-270 μg) | (810 μg) | (1620 μg) | (2150 μg) | (2860 μg) | (1620-3240 g) | (1.5-6 mg) | (1-3-6 mg) | Total |
| Preferred Term | (N = 7) | (N = 4) | (N = 6) | (N = 11) | (N = 6) | (N = 4) | (N = 1) | (N = 3) | (N = 42) |
| Subjects with at least one | 3 (42.9%) | 3 (75.0%) | 5 (83.3%) | 9 (81.8%) | 5 (83.3%) | 2 (50.0%) | 0 | 2 (66.7%) | 29 (69.0%) |
| Related TEAE | | | | | | | | | |
| General disorders and | 0 | 2 (50.0%) | 1 (16.7%) | 5 (45.5%) | 0 | 1 (25.0%) | 0 | 1 (33.3%) | 10 (23.8%) |
| administration site conditions | | | | | | | | | |
| Fatigue | 0 | 1 (25.0%) | 1 (16.7%) | 2 (18.2%) | 0 | 1 (25.0%) | 0 | 1 (33.3%) | 6 (14.3%) |
| Chills | 0 | 1 (25.0%) | 0 | 3 (27.3%) | 0 | 0 | 0 | 0 | 4 (9.5%) |
| Asthenia | 0 | 0 | 0 | 1 (9.1%) | 0 | 0 | 0 | 0 | 1 (2.4%) |
| Pyrexia | 0 | 0 | 0 | 0 | 0 | 0 | 0 | 1 (33.3%) | 1 (2.4%) |
| Immune system disorders | 0 | 0 | 1 (16.7%) | 6 (54.5%) | 2 (33.3%) | 0 | 0 | 0 | 9 (21.4%) |
| Cytokine release syndrome | 0 | 0 | 1 (16.7%) | 6 (54.5%) | 2 (33.3%) | 0 | 0 | 0 | 9 (21.4%) |
| Investigations | 0 | 2 (50.0%) | 1 (16.7%) | 2 (18.2%) | 3 (50.0%) | 1 (25.0%) | 0 | 0 | 9 (21.4%) |
| Alanine aminotransferase | 0 | 1 (25.0%) | 0 | 2 (18.2%) | 2 (33.3%) | 0 | 0 | 0 | 5 (11.9%) |
| increased | | | | | | | | | |
| Aspartate aminotransferase | 0 | 1 (25.0%) | 0 | 2 (18.2%) | 2 (33.3%) | 0 | 0 | 0 | 5 (11.9%) |
| increased | | | | | | | | | |
| Lipase increased | 0 | 1 (25.0%) | 1 (16.7%) | 0 | 0 | 0 | 0 | 0 | 2 (4.8%) |
| Bilirubin conjugated | 0 | 0 | 0 | 1 (9.1%) | 0 | 0 | 0 | 0 | 1 (2.4%) |
| increased | | | | | | | | | |
| Blood lactate dehydrogenase | 0 | 1 (25.0%) | 0 | 0 | 0 | 0 | 0 | 0 | 1 (2.4%) |
| increased | | | | | | | | | |
| C-reactive protein increased | 0 | 1 (25.0%) | 0 | 0 | 0 | 0 | 0 | 0 | 1 (2.4%) |
| Neutrophil count decreased | 0 | 0 | 0 | 0 | 0 | 1 (25.0%) | 0 | 0 | 1 (2.4%) |
| Platelet count decreased | 0 | 0 | 0 | 0 | 0 | 1 (25.0%) | 0 | 0 | 1 (2.4%) |
| Serum ferritin increased | 0 | 1 (25.0%) | 0 | 0 | 0 | 0 | 0 | 0 | 1 (2.4%) |
| Transaminases increased | 0 | 0 | 0 | 0 | 1 (16.7%) | 0 | 0 | 0 | 1 (2.4%) |
| White blood cell count | 0 | 1 (25.0%) | 0 | 0 | 0 | 0 | 0 | 0 | 1 (2.4%) |
| decreased | | | | | | | | | |
| Metabolism and nutrition | 2 (28.6%) | 1 (25.0%) | 0 | 1 (9.1%) | 1 (16.7%) | 1 (25.0%) | 0 | 1 (33.3%) | 7 (16.7%) |
| disorders | | | | | | | | | |
| Hypomagnesaemia | 1 (14.3%) | 0 | 0 | 0 | 0 | 1 (25.0%) | 0 | 1 (33.3%) | 3 (7.1%) |
| Decreased appetite | 0 | 0 | 0 | 1 (9.1%) | 0 | 1 (25.0%) | 0 | 0 | 2 (4.8%) |
| Hypophosphataemia | 0 | 1 (25.0%) | 0 | 0 | 1 (16.7%) | 0 | 0 | 0 | 2 (4.8%) |
| Dehydration | 0 | 0 | 0 | 0 | 0 | 1 (25.0%) | 0 | 0 | 1 (2.4%) |
| Hyperphosphataemia | 1 (14.3%) | 0 | 0 | 0 | 0 | 0 | 0 | 0 | 1 (2.4%) |
| Hypokalaemia | 1 (14.3%) | 0 | 0 | 0 | 0 | 0 | 0 | 0 | 1 (2.4%) |
| Gastrointestinal disorders | 0 | 1 (25.0%) | 1 (16.7%) | 1 (9.1%) | 1 (16.7%) | 1 (25.0%) | 0 | 0 | 5 (11.9%) |
| Nausea | 0 | 1 (25.0%) | 0 | 0 | 1 (16.7%) | 1 (25.0%) | 0 | 0 | 3 (7.1%) |
| Diarrhoea | 0 | 0 | 1 (16.7%) | 1 (9.1%) | 0 | 0 | 0 | 0 | 2 (4.8%) |
| Nervous system disorders | 0 | 2 (50.0%) | 1 (16.7%) | 0 | 0 | 1 (25.0%) | 0 | 1 (33.3%) | 5 (11.9%) |
| Headache | 0 | 2 (50.0%) | 1 (16.7%) | 0 | 0 | 0 | 0 | 1 (33.3%) | 4 (9.5%) |
| Akathisia | 0 | 0 | 0 | 0 | 0 | 1 (25.0%) | 0 | 0 | 1 (2.4%) |
| Blood and lymphatic system | 2 (28.6%) | 1 (25.0%) | 0 | 0 | 1 (16.7%) | 0 | 0 | 0 | 4 (9.5%) |
| disorders | | | | | | | | | |
| Anaemia | 2 (28.6%) | 0 | 0 | 0 | 1 (16.7%) | 0 | 0 | 0 | 3 (7.1%) |
| Neutropenia | 1 (14.3%) | 1 (25.0%) | 0 | 0 | 0 | 0 | 0 | 0 | 2 (4.8%) |
| Lymphopenia | 0 | 0 | 0 | 0 | 1 (16.7%) | 0 | 0 | 0 | 1 (2.4%) |
| Thrombocytopenia | 1 (14.3%) | 0 | 0 | C | 0 | 0 | 0 | 0 | 1 (2.4%) |
| Cardiac disorders | 1 (14.3%) | 0 | 0 | 0 | 1 (16.7%) | 0 | 0 | 0 | 2 (4.8%) |
| Bradycardia | 1 (14.3%) | 0 | 0 | 0 | 0 | 0 | 0 | 0 | 1 (2.4%) |
| Sinus bradycardia | 0 | 0 | 0 | 0 | 1 (16.7%) | 0 | 0 | 0 | 1 (2.4%) |
| Musculoskeletal and | 0 | 1 (25.0%) | 0 | 1 (9.1%) | 0 | 0 | 0 | 0 | 2 (4.8%) |
| connective tissue disorders | | | | | | | | | |
| Back pain | 0 | 1 (25.0%) | 0 | 0 | 0 | 0 | 0 | 0 | 1 (2.4%) |
| Muscle spasms | 0 | 0 | 0 | 1 (9.1%) | 0 | 0 | 0 | 0 | 1 (2.4%) |
| Myalgia | 0 | 1 (25.0%) | 0 | 0 | 0 | 0 | 0 | 0 | 1 (2.4%) |
| Pain in extremity | 0 | 1 (25.0%) | 0 | 0 | 0 | 0 | 0 | 0 | 1 (2.4%) |
| Psychiatric disorders | 0 | 1 (25.0%) | 0 | 1 (9.1%) | 0 | 0 | 0 | 0 | 2 (4.8%) |
| Confusional state | 0 | 0 | 0 | 1 (9.1%) | 0 | 0 | 0 | 0 | 1 (2.4%) |
| Insomnia | 0 | 1 (25.0%) | 0 | 0 | 0 | 0 | 0 | 0 | 1 (2.4%) |
| Respiratory, thoracic and | 0 | 1 (25.0%) | 1 (16.7%) | 0 | 0 | 0 | 0 | 0 | 2 (4.8%) |
| mediastinal disorders | | | | | | | | | |
| Epistaxis | 0 | 1 (25.0%) | 1 (16.7%) | 0 | 0 | 0 | 0 | 0 | 2 (4.8%) |
| Vascular disorders | 0 | 1 (25.0%) | 0 | 0 | 0 | 0 | 0 | 1 (33.3%) | 2 (4.8%) |
| Flushing | 0 | 0 | 0 | 0 | 0 | 0 | 0 | 1 (33.3%) | 1 (2.4%) |
| Hot flush | 0 | 1 (25.0%) | 0 | 0 | 0 | 0 | 0 | 0 | 1 (2.4%) |
| Hypertension | 0 | 1 (25.0%) | 0 | 0 | 0 | 0 | 0 | 0 | 1 (2.4%) |
| Injury, poisoning and | 0 | 0 | 0 | 1 (9.1%) | 0 | 0 | 0 | 0 | 1 (2.4%) |
| procedural complications | | | | | | | | | |
| Infusion related reaction | 0 | 0 | 0 | 1 (9.1%) | 0 | 0 | 0 | 0 | 1 (2.4%) |
| Skin and subcutaneous tissue | 0 | 1 (25.0%) | 0 | 0 | 0 | 0 | 0 | 0 | 1 (2.4%) |
| disorders | | | | | | | | | |
| Hyperhidrosis | 0 | 1 (25.0%) | 0 | 0 | 0 | 0 | 0 | 0 | 1 (2.4%) |

TABLE 15
Treatment-Related Emergent Serious Adverse Events per Investigator's Assessment by System Organ Class and Preferred Term

| | Fixed Dosing | | | | | Step Dosing (Priming Dose-Target Dose) | | | |
|---|---|---|---|---|---|---|---|---|---|
| | Cohort 1-5 | Cohort 6 | Cohort 7 | Cohort 8 | Cohort 109 | Cohort 301 | Cohort 302 | Cohort 501 | |
| System Order Class | (5-270 μg) | (810 μg) | (1620 μg) | (2150 μg) | (2860 μg) | (1620-3240 μg) | (1.5-6 mg) | (1-3-6 mg) | Total |
| Preferred Term | (N = 7) | (N = 4) | (N = 6) | (N = 11) | (N = 6) | (N = 4) | (N = 1) | (N = 3) | (N = 42) |
| Subjects with at least one | 0 | 1 (25.0%) | 0 | 5 (45.5%) | 2 (33.3%) | 0 | 0 | 0 | 8 (19.0%) |
| Serious Related TEAE | | | | | | | | | |
| Investigations | 0 | 1 (25.0%) | 0 | 1 (9.1%) | 2 (33.3%) | 0 | 0 | 0 | 4 (9.5%) |
| Alanine aminotransferase | 0 | 1 (25.0%) | 0 | 1 (9.1%) | 1 (16.7%) | 0 | 0 | 0 | 3 (7.1%) |
| increased | | | | | | | | | |
| Aspartate aminotransferase | 0 | 1 (25.0%) | 0 | 1 (9.1%) | 1 (16.7%) | 0 | 0 | 0 | 3 (7.1%) |
| increased | | | | | | | | | |
| Transaminases increased | 0 | 0 | 0 | 0 | 1 (16.7%) | 0 | 0 | 0 | 1 (2.4%) |
| Immune system disorders | 0 | 0 | 0 | 3 (27.3%) | 0 | 0 | 0 | 0 | 3 (7.1%) |
| Cytokine release syndrome | 0 | 0 | 0 | 3 (27.3%) | 0 | 0 | 0 | 0 | 3 (7.1%) |
| General disorders and | 0 | 0 | 0 | 1 (9.1%) | 0 | 0 | 0 | 0 | 1 (2.4%) |
| administration site conditions | | | | | | | | | |
| Chills | 0 | 0 | 0 | 1 (9.1%) | 0 | 0 | 0 | 0 | 1 (2.4%) |
| Psychiatric disorders | 0 | 0 | 0 | 1 (9.1%) | 0 | 0 | 0 | 0 | 1 (2.4%) |
| Confusional state | 0 | 0 | 0 | 1 (9.1%) | 0 | 0 | 0 | 0 | 1 (2.4%) |

### Time on Treatment

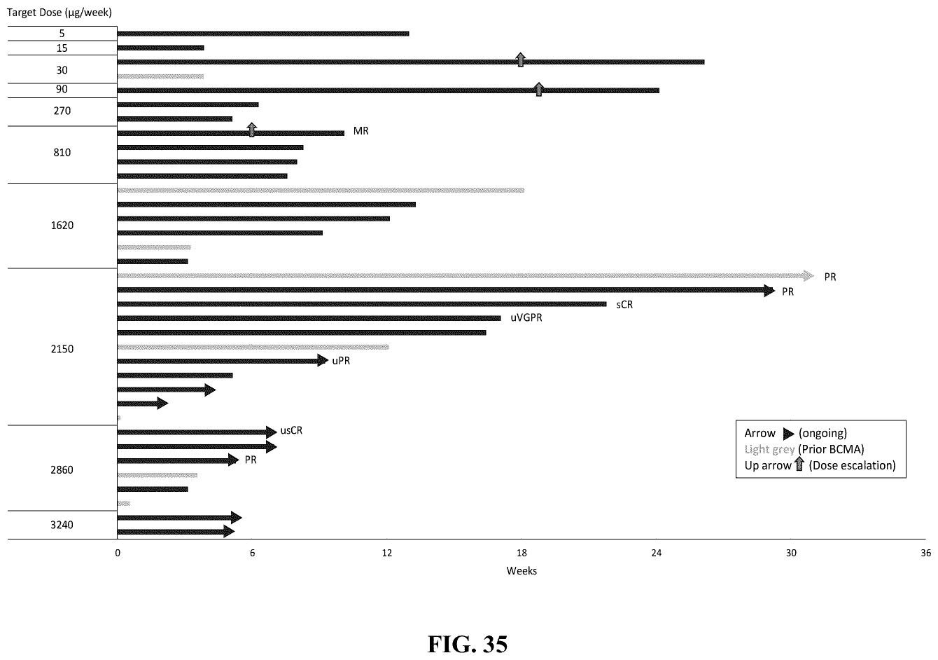

FIG. 35 shows the time on treatment for all patients treated.

### Response Assessment

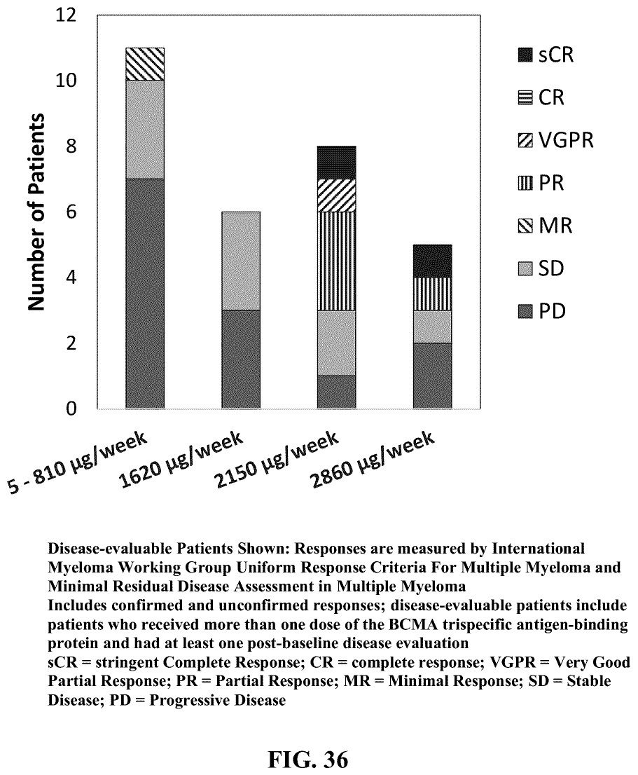

FIG. 36 shows the overall response rate. Table 16 shows the overall response and disease control rates. In 8 disease-evaluable patients enrolled at 2150 μg/wk, one stringent CR, one VGPR, and three PRs were observed, including patient with prior BCMA-targeting therapy exposure. 29% of all responders were MRD negative (as assessed by next generation flow cytometry) with stringent CR. All responders remain on study treatment.

Activity was seen at higher dose levels: 7 of 8 disease-evaluable patients in the 2150 μg/week cohort demonstrated clinical benefit, with a 63% ORR and an 88% DCR. The responders include 1 patient with prior orvacabtagene autoleucel (JCARH125) treatment. A reduction of serum B-cell maturation antigen (sBCMA) from baseline was observed in patients with PR, VGPR, or sCR.

TABLE 16
Overall Response and Disease Control Rates

| | ORR* (PR | DCR** (SD |
|---|---|---|
| Cohort | and Better) | and Better) |
| 5-810 μg/week | 0/11 (0%) | 4/11 (36%) |
| 1620 μg/week | 0/6 (0%) | 3/6 (50%) |
| 2150 μg/week | 5/8 (63%) | 7/8 (88%) |
| 2860 μg/week | 2/5 (40%) | 3/5 (60%) |

*ORR = overall response rate
**DCR = disease control rate

### Pharmacokinetics

TABLE 17
Pharmacokinetic Parameters for the BCMA trispecific antigen-binding protein Serum Concentrations^{a}

| | | | | | | | AUC₀₋₁₆₈/ | | | |
|---|---|---|---|---|---|---|---|---|---|---|
| | Dose | | | Cₘₐₓ | AUC₀₋₁₆₈ | Cₘₐₓ/Dose | Dose | Cₘᵢₙ | Cₘᵢₙ/Dose | t_{1/2} |
| Visit | (μg/week) | n | Tₘₐₓ^{b} | (ng/mL) | (h*ng/ml) | (ng/ml/μg) | (h*ng/ml/μg) | (ng/mL) | (ng/ml/μg) | (h) |
| C1D1 | 5 | 1 | EOI to 5 | 1.87 | 133 | 0.375 | 26.6 | 0.444 | 0.0888 | 129 |
| | 15 | 1 | EOI to 5 | 4.34 | 454 | 0.289 | 30.3 | 2.14 | 0.142 | 197 |
| | 30 | 2 | EOI to 24 | 10.7 | 872 | 0.358 | 29.1 | 2.79 | 0.0929 | 118 |
| | 90 | 1 | EOI to 5 | 13.9 | 1198 | 0.154 | 13.3 | 3.92 | 0.0436 | 113 |
| | 270 | 2 | EOI to 5 | 40.7 | 3306 | 0.151 | 12.2 | 7.32 | 0.0271 | 78.0 |
| | 810 | 4 | EOI to 24 | 198 (121) | 9904 (2280) | 0.244 (0.150) | 12.2 (2.82) | 15.1(4.81) | 0.0186 (0.00593) | 48.6 (9.66) |
| | 1620 | 6 | EOI to 5 | 394 (132) | 23950 (8394) | 0.243 (0.0815) | 14.8 (5.18) | 66.6 (28.0) | 0.0411 (0.0173) | 83.5 (40.4) |
| | 2150 | 6 | EOI to 5 | 831 (426) | 41086 (7601) | 0.387 (0.198) | 19.1 (3.54) | 138 (78.7) | 0.0640 (0.0366) | 73.1 (12.0) |
| C2D15 | 5 | 1 | EOI to 2 | 2.14 | 217 | 0.428 | 43.4 | 0.841 | 0.168 | 141 |
| | 30 | 1 | EOI to 2 | 95.5 | 1848 | 3.18 | 61.6 | 3.66 | 0.122 | 85.5 |
| | 90 | 1 | EOI to 2 | 17.8 | 1614 | 0.198 | 17.9 | 6.03 | 0.0670 | 153 |
| | 270 | 2 | EOI to 2 | 110 | 3408 | 0.406 | 12.6 | 34.9 | 0.129 | 109 |
| | 810 | 3 | EOI to 2 | 294 (125) | 26238 (10794) | 0.363 (0.155) | 32.4(13.3) | 43.7 (25.5) | 0.0539 (0.0315) | 50.2 |
| | 1620 | 4 | EOI to 48 | 1007 (522) | 66778 (49710) | 0.622 (0.322) | 41.2 (30.7) | 376 (264) | 0.232 (0.163) | 122 |
| | 2150 | 4 | EOI to 24 | 650 (142) | 50042 (6335) | 0.302 (0.0662) | 23.3 (2.9) | 170 (38.5) | 0.0790 (0.0179) | 101 |

AUC₀₋₁₆₈ = Area under the curve for time 0-168 hours; AUC₀₋₁₆₈/Dose = Dose normalized AUC₀₋₁₆₈; Cₘₐₓ = maximum serum concentration; Cₘₐₓ/Dose = Dose normalized max serum concentration; Cₘᵢₙ = minimum serum concentration; Cₘᵢₙ/Dose = Dose normalized min serum concentration; CxDx = Cycle x Day x; EOI = end of infusion; t_{1/2} = half-life; Tₘₐₓ = time to Cₘₐₓ
^{a}Mean (standard deviation) unless otherwise noted. When n = 1, individual observed values reports. Standard deviation reported for doses where n > 2.
^{b}Minimum to maximum for Tₘₐₓ.

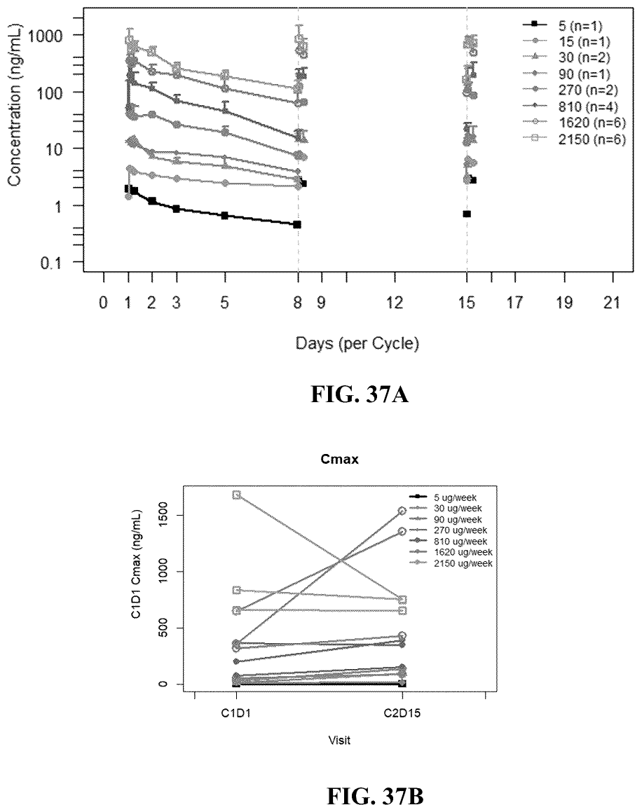

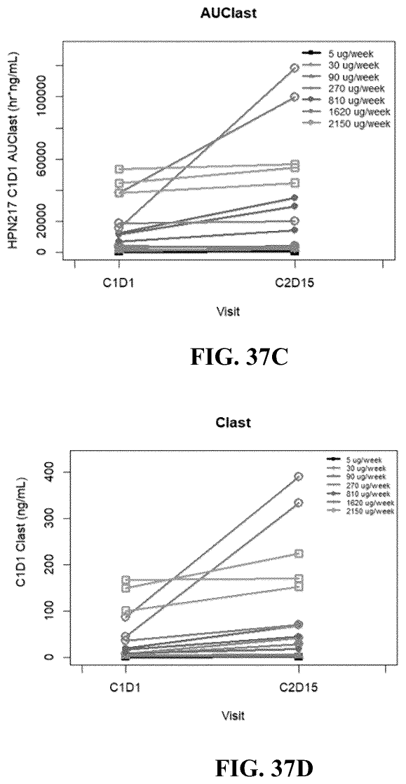

FIG. 37A illustrates the pharmacokinetic data of the BCMA trispecific antigen-binding protein for the different dosing cohorts. Linear pharmacokinetic (PK) profile shows dose-proportional increase in Cmax and AUC, dose-independent clearance and volume of distribution. Median half-life (T_{1/2}) is 74 hours. Evidence for BCMA trispecific accumulation is shown by comparing C1D1 and C2D15: about 1.5-2-fold increase in Cmax (FIG. 37B) and AUC (FIG. 37C) and about 2-3-fold increase in Clast (FIG. 37D).

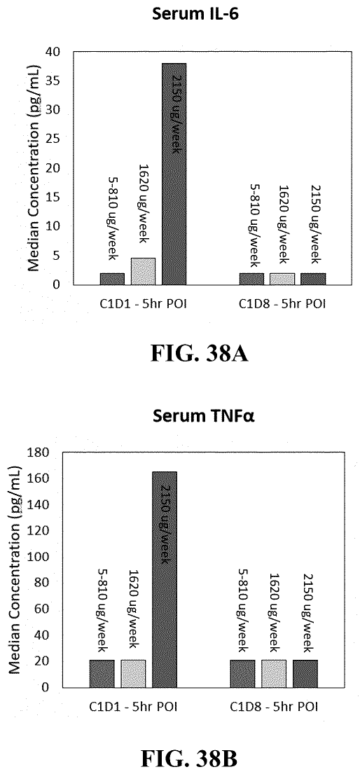

FIG. 38A shows serum cytokine concentrations 5 hours after first (C1D1) and second (C1D8) dose for serum IL-6. FIG. 38A shows serum cytokine concentrations 5 hours after first (C1D1) and second (C1D8) dose for serum TNFα.

### Serum BCMA

Measurements were available from 23 subjects who received fixed doses of 5, 15, 30, 90, 270, 810, 1620, or 2150 μg/week the BCMA trispecific antigen-binding protein. Serum specimens for BCMA (sBCMA) analysis were collected pre- and post-infusion, and samples were analyzed for sBCMA concentrations using a validated BCMA enzyme-linked immunoassay kit (hBCMA/TNFRSF17 Duo Set. RnD Systems).

In 23 patients, baseline sBCMA concentration ranged from 176 ng/ml (90 μg/week dose group, n=1) to 1213 ng/ml (15 μg/week dose group, n=1). At the higher dose groups where more than single patients were enrolled, mean sBCMA concentrations were 540 ng/mL. 588 ng/ml and 609 ng/ml, respectively, for 810 μg/week. 1620 μg/week and 2150 μg/week dose groups showing comparable levels of baseline sBCMA among these cohorts.

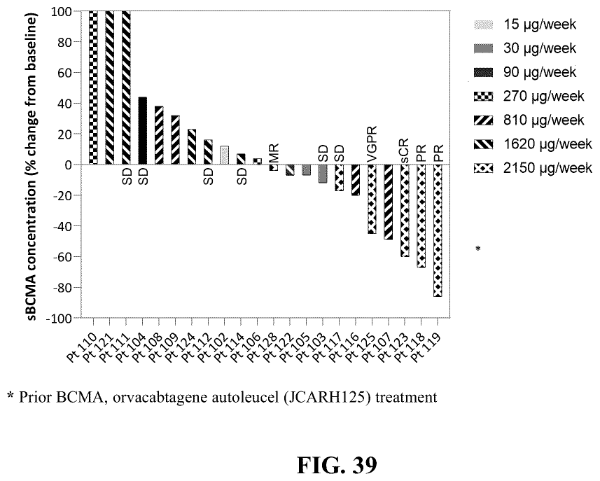

Concentrations of sBCMA (change from C1D15 to baseline) are shown in FIG. 39. Following BCMA trispecific binding protein administration. 11 out of 21 patients evaluated (52.4%) showed increases in sBCMA from 3.72 to 268%. These patients were in the 1620 μg/week or lower dose group. In 10 patients (47.6%), sBCMA concentrations showed a reduction of 1.08 to 85.6% at Cycle 1 Day 15 predose. All patients in the 2150 μg/week dose group showed reduction in sBCMA concentrations on treatment. Four patients who showed response (PR or better) to the treatment (2150 μg/week dose group) were also among the patients with reduction in sBCMA concentrations on treatment.

### Pharmacokinetics

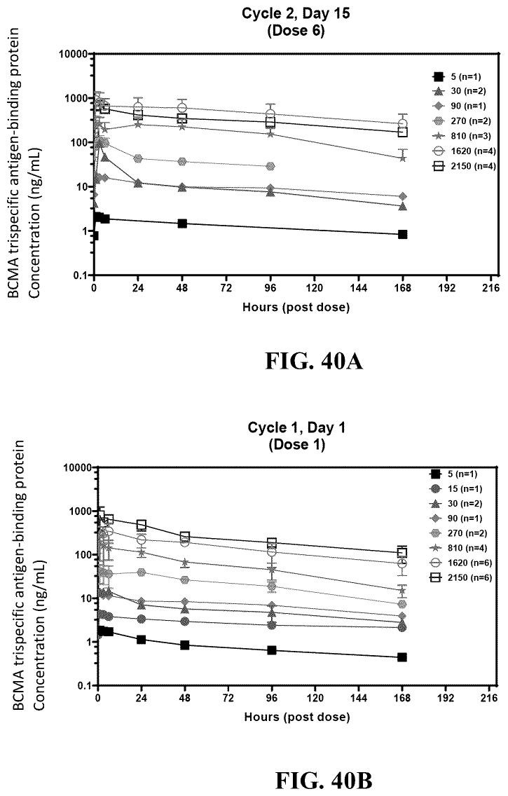

FIG. 40 shows the concentration-time profiles of the BCMA trispecific antigen-binding protein. FIG. 40A shows the concentration-time profiles after the sixth dose and FIG. 40B shows the concentration-time profiles after the first dose.

### Serum Cytokine Levels

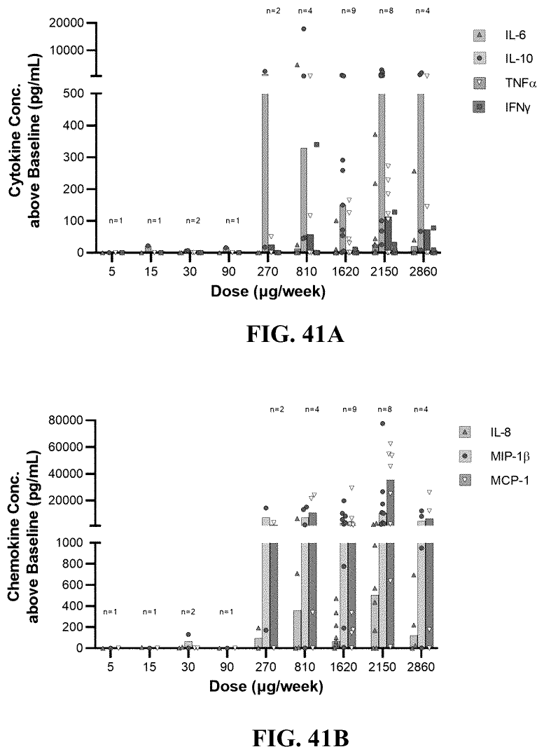

Measurements were available from 29 patients who received 5, 15, 30, 90, 270, 810, 1620, 2150, or 2860 μg/week BCMA trispecific antigen-binding protein, and from 3 subjects who received 1620 μg/week as priming doses and 3240 μg/week as target doses. Dexamethasone was given prior to infusions in Cycle 1, and as needed in subsequent cycles at the discretion of each investigator. Serum samples were collected before (baseline) and 5 hours after the infusion (5 h EOI). The Myriad RBM (Austin, TX) HMPCORE1 human multiplex cytokine panel was used to evaluate serum changes in 12 cytokines (GM-CSF, IFNγ, IL-2, IL-3, IL-4, IL-5, IL-6, IL-7, IL-10, IL-18, TNFα, and TNFβ) and 4 chemokines (IL-8, MIP-1α, MIP-1β, and MCP-1). In the 5 patients treated with 5 to 90 μg BCMA trispecific antigen-binding protein, the 5 h EOI concentrations of all 16 cytokines were either below assay LLOQ or had no significant changes from baselines (FIGS. 41A and 41B). Among patients treated with 270 to 2860 μg there was at least one patient in each cohort who also had no significant changes. Other patients had increases in IL-6, IL-10, TNFα, IFNγ, IL-8, MIP-1β, and MCP-1 to different degrees. The variability in response to the BCMA trispecific antigen-binding protein from one patient to another within each cohort is apparent. However, there was good concordance among the 7 cytokines in any given individual that concentrations tended to increase and decrease as a group, suggesting a common mechanism of induction.

There was an increase in the number of patients with cytokine spikes at 5 h EOI as dose escalated. For example, none of the patients in the 5 to 90 μg cohorts showed cytokine increase. The 270 and 810 μg dose cohorts each had 50% of patients with cytokine spikes. The majority of the patients who received 1620, 2150, or 2860 μg had cytokine spikes. Nevertheless, median cytokine concentrations did not appear to rise dose-dependently from 270 to 2860 μg/week (FIGS. 41A and 41B).

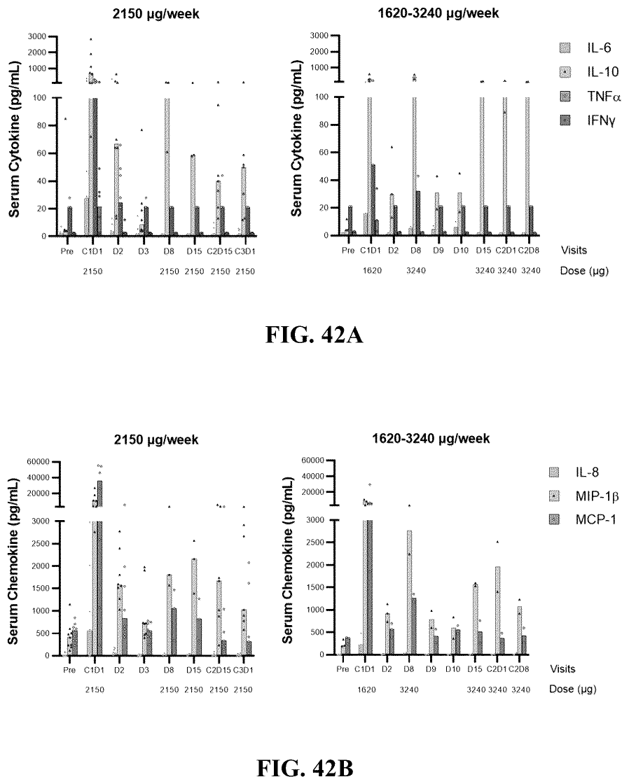

The kinetics of changes in cytokine level throughout the dosing cycle was characterized by a peak concentration at 5 h EOI on Cycle 1 Day 1, followed by a trend back to baseline in the next 24 to 48 hours. Cytokine spikes of equal or lower magnitude were observed at the 5 h EOI time points of subsequent weekly infusions. The 2150 μg cohort is given as an example for fixed dosing cohorts (FIGS. 42A and 42B, left panels). In the 1620-3240 μg step-dosing cohort, peak concentrations of IL-6, TNFα, IFNγ, IL-8, MIP-1B, and MCP-1 were recorded at 5 h EOI after the priming dose (1620 μg) on Cycle 1 Day 1. Subsequent infusions with the target dose (3240 μg) induced much lower levels of these cytokines (FIGS. 42A and 42B, right panels).

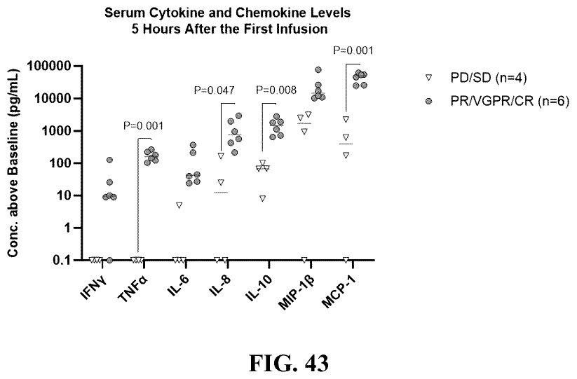

Preliminary analysis of the limited number of patients in the two highest fixed-dose cohorts (2150) and 2860 μg/week) suggested that robust cytokine and chemokine spikes at 5 h EOI on C1D1 correlate with clinical responses (FIG. 43).

### Changes in Circulating T Cells

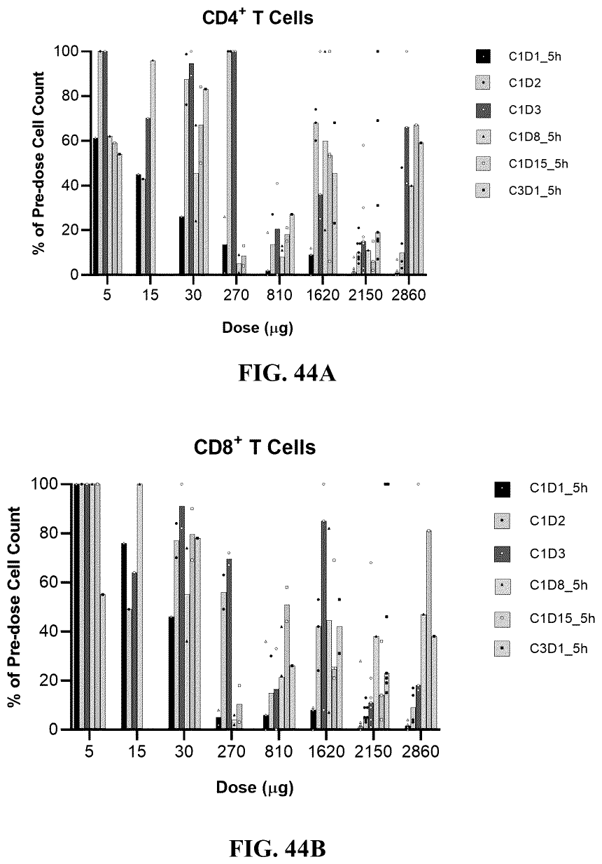

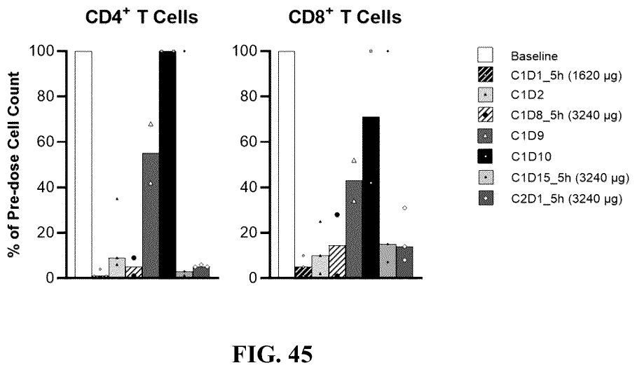

Whole blood samples collected before and after the BCMA trispecific antigen-binding protein infusions were analyzed by flow cytometry. T cells were monitored based on the expression of the following markers: CD45+CD3+CD4+ (helper T cells). CD45+CD3+CD8+ (cytotoxic T cells). A profound but transient drop in CD4+ and CD8+ T cell counts was observed after the first infusion in a dose dependent manner from 15 μg to 2860 μg. T cells reached the lowest numbers at 5 h EOI on Cycle 1 Day 1 and gradually recovered in the next 24 to 48 hours. There was a general trend of weaker recovery of T cell numbers by 48 h EOI (Cycle 1 Day 3) among patients who received higher doses. The decline in T cell count at the 5 h EOI time points on Cycle 1 Day 8. Cycle 1 Day 15, and Cycle 3 Day 1 was less profound than it was on C1D1 (FIGS. 44A and 44B). This also appeared to be the case in the 1620-3240 μg step-dosing cohort (FIG. 45), although there were data for only 3 subjects in this cohort at the time of the analysis.

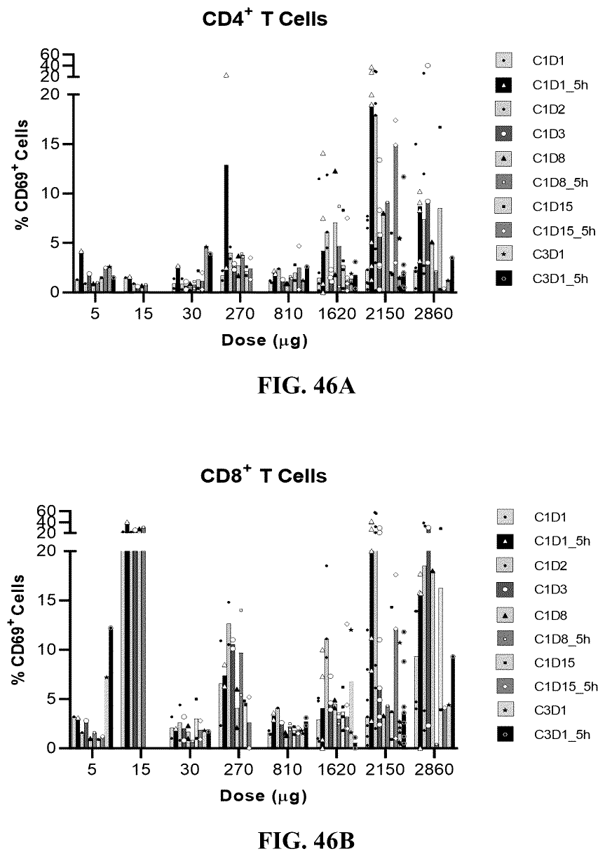

With regard to T cell activation in response to the BCMA trispecific antigen-binding protein. CD4+ and CD8+ T cells in subjects of all fixed-dose cohorts (5 to 2860 μg) upregulated the cell surface expression of CD69, a marker for T cell activation. This is consistent with the CD3-engaging property of the TriTAC platform. However, there is no evidence of a dose-dependent increase in CD69 across the cohorts. The percentages of CD69+ T cells peaked between 5 h and 24 h after the first infusion and remained higher than baseline at 48 h. The magnitude of CD69 upregulation in response to subsequent BCMA trispecific antigen-binding protein infusions on C1D8, C1D15, and C3D1 was modest in comparison to the first dose (FIGS. 46A and 46B). This also appears to be the case with the 1620-3240 μg step-dosing regimen (data not shown), although there were data for only 3 subjects in this cohort at the time of the analysis.

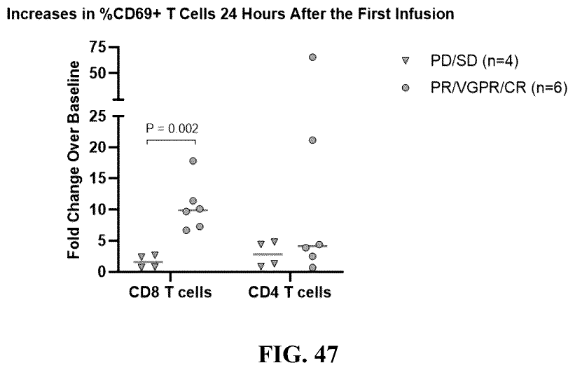

Preliminary analysis of the 2150 and 2860 μg/week dose cohorts suggested a correlation between higher expression of CD69 on CD8 T cells with clinical responses (FIG. 47). This result, corroborated by a similar finding in cytokine spikes (FIG. 43), suggested BCMA trispecific antigen-binding protein as being highly effective in engaging the target molecule (BCMA) on myeloma cells and in activating T cells.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

| SEQ | | |
|---|---|---|
| ID | | |
| NO: | Description | Sequence |
| 1 | Exemplary CDR1 | X₁X₂X₃X₄X₅X₆X₇PX₈G |
| | | where X₁ is T or S; |
| | | X₂ is N, D, or S; |
| | | X₃ is I, D, Q, H, V, |
| | | or E; X₄ is F, S, E, |
| | | A, T, M, V, I, D, |
| | | Q, P, R, or G; |
| | | X₅ is S, M, R, or |
| | | N; X₆ is I, K, S, |
| | | T, R, E, D, N, V, |
| | | H, L, A, Q, or G; |
| | | X₇ is S, T, Y, R, |
| | | or N; and X₈ is M, |
| | | G, or Y |
| 2 | Exemplary CDR2 | AIX₉GX₁₀X₁₁TX₁₂YADSVK |
| | | where X₉ is H, N, |
| | | or S; |
| | | X₁₀ is F, G, K, R, P, |
| | | D, Q, H, E, N, T, S, |
| | | A, I, L, or V; |
| | | X₁₁ is S, Q, |
| | | E, T, K, or D; and |
| | | X₁₂ is L, V, I, |
| | | F, Y, or W |
| 3 | Exemplary CDR3 | VPWGX₁₃YHPX₁₄X₁₅VX₁₆ |
| | | where X₁₃ is D, I, |
| | | T, K, R, A, E, S, |
| | | or Y; X₁₄ is R, |
| | | G, L, K, T, Q, |
| | | S, or N; X₁₅ is |
| | | N, K, E, |
| | | V, R, M, or D; |
| | | and X₁₆ is Y, A, |
| | | V, K, H, L, M, |
| | | T, R, Q, C, |
| | | S, or N |
| SEQ | | |
| ID | | |
| NO: | Name | HCDR1 |
| 4 | 01A01 | TDIFSISPMG |
| 5 | 01A02 | TNIFSSSPMG |
| 6 | 01A03 | TNIFSISPGG |
| 7 | 01A04 | TNIFMISPMG |
| 8 | 01A05 | TNIFSSSPMG |
| 9 | 01A06 | TNIFSIRPMG |
| 10 | 01A07 | TNISSISPMG |
| 11 | 01A08 | TNIFSSSPMG |
| 12 | 01A09 | TNIFSITPMG |
| 13 | 01B01 | TNIPSISPMG |
| 14 | 01B02 | TNITSISPMG |
| 15 | 01B03 | TNIFSKSPMG |
| 16 | 01B04 | TNDFSISPMG |
| 17 | 01B05 | TNITSISPMG |
| 18 | 01B06 | TNIFSISPMG |
| 19 | 01B07 | TNIFSRSPMG |
| 20 | 01B08 | TNIESISPMG |
| 21 | 01B09 | SNIFSISPMG |
| 22 | 01B12 | TNIFSTSPMG |
| 23 | 01C01 | TNIVSISPMG |
| 24 | 01C02 | TNIESISPMG |
| 25 | 01C04 | TNIPSISPMG |
| SEQ | | |
| ID | | |
| NO: | Description | Sequence |
| 26 | 01C05 | TNIFSSSPMG |
| 27 | 01C06 | TNIFSISPMG |
| 28 | 01C07 | TNIFSIYPMG |
| 29 | 01C08 | TNIFSNSPMG |
| 30 | 01C10 | TNISSISPMG |
| 31 | 01D02 | TNIVSISPMG |
| 32 | 01D03 | TNIFSNSPMG |
| 33 | 01D04 | TNITSISPMG |
| 34 | 01D05 | TNIFSDSPMG |
| 35 | 01D06 | TNIFSRSPMG |
| 36 | 01D07 | TNIFSASPMG |
| 37 | 01D10 | TNIFSASPMG |
| 38 | 01E03 | TNITSISPMG |
| 39 | 01E04 | TNIASISPMG |
| 40 | 01E05 | TNIFSRSPMG |
| 41 | 01E06 | TNIFSLSPMG |
| 42 | 01E07 | TNIPSISPMG |
| 43 | 01E08 | TNIFSQSPMG |
| 44 | 01E10 | TNIESISPMG |
| 45 | 01F02 | TNIFSHSPMG |
| 46 | 01F03 | TNIFSESPMG |
| 47 | 01F04 | TNIDSISPMG |
| 48 | 01F05 | TNIFSSSPMG |
| 49 | 01F07 | TNIFSTSPMG |
| 50 | 01F08 | TNITSVSPMG |
| 51 | 01F09 | TNISSISPMG |
| 52 | 01F10 | SNIFSISPMG |
| 53 | 01F12 | TNIFRISPMG |
| 54 | 01G01 | TNIVSISPMG |
| 55 | 01G04 | TNIDSISPMG |
| 56 | 01G06 | TNIFSRSPMG |
| 57 | 01G08 | TNIQSISPMG |
| 58 | 01G09 | TNIFNISPMG |
| 59 | 01G10 | TNEFSISPMG |
| 60 | 01G11 | TNIPSISPMG |
| 61 | 01H01 | TNIGSISPMG |
| 62 | 01H04 | TNIFSKSPMG |
| 63 | 01H05 | TNIFSITPMG |
| 64 | 01H06 | TSDFSISPMG |
| 65 | 01H08 | TNIMSISPMG |
| 66 | 01H09 | TNIMSISPMG |
| 67 | 01H10 | TNIPSISPMG |
| 68 | 01H11 | TNIFSTSPMG |
| 69 | 02A04 | TNIFSQSPMG |
| 70 | 02A05 | TNIASISPMG |
| 71 | 02A07 | TNIFSKSPMG |
| 72 | 02A08 | TNIFSRSPMG |
| 73 | 02A11 | TNHFSISPMG |
| 74 | 02B01 | TNIFSNSPMG |
| 75 | 02B04 | TNIFSTSPMG |
| 76 | 02B05 | TNIFSISPYG |
| 77 | 02B06 | TNIFSNSPMG |
| 78 | 02B07 | TNIFSSSPMG |
| 79 | 02B11 | TNIVSISPMG |
| 80 | 02B12 | TNISSISPMG |
| 81 | 02C01 | TNIISISPMG |
| 82 | 02C03 | TNIASISPMG |
| 83 | 02C05 | TNIFSESPMG |
| 84 | 02C06 | TNIFSTSPMG |
| 85 | 02D06 | TNISSISPMG |
| 86 | 02D09 | TNVVSISPMG |
| 87 | 02D11 | TNEFSISPMG |
| 88 | 02E03 | TNIFSNSPMG |
| 89 | 02E05 | TNIFSRSPMG |
| 90 | 02E06 | TNIFSDSPMG |
| 91 | 02E09 | TNDFSISPMG |
| 92 | 02F02 | TNIFSKSPMG |
| 93 | 02F03 | TNIFSIYPMG |
| 94 | 02F04 | TNIFSSSPMG |
| 95 | 02F05 | TNIFSVSPMG |
| 96 | 02F06 | TNIFSITPMG |
| 97 | 02F07 | TNIESISPMG |
| 98 | 02F11 | TNIFSTSPMG |
| 99 | 02F12 | TNIESISPMG |
| 100 | 02G01 | TNIFSINPMG |
| 101 | 02G02 | TNIFSITPMG |
| 102 | 02G05 | TNITSISPMG |
| 103 | 02G06 | TNIFSGSPMG |
| 104 | 02G07 | TNIFSITPMG |
| 105 | 02G08 | TNIDSISPMG |
| 106 | 02G09 | TNIFSDSPMG |
| 107 | 02G11 | TNIDSISPMG |
| 108 | 02H01 | TNIFSKSPMG |
| 109 | 02H04 | TNIFSVSPMG |
| 110 | 02H05 | TNQFSISPMG |
| 111 | 02H06 | TNIRSISPMG |
| 112 | 02H09 | TNIFSRSPMG |
| 113 | 02H11 | TNITSISPMG |
| 114 | 01F07-M34Y | TNIFSTSPYG |
| 115 | 01F01-M34G | TNIFSTSPGG |
| 116 | 02G02-M34Y | TNIFSITPYG |
| 17 | 02G02-M34G | TNIFSITPGG |
| | Name | CDR2 |
| 118 | 01A01 | AIHGGSTLYADSVK |
| 119 | 01A02 | AINGFSTLYADSVK |
| 120 | 01A03 | AIHGSSTLYADSVK |
| 121 | 01A04 | AIHGDSTLYADSVK |
| 122 | 01A05 | AIHGFSTLYADSVK |
| 123 | 01A06 | AIHGFSTVYADSVK |
| 124 | 01A07 | AIHGTSTLYADSVK |
| 125 | 01A08 | AIHGESTLYADSVK |
| 126 | 01A09 | AIHGRSTLYADSVK |
| 127 | 01B01 | AIHGESTLYADSVK |
| 128 | 01B02 | AISGFSTLYADSVK |
| 129 | 01B03 | AIHGKSTLYADSVK |
| 130 | 01B04 | AIHGKSTLYADSVK |
| 131 | 01B05 | AIHGFETLYADSVK |
| 132 | 01B06 | AIHGDSTLYADSVK |
| SEQ | | |
| ID | | |
| NO: | Description | Sequence |
| 133 | 01B07 | AIHGNSTLYADSVK |
| 134 | 01B08 | AIHGSSTLYADSVK |
| 135 | 01B09 | AIHGSSTLYADSVK |
| 136 | 01B12 | AIHGFQTLYADSVK |
| 137 | 01C01 | AIHGHSTLYADSVK |
| 138 | 01C02 | AIHGNSTLYADSVK |
| 139 | 01C04 | AIHGDSTLYADSVK |
| 140 | 01C05 | AIHGFKTLYADSVK |
| 141 | 01C06 | AIHGDSTLYADSVK |
| 142 | 01C07 | AIHGFSTYYADSVK |
| 143 | 01C08 | AIHGGSTLYADSVK |
| 144 | 01C10 | AIHGFSTLYADSVK |
| 145 | 01D02 | AIHGKSTLYADSVK |
| 146 | 01D03 | AIHGDSTLYADSVK |
| 147 | 01D04 | AIHGVSTLYADSVK |
| 148 | 01D05 | AIHGTSTLYADSVK |
| 149 | 01D06 | AIHGDSTLYADSVK |
| 150 | 01D07 | AIHGSSTLYADSVK |
| 151 | 01D10 | AIHGSSTLYADSVK |
| 152 | 01E03 | AIHGDSTLYADSVK |
| 153 | 01E04 | AIHGTSTLYADSVK |
| 154 | 01E05 | AIHGTSTLYADSVK |
| 155 | 01E06 | AIHGDSTLYADSVK |
| 156 | 01E07 | AIHGQSTLYADSVK |
| 157 | 01E08 | AIHGDSTLYADSVK |
| 158 | 01E10 | AIHGKSTLYADSVK |
| 159 | 01F02 | AIHGTSTLYADSVK |
| 160 | 01F03 | AIHGNSTLYADSVK |
| 161 | 01F04 | AIHGFQTLYADSVK |
| 162 | 01F05 | AIHGFSTWYADSVK |
| 163 | 01F07 | AIHGFSTIYADSVK |
| 164 | 01F08 | AIHGPSTLYADSVK |
| 165 | 01F09 | AIHGHSTLYADSVK |
| 166 | 01F10 | AIHGESTLYADSVK |
| 167 | 01F12 | AIHGDSTLYADSVK |
| 168 | 01G01 | AIHGDSTLYADSVK |
| 169 | 01G04 | AIHGNSTLYADSVK |
| 170 | 01G06 | AIHGFETLYADSVK |
| 171 | 01G08 | AIHGFETLYADSVK |
| 172 | 01G09 | AIHGFSTYYADSVK |
| 173 | 01G10 | AIHGLSTLYADSVK |
| 174 | 01G11 | AIHGASTLYADSVK |
| 175 | 01H01 | AIHGQSTLYADSVK |
| 176 | 01H04 | AIHGQSTLYADSVK |
| 177 | 01H05 | AIHGTSTLYADSVK |
| 178 | 01H06 | AIHGFETLYADSVK |
| 179 | 01H08 | AIHGFSTVYADSVK |
| 180 | 01H09 | AIHGNSTLYADSVK |
| 181 | 01H10 | AIHGESTLYADSVK |
| 182 | 01H11 | AIHGFSTLYADSVK |
| 183 | 02A04 | AIHGKSTLYADSVK |
| 184 | 02A05 | AIHGKSTLYADSVK |
| 185 | 02A07 | AIHGNSTLYADSVK |
| 186 | 02A08 | AIHGESTLYADSVK |
| 187 | 02A11 | AIHGSSTLYADSVK |
| 188 | 02B01 | AIHGRSTLYADSVK |
| 189 | 02B04 | AIHGFSTIYADSVK |
| 190 | 02B05 | AIHGTSTLYADSVK |
| 191 | 02B06 | AIHGFSTLYADSVK |
| 192 | 02B07 | AIHGHSTLYADSVK |
| 193 | 02B11 | AIHGDSTLYADSVK |
| 194 | 02B12 | AIHGFDTLYADSVK |
| 195 | 02C01 | AIHGASTLYADSVK |
| 196 | 02C03 | AIHGSSTLYADSVK |
| 197 | 02C05 | AIHGFTTLYADSVK |
| 198 | 02C06 | AIHGTSTLYADSVK |
| 199 | 02D06 | AIHGFSTVYADSVK |
| 200 | 02D09 | AIHGKSTLYADSVK |
| 201 | 02D11 | AIHGESTLYADSVK |
| 202 | 02E03 | AIHGPSTLYADSVK |
| 203 | 02E05 | AIHGISTLYADSVK |
| 204 | 02E06 | AIHGFSTFYADSVK |
| 205 | 02E09 | AIHGGSTLYADSVK |
| 206 | 02F02 | AIHGSSTLYADSVK |
| 207 | 02F03 | AIHGSSTLYADSVK |
| 208 | 02F04 | AIHGFSTLYADSVK |
| 209 | 02F05 | AIHGNSTLYADSVK |
| 210 | 02F06 | AIHGESTLYADSVK |
| 211 | 02F07 | AIHGFSTLYADSVK |
| 212 | 02F11 | AIHGTSTLYADSVK |
| 213 | 02F12 | AIHGTSTLYADSVK |
| 214 | 02G01 | AIHGFDTLYADSVK |
| 215 | 02G02 | AIHGASTLYADSVK |
| 216 | 02G05 | AIHGNSTLYADSVK |
| 217 | 02G06 | AIHGNSTLYADSVK |
| 218 | 02G07 | AIHGESTLYADSVK |
| 219 | 02G08 | AIHGESTLYADSVK |
| 220 | 02G09 | AIHGFSTLYADSVK |
| 221 | 02G11 | AIHGSSTLYADSVK |
| 222 | 02H01 | AIHGSSTLYADSVK |
| 223 | 02H04 | AIHGNSTLYADSVK |
| 224 | 02H05 | AIHGKSTLYADSVK |
| 225 | 02H06 | AIHGSSTLYADSVK |
| 226 | 02H09 | AIHGSSTLYADSVK |
| 227 | 02H11 | AIHGESTLYADSVK |
| 228 | 01F07-M34Y | AIHGFSTIYADSVK |
| 229 | 01F01-M34G | AIHGFSTIYADSVK |
| 230 | 02G02-M34Y | AIHGASTLYADSVK |
| 231 | 02G02-M34G | AIHGASTLYADSVK |
| | Name | CDR3 |
| 232 | 01A01 | VPWGDYHPRNVA |
| 233 | 01A02 | VPWGDYHPRNVH |
| 234 | 01A03 | VPWGDYHPRNVY |
| 235 | 01A04 | VPWGRYHPRNVY |
| 236 | 01A05 | VPWGDYHPRNVY |
| 237 | 01A06 | VPWGDYHPRNVY |
| 238 | 01A07 | VPWGDYHPGNVY |
| 239 | 01A08 | VPWGDYHPRKVY |
| SEQ | | |
| ID | | |
| NO: | Description | Sequence |
| 240 | 01A09 | VPWGSYHPRNVY |
| 241 | 01B01 | VPWGDYHPRNVA |
| 242 | 01B02 | VPWGDYHPRNVY |
| 243 | 01B03 | VPWGDYHPRNVV |
| 244 | 01B04 | VPWGDYHPRNVK |
| 245 | 01B05 | VPWGDYHPGNVY |
| 246 | 01B06 | VPWGEYHPRNVY |
| 247 | 01B07 | VPWGIYHPRNVY |
| 248 | 01B08 | VPWGRYHPRNVY |
| 249 | 01B09 | VPWGDYHPGNVY |
| 250 | 01B12 | VPWGDYHPRNVV |
| 251 | 01C01 | VPWGDYHPGNVY |
| 252 | 01C02 | VPWGRYHPRNVY |
| 253 | 01C04 | VPWGDYHPRNVY |
| 254 | 01C05 | VPWGDYHPGNVY |
| 255 | 01C06 | VPWGKYHPRNVY |
| 256 | 01C07 | VPWGSYHPRNVY |
| 257 | 01C08 | VPWGDYHPRNVH |
| 258 | 01C10 | VPWGYYHPRNVY |
| 259 | 01D02 | VPWGDYHPGNVY |
| 260 | 01D03 | VPWGDYHPRNVR |
| 261 | 01D04 | VPWGDYHPRNVQ |
| 262 | 01D05 | VPWGDYHPRNVY |
| 263 | 01D06 | VPWGDYHPRNVT |
| 264 | 01D07 | VPWGDYHPRNVN |
| 265 | 01D10 | VPWGRYHPRNVY |
| 266 | 01E03 | VPWGDYHPGNVY |
| 267 | 01E04 | VPWGDYHPGNVY |
| 268 | 01E05 | VPWGKYHPRNVY |
| 269 | 01E06 | VPWGDYHPRNVY |
| 270 | 01E07 | VPWGDYHPRNVQ |
| 271 | 01E08 | VPWGDYHPGNVC |
| 272 | 01E10 | VPWGDYHPRRVY |
| 273 | 01F02 | VPWGRYHPRNVY |
| 274 | 01F03 | VPWGTYHPRNVY |
| 275 | 01F04 | VPWGDYHPGNVY |
| 276 | 01F05 | VPWGRYHPRNVY |
| 277 | 01F07 | VPWGDYHPGNVY |
| 278 | 01F08 | VPWGDYHPTNVY |
| 279 | 01F09 | VPWGRYHPRNVY |
| 280 | 01F10 | VPWGDYHPRNVT |
| 281 | 01F12 | VPWGRYHPRNVY |
| 282 | 01G01 | VPWGDYHPRRVY |
| 283 | 01G04 | VPWGDYHPRMVY |
| 284 | 01G06 | VPWGDYHPRNVL |
| 285 | 01G08 | VPWGDYHPGNVY |
| 286 | 01G09 | VPWGRYHPRNVY |
| 287 | 01G10 | VPWGAYHPRNVY |
| 288 | 01G11 | VPWGDYHPRNVA |
| 289 | 01H01 | VPWGDYHPQNVY |
| 290 | 01H04 | VPWGDYHPRNVT |
| 291 | 01H05 | VPWGRYHPRNVY |
| 292 | 01H06 | VPWGDYHPGNVY |
| 293 | 01H08 | VPWGDYHPGNVY |
| 294 | 01H09 | VPWGDYHPGNVY |
| 295 | 01H10 | VPWGDYHPRNVY |
| 296 | 01H11 | VPWGDYHPGNVY |
| 297 | 02A04 | VPWGDYHPSNVY |
| 298 | 02A05 | VPWGDYHPGNVY |
| 299 | 02A07 | VPWGDYHPREVY |
| 300 | 02A08 | VPWGRYHPGNVY |
| 301 | 02A11 | VPWGDYHPRVVY |
| 302 | 02B01 | VPWGDYHPRNVM |
| 303 | 02B04 | VPWGDYHPLNVY |
| 304 | 02B05 | VPWGDYHPGNVY |
| 305 | 02B06 | VPWGDYHPGNVY |
| 306 | 02B07 | VPWGDYHPRNVT |
| 307 | 02B11 | VPWGDYHPRNVS |
| 308 | 02B12 | VPWGDYHPRNVY |
| 309 | 02C01 | VPWGDYHPGNVY |
| 310 | 02C03 | VPWGDYHPGNVY |
| 311 | 02C05 | VPWGDYHPRNVT |
| 312 | 02C06 | VPWGDYHPGNVY |
| 313 | 02D06 | VPWGRYHPRNVY |
| 314 | 02D09 | VPWGDYHPNNVY |
| 315 | 02D11 | VPWGDYHPGNVY |
| 316 | 02E03 | VPWGDYHPRNVT |
| 317 | 02E05 | VPWGDYHPGNVY |
| 318 | 02E06 | VPWGDYHPGNVY |
| 319 | 02E09 | VPWGDYHPRNVA |
| 320 | 02F02 | VPWGDYHPGNVY |
| 321 | 02F03 | VPWGDYHPKNVY |
| 322 | 02F04 | VPWGDYHPGNVY |
| 323 | 02F05 | VPWGKYHPRNVY |
| 324 | 02F06 | VPWGRYHPRNVY |
| 325 | 02F07 | VPWGDYHPGNVY |
| 326 | 02F11 | VPWGDYHPRNVQ |
| 327 | 02F12 | VPWGDYHPGNVY |
| 328 | 02G01 | VPWGDYHPRNVS |
| 329 | 02G02 | VPWGDYHPGNVY |
| 330 | 02G05 | VPWGDYHPGNVY |
| 331 | 02G06 | VPWGDYHPGNVY |
| 332 | 02G07 | VPWGDYHPRDVY |
| 333 | 02G08 | VPWGDYHPRNVT |
| 334 | 02G09 | VPWGDYHPRNVA |
| 335 | 02G11 | VPWGDYHPRNVT |
| 336 | 02H01 | VPWGDYHPRNVY |
| 337 | 02H04 | VPWGDYHPRNVY |
| 338 | 02H05 | VPWGDYHPRNVV |
| 339 | 02H06 | VPWGDYHPRNVV |
| 340 | 02H09 | VPWGDYHPGNVY |
| 341 | 02H11 | VPWGDYHPRNVY |
| 342 | 01F07-M34Y | VPWGDYHPGNVY |
| 343 | 01F01-M34G | VPWGDYHPGNVY |
| 344 | 02G02-M34Y | VPWGDYHPGNVY |
| 345 | 02G02-M34G | VPWGDYHPGNVY |
| SEQ ID | Construct | VHH |
| NO | Name | Sequences |
| 346 | BH2T | EVQLVESGGGLVQPGRSLTLSCAASTNIFSISPMGWYRQAPGKQRE |
| | | LVAAIHGFSTLYADSVKGRFTISRDNAKNSIYLQMNSLRPEDTALYY |
| | | CNKVPWGDYHPRNVYWGQGTQVTVSS |
| 347 | 01A01 | EVQLVESGGGLVQPGRSLTLSCAASTDIFSISPMGWYRQAPGKQRE |
| | | LVAAIHGGSTLYADSVKGRFTISRDNAKNSIYLQMNSLRPEDTALY |
| | | YCNKVPWGDYHPRNVAWGQGTQVTVSS |
| 348 | 02E09 | EVQLVESGGGLVQPGRSLTLSCAASTNDFSISPMGWYRQAPGKQRE |
| | | LVAAIHGGSTLYADSVKGRFTISRDNAKNSIYLQMNSLRPEDTALY |
| | | YCNKVPWGDYHPRNVAWGQGTQVTVSS |
| 349 | 01B03 | EVQLVESGGGLVQPGRSLTLSCAASTNIFSKSPMGWYRQAPGKQRE |
| | | LVAAIHGKSTLYADSVKGRFTISRDNAKNSIYLQMNSLRPEDTALY |
| | | YCNKVPWGDYHPRNVVWGQGTQVTVSS |
| 350 | 01B04 | EVQLVESGGGLVQPGRSLTLSCAASTNDFSISPMGWYRQAPGKQRE |
| | | LVAAIHGKSTLYADSVKGRFTISRDNAKNSIYLQMNSLRPEDTALY |
| | | YCNKVPWGDYHPRNVKWGQGTQVTVSS |
| 351 | 02H05 | EVQLVESGGGLVQPGRSLTLSCAASTNQFSISPMGWYRQAPGKQRE |
| | | LVAAIHGKSTLYADSVKGRFTISRDNAKNSIYLQMNSLRPEDTALY |
| | | YCNKVPWGDYHPRNVVWGQGTQVTVSS |
| 352 | 01A02 | EVQLVESGGGLVQPGRSLTLSCAASTNIFSSSPMGWYRQAPGKQRE |
| | | LVAAINGFSTLYADSVKGRFTISRDNAKNSIYLQMNSLRPEDTALYY |
| | | CNKVPWGDYHPRNVHWGQGTQVTVSS |
| 353 | 01A05 | EVQLVESGGGLVQPGRSLTLSCAASTNIFSSSPMGWYRQAPGKQRE |
| | | LVAAIHGFSTLYADSVKGRFTISRDNAKNSIYLQMNSLRPEDTALYY |
| | | CNKVPWGDYHPRNVYWGQGTQVTVSS |
| 354 | 01B12 | EVQLVESGGGLVQPGRSLTLSCAASTNIFSTSPMGWYRQAPGKQRE |
| | | LVAAIHGFQTLYADSVKGRFTISRDNAKNSIYLQMNSLRPEDTALY |
| | | YCNKVPWGDYHPRNVVWGQGTQVTVSS |
| 355 | 01G06 | EVQLVESGGGLVQPGRSLTLSCAASTNIFSRSPMGWYRQAPGKQRE |
| | | LVAAIHGFETLYADSVKGRFTISRDNAKNSIYLQMNSLRPEDTALYY |
| | | CNKVPWGDYHPRNVLWGQGTQVTVSS |
| 356 | 02C05 | EVQLVESGGGLVQPGRSLTLSCAASTNIFSESPMGWYRQAPGKQRE |
| | | LVAAIHGFTTLYADSVKGRFTISRDNAKNSIYLQMNSLRPEDTALYY |
| | | CNKVPWGDYHPRNVTWGQGTQVTVSS |
| 357 | 02G09 | EVQLVESGGGLVQPGRSLTLSCAASTNIFSDSPMGWYRQAPGKQRE |
| | | LVAAIHGFSTLYADSVKGRFTISRDNAKNSIYLQMNSLRPEDTALYY |
| | | CNKVPWGDYHPRNVAWGQGTQVTVSS |
| 358 | 01C08 | EVQLVESGGGLVQPGRSLTLSCAASTNIFSNSPMGWYRQAPGKQRE |
| | | LVAAIHGGSTLYADSVKGRFTISRDNAKNSIYLQMNSLRPEDTALY |
| | | YCNKVPWGDYHPRNVHWGQGTQVTVSS |
| 359 | 02B01 | EVQLVESGGGLVQPGRSLTLSCAASTNIFSNSPMGWYRQAPGKQRE |
| | | LVAAIHGRSTLYADSVKGRFTISRDNAKNSIYLQMNSLRPEDTALY |
| | | YCNKVPWGDYHPRNVMWGQGTQVTVSS |
| 360 | 02E03 | EVQLVESGGGLVQPGRSLTLSCAASTNIFSNSPMGWYRQAPGKQRE |
| | | LVAAIHGPSTLYADSVKGRFTISRDNAKNSIYLQMNSLRPEDTALYY |
| | | CNKVPWGDYHPRNVTWGQGTQVTVSS |
| 361 | 01D03 | EVQLVESGGGLVQPGRSLTLSCAASTNIFSNSPMGWYRQAPGKQRE |
| | | LVAAIHGDSTLYADSVKGRFTISRDNAKNSIYLQMNSLRPEDTALY |
| | | YCNKVPWGDYHPRNVRWGQGTQVTVSS |
| 362 | 01D06 | EVQLVESGGGLVQPGRSLTLSCAASTNIFSRSPMGWYRQAPGKQRE |
| | | LVAAIHGDSTLYADSVKGRFTISRDNAKNSIYLQMNSLRPEDTALY |
| | | YCNKVPWGDYHPRNVTWGQGTQVTVSS |
| 363 | 01H04 | EVQLVESGGGLVQPGRSLTLSCAASTNIFSKSPMGWYRQAPGKQRE |
| | | LVAAIHGQSTLYADSVKGRFTISRDNAKNSIYLQMNSLRPEDTALY |
| | | YCNKVPWGDYHPRNVTWGQGTQVTVSS |
| 364 | 02B07 | EVQLVESGGGLVQPGRSLTLSCAASTNIFSSSPMGWYRQAPGKQRE |
| | | LVAAIHGHSTLYADSVKGRFTISRDNAKNSIYLQMNSLRPEDTALY |
| | | YCNKVPWGDYHPRNVTWGQGTQVTVSS |
| 365 | 01A08 | EVQLVESGGGLVQPGRSLTLSCAASTNIFSSSPMGWYRQAPGKQRE |
| | | LVAAIHGESTLYADSVKGRFTISRDNAKNSIYLQMNSLRPEDTALYY |
| | | CNKVPWGDYHPRKVYWGQGTQVTVSS |
| 366 | 01B07 | EVQLVESGGGLVQPGRSLTLSCAASTNIFSRSPMGWYRQAPGKQRE |
| | | LVAAIHGNSTLYADSVKGRFTISRDNAKNSIYLQMNSLRPEDTALY |
| | | YCNKVPWGIYHPRNVYWGQGTQVTVSS |
| 367 | 01F03 | EVQLVESGGGLVQPGRSLTLSCAASTNIFSESPMGWYRQAPGKQRE |
| | | LVAAIHGNSTLYADSVKGRFTISRDNAKNSIYLQMNSLRPEDTALY |
| | | YCNKVPWGTYHPRNVYWGQGTQVTVSS |
| 368 | 02F05 | EVQLVESGGGLVQPGRSLTLSCAASTNIFSVSPMGWYRQAPGKQRE |
| | | LVAAIHGNSTLYADSVKGRFTISRDNAKNSIYLQMNSLRPEDTALY |
| | | YCNKVPWGKYHPRNVYWGQGTQVTVSS |
| 369 | 02H04 | EVQLVESGGGLVQPGRSLTLSCAASTNIFSVSPMGWYRQAPGKQRE |
| | | LVAAIHGNSTLYADSVKGRFTISRDNAKNSIYLQMNSLRPEDTALY |
| | | YCNKVPWGDYHPRNVYWGQGTQVTVSS |
| 370 | 02A07 | EVQLVESGGGLVQPGRSLTLSCAASTNIFSKSPMGWYRQAPGKQRE |
| | | LVAAIHGNSTLYADSVKGRFTISRDNAKNSIYLQMNSLRPEDTALY |
| | | YCNKVPWGDYHPREVYWGQGTQVTVSS |
| 371 | 01D05 | EVQLVESGGGLVQPGRSLTLSCAASTNIFSDSPMGWYRQAPGKQRE |
| | | LVAAIHGTSTLYADSVKGRFTISRDNAKNSIYLQMNSLRPEDTALYY |
| | | CNKVPWGDYHPRNVYWGQGTQVTVSS |
| 372 | 01E05 | EVQLVESGGGLVQPGRSLTLSCAASTNIFSRSPMGWYRQAPGKQRE |
| | | LVAAIHGTSTLYADSVKGRFTISRDNAKNSIYLQMNSLRPEDTALYY |
| | | CNKVPWGKYHPRNVYWGQGTQVTVSS |
| 373 | 01F02 | EVQLVESGGGLVQPGRSLTLSCAASTNIFSHSPMGWYRQAPGKQRE |
| | | LVAAIHGTSTLYADSVKGRFTISRDNAKNSIYLQMNSLRPEDTALYY |
| | | CNKVPWGRYHPRNVYWGQGTQVTVSS |
| 374 | 02C06 | EVQLVESGGGLVQPGRSLTLSCAASTNIFSTSPMGWYRQAPGKQRE |
| | | LVAAIHGTSTLYADSVKGRFTISRDNAKNSIYLQMNSLRPEDTALYY |
| | | CNKVPWGDYHPGNVYWGQGTQVTVSS |
| 375 | 02F11 | EVQLVESGGGLVQPGRSLTLSCAASTNIFSTSPMGWYRQAPGKQRE |
| | | LVAAIHGTSTLYADSVKGRFTISRDNAKNSIYLQMNSLRPEDTALYY |
| | | CNKVPWGDYHPRNVQWGQGTQVTVSS |
| 376 | 01E06 | EVQLVESGGGLVQPGRSLTLSCAASTNIFSLSPMGWYRQAPGKQRE |
| | | LVAAIHGDSTLYADSVKGRFTISRDNAKNSIYLQMNSLRPEDTALY |
| | | YCNKVPWGDYHPRNVYWGQGTQVTVSS |
| 377 | 01A03 | EVQLVESGGGLVQPGRSLTLSCAASTNIFSISPGGWYRQAPGKQREL |
| | | VAAIHGSSTLYADSVKGRFTISRDNAKNSIYLQMNSLRPEDTALYYC |
| | | NKVPWGDYHPRNVYWGQGTQVTVSS |
| 378 | 02A11 | EVQLVESGGGLVQPGRSLTLSCAASTNHFSISPMGWYRQAPGKQRE |
| | | LVAAIHGSSTLYADSVKGRFTISRDNAKNSIYLQMNSLRPEDTALYY |
| | | CNKVPWGDYHPRVVYWGQGTQVTVSS |
| 379 | 01D07 | EVQLVESGGGLVQPGRSLTLSCAASTNIFSASPMGWYRQAPGKQRE |
| | | LVAAIHGSSTLYADSVKGRFTISRDNAKNSIYLQMNSLRPEDTALYY |
| | | CNKVPWGDYHPRNVNWGQGTQVTVSS |
| 380 | 01D10 | EVQLVESGGGLVQPGRSLTLSCAASTNIFSASPMGWYRQAPGKQRE |
| | | LVAAIHGSSTLYADSVKGRFTISRDNAKNSIYLQMNSLRPEDTALYY |
| | | CNKVPWGRYHPRNVYWGQGTQVTVSS |
| 381 | 01A07 | EVQLVESGGGLVQPGRSLTLSCAASTNISSISPMGWYRQAPGKQRE |
| | | LVAAIHGTSTLYADSVKGRFTISRDNAKNSIYLQMNSLRPEDTALYY |
| | | CNKVPWGDYHPGNVYWGQGTQVTVSS |
| 382 | 02F12 | EVQLVESGGGLVQPGRSLTLSCAASTNIESISPMGWYRQAPGKQRE |
| | | LVAAIHGTSTLYADSVKGRFTISRDNAKNSIYLQMNSLRPEDTALYY |
| | | CNKVPWGDYHPGNVYWGQGTQVTVSS |
| 383 | 02B05 | EVQLVESGGGLVQPGRSLTLSCAASTNIFSISPYGWYRQAPGKQREL |
| | | VAAIHGTSTLYADSVKGRFTISRDNAKNSIYLQMNSLRPEDTALYY |
| | | CNKVPWGDYHPGNVYWGQGTQVTVSS |
| 384 | 01E04 | EVQLVESGGGLVQPGRSLTLSCAASTNIASISPMGWYRQAPGKQRE |
| | | LVAAIHGTSTLYADSVKGRFTISRDNAKNSIYLQMNSLRPEDTALYY |
| | | CNKVPWGDYHPGNVYWGQGTQVTVSS |
| 385 | 02A05 | EVQLVESGGGLVQPGRSLTLSCAASTNIASISPMGWYRQAPGKQRE |
| | | LVAAIHGKSTLYADSVKGRFTISRDNAKNSIYLQMNSLRPEDTALY |
| | | YCNKVPWGDYHPGNVYWGQGTQVTVSS |
| 386 | 02C03 | EVQLVESGGGLVQPGRSLTLSCAASTNIASISPMGWYRQAPGKQRE |
| | | LVAAIHGSSTLYADSVKGRFTISRDNAKNSIYLQMNSLRPEDTALYY |
| | | CNKVPWGDYHPGNVYWGQGTQVTVSS |
| 387 | 01E03 | EVQLVESGGGLVQPGRSLTLSCAASTNITSISPMGWYRQAPGKQRE |
| | | LVAAIHGDSTLYADSVKGRFTISRDNAKNSIYLQMNSLRPEDTALY |
| | | YCNKVPWGDYHPGNVYWGQGTQVTVSS |
| 388 | 01H09 | EVQLVESGGGLVQPGRSLTLSCAASTNIMSISPMGWYRQAPGKQRE |
| | | LVAAIHGNSTLYADSVKGRFTISRDNAKNSIYLQMNSLRPEDTALY |
| | | YCNKVPWGDYHPGNVYWGQGTQVTVSS |
| 389 | 02G05 | EVQLVESGGGLVQPGRSLTLSCAASTNITSISPMGWYRQAPGKQRE |
| | | LVAAIHGNSTLYADSVKGRFTISRDNAKNSIYLQMNSLRPEDTALY |
| | | YCNKVPWGDYHPGNVYWGQGTQVTVSS |
| 390 | 01C01 | EVQLVESGGGLVQPGRSLTLSCAASTNIVSISPMGWYRQAPGKQRE |
| | | LVAAIHGHSTLYADSVKGRFTISRDNAKNSIYLQMNSLRPEDTALY |
| | | YCNKVPWGDYHPGNVYWGQGTQVTVSS |
| 391 | 01D02 | EVQLVESGGGLVQPGRSLTLSCAASTNIVSISPMGWYRQAPGKQRE |
| | | LVAAIHGKSTLYADSVKGRFTISRDNAKNSIYLQMNSLRPEDTALY |
| | | YCNKVPWGDYHPGNVYWGQGTQVTVSS |
| 392 | 02D09 | EVQLVESGGGLVQPGRSLTLSCAASTNVVSISPMGWYRQAPGKQRE |
| | | LVAAIHGKSTLYADSVKGRFTISRDNAKNSIYLQMNSLRPEDTALY |
| | | YCNKVPWGDYHPNNVYWGQGTQVTVSS |
| 393 | 02C01 | EVQLVESGGGLVQPGRSLTLSCAASTNIISISPMGWYRQAPGKQREL |
| | | VAAIHGASTLYADSVKGRFTISRDNAKNSIYLQMNSLRPEDTALYY |
| | | CNKVPWGDYHPGNVYWGQGTQVTVSS |
| 394 | 02G02 | EVQLVESGGGLVQPGRSLTLSCAASTNIFSITPMGWYRQAPGKQRE |
| | | LVAAIHGASTLYADSVKGRFTISRDNAKNSIYLQMNSLRPEDTALY |
| | | YCNKVPWGDYHPGNVYWGQGTQVTVSS |
| 395 | 01B05 | EVQLVESGGGLVQPGRSLTLSCAASTNITSISPMGWYRQAPGKQRE |
| | | LVAAIHGFETLYADSVKGRFTISRDNAKNSIYLQMNSLRPEDTALYY |
| | | CNKVPWGDYHPGNVYWGQGTQVTVSS |
| 396 | 01G08 | EVQLVESGGGLVQPGRSLTLSCAASTNIQSISPMGWYRQAPGKQRE |
| | | LVAAIHGFETLYADSVKGRFTISRDNAKNSIYLQMNSLRPEDTALYY |
| | | CNKVPWGDYHPGNVYWGQGTQVTVSS |
| 397 | 01H06 | EVQLVESGGGLVQPGRSLTLSCAASTSDFSISPMGWYRQAPGKQRE |
| | | LVAAIHGFETLYADSVKGRFTISRDNAKNSIYLQMNSLRPEDTALYY |
| | | CNKVPWGDYHPGNVYWGQGTQVTVSS |
| 398 | 01F04 | EVQLVESGGGLVQPGRSLTLSCAASTNIDSISPMGWYRQAPGKQRE |
| | | LVAAIHGFQTLYADSVKGRFTISRDNAKNSIYLQMNSLRPEDTALY |
| | | YCNKVPWGDYHPGNVYWGQGTQVTVSS |
| 399 | 01H08 | EVQLVESGGGLVQPGRSLTLSCAASTNIMSISPMGWYRQAPGKQRE |
| | | LVAAIHGFSTVYADSVKGRFTISRDNAKNSIYLQMNSLRPEDTALY |
| | | YCNKVPWGDYHPGNVYWGQGTQVTVSS |
| 400 | 02F07 | EVQLVESGGGLVQPGRSLTLSCAASTNIESISPMGWYRQAPGKQRE |
| | | LVAAIHGFSTLYADSVKGRFTISRDNAKNSIYLQMNSLRPEDTALYY |
| | | CNKVPWGDYHPGNVYWGQGTQVTVSS |
| 401 | 01C05 | EVQLVESGGGLVQPGRSLTLSCAASTNIFSSSPMGWYRQAPGKQRE |
| | | LVAAIHGFKTLYADSVKGRFTISRDNAKNSIYLQMNSLRPEDTARY |
| | | YCNKVPWGDYHPGNVYWGQGTQVTVSS |
| 402 | 02F04 | EVQLVESGGGLVQPGRSLTLSCAASTNIFSSSPMGWYRQAPGKQRE |
| | | LVAAIHGFSTLYADSVKGRFTISRDNAKNSIYLQMNSLRPEDTALYY |
| | | CNKVPWGDYHPGNVYWGQGTQVTVSS |
| 403 | 02B06 | EVQLVESGGGLVQPGRSLTLSCAASTNIFSNSPMGWYRQAPGKQRE |
| | | LVAAIHGFSTLYADSVKGRFTISRDNAKNSIYLQMNSLRPEDTALYY |
| | | CNKVPWGDYHPGNVYWGQGTQVTVSS |
| 404 | 01F07 | EVQLVESGGGLVQPGRSLTLSCAASTNIFSTSPMGWYRQAPGKQRE |
| | | LVAAIHGFSTIYADSVKGRFTISRDNAKNSIYLQMNSLRPEDTALYY |
| | | CNKVPWGDYHPGNVYWGQGTQVTVSS |
| 405 | 02B04 | EVQLVESGGGLVQPGRSLTLSCAASTNIFSTSPMGWYRQAPGKQRE |
| | | LVAAIHGFSTIYADSVKGRFTISRDNAKNSIYLQMNSLRPEDTALYY |
| | | CNKVPWGDYHPLNVYWGQGTQVTVSS |
| 406 | 01H11 | EVQLVESGGGLVQPGRSLTLSCVASTNIFSTSPMGWYRQAPGKQRE |
| | | LVAAIHGFSTLYADSVKGRFTISRDNAKNSIYLQMNSLRPEDTALYY |
| | | CNKVPWGDYHPGNVYWGQGTQVTVSS |
| 407 | 02E06 | EVQLVESGGGLVQPGRSLTLSCAASTNIFSDSPMGWYRQAPGKQRE |
| | | LVAAIHGFSTFYADSVKGRFTISRDNAKNSIYLQMNSLRPEDTALYY |
| | | CNKVPWGDYHPGNVYWGQGTQVTVSS |
| 408 | 01E08 | EVQLVESGGGLVQPGRSLTLSCAASTNIFSQSPMGWYRQAPGKQRE |
| | | LVAAIHGDSTLYADSVKGRFTISRDNAKNSIYLQMNSLRPEDTALY |
| | | YCNKVPWGDYHPGNVCWGQGTQVTVSS |
| 409 | 02A04 | EVQLVESGGGLVQPGRSLTLSCAASTNIFSQSPMGWYRQAPGKQRE |
| | | LVAAIHGKSTLYADSVKGRFTISRDNAKNSIYLQMNSLRPEDTALY |
| | | YCNKVPWGDYHPSNVYWGKGTQVTVSS |
| 410 | 02A08 | EVQLVESGGGLVQPGRSLTLSCAASTNIFSRSPMGWYRQAPGKQRE |
| | | LVAAIHGESTLYADSVKGRFTISRDNAKNSIYLQMNSLRPEDTALYY |
| | | CNKVPWGRYHPGNVYWGQGTQVTVSS |
| 411 | 02E05 | EVQLVESGGGLVQPGRSLTLSCAASTNIFSRSPMGWYRQAPGKQRE |
| | | LVAAIHGISTLYADSVKGRFTISRDNAKNSIYLQMNSLRPEDTALYY |
| | | CNKVPWGDYHPGNVYWGQGTQVTVSS |
| 412 | 02H09 | EVQLVESGGGLVQPGRSLTLSCAASTNIFSRSPMGWYRQAPGKQRE |
| | | LVAAIHGSSTLYADSVKGRFTISRDNAKNSIYLQMNSLRPEDTALYY |
| | | CNKVPWGDYHPGNVYWGQGTQVTVSS |
| 413 | 02G06 | EVQLVESGGGLVQPGRSLTLSCAASTNIFSGSPMGWYRQAPGKQRE |
| | | LVAAIHGNSTLYADSVKGRFTISRDNAKNSIYLQMNSLRPEDTALY |
| | | YCNKVPWGDYHPGNVYWGQGTQVTVSS |
| 414 | 01B09 | EVQLVESGGGLVQPGRSLTLSCAASSNIFSISPMGWYRQAPGKQREL |
| | | VAAIHGSSTLYADSVKGRFTISRDNAKNSIYLQMNSLRPEDTALYYC |
| | | NKVPWGDYHPGNVYWGQGTQVTVSS |
| 415 | 02F03 | EVQLVESGGGLVQPGRSLTLSCAASTNIFSIYPMGWYRQAPGKQRE |
| | | LVAAIHGSSTLYADSVKGRFTISRDNAKNSIYLQMNSLRPEDTALYY |
| | | CNKVPWGDYHPKNVYWGQGTQVTVSS |
| 416 | 02F02 | EVQLVESGGGLVQPGRSLTLSCAASTNIFSKSPMGWYRQAPGKQRE |
| | | LVAAIHGSSTLYADSVKGRFTISRDNAKNSIYLQMNSLRPEDTALYY |
| | | CNKVPWGDYHPGNVYWGQGTQVTVSS |
| 417 | 02H01 | EVQLVESGGGLVQPGRSLTLSCAASTNIFSKSPMGWYRQAPGKQRE |
| | | LVAAIHGSSTLYADSVKGRFTISRDNAKNSIYLQMNSLRPEDTALYY |
| | | CNKVPWGDYHPRNVYWGQGTQVTVSS |
| 418 | 01G10 | EVQLVESGGGLVQPGRSLTLSCAASTNEFSISPMGWYRQAPGKQRE |
| | | LVAAIHGLSTLYADSVKGRFTISRDNAKNSIYLQMNSLRPEDTALYY |
| | | CNKVPWGAYHPRNVYWGQGTQVTVSS |
| 419 | 02D11 | EVQLVESGGGLVQPGRSLTLSCAASTNEFSISPMGWYRQAPGKQRE |
| | | LVAAIHGESTLYADSVKGRFTISRDNAKNSIYLQMNSLRPEDTALYY |
| | | CNKVPWGDYHPGNVYWGQGTQVTVSS |
| 420 | 01B01 | EVQLVESGGGLVQPGRSLTLSCAASTNIPSISPMGWYRQAPGKQRE |
| | | LVAAIHGESTLYADSVKGRFTISRDNAKNSIYLQMNSLRPEDTALYY |
| | | CNKVPWGDYHPRNVAWGQGTQVTVSS |
| 421 | 01G11 | EVQLVESGGGLVQPGRSLTLSCAASTNIPSISPMGWYRQAPGKQRE |
| | | LVAAIHGASTLYADSVKGRFTISRDNAKNSIYLQMNSLRPEDTALY |
| | | YCNKVPWGDYHPRNVAWGQGTQVTVSS |
| 422 | 01H10 | EVQLVESGGGLVQPGRSLTLSCAASTNIPSISPMGWYRQAPGKQRE |
| | | LVAAIHGESTLYADSVKGRFTISRDNAKNSIYLQMNSLRPEDTALYY |
| | | CNKVPWGDYHPRNVYWGQGTQVTVSS |
| 423 | 01C04 | EVQLVESGGGLVQPGRSLTLSCAASTNIPSISPMGWYRQAPGKQRE |
| | | LVAAIHGDSTLYADSVKGRFTISRDNAKNSIYLQMNSLRPEDTALY |
| | | YCNKVPWGDYHPRNVYWGQGTQVTVSS |
| 424 | 01D04 | EVQLVESGGGLVQPGRSLTLSCAASTNITSISPMGWYRQAPGKQRE |
| | | LVAAIHGVSTLYADSVKGRFTISRDNAKNSIYLQMNSLRPEDTALY |
| | | YCNKVPWGDYHPRNVQWGQGTQVTVSS |
| 425 | 01E07 | EVQLVESGGGLVQPGRSLTLSCAASTNIPSISPMGWYRQAPGKQRE |
| | | LVAAIHGQSTLYADSVKGRFTISRDNAKNSIYLQMNSLRPEDTALY |
| | | YCNKVPWGDYHPRNVQWGQGTQVTVSS |
| 426 | 02B11 | EVQLVESGGGLVQPGRSLTLSCAASTNIVSISPMGWYRQAPGKQRE |
| | | LVAAIHGDSTLYADSVKGRFTISRDNAKNSIYLQMNSLRPEDTALY |
| | | YCNKVPWGDYHPRNVSWGQGTQVTVSS |
| 427 | 01F10 | EVQLVESGGGLVQPGRSLTLSCAASSNIFSISPMGWYRQAPGKQREL |
| | | VAAIHGESTLYADSVKGRFTISRDNAKNSIYLQMNSLRPEDTALYY |
| | | CNKVPWGDYHPRNVTWGQGTQVTVSS |
| 428 | 02G08 | EVQLVESGGGLVQPGRSLTLSCAASTNIDSISPMGWYRQAPGKQRE |
| | | LVAAIHGESTLYADSVKGRFTISRDNAKNSIYLQMNSLRPEDTALYY |
| | | CNKVPWGDYHPRNVTWGQGTQVTVSS |
| 429 | 02G11 | EVQLVESGGGLVQPGRSLTLSCAASTNIDSISPMGWYRQAPGKQRE |
| | | LVAAIHGSSTLYADSVKGRFTISRDNAKNSIYLQMNSLRPEDTALYY |
| | | CNKVPWGDYHPRNVTWGQGTQVTVSS |
| 430 | 02H06 | EVQLVESGGGLVQPGRSLTLSCAASTNIRSISPMGWYRQAPGKQRE |
| | | LVAAIHGSSTLYADSVKGRFTISRDNAKNSIYLQMNSLRPEDTALYY |
| | | CNKVPWGDYHPRNVVWGQGTQVTVSS |
| 431 | 01B02 | EVQLVESGGGLVQPGRSLTLSCAASTNITSISPMGWYRQAPGKQRE |
| | | LVAAISGFSTLYADSVKGRFTISRDNAKNSIYLQMNSLRPEDTALYY |
| | | CNEVPWGDYHPRNVYWGQGTQVTVSS |
| 432 | 02H11 | EVQLVESGGGLVQPGRSLTLSCAASTNITSISPMGWYRQAPGKQRE |
| | | LVAAIHGESTLYADSVKGRFTISRDNAKNSIYLQMNSLRPEDTALYY |
| | | CNKVPWGDYHPRNVYWGQGTQVTVSS |
| 433 | 01F08 | EVQLVESGGGLVQPGRSLTLSCAASTNITSVSPMGWYRQAPGKQRE |
| | | LVAAIHGPSTLYADSVKGRFTISRDNAKNSIYLQMNSLRPEDTALYY |
| | | CNKVPWGDYHPTNVYWGQGTQVTVSS |
| 434 | 01H01 | EVQLVESGGGLVQPGRSLTLSCAASTNIGSISPMGWYRQAPGKQRE |
| | | LVAAIHGQSTLYADSVKGRFTISRDNAKNSIYLQMNSLRPEDTALY |
| | | YCNKVPWGDYHPQNVYWGQGTQVTVSS |
| 435 | 01E10 | EVQLVESGGGLVQPGRSLTLSCAASTNIESISPMGWYRQAPGKQRE |
| | | LVAAIHGKSTLYADSVKGRFTISRDNAKNSIYLQMNSLRPEDTALY |
| | | YCNKVPWGDYHPRRVYWGQGTQVTVSS |
| 436 | 01G01 | EVQLVESGGGLVQPGRSLTLSCAASTNIVSISPMGWYRQAPGKQRE |
| | | LVAAIHGDSTLYADSVKGRFTISRDNAKNSIYLQMNSLRPEDTALY |
| | | YCNKVPWGDYHPRRVYWGQGTQVTVSS |
| 437 | 01G04 | EVQLVESGGGLVQPGRSLTLSCAASTNIDSISPMGWYRQAPGKQRE |
| | | LVAAIHGNSTLYADSVKGRFTISRDNAKNSIYLQMNSLRPEDTALY |
| | | YCNKVPWGDYHPRMVYWGQGTQVTVSS |
| 438 | 01A04 | EVQLVESGGGLVQPGRSLTLSCAASTNIFMISPMGWYRQAPGKQRE |
| | | LVAAIHGDSTLYADSVKGRFTISRDNAKNSIYLQMNSLRPEDTALY |
| | | YCNKVPWGRYHPRNVYWGQGTQVTVSS |
| 439 | 01F12 | EVQLVESGGGLVQPGRSLTLSCAASTNIFRISPMGWYRQAPGKQRE |
| | | LVAAIHGDSTLYADSVKGRFTISRDNAKNSIYLQMNSLRPEDTALY |
| | | YCNKVPWGRYHPRNVYWGQGTQVTVSS |
| 440 | 01B06 | EVQLVESGGGLVQPGRSLTLSCAASTNIFSISPMGWYRQAPGKQRE |
| | | LVAAIHGDSTLYADSVKGRFTISRDNAKNSIYLQMNSLRPEDTALY |
| | | YCNKVPWGEYHPRNVYWGQGTQVTVSS |
| 441 | 01C06 | EVQLVESGGGLVQPGRSLTLSCAASTNIFSISPMGWYRQAPGKQRE |
| | | LVAAIHGDSTLYADSVKGRFTISRDNAKNSIYLQMNSLRPEDTALY |
| | | YCNKVPWGKYHPRNVYWGQGTQVTVSS |
| 442 | 01B08 | EVQLVESGGGLVQPGRSLTLSCAASTNIESISPMGWYRQAPGKQRE |
| | | LVAAIHGSSTLYADSVKGRFTISRDNAKNSIYLQMNSLRPEDTALYY |
| | | CNKVPWGRYHPRNVYWGQGTQVTVSS |
| 443 | 01C02 | EVQLVESGGGLVQPGRSLTLSCAASTNIESISPMGWYRQAPGKQRE |
| | | LVAAIHGNSTLYADSVKGRFTISRDNAKNSIYLQMNSLRPEDTALY |
| | | YCNKVPWGRYHPRNVYWGQGTQVTVSS |
| 444 | 01C10 | EVQLVESGGGLVQPGRSLTLSCAASTNISSISPMGWYRQAPGKQRE |
| | | LVAAIHGFSTLYADSVKGRFTISRDNAKNSIYLQMNSLRPEDTALYY |
| | | CNKVPWGYYHPRNVYWGQGTQVTVSS |
| 445 | 01F09 | EVQLVESGGGLVQPGRSLTLSCAASTNISSISPMGWYRQAPGKQRE |
| | | LVAAIHGHSTLYADSVKGRFTISRDNAKNSIYLQMNSLRPEDTALY |
| | | YCNKVPWGRYHPRNVYWGQGTQVTVSS |
| 446 | 02D06 | EVQLVESGGGLVQPGRSLTLSCAASTNISSISPMGWYRQAPGKQRE |
| | | LVAAIHGFSTVYADSVKGRFTISRDNAKNSIYLQMNSLRPEDTALY |
| | | YCNKVPWGRYHPRNVYWGQGTQVTVSS |
| 447 | 01A06 | EVQLVESGGGLVQPGRSLTLSCAASTNIFSIRPMGWYRQAPGKQRE |
| | | LVAAIHGFSTVYADSVKGRFTISRDNAKNSIYLQMNSLRPEDTALY |
| | | YCNKVPWGDYHPRNVYWGQGTQVTVSS |
| 448 | 01C07 | EVQLVESGGGLVQPGRSLTLSCAASTNIFSIYPMGWYRQAPGKQRE |
| | | LVAAIHGFSTYYADSVKGRFTISRDNAKNSIYLQMNSLRPEDTALY |
| | | YCNKVPWGSYHPRNVYWGQGTQVTVSS |
| 449 | 01G09 | EVQLVESGGGLVQPGRSLTLSCAASTNIFNISPMGWYRQAPGKQRE |
| | | LVAAIHGFSTYYADSVKGRFTISRDNAKNSIYLQMNSLRPEDTALY |
| | | YCNKVPWGRYHPRNVYWGQGTQVTVSS |
| 450 | 01F05 | EVQLVESGGGLVQPGRSLTLSCAASTNIFSSSPMGWYRQAPGKQRE |
| | | LVAAIHGFSTWYADSVKGRFTISRDNAKNSIYLQMNSLRPEDTALY |
| | | YCNKVPWGRYHPRNVYWGQGTQVTVSS |
| 451 | 02B12 | EVQLVESGGGLVQPGRSLTLSCAASTNISSISPMGWYRQAPGKQRE |
| | | LVAAIHGFDTLYADSVKGRFTISRDNAKNSIYLQMNSLRPEDTALY |
| | | YCNKVPWGDYHPRNVYWGQGTQVTVSS |
| 452 | 02G01 | EVQLVESGGGLVQPGRSLTLSCAASTNIFSINPMGWYRQAPGKQRE |
| | | LVAAIHGFDTLYADSVKGRFTISRDNAKNSIYLQMNSLRPEDTALY |
| | | YCNKVPWGDYHPRNVSWGQGTQVTVSS |
| 453 | 01A09 | EVQLVESGGGLVQPGRSLTLSCAASTNIFSITPMGWYRQAPGKQRE |
| | | LVAAIHGRSTLYADSVKGRFTISRDNAKNSIYLQMNSLRPEDTALY |
| | | YCNKVPWGSYHPRNVYWGQGTQVTVSS |
| 454 | 01H05 | EVQLVESGGGLVQPGRSLTLSCAASTNIFSITPMGWYRQAPGKQRE |
| | | LVAAIHGTSTLYADSVKGRFTISRDNAKNSIYLQMNSLRPEDTALYY |
| | | CNKVPWGRYHPRNVYWGQGTQVTVSS |
| 455 | 02F06 | EVQLVESGGGLVQPGRSLTLSCAASTNIFSITPMGWYRQAPGKQRE |
| | | LVAAIHGESTLYADSVKGRFTISRDNAKNSIYLQMNSLRPEDTALYY |
| | | CNKVPWGRYHPRNVYWGQGTQVTVSS |
| 456 | 02G07 | EVQLVESGGGLVQPGRSLTLSCAASTNIFSITPMGWYRQAPGKQRE |
| | | LVAAIHGESTLYADSVKGRFTISRDNAKNSIYLQMNSLRPEDTALYY |
| | | CNKVPWGDYHPRDVYWGQGTQVTVSS |
| 457 | 01F07- | EVQLVESGGGLVQPGRSLTLSCAASTNIFSTSPYGWYRQAPGKQRE |
| | M34Y | LVAAIHGFSTIYADSVKGRFTISRDNAKNSIYLQMNSLRPEDTALYY |
| | | CNKVPWGDYHPGNVYWGQGTQVTVSS |
| 458 | 01F01- | EVQLVESGGGLVQPGRSLTLSCAASTNIFSTSPGGWYRQAPGKQRE |
| | M34G | LVAAIHGFSTIYADSVKGRFTISRDNAKNSIYLQMNSLRPEDTALYY |
| | | CNKVPWGDYHPGNVYWGQGTQVTVSS |
| 459 | 02G02- | EVQLVESGGGLVQPGRSLTLSCAASTNIFSITPYGWYRQAPGKQREL |
| | M34Y | VAAIHGASTLYADSVKGRFTISRDNAKNSIYLQMNSLRPEDTALYY |
| | | CNKVPWGDYHPGNVYWGQGTQVTVSS |
| 460 | 02G02- | EVQLVESGGGLVQPGRSLTLSCAASTNIFSITPGGWYRQAPGKQREL |
| | M34G | VAAIHGASTLYADSVKGRFTISRDNAKNSIYLQMNSLRPEDTALYY |
| | | CNKVPWGDYHPGNVYWGQGTQVTVSS |
| 461 | F1 | EVQLVESGGGLVQPGRSLTLSCAAS |
| 462 | F1 | EVQLVESGGGLVQPGRSLTLSCVAS |
| 463 | F2 | WYRQAPGKQRELVA |
| 464 | F3 | GRFTISRDNAKNSIYLQMNSLRPEDTALYYCNK |
| 465 | F3 | GRFTISRDNAKNSIYLQMNSLRPEDTALYYCNE |
| 466 | F4 | WGQGTQVTVSS |
| 467 | F4 | WGKGTQVTVSS |
| 468 | Human | MLQMAGQCSQNEYFDSLLHACIPCQLRCSSNTPPLTCQRYCNASVT |
| | BCMA | NSVKGTNAILWTCLGLSLIISLAVFVLMFLLRKINSEPLKDEFKNTGS |
| | | GLLGMANIDLEKSRTGDEIILPRGLEYTVEECTCEDCIKSKPKVDSD |
| | | HCFPLPAMEEGATILVTTKTNDYCKSLPAALSATEIEKSISAR |
| 469 | Murine | MAQQCFHSEYFDSLLHACKPCHLRCSNPPATCQPYCDPSVTSSVKG |
| | BCMA | TYTVLWIFLGLTLVLSLALFTISFLLRKMNPEALKDEPQSPGQLDGS |
| | | AQLDKADTELTRIRAGDDRIFPRSLEYTVEECTCEDCVKSKPKGDSD |
| | | HFFPLPAMEEGATILVTTKTGDYGKSSVPTALQSVMGMEKPTHTR |
| 470 | Cynomolgus | MLQMARQCSQNEYFDSLLHDCKPCQLRCSSTPPLTCQRYCNASMT |
| | BCMA | NSVKGMNAILWTCLGLSLIISLAVFVLTFLLRKMSSEPLKDEFKNTG |
| | | SGLLGMANIDLEKGRTGDEIVLPRGLEYTVEECTCEDCIKNKPKVDS |
| | | DHCFPLPAMEEGATILVTTKTNDYCNSLSAALSVTEIEKSISAR |
| 471 | 6x His tag | His-His-His-His-His-His |
| SEQ ID | | |
| NO | Construct Name | Sequence |
| 472 | Exemplary linker | (GS)n |
| | sequence | |
| 473 | Exemplary linker | (GGS)n |
| | sequence | |
| 474 | Exemplary linker | (GGGS)n |
| | sequence | |
| 475 | Exemplary linker | (GGSG)n |
| | sequence | |
| 476 | Exemplary linker | (GGSGG)n |
| | sequence | |
| 477 | Exemplary linker | (GGGGS)n |
| | sequence | |
| 478 | Exemplary linker | (GGGGG)n |
| | sequence | |
| 479 | Exemplary linker | (GGG)n |
| | sequence | |
| 480 | Exemplary linker | (GGGGS)4 |
| | sequence | |
| 481 | Exemplary linker | (GGGGS)3 |
| | sequence | |
| 482 | Exemplary linker | LPETG |
| | sequence | |
| 483 | Exemplary BH2T | EVQLVESGGGLVQPGRSLTLSCAASTNIFSISPMGWYRQAPGK |
| | TriTAC sequence | QRELVAAIHGFSTLYADSVKGRFTISRDNAKNSIYLQMNSLRP |
| | | EDTALYYCNKVPWGDYHPRNVYWGQGTQVTVSSGGGGSGG |
| | | GSEVQLVESGGGLVQPGNSLRLSCAASGFTFSKFGMSWVRQA |
| | | PGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 484 | Exemplary 01A01 | EVQLVESGGGLVQPGRSLTLSCAASTDIFSISPMGWYRQAPGK |
| | TriTAC sequence | QRELVAAIHGGSTLYADSVKGRFTISRDNAKNSIYLQMNSLRP |
| | | EDTALYYCNKVPWGDYHPRNVAWGQGTQVTVSSGGGGSGG |
| | | GSEVQLVESGGGLVQPGNSLRLSCAASGFTFSKFGMSWVRQA |
| | | PGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 485 | Exemplary 02E09 | EVQLVESGGGLVQPGRSLTLSCAASTNDFSISPMGWYRQAPG |
| | TriTAC sequence | KQRELVAAIHGGSTLYADSVKGRFTISRDNAKNSIYLQMNSLR |
| | | PEDTALYYCNKVPWGDYHPRNVAWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 486 | Exemplary 01B03 | EVQLVESGGGLVQPGRSLTLSCAASTNIFSKSPMGWYRQAPG |
| | TriTAC sequence | KQRELVAAIHGKSTLYADSVKGRFTISRDNAKNSIYLQMNSLR |
| | | PEDTALYYCNKVPWGDYHPRNVVWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 487 | Exemplary 01B04 | EVQLVESGGGLVQPGRSLTLSCAASTNDFSISPMGWYRQAPG |
| | TriTAC sequence | KQRELVAAIHGKSTLYADSVKGRFTISRDNAKNSIYLQMNSLR |
| | | PEDTALYYCNKVPWGDYHPRNVKWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 488 | Exemplary 02H05 | EVQLVESGGGLVQPGRSLTLSCAASTNQFSISPMGWYRQAPG |
| | TriTAC sequence | KQRELVAAIHGKSTLYADSVKGRFTISRDNAKNSIYLQMNSLR |
| | | PEDTALYYCNKVPWGDYHPRNVVWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 489 | Exemplary 01A02 | EVQLVESGGGLVQPGRSLTLSCAASTNIFSSSPMGWYRQAPG |
| | TriTAC sequence | KQRELVAAINGFSTLYADSVKGRFTISRDNAKNSIYLQMNSLR |
| | | PEDTALYYCNKVPWGDYHPRNVHWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 490 | Exemplary 01A05 | EVQLVESGGGLVQPGRSLTLSCAASTNIFSSSPMGWYRQAPG |
| | TriTAC sequence | KQRELVAAIHGFSTLYADSVKGRFTISRDNAKNSIYLQMNSLR |
| | | PEDTALYYCNKVPWGDYHPRNVYWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 491 | Exemplary 01B12 | EVQLVESGGGLVQPGRSLTLSCAASTNIFSTSPMGWYRQAPG |
| | TriTAC sequence | KQRELVAAIHGFQTLYADSVKGRFTISRDNAKNSIYLQMNSLR |
| | | PEDTALYYCNKVPWGDYHPRNVVWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 492 | Exemplary 01G06 | EVQLVESGGGLVQPGRSLTLSCAASTNIFSRSPMGWYRQAPG |
| | TriTAC sequence | KQRELVAAIHGFETLYADSVKGRFTISRDNAKNSIYLQMNSLR |
| | | PEDTALYYCNKVPWGDYHPRNVLWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 493 | Exemplary 02C05 | EVQLVESGGGLVQPGRSLTLSCAASTNIFSESPMGWYRQAPG |
| | TriTAC sequence | KQRELVAAIHGFTTLYADSVKGRFTISRDNAKNSIYLQMNSLR |
| | | PEDTALYYCNKVPWGDYHPRNVTWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 494 | Exemplary 02G09 | EVQLVESGGGLVQPGRSLTLSCAASTNIFSDSPMGWYRQAPG |
| | TriTAC sequence | KQRELVAAIHGFSTLYADSVKGRFTISRDNAKNSIYLQMNSLR |
| | | PEDTALYYCNKVPWGDYHPRNVAWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 495 | Exemplary 01C08 | EVQLVESGGGLVQPGRSLTLSCAASTNIFSNSPMGWYRQAPG |
| | TriTAC sequence | KQRELVAAIHGGSTLYADSVKGRFTISRDNAKNSIYLQMNSLR |
| | | PEDTALYYCNKVPWGDYHPRNVHWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 496 | Exemplary 02B01 | EVQLVESGGGLVQPGRSLTLSCAASTNIFSNSPMGWYRQAPG |
| | TriTAC sequence | KQRELVAAIHGRSTLYADSVKGRFTISRDNAKNSIYLQMNSLR |
| | | PEDTALYYCNKVPWGDYHPRNVMWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 497 | Exemplary 02E03 | EVQLVESGGGLVQPGRSLTLSCAASTNIFSNSPMGWYRQAPG |
| | TriTAC sequence | KQRELVAAIHGPSTLYADSVKGRFTISRDNAKNSIYLQMNSLR |
| | | PEDTALYYCNKVPWGDYHPRNVTWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 498 | Exemplary 01D03 | EVQLVESGGGLVQPGRSLTLSCAASTNIFSNSPMGWYRQAPG |
| | TriTAC sequence | KQRELVAAIHGDSTLYADSVKGRFTISRDNAKNSIYLQMNSLR |
| | | PEDTALYYCNKVPWGDYHPRNVRWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 499 | Exemplary 01D06 | EVQLVESGGGLVQPGRSLTLSCAASTNIFSRSPMGWYRQAPG |
| | TriTAC sequence | KQRELVAAIHGDSTLYADSVKGRFTISRDNAKNSIYLQMNSLR |
| | | PEDTALYYCNKVPWGDYHPRNVTWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 500 | Exemplary 01H04 | EVQLVESGGGLVQPGRSLTLSCAASTNIFSKSPMGWYRQAPG |
| | TriTAC sequence | KQRELVAAIHGQSTLYADSVKGRFTISRDNAKNSIYLQMNSLR |
| | | PEDTALYYCNKVPWGDYHPRNVTWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 501 | Exemplary 02B07 | EVQLVESGGGLVQPGRSLTLSCAASTNIFSSSPMGWYRQAPG |
| | TriTAC sequence | KQRELVAAIHGHSTLYADSVKGRFTISRDNAKNSIYLQMNSLR |
| | | PEDTALYYCNKVPWGDYHPRNVTWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 502 | Exemplary 01A08 | EVQLVESGGGLVQPGRSLTLSCAASTNIFSSSPMGWYRQAPG |
| | TriTAC sequence | KQRELVAAIHGESTLYADSVKGRFTISRDNAKNSIYLQMNSLR |
| | | PEDTALYYCNKVPWGDYHPRKVYWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 503 | Exemplary 01B07 | EVQLVESGGGLVQPGRSLTLSCAASTNIFSRSPMGWYRQAPG |
| | TriTAC sequence | KQRELVAAIHGNSTLYADSVKGRFTISRDNAKNSIYLQMNSLR |
| | | PEDTALYYCNKVPWGIYHPRNVYWGQGTQVTVSSGGGGSGG |
| | | GSEVQLVESGGGLVQPGNSLRLSCAASGFTFSKFGMSWVRQA |
| | | PGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 504 | Exemplary 01F03 | EVQLVESGGGLVQPGRSLTLSCAASTNIFSESPMGWYRQAPG |
| | TriTAC sequence | KQRELVAAIHGNSTLYADSVKGRFTISRDNAKNSIYLQMNSLR |
| | | PEDTALYYCNKVPWGTYHPRNVYWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 505 | Exemplary 02F05 | EVQLVESGGGLVQPGRSLTLSCAASTNIFSVSPMGWYRQAPG |
| | TriTAC sequence | KQRELVAAIHGNSTLYADSVKGRFTISRDNAKNSIYLQMNSLR |
| | | PEDTALYYCNKVPWGKYHPRNVYWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 506 | Exemplary 02H04 | EVQLVESGGGLVQPGRSLTLSCAASTNIFSVSPMGWYRQAPG |
| | TriTAC sequence | KQRELVAAIHGNSTLYADSVKGRFTISRDNAKNSIYLQMNSLR |
| | | PEDTALYYCNKVPWGDYHPRNVYWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 507 | Exemplary 02A07 | EVQLVESGGGLVQPGRSLTLSCAASTNIFSKSPMGWYRQAPG |
| | TriTAC sequence | KQRELVAAIHGNSTLYADSVKGRFTISRDNAKNSIYLQMNSLR |
| | | PEDTALYYCNKVPWGDYHPREVYWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 508 | Exemplary 01D05 | EVQLVESGGGLVQPGRSLTLSCAASTNIFSDSPMGWYRQAPG |
| | TriTAC sequence | KQRELVAAIHGTSTLYADSVKGRFTISRDNAKNSIYLQMNSLR |
| | | PEDTALYYCNKVPWGDYHPRNVYWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 509 | Exemplary 01E05 | EVQLVESGGGLVQPGRSLTLSCAASTNIFSRSPMGWYRQAPG |
| | TriTAC sequence | KQRELVAAIHGTSTLYADSVKGRFTISRDNAKNSIYLQMNSLR |
| | | PEDTALYYCNKVPWGKYHPRNVYWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 510 | Exemplary 01F02 | EVQLVESGGGLVQPGRSLTLSCAASTNIFSHSPMGWYRQAPG |
| | TriTAC sequence | KQRELVAAIHGTSTLYADSVKGRFTISRDNAKNSIYLQMNSLR |
| | | PEDTALYYCNKVPWGRYHPRNVYWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 511 | Exemplary 02C06 | EVQLVESGGGLVQPGRSLTLSCAASTNIFSTSPMGWYRQAPG |
| | TriTAC sequence | KQRELVAAIHGTSTLYADSVKGRFTISRDNAKNSIYLQMNSLR |
| | | PEDTALYYCNKVPWGDYHPGNVYWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 512 | Exemplary 02F11 | EVQLVESGGGLVQPGRSLTLSCAASTNIFSTSPMGWYRQAPG |
| | TriTAC sequence | KQRELVAAIHGTSTLYADSVKGRFTISRDNAKNSIYLQMNSLR |
| | | PEDTALYYCNKVPWGDYHPRNVQWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 513 | Exemplary 01E06 | EVQLVESGGGLVQPGRSLTLSCAASTNIFSLSPMGWYRQAPG |
| | TriTAC sequence | KQRELVAAIHGDSTLYADSVKGRFTISRDNAKNSIYLQMNSLR |
| | | PEDTALYYCNKVPWGDYHPRNVYWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 514 | Exemplary 01A03 | EVQLVESGGGLVQPGRSLTLSCAASTNIFSISPGGWYRQAPGK |
| | TriTAC sequence | QRELVAAIHGSSTLYADSVKGRFTISRDNAKNSIYLQMNSLRP |
| | | EDTALYYCNKVPWGDYHPRNVYWGQGTQVTVSSGGGGSGG |
| | | GSEVQLVESGGGLVQPGNSLRLSCAASGFTFSKFGMSWVRQA |
| | | PGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 515 | Exemplary 02A11 | EVQLVESGGGLVQPGRSLTLSCAASTNHFSISPMGWYRQAPG |
| | TriTAC sequence | KQRELVAAIHGSSTLYADSVKGRFTISRDNAKNSIYLQMNSLR |
| | | PEDTALYYCNKVPWGDYHPRVVYWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 516 | Exemplary 01D07 | EVQLVESGGGLVQPGRSLTLSCAASTNIFSASPMGWYRQAPG |
| | TriTAC sequence | KQRELVAAIHGSSTLYADSVKGRFTISRDNAKNSIYLQMNSLR |
| | | PEDTALYYCNKVPWGDYHPRNVNWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 517 | Exemplary 01D10 | EVQLVESGGGLVQPGRSLTLSCAASTNIFSASPMGWYRQAPG |
| | TriTAC sequence | KQRELVAAIHGSSTLYADSVKGRFTISRDNAKNSIYLQMNSLR |
| | | PEDTALYYCNKVPWGRYHPRNVYWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 518 | Exemplary 01A07 | EVQLVESGGGLVQPGRSLTLSCAASTNISSISPMGWYRQAPGK |
| | TriTAC sequence | QRELVAAIHGTSTLYADSVKGRFTISRDNAKNSIYLQMNSLRP |
| | | EDTALYYCNKVPWGDYHPGNVYWGQGTQVTVSSGGGGSGG |
| | | GSEVQLVESGGGLVQPGNSLRLSCAASGFTFSKFGMSWVRQA |
| | | PGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 519 | Exemplary 02F12 | EVQLVESGGGLVQPGRSLTLSCAASTNIESISPMGWYRQAPGK |
| | TriTAC sequence | QRELVAAIHGTSTLYADSVKGRFTISRDNAKNSIYLQMNSLRP |
| | | EDTALYYCNKVPWGDYHPGNVYWGQGTQVTVSSGGGGSGG |
| | | GSEVQLVESGGGLVQPGNSLRLSCAASGFTFSKFGMSWVRQA |
| | | PGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 520 | Exemplary 02B05 | EVQLVESGGGLVQPGRSLTLSCAASTNIFSISPYGWYRQAPGK |
| | TriTAC sequence | QRELVAAIHGTSTLYADSVKGRFTISRDNAKNSIYLQMNSLRP |
| | | EDTALYYCNKVPWGDYHPGNVYWGQGTQVTVSSGGGGSGG |
| | | GSEVQLVESGGGLVQPGNSLRLSCAASGFTFSKFGMSWVRQA |
| | | PGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 521 | Exemplary 01E04 | EVQLVESGGGLVQPGRSLTLSCAASTNIASISPMGWYRQAPG |
| | TriTAC sequence | KQRELVAAIHGTSTLYADSVKGRFTISRDNAKNSIYLQMNSLR |
| | | PEDTALYYCNKVPWGDYHPGNVYWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 522 | Exemplary 02A05 | EVQLVESGGGLVQPGRSLTLSCAASTNIASISPMGWYRQAPG |
| | TriTAC sequence | KQRELVAAIHGKSTLYADSVKGRFTISRDNAKNSIYLQMNSLR |
| | | PEDTALYYCNKVPWGDYHPGNVYWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 523 | Exemplary 02C03 | EVQLVESGGGLVQPGRSLTLSCAASTNIASISPMGWYRQAPG |
| | TriTAC sequence | KQRELVAAIHGSSTLYADSVKGRFTISRDNAKNSIYLQMNSLR |
| | | PEDTALYYCNKVPWGDYHPGNVYWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 524 | Exemplary 01E03 | EVQLVESGGGLVQPGRSLTLSCAASTNITSISPMGWYRQAPGK |
| | TriTAC sequence | QRELVAAIHGDSTLYADSVKGRFTISRDNAKNSIYLQMNSLRP |
| | | EDTALYYCNKVPWGDYHPGNVYWGQGTQVTVSSGGGGSGG |
| | | GSEVQLVESGGGLVQPGNSLRLSCAASGFTFSKFGMSWVRQA |
| | | PGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 525 | Exemplary 01H09 | EVQLVESGGGLVQPGRSLTLSCAASTNIMSISPMGWYRQAPG |
| | TriTAC sequence | KQRELVAAIHGNSTLYADSVKGRFTISRDNAKNSIYLQMNSLR |
| | | PEDTALYYCNKVPWGDYHPGNVYWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 526 | Exemplary 02G05 | EVQLVESGGGLVQPGRSLTLSCAASTNITSISPMGWYRQAPGK |
| | TriTAC sequence | QRELVAAIHGNSTLYADSVKGRFTISRDNAKNSIYLQMNSLRP |
| | | EDTALYYCNKVPWGDYHPGNVYWGQGTQVTVSSGGGGSGG |
| | | GSEVQLVESGGGLVQPGNSLRLSCAASGFTFSKFGMSWVRQA |
| | | PGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 527 | Exemplary 01C01 | EVQLVESGGGLVQPGRSLTLSCAASTNIVSISPMGWYRQAPG |
| | TriTAC sequence | KQRELVAAIHGHSTLYADSVKGRFTISRDNAKNSIYLQMNSLR |
| | | PEDTALYYCNKVPWGDYHPGNVYWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 528 | Exemplary 01D02 | EVQLVESGGGLVQPGRSLTLSCAASTNIVSISPMGWYRQAPG |
| | TriTAC sequence | KQRELVAAIHGKSTLYADSVKGRFTISRDNAKNSIYLQMNSLR |
| | | PEDTALYYCNKVPWGDYHPGNVYWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 529 | Exemplary 02D09 | EVQLVESGGGLVQPGRSLTLSCAASTNVVSISPMGWYRQAPG |
| | TriTAC sequence | KQRELVAAIHGKSTLYADSVKGRFTISRDNAKNSIYLQMNSLR |
| | | PEDTALYYCNKVPWGDYHPNNVYWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 530 | Exemplary 02C01 | EVQLVESGGGLVQPGRSLTLSCAASTNIISISPMGWYRQAPGK |
| | TriTAC sequence | QRELVAAIHGASTLYADSVKGRFTISRDNAKNSIYLQMNSLRP |
| | | EDTALYYCNKVPWGDYHPGNVYWGQGTQVTVSSGGGGSGG |
| | | GSEVQLVESGGGLVQPGNSLRLSCAASGFTFSKFGMSWVRQA |
| | | PGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 531 | Exemplary 02G02 | EVQLVESGGGLVQPGRSLTLSCAASTNIFSITPMGWYRQAPGK |
| | TriTAC sequence | QRELVAAIHGASTLYADSVKGRFTISRDNAKNSIYLQMNSLRP |
| | | EDTALYYCNKVPWGDYHPGNVYWGQGTQVTVSSGGGGSGG |
| | | GSEVQLVESGGGLVQPGNSLRLSCAASGFTFSKFGMSWVRQA |
| | | PGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 532 | Exemplary 01B05 | EVQLVESGGGLVQPGRSLTLSCAASTNITSISPMGWYRQAPGK |
| | TriTAC sequence | QRELVAAIHGFETLYADSVKGRFTISRDNAKNSIYLQMNSLRP |
| | | EDTALYYCNKVPWGDYHPGNVYWGQGTQVTVSSGGGGSGG |
| | | GSEVQLVESGGGLVQPGNSLRLSCAASGFTFSKFGMSWVRQA |
| | | PGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 533 | Exemplary 01G08 | EVQLVESGGGLVQPGRSLTLSCAASTNIQSISPMGWYRQAPG |
| | TriTAC sequence | KQRELVAAIHGFETLYADSVKGRFTISRDNAKNSIYLQMNSLR |
| | | PEDTALYYCNKVPWGDYHPGNVYWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 534 | Exemplary 01H06 | EVQLVESGGGLVQPGRSLTLSCAASTSDFSISPMGWYRQAPG |
| | TriTAC sequence | KQRELVAAIHGFETLYADSVKGRFTISRDNAKNSIYLQMNSLR |
| | | PEDTALYYCNKVPWGDYHPGNVYWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 535 | Exemplary 01F04 | EVQLVESGGGLVQPGRSLTLSCAASTNIDSISPMGWYRQAPG |
| | TriTAC sequence | KQRELVAAIHGFQTLYADSVKGRFTISRDNAKNSIYLQMNSLR |
| | | PEDTALYYCNKVPWGDYHPGNVYWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 536 | Exemplary 01H08 | EVQLVESGGGLVQPGRSLTLSCAASTNIMSISPMGWYRQAPG |
| | TriTAC sequence | KQRELVAAIHGFSTVYADSVKGRFTISRDNAKNSIYLQMNSLR |
| | | PEDTALYYCNKVPWGDYHPGNVYWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 537 | Exemplary 02F07 | EVQLVESGGGLVQPGRSLTLSCAASTNIESISPMGWYRQAPGK |
| | TriTAC sequence | QRELVAAIHGFSTLYADSVKGRFTISRDNAKNSIYLQMNSLRP |
| | | EDTALYYCNKVPWGDYHPGNVYWGQGTQVTVSSGGGGSGG |
| | | GSEVQLVESGGGLVQPGNSLRLSCAASGFTFSKFGMSWVRQA |
| | | PGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 538 | Exemplary 01C05 | EVQLVESGGGLVQPGRSLTLSCAASTNIFSSSPMGWYRQAPG |
| | TriTAC sequence | KQRELVAAIHGFKTLYADSVKGRFTISRDNAKNSIYLQMNSLR |
| | | PEDTARYYCNKVPWGDYHPGNVYWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 539 | Exemplary 02F04 | EVQLVESGGGLVQPGRSLTLSCAASTNIFSSSPMGWYRQAPG |
| | TriTAC sequence | KQRELVAAIHGFSTLYADSVKGRFTISRDNAKNSIYLQMNSLR |
| | | PEDTALYYCNKVPWGDYHPGNVYWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 540 | Exemplary 02B06 | EVQLVESGGGLVQPGRSLTLSCAASTNIFSNSPMGWYRQAPG |
| | TriTAC sequence | KQRELVAAIHGFSTLYADSVKGRFTISRDNAKNSIYLQMNSLR |
| | | PEDTALYYCNKVPWGDYHPGNVYWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 541 | Exemplary 01F07 | EVQLVESGGGLVQPGRSLTLSCAASTNIFSTSPMGWYRQAPG |
| | TriTAC sequence | KQRELVAAIHGFSTIYADSVKGRFTISRDNAKNSIYLQMNSLR |
| | | PEDTALYYCNKVPWGDYHPGNVYWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 542 | Exemplary 02B04 | EVQLVESGGGLVQPGRSLTLSCAASTNIFSTSPMGWYRQAPG |
| | TriTAC sequence | KQRELVAAIHGFSTIYADSVKGRFTISRDNAKNSIYLQMNSLR |
| | | PEDTALYYCNKVPWGDYHPLNVYWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 543 | Exemplary 01H11 | EVQLVESGGGLVQPGRSLTLSCVASTNIFSTSPMGWYRQAPG |
| | TriTAC sequence | KQRELVAAIHGFSTLYADSVKGRFTISRDNAKNSIYLQMNSLR |
| | | PEDTALYYCNKVPWGDYHPGNVYWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 544 | Exemplary 02E06 | EVQLVESGGGLVQPGRSLTLSCAASTNIFSDSPMGWYRQAPG |
| | TriTAC sequence | KQRELVAAIHGFSTFYADSVKGRFTISRDNAKNSIYLQMNSLR |
| | | PEDTALYYCNKVPWGDYHPGNVYWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 545 | Exemplary 01E08 | EVQLVESGGGLVQPGRSLTLSCAASTNIFSQSPMGWYRQAPG |
| | TriTAC sequence | KQRELVAAIHGDSTLYADSVKGRFTISRDNAKNSIYLQMNSLR |
| | | PEDTALYYCNKVPWGDYHPGNVCWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 546 | Exemplary 02A04 | EVQLVESGGGLVQPGRSLTLSCAASTNIFSQSPMGWYRQAPG |
| | TriTAC sequence | KQRELVAAIHGKSTLYADSVKGRFTISRDNAKNSIYLQMNSLR |
| | | PEDTALYYCNKVPWGDYHPSNVYWGKGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 547 | Exemplary 02A08 | EVQLVESGGGLVQPGRSLTLSCAASTNIFSRSPMGWYRQAPG |
| | TriTAC sequence | KQRELVAAIHGESTLYADSVKGRFTISRDNAKNSIYLQMNSLR |
| | | PEDTALYYCNKVPWGRYHPGNVYWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 548 | Exemplary 02E05 | EVQLVESGGGLVQPGRSLTLSCAASTNIFSRSPMGWYRQAPG |
| | TriTAC sequence | KQRELVAAIHGISTLYADSVKGRFTISRDNAKNSIYLQMNSLR |
| | | PEDTALYYCNKVPWGDYHPGNVYWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 549 | Exemplary 02H09 | EVQLVESGGGLVQPGRSLTLSCAASTNIFSRSPMGWYRQAPG |
| | TriTAC sequence | KQRELVAAIHGSSTLYADSVKGRFTISRDNAKNSIYLQMNSLR |
| | | PEDTALYYCNKVPWGDYHPGNVYWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 550 | Exemplary 02G06 | EVQLVESGGGLVQPGRSLTLSCAASTNIFSGSPMGWYRQAPG |
| | TriTAC sequence | KQRELVAAIHGNSTLYADSVKGRFTISRDNAKNSIYLQMNSLR |
| | | PEDTALYYCNKVPWGDYHPGNVYWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 551 | Exemplary 01B09 | EVQLVESGGGLVQPGRSLTLSCAASSNIFSISPMGWYRQAPGK |
| | TriTAC sequence | QRELVAAIHGSSTLYADSVKGRFTISRDNAKNSIYLQMNSLRP |
| | | EDTALYYCNKVPWGDYHPGNVYWGQGTQVTVSSGGGGSGG |
| | | GSEVQLVESGGGLVQPGNSLRLSCAASGFTFSKFGMSWVRQA |
| | | PGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 552 | Exemplary 02F03 | EVQLVESGGGLVQPGRSLTLSCAASTNIFSIYPMGWYRQAPG |
| | TriTAC sequence | KQRELVAAIHGSSTLYADSVKGRFTISRDNAKNSIYLQMNSLR |
| | | PEDTALYYCNKVPWGDYHPKNVYWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 553 | Exemplary 02F02 | EVQLVESGGGLVQPGRSLTLSCAASTNIFSKSPMGWYRQAPG |
| | TriTAC sequence | KQRELVAAIHGSSTLYADSVKGRFTISRDNAKNSIYLQMNSLR |
| | | PEDTALYYCNKVPWGDYHPGNVYWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 554 | Exemplary 02H01 | EVQLVESGGGLVQPGRSLTLSCAASTNIFSKSPMGWYRQAPG |
| | TriTAC sequence | KQRELVAAIHGSSTLYADSVKGRFTISRDNAKNSIYLQMNSLR |
| | | PEDTALYYCNKVPWGDYHPRNVYWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 555 | Exemplary 01G10 | EVQLVESGGGLVQPGRSLTLSCAASTNEFSISPMGWYRQAPG |
| | TriTAC sequence | KQRELVAAIHGLSTLYADSVKGRFTISRDNAKNSIYLQMNSLR |
| | | PEDTALYYCNKVPWGAYHPRNVYWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 556 | Exemplary 02D11 | EVQLVESGGGLVQPGRSLTLSCAASTNEFSISPMGWYRQAPG |
| | TriTAC sequence | KQRELVAAIHGESTLYADSVKGRFTISRDNAKNSIYLQMNSLR |
| | | PEDTALYYCNKVPWGDYHPGNVYWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 557 | Exemplary 01B01 | EVQLVESGGGLVQPGRSLTLSCAASTNIPSISPMGWYRQAPGK |
| | TriTAC sequence | QRELVAAIHGESTLYADSVKGRFTISRDNAKNSIYLQMNSLRP |
| | | EDTALYYCNKVPWGDYHPRNVAWGQGTQVTVSSGGGGSGG |
| | | GSEVQLVESGGGLVQPGNSLRLSCAASGFTFSKFGMSWVRQA |
| | | PGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 558 | Exemplary 01G11 | EVQLVESGGGLVQPGRSLTLSCAASTNIPSISPMGWYRQAPGK |
| | TriTAC sequence | QRELVAAIHGASTLYADSVKGRFTISRDNAKNSIYLQMNSLRP |
| | | EDTALYYCNKVPWGDYHPRNVAWGQGTQVTVSSGGGGSGG |
| | | GSEVQLVESGGGLVQPGNSLRLSCAASGFTFSKFGMSWVRQA |
| | | PGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 559 | Exemplary 01H10 | EVQLVESGGGLVQPGRSLTLSCAASTNIPSISPMGWYRQAPGK |
| | TriTAC sequence | QRELVAAIHGESTLYADSVKGRFTISRDNAKNSIYLQMNSLRP |
| | | EDTALYYCNKVPWGDYHPRNVYWGQGTQVTVSSGGGGSGG |
| | | GSEVQLVESGGGLVQPGNSLRLSCAASGFTFSKFGMSWVRQA |
| | | PGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 560 | Exemplary 01C04 | EVQLVESGGGLVQPGRSLTLSCAASTNIPSISPMGWYRQAPGK |
| | TriTAC sequence | QRELVAAIHGDSTLYADSVKGRFTISRDNAKNSIYLQMNSLRP |
| | | EDTALYYCNKVPWGDYHPRNVYWGQGTQVTVSSGGGGSGG |
| | | GSEVQLVESGGGLVQPGNSLRLSCAASGFTFSKFGMSWVRQA |
| | | PGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 561 | Exemplary 01D04 | EVQLVESGGGLVQPGRSLTLSCAASTNITSISPMGWYRQAPGK |
| | TriTAC sequence | QRELVAAIHGVSTLYADSVKGRFTISRDNAKNSIYLQMNSLRP |
| | | EDTALYYCNKVPWGDYHPRNVQWGQGTQVTVSSGGGGSGG |
| | | GSEVQLVESGGGLVQPGNSLRLSCAASGFTFSKFGMSWVRQA |
| | | PGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 562 | Exemplary 01E07 | EVQLVESGGGLVQPGRSLTLSCAASTNIPSISPMGWYRQAPGK |
| | TriTAC sequence | QRELVAAIHGQSTLYADSVKGRFTISRDNAKNSIYLQMNSLRP |
| | | EDTALYYCNKVPWGDYHPRNVQWGQGTQVTVSSGGGGSGG |
| | | GSEVQLVESGGGLVQPGNSLRLSCAASGFTFSKFGMSWVRQA |
| | | PGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 563 | Exemplary 02B11 | EVQLVESGGGLVQPGRSLTLSCAASTNIVSISPMGWYRQAPG |
| | TriTAC sequence | KQRELVAAIHGDSTLYADSVKGRFTISRDNAKNSIYLQMNSLR |
| | | PEDTALYYCNKVPWGDYHPRNVSWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 564 | Exemplary 01F10 | EVQLVESGGGLVQPGRSLTLSCAASSNIFSISPMGWYRQAPGK |
| | TriTAC sequence | QRELVAAIHGESTLYADSVKGRFTISRDNAKNSIYLQMNSLRP |
| | | EDTALYYCNKVPWGDYHPRNVTWGQGTQVTVSS |
| | | GGGGSGGGSEVQLVESGGGLVQPGNSLRLSCAASGFTFSKFG |
| | | MSWVRQAPGKGLEWVSSISGSGRDTLYADSVKGRFTISRDNA |
| | | KTTLYLQMNSLRPEDTAVYYCTIGGSLSVSSQGTLVTVSSGGG |
| | | GSGGGSEVQLVESGGGLVQPGGSLKLSCAASGFTFNKYAINW |
| | | VRQAPGKGLEWVARIRSKYNNYATYYADQVKDRFTISRDDS |
| | | KNTAYLQMNNLKTEDTAVYYCVRHANFGNSYISYWAYWGQ |
| | | GTLVTVSSGGGGSGGGGSGGGGSQTVVTQEPSLTVSPGGTVT |
| | | LTCASSTGAVTSGNYPNWVQQKPGQAPRGLIGGTKFLVPGTP |
| | | ARFSGSLLGGKAALTLSGVQPEDEAEYYCTLWYSNRWVFGG |
| | | GTKLTVLHHHHHH |
| 565 | Exemplary 02G08 | EVQLVESGGGLVQPGRSLTLSCAASTNIDSISPMGWYRQAPG |
| | TriTAC sequence | KQRELVAAIHGESTLYADSVKGRFTISRDNAKNSIYLQMNSLR |
| | | PEDTALYYCNKVPWGDYHPRNVTWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 566 | Exemplary 02G11 | EVQLVESGGGLVQPGRSLTLSCAASTNIDSISPMGWYRQAPG |
| | TriTAC sequence | KQRELVAAIHGSSTLYADSVKGRFTISRDNAKNSIYLQMNSLR |
| | | PEDTALYYCNKVPWGDYHPRNVTWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 567 | Exemplary 02H06 | EVQLVESGGGLVQPGRSLTLSCAASTNIRSISPMGWYRQAPGK |
| | TriTAC sequence | QRELVAAIHGSSTLYADSVKGRFTISRDNAKNSIYLQMNSLRP |
| | | EDTALYYCNKVPWGDYHPRNVVWGQGTQVTVSSGGGGSGG |
| | | GSEVQLVESGGGLVQPGNSLRLSCAASGFTFSKFGMSWVRQA |
| | | PGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 568 | Exemplary 01B02 | EVQLVESGGGLVQPGRSLTLSCAASTNITSISPMGWYRQAPGK |
| | TriTAC sequence | QRELVAAISGFSTLYADSVKGRFTISRDNAKNSIYLQMNSLRP |
| | | EDTALYYCNEVPWGDYHPRNVYWGQGTQVTVSSGGGGSGG |
| | | GSEVQLVESGGGLVQPGNSLRLSCAASGFTFSKFGMSWVRQA |
| | | PGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 569 | Exemplary 02H11 | EVQLVESGGGLVQPGRSLTLSCAASTNITSISPMGWYRQAPGK |
| | TriTAC sequence | QRELVAAIHGESTLYADSVKGRFTISRDNAKNSIYLQMNSLRP |
| | | EDTALYYCNKVPWGDYHPRNVYWGQGTQVTVSSGGGGSGG |
| | | GSEVQLVESGGGLVQPGNSLRLSCAASGFTFSKFGMSWVRQA |
| | | PGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 570 | Exemplary 01F08 | EVQLVESGGGLVQPGRSLTLSCAASTNITSVSPMGWYRQAPG |
| | TriTAC sequence | KQRELVAAIHGPSTLYADSVKGRFTISRDNAKNSIYLQMNSLR |
| | | PEDTALYYCNKVPWGDYHPTNVYWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 571 | Exemplary 01H01 | EVQLVESGGGLVQPGRSLTLSCAASTNIGSISPMGWYRQAPG |
| | TriTAC sequence | KQRELVAAIHGQSTLYADSVKGRFTISRDNAKNSIYLQMNSLR |
| | | PEDTALYYCNKVPWGDYHPQNVYWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 572 | Exemplary 01E10 | EVQLVESGGGLVQPGRSLTLSCAASTNIESISPMGWYRQAPGK |
| | TriTAC sequence | QRELVAAIHGKSTLYADSVKGRFTISRDNAKNSIYLQMNSLRP |
| | | EDTALYYCNKVPWGDYHPRRVYWGQGTQVTVSSGGGGSGG |
| | | GSEVQLVESGGGLVQPGNSLRLSCAASGFTFSKFGMSWVRQA |
| | | PGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 573 | Exemplary 01G01 | EVQLVESGGGLVQPGRSLTLSCAASTNIVSISPMGWYRQAPG |
| | TriTAC sequence | KQRELVAAIHGDSTLYADSVKGRFTISRDNAKNSIYLQMNSLR |
| | | PEDTALYYCNKVPWGDYHPRRVYWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 574 | Exemplary 01G04 | EVQLVESGGGLVQPGRSLTLSCAASTNIDSISPMGWYRQAPG |
| | TriTAC sequence | KQRELVAAIHGNSTLYADSVKGRFTISRDNAKNSIYLQMNSLR |
| | | PEDTALYYCNKVPWGDYHPRMVYWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 575 | Exemplary 01A04 | EVQLVESGGGLVQPGRSLTLSCAASTNIFMISPMGWYRQAPG |
| | TriTAC sequence | KQRELVAAIHGDSTLYADSVKGRFTISRDNAKNSIYLQMNSLR |
| | | PEDTALYYCNKVPWGRYHPRNVYWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 576 | Exemplary 01F12 | EVQLVESGGGLVQPGRSLTLSCAASTNIFRISPMGWYRQAPGK |
| | TriTAC sequence | QRELVAAIHGDSTLYADSVKGRFTISRDNAKNSIYLQMNSLRP |
| | | EDTALYYCNKVPWGRYHPRNVYWGQGTQVTVSSGGGGSGG |
| | | GSEVQLVESGGGLVQPGNSLRLSCAASGFTFSKFGMSWVRQA |
| | | PGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 577 | Exemplary 01B06 | EVQLVESGGGLVQPGRSLTLSCAASTNIFSISPMGWYRQAPGK |
| | TriTAC sequence | QRELVAAIHGDSTLYADSVKGRFTISRDNAKNSIYLQMNSLRP |
| | | EDTALYYCNKVPWGEYHPRNVYWGQGTQVTVSSGGGGSGG |
| | | GSEVQLVESGGGLVQPGNSLRLSCAASGFTFSKFGMSWVRQA |
| | | PGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 578 | Exemplary 01C06 | EVQLVESGGGLVQPGRSLTLSCAASTNIFSISPMGWYRQAPGK |
| | TriTAC sequence | QRELVAAIHGDSTLYADSVKGRFTISRDNAKNSIYLQMNSLRP |
| | | EDTALYYCNKVPWGKYHPRNVYWGQGTQVTVSSGGGGSGG |
| | | GSEVQLVESGGGLVQPGNSLRLSCAASGFTFSKFGMSWVRQA |
| | | PGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 579 | Exemplary 01B08 | EVQLVESGGGLVQPGRSLTLSCAASTNIESISPMGWYRQAPGK |
| | TriTAC sequence | QRELVAAIHGSSTLYADSVKGRFTISRDNAKNSIYLQMNSLRP |
| | | EDTALYYCNKVPWGRYHPRNVYWGQGTQVTVSSGGGGSGG |
| | | GSEVQLVESGGGLVQPGNSLRLSCAASGFTFSKFGMSWVRQA |
| | | PGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 580 | Exemplary 01C02 | EVQLVESGGGLVQPGRSLTLSCAASTNIESISPMGWYRQAPGK |
| | TriTAC sequence | QRELVAAIHGNSTLYADSVKGRFTISRDNAKNSIYLQMNSLRP |
| | | EDTALYYCNKVPWGRYHPRNVYWGQGTQVTVSSGGGGSGG |
| | | GSEVQLVESGGGLVQPGNSLRLSCAASGFTFSKFGMSWVRQA |
| | | PGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 581 | Exemplary 01C10 | EVQLVESGGGLVQPGRSLTLSCAASTNISSISPMGWYRQAPGK |
| | TriTAC sequence | QRELVAAIHGFSTLYADSVKGRFTISRDNAKNSIYLQMNSLRP |
| | | EDTALYYCNKVPWGYYHPRNVYWGQGTQVTVSSGGGGSGG |
| | | GSEVQLVESGGGLVQPGNSLRLSCAASGFTFSKFGMSWVRQA |
| | | PGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 582 | Exemplary 01F09 | EVQLVESGGGLVQPGRSLTLSCAASTNISSISPMGWYRQAPGK |
| | TriTAC sequence | QRELVAAIHGHSTLYADSVKGRFTISRDNAKNSIYLQMNSLRP |
| | | EDTALYYCNKVPWGRYHPRNVYWGQGTQVTVSSGGGGSGG |
| | | GSEVQLVESGGGLVQPGNSLRLSCAASGFTFSKFGMSWVRQA |
| | | PGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 583 | Exemplary 02D06 | EVQLVESGGGLVQPGRSLTLSCAASTNISSISPMGWYRQAPGK |
| | TriTAC sequence | QRELVAAIHGFSTVYADSVKGRFTISRDNAKNSIYLQMNSLRP |
| | | EDTALYYCNKVPWGRYHPRNVYWGQGTQVTVSSGGGGSGG |
| | | GSEVQLVESGGGLVQPGNSLRLSCAASGFTFSKFGMSWVRQA |
| | | PGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 584 | Exemplary 01A06 | EVQLVESGGGLVQPGRSLTLSCAASTNIFSIRPMGWYRQAPGK |
| | TriTAC sequence | QRELVAAIHGFSTVYADSVKGRFTISRDNAKNSIYLQMNSLRP |
| | | EDTALYYCNKVPWGDYHPRNVYWGQGTQVTVSSGGGGSGG |
| | | GSEVQLVESGGGLVQPGNSLRLSCAASGFTFSKFGMSWVRQA |
| | | PGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 585 | Exemplary 01C07 | EVQLVESGGGLVQPGRSLTLSCAASTNIFSIYPMGWYRQAPG |
| | TriTAC sequence | KQRELVAAIHGFSTYYADSVKGRFTISRDNAKNSIYLQMNSLR |
| | | PEDTALYYCNKVPWGSYHPRNVYWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 586 | Exemplary 01G09 | EVQLVESGGGLVQPGRSLTLSCAASTNIFNISPMGWYRQAPG |
| | TriTAC sequence | KQRELVAAIHGFSTYYADSVKGRFTISRDNAKNSIYLQMNSLR |
| | | PEDTALYYCNKVPWGRYHPRNVYWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 587 | Exemplary 01F05 | EVQLVESGGGLVQPGRSLTLSCAASTNIFSSSPMGWYRQAPG |
| | TriTAC sequence | KQRELVAAIHGFSTWYADSVKGRFTISRDNAKNSIYLQMNSL |
| | | RPEDTALYYCNKVPWGRYHPRNVYWGQGTQVTVSSGGGGS |
| | | GGGSEVQLVESGGGLVQPGNSLRLSCAASGFTFSKFGMSWVR |
| | | QAPGKGLEWVSSISGSGRDTLYADSVKGRFTISRDNAKTTLYL |
| | | QMNSLRPEDTAVYYCTIGGSLSVSSQGTLVTVSSGGGGSGGG |
| | | SEVQLVESGGGLVQPGGSLKLSCAASGFTFNKYAINWVRQAP |
| | | GKGLEWVARIRSKYNNYATYYADQVKDRFTISRDDSKNTAY |
| | | LQMNNLKTEDTAVYYCVRHANFGNSYISYWAYWGQGTLVT |
| | | VSSGGGGSGGGGSGGGGSQTVVTQEPSLTVSPGGTVTLTCAS |
| | | STGAVTSGNYPNWVQQKPGQAPRGLIGGTKFLVPGTPARFSG |
| | | SLLGGKAALTLSGVQPEDEAEYYCTLWYSNRWVFGGGTKLT |
| | | VLHHHHHH |
| 588 | Exemplary 02B12 | EVQLVESGGGLVQPGRSLTLSCAASTNISSISPMGWYRQAPGK |
| | TriTAC sequence | QRELVAAIHGFDTLYADSVKGRFTISRDNAKNSIYLQMNSLRP |
| | | EDTALYYCNKVPWGDYHPRNVYWGQGTQVTVSSGGGGSGG |
| | | GSEVQLVESGGGLVQPGNSLRLSCAASGFTFSKFGMSWVRQA |
| | | PGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 589 | Exemplary 02G01 | EVQLVESGGGLVQPGRSLTLSCAASTNIFSINPMGWYRQAPG |
| | TriTAC sequence | KQRELVAAIHGFDTLYADSVKGRFTISRDNAKNSIYLQMNSLR |
| | | PEDTALYYCNKVPWGDYHPRNVSWGQGTQVTVSSGGGGSG |
| | | GGSEVQLVESGGGLVQPGNSLRLSCAASGFTFSKFGMSWVRQ |
| | | APGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 590 | Exemplary 01A09 | EVQLVESGGGLVQPGRSLTLSCAASTNIFSITPMGWYRQAPGK |
| | TriTAC sequence | QRELVAAIHGRSTLYADSVKGRFTISRDNAKNSIYLQMNSLRP |
| | | EDTALYYCNKVPWGSYHPRNVYWGQGTQVTVSSGGGGSGG |
| | | GSEVQLVESGGGLVQPGNSLRLSCAASGFTFSKFGMSWVRQA |
| | | PGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 591 | Exemplary 01H05 | EVQLVESGGGLVQPGRSLTLSCAASTNIFSITPMGWYRQAPGK |
| | TriTAC sequence | QRELVAAIHGTSTLYADSVKGRFTISRDNAKNSIYLQMNSLRP |
| | | EDTALYYCNKVPWGRYHPRNVYWGQGTQVTVSSGGGGSGG |
| | | GSEVQLVESGGGLVQPGNSLRLSCAASGFTFSKFGMSWVRQA |
| | | PGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 592 | Exemplary 02F06 | EVQLVESGGGLVQPGRSLTLSCAASTNIFSITPMGWYRQAPGK |
| | TriTAC sequence | QRELVAAIHGESTLYADSVKGRFTISRDNAKNSIYLQMNSLRP |
| | | EDTALYYCNKVPWGRYHPRNVYWGQGTQVTVSSGGGGSGG |
| | | GSEVQLVESGGGLVQPGNSLRLSCAASGFTFSKFGMSWVRQA |
| | | PGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 593 | Exemplary 02G07 | EVQLVESGGGLVQPGRSLTLSCAASTNIFSITPMGWYRQAPGK |
| | TriTAC sequence | QRELVAAIHGESTLYADSVKGRFTISRDNAKNSIYLQMNSLRP |
| | | EDTALYYCNKVPWGDYHPRDVYWGQGTQVTVSSGGGGSGG |
| | | GSEVQLVESGGGLVQPGNSLRLSCAASGFTFSKFGMSWVRQA |
| | | PGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 594 | Exemplary 01F07- | EVQLVESGGGLVQPGRSLTLSCAASTNIFSTSPYGWYRQAPGK |
| | M34Y TriTAC | QRELVAAIHGFSTIYADSVKGRFTISRDNAKNSIYLQMNSLRPE |
| | sequence | DTALYYCNKVPWGDYHPGNVYWGQGTQVTVSSGGGGSGGG |
| | | SEVQLVESGGGLVQPGNSLRLSCAASGFTFSKFGMSWVRQAP |
| | | GKGLEWVSSISGSGRDTLYADSVKGRFTISRDNAKTTLYLQM |
| | | NSLRPEDTAVYYCTIGGSLSVSSQGTLVTVSSGGGGSGGGSEV |
| | | QLVESGGGLVQPGGSLKLSCAASGFTFNKYAINWVRQAPGKG |
| | | LEWVARIRSKYNNYATYYADQVKDRFTISRDDSKNTAYLQM |
| | | NNLKTEDTAVYYCVRHANFGNSYISYWAYWGQGTLVTVSSG |
| | | GGGSGGGGSGGGGSQTVVTQEPSLTVSPGGTVTLTCASSTGA |
| | | VTSGNYPNWVQQKPGQAPRGLIGGTKFLVPGTPARFSGSLLG |
| | | GKAALTLSGVQPEDEAEYYCTLWYSNRWVFGGGTKLTVLHH |
| | | HHHH |
| 595 | Exemplary 01F01- | EVQLVESGGGLVQPGRSLTLSCAASTNIFSTSPGGWYRQAPGK |
| | M34G TriTAC | QRELVAAIHGFSTIYADSVKGRFTISRDNAKNSIYLQMNSLRPE |
| | sequence | DTALYYCNKVPWGDYHPGNVYWGQGTQVTVSSGGGGSGGG |
| | | SEVQLVESGGGLVQPGNSLRLSCAASGFTFSKFGMSWVRQAP |
| | | GKGLEWVSSISGSGRDTLYADSVKGRFTISRDNAKTTLYLQM |
| | | NSLRPEDTAVYYCTIGGSLSVSSQGTLVTVSSGGGGSGGGSEV |
| | | QLVESGGGLVQPGGSLKLSCAASGFTFNKYAINWVRQAPGKG |
| | | LEWVARIRSKYNNYATYYADQVKDRFTISRDDSKNTAYLQM |
| | | NNLKTEDTAVYYCVRHANFGNSYISYWAYWGQGTLVTVSSG |
| | | GGGSGGGGSGGGGSQTVVTQEPSLTVSPGGTVTLTCASSTGA |
| | | VTSGNYPNWVQQKPGQAPRGLIGGTKFLVPGTPARFSGSLLG |
| | | GKAALTLSGVQPEDEAEYYCTLWYSNRWVFGGGTKLTVLHH |
| | | HHHH |
| 596 | Exemplary 02G02- | EVQLVESGGGLVQPGRSLTLSCAASTNIFSITPYGWYRQAPGK |
| | M34Y TriTAC | QRELVAAIHGASTLYADSVKGRFTISRDNAKNSIYLQMNSLRP |
| | sequence | EDTALYYCNKVPWGDYHPGNVYWGQGTQVTVSSGGGGSGG |
| | | GSEVQLVESGGGLVQPGNSLRLSCAASGFTFSKFGMSWVRQA |
| | | PGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 597 | Exemplary 02G02- | EVQLVESGGGLVQPGRSLTLSCAASTNIFSITPGGWYRQAPGK |
| | M34G TriTAC | QRELVAAIHGASTLYADSVKGRFTISRDNAKNSIYLQMNSLRP |
| | sequence | EDTALYYCNKVPWGDYHPGNVYWGQGTQVTVSSGGGGSGG |
| | | GSEVQLVESGGGLVQPGNSLRLSCAASGFTFSKFGMSWVRQA |
| | | PGKGLEWVSSISGSGRDTLYADSVKGRFTISRDNAKTTLYLQ |
| | | MNSLRPEDTAVYYCTIGGSLSVSSQGTLVTVSSGGGGSGGGSE |
| | | VQLVESGGGLVQPGGSLKLSCAASGFTFNKYAINWVRQAPGK |
| | | GLEWVARIRSKYNNYATYYADQVKDRFTISRDDSKNTAYLQ |
| | | MNNLKTEDTAVYYCVRHANFGNSYISYWAYWGQGTLVTVS |
| | | SGGGGSGGGGSGGGGSQTVVTQEPSLTVSPGGTVTLTCASST |
| | | GAVTSGNYPNWVQQKPGQAPRGLIGGTKFLVPGTPARFSGSL |
| | | LGGKAALTLSGVQPEDEAEYYCTLWYSNRWVFGGGTKLTVL |
| | | HHHHHH |
| 598 | 253BH10 (llama | QVQLVESGGGLVQPGESLRLSCAASTNIFSISPMGWYRQAPGK |
| | anti-BCMA | QRELVAAIHGFSTLYADSVKGRFTISRDNAKNTIYLQMNSLKP |
| | antibody) | EDTAVYYCNKVPWGDYHPRNVYWGQGTQVTVSS |
| 599 | 253BH10 CDR1 | TNIFSISPMG |
| 600 | 253BH10 CDR2 | AIHGFSTLYADSVK |
| 601 | 253BH10 CDR3 | VPWGDYHPRNVY |
| 602 | Exemplary linker | GGGGSGGGS |
| | sequence | |

1-84. (canceled)

85. A method of treating cancer, the method comprising administration of an effective amount of a B cell maturation agent (BCMA) targeting trispecific protein to a subject, wherein said protein comprises:
(a) a first domain (A) which specifically binds to human CD3;
(b) a second domain (B) which is a half-life extension domain; and
(c) a third domain (C) which specifically binds to BCMA,
wherein the domains are linked in the order H₂N-(C)-(B)-(A)-COOH, or by linkers L1 and L2, and wherein the BCMA targeting trispecific protein is administered at a dosage of from about 800 μg to about 8000 μg.

86. The method of claim 85, wherein the BCMA targeting trispecific protein is administered at a dosage of from about 1500 μg to about 6000 μg.

87. The method of claim 86, wherein the BCMA targeting trispecific protein is administered at a dosage of from about 2000 μg to about 3000 μg.

88. The method of claim 87, wherein the BCMA targeting trispecific protein is administered at a dosage of 2150 μg or 2860 μg.

89. The method of claim 85, wherein the BCMA targeting trispecific protein is administered once a week.

90. The method of claim 85, wherein the BCMA targeting trispecific protein is administered intravenously, intraperitoneally, subcutaneously, intramuscularly, topically or intradermally.

91. The method of claim 85, wherein the method comprises administering a first dose of the BCMA targeting trispecific protein and a second dose of the BCMA targeting trispecific protein, and wherein the second dose is higher than the first dose.

92. The method of claim 91, wherein the first dose of the BCMA targeting trispecific protein is from about 1000 μg to about 1700 μg.

93. The method of claim 92, wherein the first dose of the BCMA targeting trispecific protein is 1000 μg, 1500 μg, or 1620 μg.

94. The method of claim 91, wherein the second dose of the BCMA targeting trispecific protein is from about 3000 μg to about 6000 μg.

95. The method of claim 94, wherein the second dose of the BCMA targeting trispecific protein is 3000 μg, 3240 μg, or 6000 μg.

96. The method of claim 91, wherein the first dose of the BCMA targeting trispecific protein is 1620 μg, and wherein the second dose of the BCMA targeting trispecific protein is 3240 μg.

97. The method of claim 91, wherein the method further comprises a third dose of the BCMA trispecific protein, and wherein the third dose is higher than the second dose.

98. The method of claim 97, wherein the third dose is about 6000 μg.

99. The method of claim 91, wherein the first dose or the second dose is administered once a week.

100. The method of claim 85, wherein the third domain comprises a VHH domain, and wherein the VHH domain comprises one or more sequences selected from the group consisting of SEQ ID NOS: 346-460.

101. The method of claim 85, wherein the second domain binds human serum albumin and comprises a scFv, a variable heavy domain (VH), a variable light domain (VL), a peptide, a ligand, or a small molecule.

102. The method of claim 85, wherein the BCMA targeting trispecific protein comprises a sequence selected from the group consisting of SEQ ID NO: 483-597.

103. The method of claim 102, wherein the BCMA targeting trispecific protein comprises a sequence of SEQ ID NO: 520.

104. The method of claim 85, wherein the cancer is a multiple myeloma, a leukemia, a lymphoma, or a metastasis thereof.

105. A method of treating cancer, the method comprising administration of an effective amount of a B cell maturation agent (BCMA) targeting trispecific protein to a subject, wherein said protein comprises:
(a) a first domain (A) which specifically binds to human CD3;
(b) a second domain (B) which is a half-life extension domain; and
(c) a third domain (C) which specifically binds to BCMA,
wherein the domains are linked in the order H₂N-(C)-(B)-(A)-COOH, or by linkers L1 and L2, and wherein the BCMA targeting trispecific protein is administered according to a schedule comprising the following steps:
(i) administration of a first dose of the BCMA targeting trispecific protein, and
(ii) administration of a second dose of the BCMA targeting trispecific protein, wherein the second dose is higher than the first dose.
